SEQUENCE LISTING (110 sequences)

SEQ ID NO: 1 - DNA, 20 bp, synthetic construct
  misc_feature 1..20: note = Synthetic
  source 1..20: mol_type = other DNA
ctactgtgccccaccggcgc

SEQ ID NO: 2 - DNA, 20 bp, synthetic construct
  misc_feature 1..20: note = Synthetic
  source 1..20: mol_type = other DNA
tggacgcgcaggctgccggt

SEQ ID NO: 3 - DNA, 20 bp, synthetic construct
  misc_feature 1..20: note = Synthetic
  source 1..20: mol_type = other DNA
ggatcttccgatggggctcg

SEQ ID NO: 4 - DNA, 21 bp, synthetic construct
  misc_feature 1..21: note = Synthetic
  source 1..21: mol_type = other DNA
gagcagcagcggcggcaacag

SEQ ID NO: 5 - DNA, 20 bp, synthetic construct
  misc_feature 1..20: note = Synthetic
  source 1..20: mol_type = other DNA
agcagcagcggcggcaacag

SEQ ID NO: 6 - DNA, 20 bp, synthetic construct
  misc_feature 1..20: note = Synthetic
  source 1..20: mol_type = other DNA
ggctgatgaggccgcacatg

SEQ ID NO: 7 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
aactactctttactgaccccgttttagagctagaaatagcaagttaaaataaggctagtccgttatcaacttgaaaaagtggcaccgagtcggtgctttt

SEQ ID NO: 8 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
tactctttactgacccctgggttttagagctagaaatagcaagttaaaataaggctagtccgttatcaacttgaaaaagtggcaccgagtcggtgctttt

SEQ ID NO: 9 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
tttgttgctgaggtgccaccgttttagagctagaaatagcaagttaaaataaggctagtccgttatcaacttgaaaaagtggcaccgagtcggtgctttt

SEQ ID NO: 10 - DNA, 20 bp, synthetic construct
  misc_feature 1..20: note = Synthetic
  source 1..20: mol_type = other DNA
agatttctcacttgttgtac

SEQ ID NO: 11 - DNA, 20 bp, synthetic construct
  misc_feature 1..20: note = Synthetic
  source 1..20: mol_type = other DNA
gttgtactggtgccctctgc

SEQ ID NO: 12 - DNA, 20 bp, synthetic construct
  misc_feature 1..20: note = Synthetic
  source 1..20: mol_type = other DNA
gactacaatggaccagcaga

SEQ ID NO: 13 - DNA, 20 bp, synthetic construct
  misc_feature 1..20: note = Synthetic
  source 1..20: mol_type = other DNA
tctaggagcagtgactacaa

SEQ ID NO: 14 - DNA, 20 bp, synthetic construct
  misc_feature 1..20: note = Synthetic
  source 1..20: mol_type = other DNA
tatgaccagaagatggcgtc

SEQ ID NO: 15 - DNA, 20 bp, synthetic construct
  misc_feature 1..20: note = Synthetic
  source 1..20: mol_type = other DNA
ggttgccagacgccatcttc

SEQ ID NO: 16 - DNA, 20 bp, synthetic construct
  misc_feature 1..20: note = Synthetic
  source 1..20: mol_type = other DNA
ccataactatgaccagaaga

SEQ ID NO: 17 - DNA, 20 bp, synthetic construct
  misc_feature 1..20: note = Synthetic
  source 1..20: mol_type = other DNA
ccatcttctggtcatagtta

SEQ ID NO: 18 - DNA, 20 bp, synthetic construct
  misc_feature 1..20: note = Synthetic
  source 1..20: mol_type = other DNA
aactactctttactgacccc

SEQ ID NO: 19 - DNA, 20 bp, synthetic construct
  misc_feature 1..20: note = Synthetic
  source 1..20: mol_type = other DNA
tactctttactgacccctgg

SEQ ID NO: 20 - DNA, 20 bp, synthetic construct
  misc_feature 1..20: note = Synthetic
  source 1..20: mol_type = other DNA
tttgttgctgaggtgccacc

SEQ ID NO: 21 - RNA, 100 nt, synthetic construct
  misc_feature 1..100: note = Synthetic
  modified_base 1..3: mod_base = OTHER; note = 2'-O-methyl and phosphorothioate linker
  modified_base 28..39: mod_base = OTHER; note = 2'-O-methyl
  modified_base 97..99: mod_base = OTHER; note = phosphorothioate linker
  modified_base 68..100: mod_base = OTHER; note = 2'-O-methyl
  source 1..100: mol_type = other RNA
ctactgtgccccaccggcgcgttttagagctagaaatagcaagttaaaataaggctagtccgttatcaacttgaaaaagtggcaccgagtcggtgctttt

SEQ ID NO: 22 - RNA, 100 nt, synthetic construct
  misc_feature 1..100: note = Synthetic
  modified_base 1..3: mod_base = OTHER; note = 2'-O-methyl and phosphorothioate linker
  modified_base 28..39: mod_base = OTHER; note = 2'-O-methyl
  modified_base 97..99: mod_base = OTHER; note = phosphorothioate linker
  modified_base 68..100: mod_base = OTHER; note = 2'-O-methyl
  source 1..100: mol_type = other RNA
tggacgcgcaggctgccggtgttttagagctagaaatagcaagttaaaataaggctagtccgttatcaacttgaaaaagtggcaccgagtcggtgctttt

SEQ ID NO: 23 - RNA, 100 nt, synthetic construct
  misc_feature 1..100: note = Synthetic
  modified_base 1..3: mod_base = OTHER; note = 2'-O-methyl and phosphorothioate linker
  modified_base 28..39: mod_base = OTHER; note = 2'-O-methyl
  modified_base 97..99: mod_base = OTHER; note = phosphorothioate linker
  modified_base 68..100: mod_base = OTHER; note = 2'-O-methyl
  source 1..100: mol_type = other RNA
ggatcttccgatggggctcggttttagagctagaaatagcaagttaaaataaggctagtccgttatcaacttgaaaaagtggcaccgagtcggtgctttt

SEQ ID NO: 24 - RNA, 101 nt, synthetic construct
  misc_feature 1..101: note = Synthetic
  modified_base 1..3: mod_base = OTHER; note = 2'-O-methyl and phosphorothioate linker
  modified_base 28..39: mod_base = OTHER; note = 2'-O-methyl
  modified_base 97..99: mod_base = OTHER; note = phosphorothioate linker
  modified_base 68..100: mod_base = OTHER; note = 2'-O-methyl
  source 1..101: mol_type = other RNA
gagcagcagcggcggcaacaggttttagagctagaaatagcaagttaaaataaggctagtccgttatcaacttgaaaaagtggcaccgagtcggtgctttt

SEQ ID NO: 25 - RNA, 100 nt, synthetic construct
  misc_feature 1..100: note = Synthetic
  modified_base 1..3: mod_base = OTHER; note = 2'-O-methyl and phosphorothioate linker
  modified_base 28..39: mod_base = OTHER; note = 2'-O-methyl
  modified_base 97..99: mod_base = OTHER; note = phosphorothioate linker
  modified_base 68..100: mod_base = OTHER; note = 2'-O-methyl
  source 1..100: mol_type = other RNA
agcagcagcggcggcaacaggttttagagctagaaatagcaagttaaaataaggctagtccgttatcaacttgaaaaagtggcaccgagtcggtgctttt

SEQ ID NO: 26 - RNA, 100 nt, synthetic construct
  misc_feature 1..100: note = Synthetic
  modified_base 1..3: mod_base = OTHER; note = 2'-O-methyl and phosphorothioate linker
  modified_base 28..39: mod_base = OTHER; note = 2'-O-methyl
  modified_base 97..99: mod_base = OTHER; note = phosphorothioate linker
  modified_base 68..100: mod_base = OTHER; note = 2'-O-methyl
  source 1..100: mol_type = other RNA
ggctgatgaggccgcacatggttttagagctagaaatagcaagttaaaataaggctagtccgttatcaacttgaaaaagtggcaccgagtcggtgctttt

SEQ ID NO: 27 - RNA, 100 nt, synthetic construct; identical to SEQ ID NO: 21
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA

SEQ ID NO: 28 - RNA, 100 nt, synthetic construct; identical to SEQ ID NO: 22
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA

SEQ ID NO: 29 - RNA, 100 nt, synthetic construct; identical to SEQ ID NO: 23
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA

SEQ ID NO: 30 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
agatttctcacttgttgtacgttttagagctagaaatagcaagttaaaataaggctagtccgttatcaacttgaaaaagtggcaccgagtcggtgctttt

SEQ ID NO: 31 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
gttgtactggtgccctctgcgttttagagctagaaatagcaagttaaaataaggctagtccgttatcaacttgaaaaagtggcaccgagtcggtgctttt

SEQ ID NO: 32 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
gactacaatggaccagcagagttttagagctagaaatagcaagttaaaataaggctagtccgttatcaacttgaaaaagtggcaccgagtcggtgctttt

SEQ ID NO: 33 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
tctaggagcagtgactacaagttttagagctagaaatagcaagttaaaataaggctagtccgttatcaacttgaaaaagtggcaccgagtcggtgctttt

SEQ ID NO: 34 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
tatgaccagaagatggcgtcgttttagagctagaaatagcaagttaaaataaggctagtccgttatcaacttgaaaaagtggcaccgagtcggtgctttt

SEQ ID NO: 35 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
ggttgccagacgccatcttcgttttagagctagaaatagcaagttaaaataaggctagtccgttatcaacttgaaaaagtggcaccgagtcggtgctttt

SEQ ID NO: 36 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
ccataactatgaccagaagagttttagagctagaaatagcaagttaaaataaggctagtccgttatcaacttgaaaaagtggcaccgagtcggtgctttt

SEQ ID NO: 37 - RNA, 100 nt, synthetic construct
  misc_feature 1..100: note = Synthetic
  modified_base 1..3: mod_base = OTHER; note = 2'-O-methyl and phosphorothioate linker
  modified_base 97..99: mod_base = OTHER; note = phosphorothioate linker
  modified_base 98..100: mod_base = OTHER; note = 2'-O-methyl
  source 1..100: mol_type = other RNA
ccatcttctggtcatagttagttttagagctagaaatagcaagttaaaataaggctagtccgttatcaacttgaaaaagtggcaccgagtcggtgctttt

SEQ ID NO: 38 - protein, 456 aa, synthetic construct
  REGION 1..456: note = Synthetic
EWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHWDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 39 - protein, 540 aa, synthetic construct
  REGION 1..540: note = Synthetic
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENWAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMNPLEMFETVPVWRRQPVRVLSLFEDIKKELTSLGFLESGSDPGQLKHWDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 40 - DNA, 1626 bp, synthetic construct
  misc_feature 1..1626: note = Synthetic
  source 1..1626: mol_type = other DNA
aaccacgaccaggagttcgacccccccaaggtgtacccccccgtgcccgccgagaagcggaagcccatccgggtgctgagcctgttcgacggcatcgccaccggcctgctggtgctgaaggacctgggcatccaggtggaccggtacatcgccagcgaggtgtgcgaggacagcatcaccgtgggcatggtgcggcaccagggcaagatcatgtacgtgggcgacgtgcggagcgtgacccagaagcacatccaggagtggggccccttcgacctggtgatcggcggcagcccctgcaacgacctgagcatcgtgaaccccgcccggaagggcctgtacgagggcaccggccggctgttcttcgagttctaccggctgctgcacgacgcccggcccaaggagggcgacgaccggcccttcttctggctgttcgagaacgtggtggccatgggcgtgagcgacaagcgggacatcagccggttcctggagagcaaccccgtgatgatcgacgccaaggaggtgagcgccgcccaccgggcccggtacttctggggcaacctgcccggcatgaaccggcccctggccagcaccgtgaacgacaagctggagctgcaggagtgcctggagcacggccggatcgccaagttcagcaaggtgcggaccatcaccacccggagcaacagcatcaagcagggcaaggaccagcacttccccgtgttcatgaacgagaaggaggacatcctgtggtgcaccgagatggagcgggtgttcggcttccccgtgcactacaccgacgtgagcaacatgagccggctggcccggcagcggctgctgggccggagctggagcgtgcccgtgatccggcacctgttcgcccccctgaaggagtacttcgcctgcgtgagcagcggcaacagcaacgccaacagccggggccccagcttcagcagcggcctggtgcccctgagcctgcggggcagccacatgaatcctctggagatgttcgagacagtgcccgtgtggagaaggcaacccgtgagggtgctgagcctcttcgaggacattaagaaggagctgacctctctgggctttctggaatccggcagcgaccccggccagctgaaacacgtggtggacgtgaccgacacagtgaggaaggacgtggaagagtggggcccctttgacctcgtgtatggagccacacctcctctcggccacacatgcgataggcctcccagctggtatctcttccagttccacagactgctccagtacgccagacctaagcccggcagccccagacccttcttctggatgttcgtggacaatctggtgctgaacaaggaggatctggatgtggccagcagatttctggagatggaacccgtgacaatccccgacgtgcatggcggctctctgcagaacgccgtgagagtgtggtccaacatccccgccattagaagcagacactgggctctggtgagcgaggaggaactgtctctgctggcccagaataagcagtcctccaagctggccgccaagtggcccaccaagctggtgaagaactgctttctgcctctgagggagtatttcaagtatttcagcaccgaactgaccagcagcctg

SEQ ID NO: 41 - RNA, 4004 nt, synthetic construct
  misc_feature 1..4004: note = Synthetic
  source 1..4004: mol_type = other RNA
gggaaataagagagaaaagaagagtaagaagaaatataagagccaccatggccccaaagaagaagcggaaagtcggaggttcaggcggctcgcatatggtggatctgagaaccctgggttacagccagcagcagcaggaaaagattaagccgaaggtccgctcaaccgtggcccaacaccacgaggccttggtcggacatgggttcactcacgcccacatcgtggcgctctcgcaacacccggcagccctgggaaccgtggctgtgaagtaccaagacatgatcgcggccctgcctgaagcaacgcatgaggccattgtcggcgtgggaaagcgcggagctggagccagagcgctggaagccctgctgaccgtcgctggggaactgcggggaccacctctgcaactggacactggacaactgctcaagatcgctaagcgcgggggagtgactgccgtggaggcggtgcatgcctggaggaacgccctgactggagcacctctgaatctgacgcccgaacaggtggtggccattgcgtccaacaacggaggtaaacaggcccttgagactgtgcaacggttgctgccagtcctgtgccaggcccatggccttacaccagaacaagtggtcgctattgccagcaacggaggaggcaagcaggcactggagactgttcaacgcctcctgccggtcctgtgccaggcccatggattgactcctgagcaagtcgtggctatcgcgtcgcacgatggaggcaaacaggctctcgaaacggtgcaaagacttctgcccgtcctgtgtcaagcgcatggcctcacccctgaacaagtagtggccatagcctcccatgacgggggaaaacaggctttggagactgtgcagaggctgctgcctgtcctgtgccaggcccacggtttgaccccggaacaggttgtggccattgcctcgaatatcggagggaaacaagcgctagaaaccgtgcagagacttcttcctgtcctctgtcaggctcacggtctgactcctgaacaggtggtggctattgcttctaacaacggtgggaagcaggccctagaaactgtgcaaaggctgcttcccgtgctatgccaggctcatggactcaccccggagcaagtcgtggccattgcatccaatggaggggggaagcaggccctggaaactgtgcagcgcttgctgcctgtgctgtgccaagctcacggcctaacccccgaacaggtggtggccatcgcctccaacatcggcgggaagcaggccctcgaaacagtccagagattgctcccggtgctgtgtcaggcccacggactcacgccggaacaagtggtggcgattgcctcacacgatggtggcaaacaagcactggaaacggtccagagactgctcccggtcctctgccaggcgcatggcttgacgccagaacaggtcgtggcgattgcctcacacgatgggggaaagcaggctctggaaacagtgcagagattgttacctgtgctctgtcaagcccacggcttgactccagaacaggtcgtagcaattgcctcccacgatggcggaaagcaggcgttagagacagtgcaacggctgctccccgtgttgtgtcaagcacatgggctgacaccggaacaagtggtagccatcgcgagcaacattggagggaagcaagcgctcgagacagtccaacggcttctgccggtgctgtgtcaggcacatggcctgacccctgagcaagttgtcgccattgcttcccatgatggaggaaagcaagccctagagactgtccaacgcctgttgccggtactctgccaagcccatggtttgactccggaacaggtggtggcaatcgcttccaatatcggcggcaagcaagctctcgaaaccgtccagcgactcctgcccgtgctttgtcaggcgcacggactgactcccgaacaagtcgtcgcaattgccagccatgatggtggaaagcaggccttggaaactgtccagcggctcctgccagtgctttgccaagcacacggacttacccccgagcaagtggtcgcaatcgcgtcacatgacggcgggaaacaggcgctggaaaccgttcagagactcttgccggtgttgtgccaggcacacgggctcaccccagaacaggtcgtcgccatcgccagccacgacggtggaaaacaagctcttgaaacggtgcagcgcctccttccggtgctctgtcaagctcatgggcttactccagagcaagtagtcgctatcgcctcgcatgacggtggacgaccagccctggaatccatcgtggcccagctctccagaccggaccctgctctcgctgccctgaccaacgaccacttggtggccctggcctgtctgggtggtagaccggcgttggacgctgtcaagaagggactccctcacgcaccggcacttattaagcggaccaacaggcggatccctgagcgcacttcccatcgcgtggcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - DNA, 3732 bp, synthetic construct
  misc_feature 1..3732: note = Synthetic
  source 1..3732: mol_type = other DNA
atggccccaaagaagaagcggaaagtcggaggttcaggcggctcgcatatggtggatctgagaaccctgggttacagccagcagcagcaggaaaagattaagccgaaggtccgctcaaccgtggcccaacaccacgaggccttggtcggacatgggttcactcacgcccacatcgtggcgctctcgcaacacccggcagccctgggaaccgtggctgtgaagtaccaagacatgatcgcggccctgcctgaagcaacgcatgaggccattgtcggcgtgggaaagcgcggagctggagccagagcgctggaagccctgctgaccgtcgctggggaactgcggggaccacctctgcaactggacactggacaactgctcaagatcgctaagcgcgggggagtgactgccgtggaggcggtgcatgcctggaggaacgccctgactggagcacctctgaatctgacgcccgaacaggtggtggccattgcgtccaacaacggaggtaaacaggcccttgagactgtgcaacggttgctgccagtcctgtgccaggcccatggccttacaccagaacaagtggtcgctattgccagcaacggaggaggcaagcaggcactggagactgttcaacgcctcctgccggtcctgtgccaggcccatggattgactcctgagcaagtcgtggctatcgcgtcgcacgatggaggcaaacaggctctcgaaacggtgcaaagacttctgcccgtcctgtgtcaagcgcatggcctcacccctgaacaagtagtggccatagcctcccatgacgggggaaaacaggctttggagactgtgcagaggctgctgcctgtcctgtgccaggcccacggtttgaccccggaacaggttgtggccattgcctcgaatatcggagggaaacaagcgctagaaaccgtgcagagacttcttcctgtcctctgtcaggctcacggtctgactcctgaacaggtggtggctattgcttctaacaacggtgggaagcaggccctagaaactgtgcaaaggctgcttcccgtgctatgccaggctcatggactcaccccggagcaagtcgtggccattgcatccaatggaggggggaagcaggccctggaaactgtgcagcgcttgctgcctgtgctgtgccaagctcacggcctaacccccgaacaggtggtggccatcgcctccaacatcggcgggaagcaggccctcgaaacagtccagagattgctcccggtgctgtgtcaggcccacggactcacgccggaacaagtggtggcgattgcctcacacgatggtggcaaacaagcactggaaacggtccagagactgctcccggtcctctgccaggcgcatggcttgacgccagaacaggtcgtggcgattgcctcacacgatgggggaaagcaggctctggaaacagtgcagagattgttacctgtgctctgtcaagcccacggcttgactccagaacaggtcgtagcaattgcctcccacgatggcggaaagcaggcgttagagacagtgcaacggctgctccccgtgttgtgtcaagcacatgggctgacaccggaacaagtggtagccatcgcgagcaacattggagggaagcaagcgctcgagacagtccaacggcttctgccggtgctgtgtcaggcacatggcctgacccctgagcaagttgtcgccattgcttcccatgatggaggaaagcaagccctagagactgtccaacgcctgttgccggtactctgccaagcccatggtttgactccggaacaggtggtggcaatcgcttccaatatcggcggcaagcaagctctcgaaaccgtccagcgactcctgcccgtgctttgtcaggcgcacggactgactcccgaacaagtcgtcgcaattgccagccatgatggtggaaagcaggccttggaaactgtccagcggctcctgccagtgctttgccaagcacacggacttacccccgagcaagtggtcgcaatcgcgtcacatgacggcgggaaacaggcgctggaaaccgttcagagactcttgccggtgttgtgccaggcacacgggctcaccccagaacaggtcgtcgccatcgccagccacgacggtggaaaacaagctcttgaaacggtgcagcgcctccttccggtgctctgtcaagctcatgggcttactccagagcaagtagtcgctatcgcctcgcatgacggtggacgaccagccctggaatccatcgtggcccagctctccagaccggaccctgctctcgctgccctgaccaacgaccacttggtggccctggcctgtctgggtggtagaccggcgttggacgctgtcaagaagggactccctcacgcaccggcacttattaagcggaccaacaggcggatccctgagcgcacttcccatcgcgtggcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctga

SEQ ID NO: 43 - protein, 1243 aa, synthetic construct
  REGION 1..1243: note = Synthetic
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 44 - RNA, 2394 nt, synthetic construct
  misc_feature 1..2394: note = Synthetic
  source 1..2394: mol_type = other RNA
catatggtggatctgagaaccctgggttacagccagcagcagcaggaaaagattaagccgaaggtccgctcaaccgtggcccaacaccacgaggccttggtcggacatgggttcactcacgcccacatcgtggcgctctcgcaacacccggcagccctgggaaccgtggctgtgaagtaccaagacatgatcgcggccctgcctgaagcaacgcatgaggccattgtcggcgtgggaaagcgcggagctggagccagagcgctggaagccctgctgaccgtcgctggggaactgcggggaccacctctgcaactggacactggacaactgctcaagatcgctaagcgcgggggagtgactgccgtggaggcggtgcatgcctggaggaacgccctgactggagcacctctgaatctgacgcccgaacaggtggtggccattgcgtccaacaacggaggtaaacaggcccttgagactgtgcaacggttgctgccagtcctgtgccaggcccatggccttacaccagaacaagtggtcgctattgccagcaacggaggaggcaagcaggcactggagactgttcaacgcctcctgccggtcctgtgccaggcccatggattgactcctgagcaagtcgtggctatcgcgtcgcacgatggaggcaaacaggctctcgaaacggtgcaaagacttctgcccgtcctgtgtcaagcgcatggcctcacccctgaacaagtagtggccatagcctcccatgacgggggaaaacaggctttggagactgtgcagaggctgctgcctgtcctgtgccaggcccacggtttgaccccggaacaggttgtggccattgcctcgaatatcggagggaaacaagcgctagaaaccgtgcagagacttcttcctgtcctctgtcaggctcacggtctgactcctgaacaggtggtggctattgcttctaacaacggtgggaagcaggccctagaaactgtgcaaaggctgcttcccgtgctatgccaggctcatggactcaccccggagcaagtcgtggccattgcatccaatggaggggggaagcaggccctggaaactgtgcagcgcttgctgcctgtgctgtgccaagctcacggcctaacccccgaacaggtggtggccatcgcctccaacatcggcgggaagcaggccctcgaaacagtccagagattgctcccggtgctgtgtcaggcccacggactcacgccggaacaagtggtggcgattgcctcacacgatggtggcaaacaagcactggaaacggtccagagactgctcccggtcctctgccaggcgcatggcttgacgccagaacaggtcgtggcgattgcctcacacgatgggggaaagcaggctctggaaacagtgcagagattgttacctgtgctctgtcaagcccacggcttgactccagaacaggtcgtagcaattgcctcccacgatggcggaaagcaggcgttagagacagtgcaacggctgctccccgtgttgtgtcaagcacatgggctgacaccggaacaagtggtagccatcgcgagcaacattggagggaagcaagcgctcgagacagtccaacggcttctgccggtgctgtgtcaggcacatggcctgacccctgagcaagttgtcgccattgcttcccatgatggaggaaagcaagccctagagactgtccaacgcctgttgccggtactctgccaagcccatggtttgactccggaacaggtggtggcaatcgcttccaatatcggcggcaagcaagctctcgaaaccgtccagcgactcctgcccgtgctttgtcaggcgcacggactgactcccgaacaagtcgtcgcaattgccagccatgatggtggaaagcaggccttggaaactgtccagcggctcctgccagtgctttgccaagcacacggacttacccccgagcaagtggtcgcaatcgcgtcacatgacggcgggaaacaggcgctggaaaccgttcagagactcttgccggtgttgtgccaggcacacgggctcaccccagaacaggtcgtcgccatcgccagccacgacggtggaaaacaagctcttgaaacggtgcagcgcctccttccggtgctctgtcaagctcatgggcttactccagagcaagtagtcgctatcgcctcgcatgacggtggacgaccagccctggaatccatcgtggcccagctctccagaccggaccctgctctcgctgccctgaccaacgaccacttggtggccctggcctgtctgggtggtagaccggcgttggacgctgtcaagaagggactccctcacgcaccggcacttattaagcggaccaacaggcggatccctgagcgcacttcccatcgcgtg

SEQ ID NO: 45 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 46 - RNA, 1155 nt, synthetic construct
  misc_feature 1..1155: note = Synthetic
  source 1..1155: mol_type = other RNA
tccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggc

SEQ ID NO: 47 - protein, 385 aa, synthetic construct
  REGION 1..385: note = Synthetic
SKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 48 - DNA, 18 bp, synthetic construct
  misc_feature 1..18: note = Synthetic
  source 1..18: mol_type = other DNA
gtccagtacccacacccc

SEQ ID NO: 49 - RNA, 4004 nt, synthetic construct
  misc_feature 1..4004: note = Synthetic
  source 1..4004: mol_type = other RNA
gggaaataagagagaaaagaagagtaagaagaaatataagagccaccatggccccaaagaagaagcggaaagtcggaggttcaggcggctcgcacatggtggatctgcgcaccctggggtactcgcagcagcagcaggaaaagattaagccgaaggtccggtccactgtcgcccagcaccacgaagcgctcgtcggacacggtttcactcacgcacacatagtcgcactgagccagcaccctgcggcactgggaaccgtggccgtgaagtaccaggatatgatcgcagcccttcctgaggccacccacgaagccatcgtgggcgtgggaaagagaggtgccggagcaagggctcttgaggccctgctgactgtcgcgggagaactcaggggaccgcctctgcaactggacaccggacagctgctgaaaattgccaagcggggcggtgtgactgccgtggaagccgtgcacgcctggagaaacgcactcactggcgctcctctgaacctgaccccggaacaggtcgtcgcgattgcgtcgaacaatggagggaaacaggcgttggaaactgtccaacggttgctcccggtgctatgtcaagcccatggactgacgcccgaacaagtcgtagccatcgcgtcaaacatcggcggaaaacaggcgctcgaaacggtccagcgcttactccccgtgctgtgtcaagcgcatggattgactccggagcaagtcgtcgctatcgccagccacgacggaggaaagcaagctttggaaacagtgcaacgactgctgcccgtcttgtgtcaagcacacgggttgaccccagagcaagtggtggccattgcctcccatgatgggggaaagcaggccctggagactgtgcagcgcctgttgccggtgttgtgccaagcacatgggctgacacccgaacaggtcgtggcgattgcctccaacggtggcggcaaacaagcactcgagacagtgcagcgactcctcccggttctgtgccaggctcacggcctcactcccgaacaggtggtggcaatagcctccaacaacggaggcaaacaggctttagaaacggttcagcgcttgctgccagtcctgtgccaagcgcacggattgaccccggagcaagtggtggccatagccagcaacggcggagggaagcaggccttggaaaccgtacagagactcctccccgtgttatgccaggcacacggcctgacaccagaacaggtggtggcgattgcttccaacggaggcggaaagcaggccctcgaaacagtccagcgacttctgccggtgctgtgccaggctcacggtttgactccagagcaagttgtagctattgccagcaacatcggtgggaagcaggcgctggaaactgtgcaacggctcctgcccgtcctctgccaagctcatggcctcacccccgaacaggtcgtggccatcgcgagccacgatggaggaaaacaagccctggaaacggtgcagaggttgctgcccgtactttgtcaggcccacgggcttactcccgagcaagtggtggccatcgcctccaacattggcggcaagcaggcactggaaactgtgcagagactgcttcccgtgctctgccaggcacacggactcacccctgaacaggttgtggccatagcctcaaacggaggaggaaagcaggcccttgaaactgtccagagactgttgcccgtactgtgtcaggcccacggccttactcctgaacaggtagtggcaatcgcatcgaacggcggtgggaaacaagctctcgagactgtccagcggctgctacctgtgttgtgtcaggctcatggacttacgccggaacaagtagtggccattgcttcccacgacgggggtaaacaggctctggaaaccgtccaaaggctgctgccagtgttgtgccaggcgcatggcctaaccccagaacaagtggtcgcaatcgcctcgcacgatggtggtaaacaagcgctagaaaccgtgcagaggcttctcccagtgctctgtcaggcccacggtctgactccagaacaagtggtcgccatcgcatcccatgacggggggaagcaagccttggagacagtccaacggctgcttccggtgttatgtcaggcgcacggactgactcctgagcaagtcgtggctatcgcgtcgcatgatgggggcaagcaagccctggaaacggtgcaacgcctgctgcctgtcctttgccaggcacacgggctgactccggaacaggtggtggctattgcctcgcatgacggaggtaggcccgcactcgaaagcattgtcgcacagctgtcccggccagatcctgctttggccgccctgaccaacgatcacctcgtcgctctggcctgcctgggtggaaggccggctctggatgccgtgaagaaggggctgcctcatgcccctgccctgattaagagaactaaccggcgcattccggagagaacctcccacagagtggcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 50 - DNA, 3732 bp, synthetic construct
  misc_feature 1..3732: note = Synthetic
  source 1..3732: mol_type = other DNA
atggccccaaagaagaagcggaaagtcggaggttcaggcggctcgcacatggtggatctgcgcaccctggggtactcgcagcagcagcaggaaaagattaagccgaaggtccggtccactgtcgcccagcaccacgaagcgctcgtcggacacggtttcactcacgcacacatagtcgcactgagccagcaccctgcggcactgggaaccgtggccgtgaagtaccaggatatgatcgcagcccttcctgaggccacccacgaagccatcgtgggcgtgggaaagagaggtgccggagcaagggctcttgaggccctgctgactgtcgcgggagaactcaggggaccgcctctgcaactggacaccggacagctgctgaaaattgccaagcggggcggtgtgactgccgtggaagccgtgcacgcctggagaaacgcactcactggcgctcctctgaacctgaccccggaacaggtcgtcgcgattgcgtcgaacaatggagggaaacaggcgttggaaactgtccaacggttgctcccggtgctatgtcaagcccatggactgacgcccgaacaagtcgtagccatcgcgtcaaacatcggcggaaaacaggcgctcgaaacggtccagcgcttactccccgtgctgtgtcaagcgcatggattgactccggagcaagtcgtcgctatcgccagccacgacggaggaaagcaagctttggaaacagtgcaacgactgctgcccgtcttgtgtcaagcacacgggttgaccccagagcaagtggtggccattgcctcccatgatgggggaaagcaggccctggagactgtgcagcgcctgttgccggtgttgtgccaagcacatgggctgacacccgaacaggtcgtggcgattgcctccaacggtggcggcaaacaagcactcgagacagtgcagcgactcctcccggttctgtgccaggctcacggcctcactcccgaacaggtggtggcaatagcctccaacaacggaggcaaacaggctttagaaacggttcagcgcttgctgccagtcctgtgccaagcgcacggattgaccccggagcaagtggtggccatagccagcaacggcggagggaagcaggccttggaaaccgtacagagactcctccccgtgttatgccaggcacacggcctgacaccagaacaggtggtggcgattgcttccaacggaggcggaaagcaggccctcgaaacagtccagcgacttctgccggtgctgtgccaggctcacggtttgactccagagcaagttgtagctattgccagcaacatcggtgggaagcaggcgctggaaactgtgcaacggctcctgcccgtcctctgccaagctcatggcctcacccccgaacaggtcgtggccatcgcgagccacgatggaggaaaacaagccctggaaacggtgcagaggttgctgcccgtactttgtcaggcccacgggcttactcccgagcaagtggtggccatcgcctccaacattggcggcaagcaggcactggaaactgtgcagagactgcttcccgtgctctgccaggcacacggactcacccctgaacaggttgtggccatagcctcaaacggaggaggaaagcaggcccttgaaactgtccagagactgttgcccgtactgtgtcaggcccacggccttactcctgaacaggtagtggcaatcgcatcgaacggcggtgggaaacaagctctcgagactgtccagcggctgctacctgtgttgtgtcaggctcatggacttacgccggaacaagtagtggccattgcttcccacgacgggggtaaacaggctctggaaaccgtccaaaggctgctgccagtgttgtgccaggcgcatggcctaaccccagaacaagtggtcgcaatcgcctcgcacgatggtggtaaacaagcgctagaaaccgtgcagaggcttctcccagtgctctgtcaggcccacggtctgactccagaacaagtggtcgccatcgcatcccatgacggggggaagcaagccttggagacagtccaacggctgcttccggtgttatgtcaggcgcacggactgactcctgagcaagtcgtggctatcgcgtcgcatgatgggggcaagcaagccctggaaacggtgcaacgcctgctgcctgtcctttgccaggcacacgggctgactccggaacaggtggtggctattgcctcgcatgacggaggtaggcccgcactcgaaagcattgtcgcacagctgtcccggccagatcctgctttggccgccctgaccaacgatcacctcgtcgctctggcctgcctgggtggaaggccggctctggatgccgtgaagaaggggctgcctcatgcccctgccctgattaagagaactaaccggcgcattccggagagaacctcccacagagtggcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctga

SEQ ID NO: 51 - protein, 1243 aa, synthetic construct
  REGION 1..1243: note = Synthetic
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 52 - RNA, 2394 nt, synthetic construct
  misc_feature 1..2394: note = Synthetic
  source 1..2394: mol_type = other RNA
cacatggtggatctgcgcaccctggggtactcgcagcagcagcaggaaaagattaagccgaaggtccggtccactgtcgcccagcaccacgaagcgctcgtcggacacggtttcactcacgcacacatagtcgcactgagccagcaccctgcggcactgggaaccgtggccgtgaagtaccaggatatgatcgcagcccttcctgaggccacccacgaagccatcgtgggcgtgggaaagagaggtgccggagcaagggctcttgaggccctgctgactgtcgcgggagaactcaggggaccgcctctgcaactggacaccggacagctgctgaaaattgccaagcggggcggtgtgactgccgtggaagccgtgcacgcctggagaaacgcactcactggcgctcctctgaacctgaccccggaacaggtcgtcgcgattgcgtcgaacaatggagggaaacaggcgttggaaactgtccaacggttgctcccggtgctatgtcaagcccatggactgacgcccgaacaagtcgtagccatcgcgtcaaacatcggcggaaaacaggcgctcgaaacggtccagcgcttactccccgtgctgtgtcaagcgcatggattgactccggagcaagtcgtcgctatcgccagccacgacggaggaaagcaagctttggaaacagtgcaacgactgctgcccgtcttgtgtcaagcacacgggttgaccccagagcaagtggtggccattgcctcccatgatgggggaaagcaggccctggagactgtgcagcgcctgttgccggtgttgtgccaagcacatgggctgacacccgaacaggtcgtggcgattgcctccaacggtggcggcaaacaagcactcgagacagtgcagcgactcctcccggttctgtgccaggctcacggcctcactcccgaacaggtggtggcaatagcctccaacaacggaggcaaacaggctttagaaacggttcagcgcttgctgccagtcctgtgccaagcgcacggattgaccccggagcaagtggtggccatagccagcaacggcggagggaagcaggccttggaaaccgtacagagactcctccccgtgttatgccaggcacacggcctgacaccagaacaggtggtggcgattgcttccaacggaggcggaaagcaggccctcgaaacagtccagcgacttctgccggtgctgtgccaggctcacggtttgactccagagcaagttgtagctattgccagcaacatcggtgggaagcaggcgctggaaactgtgcaacggctcctgcccgtcctctgccaagctcatggcctcacccccgaacaggtcgtggccatcgcgagccacgatggaggaaaacaagccctggaaacggtgcagaggttgctgcccgtactttgtcaggcccacgggcttactcccgagcaagtggtggccatcgcctccaacattggcggcaagcaggcactggaaactgtgcagagactgcttcccgtgctctgccaggcacacggactcacccctgaacaggttgtggccatagcctcaaacggaggaggaaagcaggcccttgaaactgtccagagactgttgcccgtactgtgtcaggcccacggccttactcctgaacaggtagtggcaatcgcatcgaacggcggtgggaaacaagctctcgagactgtccagcggctgctacctgtgttgtgtcaggctcatggacttacgccggaacaagtagtggccattgcttcccacgacgggggtaaacaggctctggaaaccgtccaaaggctgctgccagtgttgtgccaggcgcatggcctaaccccagaacaagtggtcgcaatcgcctcgcacgatggtggtaaacaagcgctagaaaccgtgcagaggcttctcccagtgctctgtcaggcccacggtctgactccagaacaagtggtcgccatcgcatcccatgacggggggaagcaagccttggagacagtccaacggctgcttccggtgttatgtcaggcgcacggactgactcctgagcaagtcgtggctatcgcgtcgcatgatgggggcaagcaagccctggaaacggtgcaacgcctgctgcctgtcctttgccaggcacacgggctgactccggaacaggtggtggctattgcctcgcatgacggaggtaggcccgcactcgaaagcattgtcgcacagctgtcccggccagatcctgctttggccgccctgaccaacgatcacctcgtcgctctggcctgcctgggtggaaggccggctctggatgccgtgaagaaggggctgcctcatgcccctgccctgattaagagaactaaccggcgcattccggagagaacctcccacagagtg

SEQ ID NO: 53 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 54 - skipped

SEQ ID NO: 55 - protein, 10 aa, synthetic construct
  REGION 1..10: note = Synthetic
ASGSGGGSGG

SEQ ID NO: 56 - DNA, 18 bp, synthetic construct
  misc_feature 1..18: note = Synthetic
  source 1..18: mol_type = other DNA
gacctgttacattccccc

SEQ ID NO: 57 - RNA, 4004 nt, synthetic construct
  misc_feature 1..4004: note = Synthetic
  source 1..4004: mol_type = other RNA
gggaaataagagagaaaagaagagtaagaagaaatataagagccaccatggccccaaagaagaagcggaaagtcggaggttcaggcggctcgcacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacgggggggagtgaccgctgtggaagccgtgcacgcatggaggaacgctctcaccggcgcaccgttaaaccttacgcccgaacaggttgtggccatagcctccaacattggcgggaaacaggcattggagactgtccaacggttgcttccagtgctttgccaagcccacggattgacaccagaacaggtcgttgcaatcgccagccacgatgggggtaaacaagcgctggagacagtccaacggctgctccccgtgctgtgccaggcccatggtctgactccggaacaggtggtggccatcgccagcaacggtggaggcaaacaagccctcgaaacggtacaaagacttttgcccgtgttgtgccaggcgcatggacttacccctgagcaagtggtagctattgccagccatgatgggggaaagcaagccttggagacagtgcaacgcctgctccctgtgctgtgtcaggcgcacggcctcactccggaacaggtcgtcgctatcgcttcccatgacggcggcaagcaggctctggaaacagtgcagcggttgctgcccgtgctctgccaagcacatgggctgactcctgaacaagtcgtggcaattgccagcaacattggaggaaagcaggccttggaaactgtccagcggctccttccggtcctctgtcaagcacacggactgaccccggaacaagtggtggcgattgcttcgaacatcggtggcaagcaggcactggaaacggtgcagaggctgctgccagtgctgtgccaggcacacgggcttactccagaacaggttgtcgctattgcctccaacggaggaggcaagcaggcgctcgaaaccgtacaacgcttgctgccggtcctttgtcaagcgcacgggctcactccagagcaagtggtcgcgattgcctcccacgatggagggaaacaagctcttgagactgtgcagagactcttgcctgtgctctgtcaggcccacggtctgacccctgaacaggtggttgcgattgcaagccacgacggtggtaaacaggccctggaaactgtgcagcgcctcctgcctgtcctgtgccaagcgcatggcctcaccccggaacaagtggtggcgattgcgagcaacggagggggcaaacaggcgctggaaacggtgcagaggctgctcccggtgctttgccaagcccacggacttacacccgaacaggtcgtggccatcgccagtcatgacgggggcaagcaagctttggaaaccgtgcagcggctgttgccagtgttgtgtcaagctcacgggctgacgcctgaacaagtcgtcgccatcgcttcacacgacggaggaaagcaggccttggaaactgtccagaggctcttgccggtcctatgtcaggcacatggactgactcccgaacaagtggtggcaatcgcctcgaacggtggtgggaagcaggccctcgagactgtgcaacggcttctgcctgtgttatgccaggctcatggcctgacccccgagcaagtagtcgccatcgcctcacacgatggcggaaaacaggctctcgaaacagtccaaagactgctgcccgtcctgtgtcaagcccatggactcacccccgaacaagtggtggctatcgcgtcgaatatcggcgggaagcaggcccttgaaaccgtccagagacttctgccggtgctctgccaagctcacggcctgacgccagaacaagtagtggccattgcatctaacaacggggggaagcaagctctcgaaactgtgcaaaggctcctgcccgtcttgtgtcaggctcacggtctgacccctgaacaggtggtcgccattgcgtcacgccatggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtggcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 58 - DNA, 3732 bp, synthetic construct
  misc_feature 1..3732: note = Synthetic
  source 1..3732: mol_type = other DNA
atggccccaaagaagaagcggaaagtcggaggttcaggcggctcgcacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacgggggggagtgaccgctgtggaagccgtgcacgcatggaggaacgctctcaccggcgcaccgttaaaccttacgcccgaacaggttgtggccatagcctccaacattggcgggaaacaggcattggagactgtccaacggttgcttccagtgctttgccaagcccacggattgacaccagaacaggtcgttgcaatcgccagccacgatgggggtaaacaagcgctggagacagtccaacggctgctccccgtgctgtgccaggcccatggtctgactccggaacaggtggtggccatcgccagcaacggtggaggcaaacaagccctcgaaacggtacaaagacttttgcccgtgttgtgccaggcgcatggacttacccctgagcaagtggtagctattgccagccatgatgggggaaagcaagccttggagacagtgcaacgcctgctccctgtgctgtgtcaggcgcacggcctcactccggaacaggtcgtcgctatcgcttcccatgacggcggcaagcaggctctggaaacagtgcagcggttgctgcccgtgctctgccaagcacatgggctgactcctgaacaagtcgtggcaattgccagcaacattggaggaaagcaggccttggaaactgtccagcggctccttccggtcctctgtcaagcacacggactgaccccggaacaagtggtggcgattgcttcgaacatcggtggcaagcaggcactggaaacggtgcagaggctgctgccagtgctgtgccaggcacacgggcttactccagaacaggttgtcgctattgcctccaacggaggaggcaagcaggcgctcgaaaccgtacaacgcttgctgccggtcctttgtcaagcgcacgggctcactccagagcaagtggtcgcgattgcctcccacgatggagggaaacaagctcttgagactgtgcagagactcttgcctgtgctctgtcaggcccacggtctgacccctgaacaggtggttgcgattgcaagccacgacggtggtaaacaggccctggaaactgtgcagcgcctcctgcctgtcctgtgccaagcgcatggcctcaccccggaacaagtggtggcgattgcgagcaacggagggggcaaacaggcgctggaaacggtgcagaggctgctcccggtgctttgccaagcccacggacttacacccgaacaggtcgtggccatcgccagtcatgacgggggcaagcaagctttggaaaccgtgcagcggctgttgccagtgttgtgtcaagctcacgggctgacgcctgaacaagtcgtcgccatcgcttcacacgacggaggaaagcaggccttggaaactgtccagaggctcttgccggtcctatgtcaggcacatggactgactcccgaacaagtggtggcaatcgcctcgaacggtggtgggaagcaggccctcgagactgtgcaacggcttctgcctgtgttatgccaggctcatggcctgacccccgagcaagtagtcgccatcgcctcacacgatggcggaaaacaggctctcgaaacagtccaaagactgctgcccgtcctgtgtcaagcccatggactcacccccgaacaagtggtggctatcgcgtcgaatatcggcgggaagcaggcccttgaaaccgtccagagacttctgccggtgctctgccaagctcacggcctgacgccagaacaagtagtggccattgcatctaacaacggggggaagcaagctctcgaaactgtgcaaaggctcctgcccgtcttgtgtcaggctcacggtctgacccctgaacaggtggtcgccattgcgtcacgccatggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtggcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctga

SEQ ID NO: 59 - protein, 1243 aa, synthetic construct
  REGION 1..1243: note = Synthetic
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGRPALESIVAQLSRPDPALAALTNDHLVALACLGGRPALDAVKKGLPHAPALIKRTNRRIPERTSHRVASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 60 - RNA, 2394 nt, synthetic construct
  misc_feature 1..2394: note = Synthetic
  source 1..2394: mol_type = other RNA
cacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacgggggggagtgaccgctgtggaagccgtgcacgcatggaggaacgctctcaccggcgcaccgttaaaccttacgcccgaacaggttgtggccatagcctccaacattggcgggaaacaggcattggagactgtccaacggttgcttccagtgctttgccaagcccacggattgacaccagaacaggtcgttgcaatcgccagccacgatgggggtaaacaagcgctggagacagtccaacggctgctccccgtgctgtgccaggcccatggtctgactccggaacaggtggtggccatcgccagcaacggtggaggcaaacaagccctcgaaacggtacaaagacttttgcccgtgttgtgccaggcgcatggacttacccctgagcaagtggtagctattgccagccatgatgggggaaagcaagccttggagacagtgcaacgcctgctccctgtgctgtgtcaggcgcacggcctcactccggaacaggtcgtcgctatcgcttcccatgacggcggcaagcaggctctggaaacagtgcagcggttgctgcccgtgctctgccaagcacatgggctgactcctgaacaagtcgtggcaattgccagcaacattggaggaaagcaggccttggaaactgtccagcggctccttccggtcctctgtcaagcacacggactgaccccggaacaagtggtggcgattgcttcgaacatcggtggcaagcaggcactggaaacggtgcagaggctgctgccagtgctgtgccaggcacacgggcttactccagaacaggttgtcgctattgcctccaacggaggaggcaagcaggcgctcgaaaccgtacaacgcttgctgccggtcctttgtcaagcgcacgggctcactccagagcaagtggtcgcgattgcctcccacgatggagggaaacaagctcttgagactgtgcagagactcttgcctgtgctctgtcaggcccacggtctgacccctgaacaggtggttgcgattgcaagccacgacggtggtaaacaggccctggaaactgtgcagcgcctcctgcctgtcctgtgccaagcgcatggcctcaccccggaacaagtggtggcgattgcgagcaacggagggggcaaacaggcgctggaaacggtgcagaggctgctcccggtgctttgccaagcccacggacttacacccgaacaggtcgtggccatcgccagtcatgacgggggcaagcaagctttggaaaccgtgcagcggctgttgccagtgttgtgtcaagctcacgggctgacgcctgaacaagtcgtcgccatcgcttcacacgacggaggaaagcaggccttggaaactgtccagaggctcttgccggtcctatgtcaggcacatggactgactcccgaacaagtggtggcaatcgcctcgaacggtggtgggaagcaggccctcgagactgtgcaacggcttctgcctgtgttatgccaggctcatggcctgacccccgagcaagtagtcgccatcgcctcacacgatggcggaaaacaggctctcgaaacagtccaaagactgctgcccgtcctgtgtcaagcccatggactcacccccgaacaagtggtggctatcgcgtcgaatatcggcgggaagcaggcccttgaaaccgtccagagacttctgccggtgctctgccaagctcacggcctgacgccagaacaagtagtggccattgcatctaacaacggggggaagcaagctctcgaaactgtgcaaaggctcctgcccgtcttgtgtcaggctcacggtctgacccctgaacaggtggtcgccattgcgtcacgccatggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtg

SEQ ID NO: 61 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGRPALESIVAQLSRPDPALAALTNDHLVALACLGGRPALDAVKKGLPHAPALIKRTNRRIPERTSHRV

SEQ ID NO: 62 - protein, 13 aa, synthetic construct
  REGION 1..13: note = Synthetic
ASGSGGGSGGARD

SEQ ID NO: 63 - protein, 5 aa, synthetic construct
  REGION 1..5: note = Synthetic
GGGGS

SEQ ID NO: 64 - DNA, 18 bp, synthetic construct
  misc_feature 1..18: note = Synthetic
  source 1..18: mol_type = other DNA
actccaatcctcctcagg

SEQ ID NO: 65 - DNA, 28 bp, synthetic construct
  misc_feature 1..28: note = Synthetic
  source 1..28: mol_type = other DNA
gtttaggagaagttagttaatattgttg

SEQ ID NO: 66 - DNA, 28 bp, synthetic construct
  misc_feature 1..28: note = Synthetic
  source 1..28: mol_type = other DNA
atatctctaaaaaaccaaaaactaactc

SEQ ID NO: 67 - DNA, 18 bp, synthetic construct
  misc_feature 1..18: note = Synthetic
  source 1..18: mol_type = other DNA
agacttctgaatcaatcc

SEQ ID NO: 68 - DNA, 18 bp, synthetic construct
  misc_feature 1..18: note = Synthetic
  source 1..18: mol_type = other DNA
agaagtctcactggtcag

SEQ ID NO: 69 - DNA, 18 bp, synthetic construct
  misc_feature 1..18: note = Synthetic
  source 1..18: mol_type = other DNA
ggtggacccaggaaacac

SEQ ID NO: 70 - DNA, 18 bp, synthetic construct
  misc_feature 1..18: note = Synthetic
  source 1..18: mol_type = other DNA
aaggtggacccaggaaac

SEQ ID NO: 71 - DNA, 18 bp, synthetic construct
  misc_feature 1..18: note = Synthetic
  source 1..18: mol_type = other DNA
actggaggatcaggtttc

SEQ ID NO: 72 - DNA, 18 bp, synthetic construct
  misc_feature 1..18: note = Synthetic
  source 1..18: mol_type = other DNA
gtttaatcagataggatc

SEQ ID NO: 73 - DNA, 18 bp, synthetic construct
  misc_feature 1..18: note = Synthetic
  source 1..18: mol_type = other DNA
atctgattaaacattaac

SEQ ID NO: 74 - DNA, 18 bp, synthetic construct
  misc_feature 1..18: note = Synthetic
  source 1..18: mol_type = other DNA
aaaccttctagggtgtgg

SEQ ID NO: 75 - DNA, 18 bp, synthetic construct
  misc_feature 1..18: note = Synthetic
  source 1..18: mol_type = other DNA
aaccttctagggtgtggg

SEQ ID NO: 76 - DNA, 18 bp, synthetic construct
  misc_feature 1..18: note = Synthetic
  source 1..18: mol_type = other DNA
cacccacaccctagaagg

SEQ ID NO: 77 - DNA, 18 bp, synthetic construct
  misc_feature 1..18: note = Synthetic
  source 1..18: mol_type = other DNA
gaatcaatcctactgtgg

SEQ ID NO: 78 - DNA, 18 bp, synthetic construct
  misc_feature 1..18: note = Synthetic
  source 1..18: mol_type = other DNA
gatcctatctgattaaac

SEQ ID NO: 79 - DNA, 18 bp, synthetic construct
  misc_feature 1..18: note = Synthetic
  source 1..18: mol_type = other DNA
cacaccctagaaggtttc

SEQ ID NO: 80 - DNA, 18 bp, synthetic construct
  misc_feature 1..18: note = Synthetic
  source 1..18: mol_type = other DNA
gaaaccttctagggtgtg

SEQ ID NO: 81 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 82 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRV

SEQ ID NO: 83 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 84 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 85 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 86 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 87 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 88 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGRPALESIVAQLSRPDPALAALTNDHLVALACLGGRPALDAVKKGLPHAPALIKRTNRRIPERTSHRV

SEQ ID NO: 89 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGRPALESIVAQLSRPDPALAALTNDHLVALACLGGRPALDAVKKGLPHAPALIKRTNRRIPERTSHRV

SEQ ID NO: 90 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGRPALESIVAQLSRPDPALAALTNDHLVALACLGGRPALDAVKKGLPHAPALIKRTNRRIPERTSHRV

SEQ ID NO: 91 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGRPALESIVAQLSRPDPALAALTNDHLVALACLGGRPALDAVKKGLPHAPALIKRTNRRIPERTSHRV

SEQ ID NO: 92 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 93 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 94 - protein, 798 aa, synthetic construct
  REGION 1..798: note = Synthetic
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 95 - protein, 1367 aa, synthetic construct
  REGION 1..1367: note = Synthetic
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 96 - DNA, 4101 bp, synthetic construct
  misc_feature 1..4101: note = Synthetic
  source 1..4101: mol_type = other DNA
gacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgac

SEQ ID NO: 97 - protein, 385 aa, synthetic construct; identical to SEQ ID NO: 47
  REGION 1..385: note = Synthetic

SEQ ID NO: 98 - DNA, 1155 bp, synthetic construct
  misc_feature 1..1155: note = Synthetic
  source 1..1155: mol_type = other DNA
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 99 - protein, 386 aa, synthetic construct
  REGION 1..386: note = Synthetic
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 100 - RNA, 1155 nt, synthetic construct
  misc_feature 1..1155: note = Synthetic
  source 1..1155: mol_type = other RNA
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 101 - protein, 96 aa, synthetic construct
  REGION 1..96: note = Synthetic
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 102 - DNA, 288 bp, synthetic construct
  misc_feature 1..288: note = Synthetic
  source 1..288: mol_type = other DNA
gacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtg

SEQ ID NO: 103 - protein, 479 aa, Homo sapiens
  REGION 1..479: note = misc_feature - human DNMT1 amino acid
VDLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLWSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 104 - DNA, 1440 bp, Homo sapiens
  misc_feature 1..1440: note = human DNMT1 nucleotide
  source 1..1440: mol_type = genomic DNA
gtggatctgaggacactcgacgtgtttagcggatgcggcggactctccgaaggcttccaccaagccggaatttccgacacactctgggccattgagatgtgggaccccgccgctcaagccttcagactgaataatcccggctccaccgtgttcaccgaggactgcaacattctgctgaagctggtgatggctggcgaaaccaccaactctagaggccagaggctgccccagaagggagatgtggaaatgctctgtggaggccctccttgccaaggcttctccggcatgaacaggttcaactctagaacatacagcaagttcaagaactctctggtcgtgagctttctgagctactgcgactactatagacctaggttctttctgctggagaacgtgagaaatttcgtgtccttcaagaggagcatggtgctgaagctgacactgaggtgtctggtgaggatgggctaccagtgcacattcggagtgctgcaagctggccagtacggcgtggcccagaccagaaggagggccatcattctggctgctgcccccggcgagaaactccctctgttccccgagcccctccacgtgttcgcccctagagcttgccagctgagcgtggtggtcgacgataagaagttcgtgagcaacatcacaaggctgtccagcggacccttcagaaccattaccgtgagggataccatgtccgacctccccgaggtgaggaatggcgccagcgctctggagatttcctacaacggcgaacctcagagctggttccaaaggcagctgagaggcgctcagtatcagcccattctgagggaccacatctgcaaagatatgagcgctctggtggccgctagaatgagacatattcctctggcccccggcagcgactggagagatctgcccaatattgaggtgagactcagcgacggaacaatggctagaaaactgaggtacacccatcatgatagaaagaacggaaggagcagcagcggcgctctgagaggagtgtgtagctgcgtggaagctggcaaggcttgcgatcccgccgctaggcagttcaataccctcatcccttggtgtctgcctcacaccggcaacagacacaatcattgggctggactgtatggaaggctcgaatgggacggctttttcagcaccaccgtgaccaatcccgaacctatgggcaagcaaggaagggtgctccaccccgagcagcatagagtcgtgtccgtgagagaatgcgctagaagccaaggcttccccgacacctatagactgttcggcaacattctggataagcacagacaagtgggaaatgctgtccctcctcctctggccaaggctatcggactggagatcaagctgtgtatgctcgccaaagctagggagagcgcttccgccaagattaaggaggaggaggccgccaaggac

SEQ ID NO: 105 - DNA, 684 bp, Mus musculus
  misc_feature 1..684: note = (Fig. 1A) Mouse PCSK9 top strand
  source 1..684: mol_type = genomic DNA
cctcctgggacgggagggcgagcatcagctcttcataatctccatcctcgtcctgggcaccagcgccggtggggcacagtagcagcaacagcagcagcagcggcggcaacagcggcaacagcggccaccgcagccacgcagagcagtgggtgcccatcggggcgaggagaggtgcgcgcggcctcgggaaggacatggacgcctgggactcggggcacttggctccggggctgcgggccgtgatgtctctggggagccaaggactggctcagtcctctagcctcagaaagagagagagaacgggagcccaccggcagcctgcgcgtccacgtgaaggtggaagccttctggggtgtgggtactggacgcctgggggccgggagatcacgccaccccgagccccatcggaagatcctctctgattaaaaaggatcaggtttcggcctcgccctccccagacagcgtcaaatgacgctcagagtgaggacaaaaccccaaatcctaattgggctgcaaacgggacctgttacattcccccctcttaatccccactccaatcctcctcaggcggggtgccaactcagaggcgtgctgagccccacctctttagcatttccggggtctcccagccacccctgtacttctccctccaacaatattaactaacttctcctgggccctcct

SEQ ID NO: 106 - DNA, 684 bp, Mus musculus
  misc_feature 1..684: note = (Fig. 1A) Mouse PCSK9 bottom strand
  source 1..684: mol_type = genomic DNA
aggagggcccaggagaagttagttaatattgttggagggagaagtacaggggtggctgggagaccccggaaatgctaaagaggtggggctcagcacgcctctgagttggcaccccgcctgaggaggattggagtggggattaagaggggggaatgtaacaggtcccgtttgcagcccaattaggatttggggttttgtcctcactctgagcgtcatttgacgctgtctggggagggcgaggccgaaacctgatcctttttaatcagagaggatcttccgatggggctcggggtggcgtgatctcccggcccccaggcgtccagtacccacaccccagaaggcttccaccttcacgtggacgcgcaggctgccggtgggctcccgttctctctctctttctgaggctagaggactgagccagtccttggctccccagagacatcacggcccgcagccccggagccaagtgccccgagtcccaggcgtccatgtccttcccgaggccgcgcgcacctctcctcgccccgatgggcacccactgctctgcgtggctgcggtggccgctgttgccgctgttgccgccgctgctgctgctgttgctgctactgtgccccaccggcgctggtgcccaggacgaggatggagattatgaagagctgatgctcgccctcccgtcccaggagg

SEQ ID NO: 107 - protein, 72 aa, Mus musculus
  REGION 1..72: note = misc_feature - (Fig. 1A) Mouse PCSK9 protein
MGTHCSAWLRWPLLPLLPPLLLLLLLLCPTGAGAQDEDGDYEELMLALPSQEDGLADEAAHVATATFRRCSK

SEQ ID NO: 108 - DNA, 855 bp, Homo sapiens
  misc_feature 1..855: note = (Fig. 1B) Human PCSK9 top strand
  source 1..855: mol_type = genomic DNA
tgggcagcgaggagtccacagtaggattgattcagaagtctcactggtcagcaggagacaaggtggacccaggaaacactgaaaaggtgggcccggcagaacttggagtctggcatcccacgcagggtgagaggcgggagaggaggagcccctagggcgccggcctgccttccagcccagttaggatttgggagttttttcttccctctgcgcgtaatctgacgctgtttggggagggcgaggccgaaacctgatcctccagtccgggggttccgttaatgtttaatcagataggatcgtccgatggggctctggtggcgtgatctgcgcgccccaggcgtcaagcacccacaccctagaaggtttccgcagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccac

SEQ ID NO: 109 - DNA, 855 bp, Homo sapiens
  misc_feature 1..855: note = (Fig. 1B) Human PCSK9 bottom strand
  source 1..855: mol_type = genomic DNA
gtggaaggtggctgtggttccgtgctcgggtgcttcggccaggccgtcctcctcggaacgcaaggctagcaccagctcctcgtagtcgccgtcctcgtcctcctgcgcacgggcgcccgcgggacccaggagcagcagcagcagcagcagcagtggcagcggccaccaggaccgcctggagctgacggtgcccatgagggccaggggagaggttgctgtcctggcgaggagacctagaggccgtgcgcggtccacgccggcggcgccttgagccttgcggtggggaggactgtgcaggagctgaagttcaggagcagggcgcgtgaaggggcgcgcggaatcctggctgggagctgggagccgctgctgcaacgacgcgtcccggcccgcccgagccagtctcactgcctggctcactcctccaggctcagaccctgaactgaacggcggcgcccgcctgcaaccatgagcgcctcgacgtcgctgcggaaaccttctagggtgtgggtgcttgacgcctggggcgcgcagatcacgccaccagagccccatcggacgatcctatctgattaaacattaacggaacccccggactggaggatcaggtttcggcctcgccctccccaaacagcgtcagattacgcgcagagggaagaaaaaactcccaaatcctaactgggctggaaggcaggccggcgccctaggggctcctcctctcccgcctctcaccctgcgtgggatgccagactccaagttctgccgggcccaccttttcagtgtttcctgggtccaccttgtctcctgctgaccagtgagacttctgaatcaatcctactgtggactcctcgctgccca

SEQ ID NO: 110 - protein, 65 aa, Homo sapiens
  REGION 1..65: note = misc_feature - (Fig. 1B) Human PCSK9 protein
MGTVSSRRSWWPLPLLLLLLLLLGPAGARAQEDEDGDYEELVLALRSEEDGLAEAPEHGTTATFH

(19) United States
(12) Patent Application Publication
(10) Pub. No.: US 2026/0327587 A1
(43) Pub. Date: Oct. 22, 2026

(54) METHODS AND COMPOSITIONS FOR MODULATING PCSK9 EXPRESSION

(71) Applicant: FLAGSHIP LABS 114, INC., Cambridge, MA (US)

(72) Inventors: Chevaun Danielle Morrison-Smith, Andover, MA (US); Jeremiah D. Farelli, Marblehead, MA (US); Charles W. O'Donnell, Winchester, MA (US)

(21) Appl. No.: 19/484,264

(22) PCT Filed: May 16, 2024
(86) PCT No.: PCT/US2024/029573

§ 371 Date: Nov. 13, 2025

Related U.S. Application Data
(60) Provisional application No. 63/502,590, filed on May 16, 2023.

(51) Int. Cl. C12N 9/22; C12N 9/10; C12N 9/80; C12N 15/11; C12N 15/88; C12N 15/90

(52) U.S. Cl. CPC C12N 9/226; C12N 9/1007; C12N 9/80; C12N 15/11; C12N 15/88; C12N 15/907; C12Y 305/01098; C12N 2310/20

(57) ABSTRACT

The present disclosure is directed to compositions and methods for reducing expression of the PCSK9 gene in a cell, e.g., using an expression repressor that comprises a targeting moiety that binds a PCSK9 promoter, anchor sequence, or super-enhancer and an effector domain that represses transcription or methylates DNA. Systems comprising two or more expression repressors are also disclosed. The compositions can be used, for example, to treat a PCSK9-associated disease or disorder.

### CROSS-REFERENCE TO RELATED APPLICATIONS

This application claims the benefit of U.S. Provisional Patent Application No. 63/502,590 filed May 16, 2023, which is incorporated by reference herein in its entirety.

### BACKGROUND

Serine proteases are the largest protease family in humans and includes the proprotein convertases (PCs). PCs function in cellular processing pathways that activate, inactivate, or alter certain hormones, neuropeptides, growth factors, and receptors. As a result, they are essential for cellular homeostasis and their dysregulation can contribute to disease. PCSK9 is a PC having expression in the liver, intestines, and kidneys (see Luo, et al (2009) J. Lipid Res 50:1581). PCSK9's catalytic activity is to produce an autocleavage of its N-terminal prodomain in the ER (cleaving between Gln-152 and Ser-153 (i.e., VFAQ_{152↓})) (see Naureckiene et al., (2003) Arch. Biochem. Biophys. 420:55-67; Benjannet, et al (2004) J. Biol Chem. 279:48865; Benjannet, et al (2012) J. Biol Chem 287:33745; Seidah et al., (2003) PNAS 100:928-933), which has been shown to be a critical maturation step preceding its secretion and extracellular function (see Benjannet et al., (2012) J. Biol. Chem. 287:33745-33755). Once secreted, the prodomain remains non-covalently associated and thus, following autocatalytic processing, does not function further in proteolysis (see Seida, et al (2003) PNAS 100:928).

PCSK9 functions as a chaperone that directs the LDL receptor (LDLR) for intracellular catabolism. The LDLR facilitates LDL clearance and lowers levels of LDL-cholesterol (LDLc) (Seidah, et al (2021) J Lipid Res 62:100130). PCSK9 forms a protein-protein interaction with the epidermal growth factor-like repeat A (EGF-A) domain of the LDLR once internalized and targets it to lysosomes for degradation (Kwon et al, (2008) PNAS 105:1820-1825; Zhang, et al (2007) J. Biol. Chem. 282:18602-18612; Li et al (2007) Biochem J. 406:203-07; McNutt, et al (2007) J Biol Chem 282:20799-20803). Inhibiting the interaction between PCSK9 and LDLR using an anti-PCSK9 monoclonal antibody has been shown to increases recycling of LDL receptors, thereby increasing LDL uptake and providing lowered circulating LDLc (see Roth et al, (2012) N Engl J Med 367:1891). Circulating levels of LDLc positively corelate with risk of heart disease, and reducing levels of LDLc (e.g., using statins), has positive health benefits. The role of PCSK9 in modulating levels of circulating LDLc has implications for human health. Indeed, it has been shown that while gain of function mutations in PCSK9 are associated with autosomal dominant hypercholesterolemia (“ADH”), an inherited metabolism disorder characterized by marked elevations of LDL particles in the plasma, which can lead to premature cardiovascular failure (see Abifadel et al. (2003) Nat. Gen. 34:154-156; Timms et al. (2004) Hum. Genet. 114:349-353; Leren (2004) Clin. Genet. 65:419-422), loss of function in PCSK9 is associated with reduced risk of atherosclerotic cardiovascular disease (see Cohen, et al (2005) Nat Genet 37:161).

In view of the beneficial effects of reducing PCSK9 levels in order to decrease circulating LDLc, drug discovery efforts have focused on therapeutics to decrease expression levels of PCSK9 or antagonize its function in LDL regulation. Monoclonal anti-PCSK9 antibodies (e.g., evolocumab/Repatha and alirocumab/Praluent) and small interfering RNAs (siRNAs) targeting PCSK9 (e.g., Inclisiran) have been developed to block PCSK9 function or silence its expression. However, a drawback of these drugs is the need for frequent reinjection (e.g., on the order of one dose per every 2-4 weeks for mABs and one dose per every 6 months for siRNAs). Although gene-editing approaches are being developed to knockdown expression of PCSK9, such strategies are associated with safety risks relating to introducing permanent changes to the genome.

### SUMMARY

In some aspects, the disclosure provides an expression repressor targeting a gene encoding proprotein convertase subtilisin/kexin type 9 (PCSK9) comprising (i) a DNA targeting moiety that binds to a target sequence, wherein the target sequence is about 15-20 nucleotides of a region spanning position 55,037,859 to position 55,041,755, according to the hg38 reference genome for chromosome 1 (chr1); and (ii) an effector domain.

In some embodiments of the foregoing or related aspects, the region spans position 55,038,000 to position 55,040,500, according to the hg38 reference genome for chr1. In some embodiments, the region spans position 55,039,100 to position 55,039,600, according to the hg38 reference genome for chr1. In some embodiments, the region spans position 55,039,100 to position 55,039,200; position 55,039,150 to position 55,039,250; position 55,039,200 to position 55,039,300; position 55,039,250 to position 55,039,350; position 55,039,300 to position 55,039,400; position 55,039,350 to position 55,039,450; position 55,039,400 to position 55,039,500; position 55,039,450 to position 55,039,550; position 55,039,500 to position 55,039,600, according to the hg38 reference genome for chr1. In some embodiments, the PCSK9 target sequence is 15, 16, 17, 18, 19, or 20 nucleotides in length.

In another aspect, the disclosure provides an expression repressor targeting PCSK9 comprising (i) a DNA targeting moiety that binds to a target sequence, wherein the target sequence comprises a sequence having at least 90% identity to a sequence selected from SEQ ID NOs: 67-80; and (ii) an effector domain. In some embodiments, the target sequence comprises a sequence having at least about 91%, about 92%, about 93%, about 94%, about 95%, about 96%, about 97%, about 98%, or about 99% identity to a sequence selected from SEQ ID NOs: 67-80. In some embodiments, the target sequence comprises a sequence selected from SEQ ID NOs: 67-80. In some embodiments, the target sequence consists of a sequence selected from SEQ ID NOs: 67-80. In some embodiments, the DNA-targeting moiety comprises a zinc finger (ZF) domain. In some embodiments, the DNA-targeting moiety comprises a transcription activator-like effector (TALE) domain. In some embodiments, the DNA-targeting moiety comprises a nuclease inactive Cas polypeptide (dCas9) and a gRNA comprising a sequence complementary to the target sequence.

In another aspect, the present disclosure provides an expression repressor targeting PCSK9 comprising (i) a DNA targeting moiety that binds to a target sequence comprising an amino acid sequence having at least 90% identity to a sequence selected from SEQ ID NOs: 81-94; and (ii) an effector domain. In some embodiments, the DNA targeting moiety comprises a sequence having at least about 91%, about 92%, about 93%, about 94%, about 95%, about 96%, about 97%, about 98%, or about 99% identity to a sequence selected from SEQ ID NOs: 81-94.

In some embodiments of the foregoing or related aspects, the effector domain comprises a transcriptional repressor moiety. In some embodiments, the transcriptional repressor moiety comprises a Kruppel associated box (KRAB) domain or a functional variant or fragment thereof. In some embodiments, the transcriptional repressor moiety comprises a histone modifying enzyme selected from a histone methyltransferase, a histone deacetylase, and a histone demethylase. In some embodiments, the histone modifying enzyme is a histone deacetylase. In some embodiments, the histone deacetylase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment thereof. In some embodiments, the histone modifying enzyme is a histone methyltransferase. In some embodiments, the histone methyltransferase is SETDB1, SETDB2, EHMT2, EHMT1, SUV39H1, EZH2, EZH1, SUV39H2, SETD8, SUV420H1, SUV420H2, or a functional variant or fragment thereof. In some embodiments, the transcriptional repressor moiety comprises a DNA methyltransferase. In some embodiments, the DNA methyltransferase is MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment thereof. In some embodiments, the DNA methyltransferase is MQ1, or a functional variant or fragment thereof. In some embodiments, the DNA methyltransferase increases a percentage of methylated CpG dinucleotides in a region of PCKS9.

In another aspect, provided herein is a nucleic acid comprising a nucleotide sequence encoding the expression repressor of any one of the above aspects or embodiments.

In another aspect, provided herein is a recombinant expression vector comprising the nucleic acid of the above aspect.

In another aspect, provided herein is a messenger RNA (mRNA) encoding the expression repressor of any one of the above aspects or embodiments.

In another aspect, provided herein is a lipid nanoparticle (LNP) comprising the expression repressor, the nucleic acid, the recombinant expression vector, or the mRNA of any one of the above aspects or embodiments.

In another aspect, provided herein is a pharmaceutical composition comprising the expression repressor, the nucleic acid, the recombinant expression vector, the mRNA, or the LNP of any one of the above aspects or embodiments, and a pharmaceutically acceptable carrier.

In another aspect, the present disclosure provides a system for modulating expression of human PCSK9 comprising (i) the expression repressor according to any one of the above aspects or embodiments, and (ii) a second expression repressor comprising: a second DNA targeting moiety that binds to a second target sequence that is different from the target sequence, and a second effector domain, wherein the second effector domain is the same as or different from the effector domain. In some embodiments, the expression repressor and the second expression repressor are in the same composition. In some embodiments, the expression repressor and the second expression repressor are in different compositions.

In some embodiments of the foregoing or related aspects, the system comprises a first nucleic acid encoding the expression repressor and a second nucleic acid encoding the second expression repressor. In some embodiments, the first nucleic acid and the second nucleic acid are in the same composition. In some embodiments, the first nucleic acid and the second nucleic acid are in different compositions. In some embodiments, the first nucleic acid and the second nucleic acid are formulated in the same LNP. In some embodiments, the first nucleic acid and the second nucleic acid are formulated in different LNPs. In some embodiments, the system comprises a first recombinant expression vector comprising the first nucleic acid and a second recombinant expression vector comprising the second nucleic acid. In some embodiments, the first recombinant expression vector and the second recombinant expression vector are formulated in the same LNP. In some embodiments, the first recombinant expression vector and the second recombinant expression vector are formulated in different LNPs. In some embodiments, the system comprises a recombinant expression vector comprising the first nucleic acid and the second nucleic acid. In some embodiments, the recombinant expression vector is formulated in an LNP.

In another aspect, provided herein is a nucleic acid comprising a first nucleotide sequence encoding the expression repressor according to any one of the above aspects or embodiments, and a second nucleotide sequence encoding a second expression repressor comprising a second DNA targeting moiety that binds to a second target sequence that is different from the target sequence, and a second effector domain, wherein the second effector domain is the same as or different from the effector domain.

In another aspect, a recombinant expression vector comprising the nucleic acid of the above aspect.

In another aspect, provided herein is an mRNA that encodes: the expression repressor according to any one of the above aspects or embodiments; and a second expression repressor comprising: a second DNA targeting moiety that binds to a second target sequence that is different from the target sequence, and a second effector domain, wherein the second effector domain is the same as or different from the effector domain. In some embodiments, the ORF comprises a ribosome skipping sequence between the first nucleotide sequence and the second nucleotide sequence.

In another aspect, provided herein is an LNP comprising the nucleic acid, the recombinant expression vector, or the mRNA of the above aspects or embodiments. In some embodiments of the system, nucleic acid, recombinant expression vector, mRNA, or LNP, the second target sequence is about 15-20 nucleotides in an insulated genomic domain (IGD) comprising human PCSK9.

In some embodiments of the system, nucleic acid, recombinant expression vector, mRNA, or LNP, the second target sequence is in a region spanning position 55,037,859 to position 55,041,755, according to the hg38 reference genome for chromosome 1 (chr1). In some embodiments of the system, nucleic acid, recombinant expression vector, mRNA, or LNP, the second target sequence is in a region spanning position 55,038,000 to position 55,040,500, according to the hg38 reference genome for chr1. In some embodiments of the system, nucleic acid, recombinant expression vector, mRNA, or LNP, the second target sequence is in a region spanning position 55,039,100 to position 55,039,600, according to the hg38 reference genome for chr1. In some embodiments of the system, nucleic acid, recombinant expression vector, mRNA, or LNP, the second target sequence is in a region spans position 55,039,100 to position 55,039,200; position 55,039,150 to position 55,039,250; position 55,039,200 to position 55,039,300; position 55,039,250 to position 55,039,350; position 55,039,300 to position 55,039,400; position 55,039,350 to position 55,039,450; position 55,039,400 to position 55,039,500; position 55,039,450 to position 55,039,550; position 55,039,500 to position 55,039,600, according to the hg38 reference genome for chr1.

In some embodiments of the system, nucleic acid, recombinant expression vector, mRNA, or LNP, the second target sequence comprises a sequence having at least 90% identity to a sequence selected from SEQ ID NOs: 67-80. In some embodiments, the second target sequence comprises a sequence having at least about 91%, about 92%, about 93%, about 94%, about 95%, about 96%, about 97%, about 98%, or about 99% identity to a sequence selected from SEQ ID NOs: 67-80. In some embodiments of the system, nucleic acid, recombinant expression vector, mRNA, or LNP, the second target sequence comprises a sequence selected from SEQ ID NOs: 67-80. In some embodiments of the system, nucleic acid, recombinant expression vector, mRNA, or LNP, the second target sequence consists of a sequence selected from SEQ ID NOs: 67-80. In some embodiments of the system, nucleic acid, recombinant expression vector, mRNA, or LNP, the DNA-targeting moiety of the second fusion protein comprises a zinc finger (ZF) domain.

In some embodiments of the system, nucleic acid, recombinant expression vector, mRNA, or LNP, the DNA-targeting moiety of the second fusion protein comprises a TALE domain. In some embodiments of the system, nucleic acid, recombinant expression vector, mRNA, or LNP, the DNA targeting moiety comprises an amino acid sequence having at least 90% identity to a sequence selected from SEQ ID NOs: 81-94. In some embodiments, the DNA targeting moiety comprises an amino acid sequence having at least about 91%, about 92%, about 93%, about 94%, about 95%, about 96%, about 97%, about 98%, or about 99% identity to a sequence selected from SEQ ID NOs: 81-94. In some embodiments of the system, nucleic acid, recombinant expression vector, mRNA, or LNP, the DNA-targeting moiety of the second fusion protein comprises a dCas9 and a gRNA comprising a sequence complementary to the second target sequence. In some embodiments of the system, nucleic acid, recombinant expression vector, mRNA, or LNP, the second effector domain comprises a second transcriptional repressor moiety. In some embodiments of the system, nucleic acid, recombinant expression vector, mRNA, or LNP, the second transcriptional repressor moiety comprises a Kruppel associated box (KRAB) domain or a functional variant or fragment thereof.

In some embodiments of the system, nucleic acid, recombinant expression vector, mRNA, or LNP, the second transcriptional repressor moiety comprises a histone modifying enzyme selected from a histone methyltransferase, a histone deacetylase, and a histone demethylase.

In some embodiments of the system, nucleic acid, recombinant expression vector, mRNA, or LNP, the histone modifying enzyme is a histone deacetylase. In certain embodiments, the histone deacetylase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment thereof.

In some embodiments of the system, nucleic acid, recombinant expression vector, mRNA, or LNP, the histone modifying enzyme is a histone methyltransferase. In some embodiments of the system, nucleic acid, recombinant expression vector, mRNA, or LNP, the histone methyltransferase is SETDB1, SETDB2, EHMT2, EHMT1, SUV39H1, EZH2, EZH1, SUV39H2, SETD8, SUV420H1, SUV420H2, or a functional variant or fragment thereof.

In some embodiments of the system, nucleic acid, recombinant expression vector, mRNA, or LNP, the second transcriptional repressor moiety comprises a DNA methyltransferase. In some embodiments of the system, nucleic acid, recombinant expression vector, mRNA, or LNP, the DNA methyltransferase is MQ1, DNMT1, DNMT3A1, DNMT3A1, DNMT3B1, DNMT3B2, DNMT3B3, DNMT3B4, DNMT3B5, DNMT3B6, DNMT3L, or a functional variant or fragment thereof. In some embodiments of the system, nucleic acid, recombinant expression vector, mRNA, or LNP, the DNA methyltransferase is MQ1, or a functional variant or fragment thereof.

In another aspect, provided herein is a pharmaceutical composition comprising the system, the nucleic acid, the recombinant expression vector, the mRNA, or the LNP of any one of the above aspects or embodiments, and a pharmaceutically acceptable carrier.

In another aspect, provided herein is a cell comprising the expression repressor, the system, the nucleic acid, the recombinant expression vector, the mRNA, the LNP, or the pharmaceutical composition of the above aspects.

In another aspect, provided herein is a method of altering expression of PCSK9 in a cell, comprising contacting the cell with the expression repressor, the system, the nucleic acid, the recombinant expression vector, the mRNA, the LNP, or the pharmaceutical composition of the above aspects or embodiments. In some embodiments, expression of PCSK9 is decreased.

In another aspect, provided herein is a method of introducing one or more epigenetic modifications to PCSK9 in a cell, comprising contacting the cell with the expression repressor, the system, the nucleic acid, the recombinant expression vector, the mRNA, the LNP, or the pharmaceutical composition of the above aspects or embodiments. In some embodiments, the epigenetic modification is DNA methylation or histone methylation.

In another aspect, provided herein is a method of treating a condition associated with PCSK9 expression in a subject, comprising administering to the subject the expression repressor, the system, the nucleic acid, the recombinant expression vector, the mRNA, the LNP, or the pharmaceutical composition of the above aspects or embodiments. In some embodiments, the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

In another aspect, provided herein is a method of increasing LDL receptor-mediated clearance of LDL cholesterol in a subject, comprising administering to the subject the expression repressor, the system, the nucleic acid, the recombinant expression vector, the mRNA, the LNP, or the pharmaceutical composition of any one of the above aspects or embodiments.

In another aspect, provided herein is a method of decreasing a circulating cholesterol level in a subject, comprising administering to the subject the expression repressor, the system, the nucleic acid, the recombinant expression vector, the mRNA, the LNP, or the pharmaceutical composition of any one of the above aspects or embodiments.

In another aspect, provided herein is a kit comprising a container comprising a pharmaceutical composition comprising the expression repressor, the system, the nucleic acid, the recombinant expression vector, the mRNA, or the LNP of the above aspects or embodiments, and a pharmaceutically acceptable carrier, and instructions for use in treating a condition associated with PCSK9 expression in a subject. In some embodiments, the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

In another aspect, provided herein is a kit comprising a container comprising a pharmaceutical composition comprising the expression repressor, the nucleic acid, the recombinant expression vector, the mRNA, or the LNP of the above aspects or embodiments, and a pharmaceutically acceptable carrier, and instructions for use in increasing LDL receptor-mediated clearance of LDL cholesterol and/or decreasing a circulating cholesterol level in a subject.

In another aspect, provided herein is a method of treating a condition associated with PCSK9 expression in a subject, comprising administering to the subject (i) the expression repressor according to any one of the above aspects or embodiments, and (ii) a second expression repressor comprising: a second DNA targeting moiety that binds to a second target sequence that is different from the target sequence, and a second effector domain, wherein the second effector domain is the same as or different from the effector domain. In some embodiments, the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

In another aspect, provided herein is a method of increasing LDL receptor-mediated clearance of LDL cholesterol in a subject, comprising administering to the subject (i) the expression repressor according to any of the above aspects or embodiments, and (ii) a second expression repressor comprising: a second DNA targeting moiety that binds to a second target sequence that is different from the target sequence, and a second effector domain, wherein the second effector domain is the same as or different from the effector domain.

In another aspect, provided herein is a method of decreasing a circulating cholesterol level in a subject, comprising administering to the subject (i) the expression repressor according to any one of the above aspects or embodiments, and (ii) a second expression repressor comprising: a second DNA targeting moiety that binds to a second target sequence that is different from the target sequence, and a second effector domain, wherein the second effector domain is the same as or different from the effector domain. In some embodiments, the method comprises administering the expression repressor and the second expression repressor in the same composition or in different compositions. In some embodiments, the method comprises administering a first nucleic acid encoding the expression repressor and a second nucleic acid encoding the second repression repressor. In some embodiments, the first nucleic acid is an mRNA encoding the expression repressor. In some embodiments, the second nucleic acid is an mRNA encoding the second expression repressor. In some embodiments, the method comprises administering the first nucleic acid and the second nucleic acid in the same composition or in different compositions. In some embodiments, the method comprises administering the first nucleic acid and the second nucleic acid formulated in the same LNP or in separate LNPs. In some embodiments, the method comprises administering a first recombinant expression vector comprising the first nucleic acid and a second recombinant expression vector comprising the second nucleic acid. In some embodiments, the first recombinant expression vector and the second recombinant expression vector are formulated in the same LNP or in separate LNPs.

In some embodiments, the method comprises administering a recombinant expression vector comprising the first nucleic acid and the second nucleic acid. In some embodiments, the recombinant expression vector is formulated in an LNP.

In some embodiments, the second target sequence is about 15-20 nucleotides in an insulated genomic domain (IGD) comprising human PCSK9. In some embodiments, the second target sequence is in a region spanning position 55,037,859 to position 55,041,755, according to the hg38 reference genome for chromosome 1 (chr1). In some embodiments, the second target sequence is in a region spanning position 55,038,000 to position 55,040,500, according to the hg38 reference genome for chr1. In some embodiments, the second target sequence is in a region spanning position 55,039,100 to position 55,039,600, according to the hg38 reference genome for chr1. In some embodiments, the second target sequence is in a region spanning position 55,039,100 to position 55,039,200; position 55,039,150 to position 55,039,250; position 55,039,200 to position 55,039,300; position 55,039,250 to position 55,039,350; position 55,039,300 to position 55,039,400; position 55,039,350 to position 55,039,450; position 55,039,400 to position 55,039,500; position 55,039,450 to position 55,039,550; or position 55,039,500 to position 55,039,600, each according to the hg38 reference genome for chr1.

In some embodiments, the second target sequence comprises a sequence having at least 90% identity to a sequence selected from SEQ ID NOs: 67-80. In some embodiments, the second target sequence comprises a sequence selected from SEQ ID NOs: 67-80. In some embodiments, the second target sequence consists of a sequence selected from SEQ ID NOs: 67-80.

In some embodiments, the second DNA-targeting moiety comprises a zinc finger (ZF) domain. In some embodiments, the second DNA-targeting moiety comprises a TALE domain. In some embodiments, the second DNA targeting moiety comprises an amino acid sequence having at least 90% identity to a sequence selected from SEQ ID NOs: 81-94.

In some embodiments, the second DNA-targeting moiety comprises a dCas9 and a gRNA comprising a sequence complementary to the second target sequence. In some embodiments, the second effector domain comprises a second transcriptional repressor moiety.

In some embodiments, the second transcriptional repressor moiety comprises a Kruppel associated box (KRAB) domain or a functional variant or fragment thereof. In some embodiments, the second transcriptional repressor moiety comprises a histone modifying enzyme selected from a histone methyltransferase, a histone deacetylase, and a histone demethylase.

In some embodiments, the histone modifying enzyme is a histone deacetylase. In some embodiments, the histone deacetylase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment thereof.

In some embodiments, the histone modifying enzyme is a histone methyltransferase. In some embodiments, the histone methyltransferase is SETDB1, SETDB2, EHMT2, EHMT1, SUV39H1, EZH2, EZH1, SUV39H2, SETD8, SUV420H1, SUV420H2, or a functional variant or fragment thereof.

In some embodiments, the second transcriptional repressor moiety comprises a DNA methyltransferase. In some embodiments, the DNA methyltransferase is MQ1, DNMT1, DNMT3A1, DNMT3A1, DNMT3B1, DNMT3B2, DNMT3B3, DNMT3B4, DNMT3B5, DNMT3B6, DNMT3L, or a functional variant or fragment thereof. In some embodiments, the DNA methyltransferase is MQ1, or a functional variant or fragment thereof.

### BRIEF DESCRIPTION OF THE DRAWINGS

FIG. 1A provides a schematic depicting a region of the mouse PCSK9 (mPCSK9) gene containing a CpG island and target sequences for exemplary single guide RNAs (sgRNAs) and transcription activator-like effectors (TALEs) described herein. In the schematic, transcription occurs from right-to-left and bottom-to-top. Indicated is the primary transcript of mPCSK9, the 5′ end of which coincides with the 5′ end of exon 1. The scale provides genomic coordinates according to the GRCm38 reference genome and is annotated to show alignment to the mPCSK9 transcript (“Pcsk9”), mPCSK9 Exon 1 (“Pcsk9-001 Exon 1”), the CpG island, and target sequences (the genomic coordinates for each, as shown by the alignments in the figure, are approximate).

FIG. 1B provides a schematic depicting a region of the human PCSK9 gene containing a CpG island and target sequences for exemplary TALEs described herein. In the schematic, transcription occurs left-to-right and top-to-bottom. Indicated is the primary transcript of PCSK9, the 5′ end of which does not coincide with the 5′ end of exon 1 due to a splice event. The scale provides genomic coordinates according to the GRCh38 reference genome (genomic coordinates of the hPCSK9 transcript (“PCSK9”), hPCSK9 exon 1 (“PCSK9-201 Exon 1”), CpG island, and target sequences as shown by the alignment are approximate).

FIGS. 2A-2C provide graphs depicting serum mPCSK9 protein levels in vivo from mice intravenously administered a single dose of mRNA encoding dCas9-MQ1 (catalytically inactive Cas9 (dCas9) fused to a DNA methylator (MQ1)) and a single guide RNA (sgRNA) targeting the mPCSK9 gene region depicted in FIG. 1A, co-formulated in a lipid nanoparticle (LNP). Dosing was at 6 mg/kg (TA-1) or 3 mg/kg (TA-2). Control mice received an intravenous injection of PBS. FIG. 2A is a graph depicting mPCSK9 protein levels (ng/ml) as measured in serum collected from each mouse at the indicated day post-administration. FIG. 2B shows percent change in mPCSK9 protein in serum at the indicated day post-administration relative to baseline for each subject. FIG. 2C shows a line graph depicting change over time of mPCSK9 protein levels in serum. For FIG. 2A and FIG. 2B, data were analyzed using a two-way ANOVA with Sidak's multiple comparison tests, with comparison to control for each time point (*p<0.032 and ** p<0.021).

FIG. 3A provides an image of a Western blot showing mPCSK9 protein expression in cell lysate obtained from AML12 and Hepa1-6 murine cells treated with mRNA encoding a fusion of a TALE targeting the mPCSK9 gene region depicted in FIG. 1A and the DNA methylator MQ1 (TAL01-MQ1, TAL02-MQ1, or TAL03-MQ1) formulated in an MC3 or SSOP LNP. Control cells were untreated. The blot is labeled to identify protein bands corresponding to CTCF (used as loading control) and the TALE-MQ1 fusion.

FIGS. 3B-3C provide graphs depicting the level of mPCSK9 mRNA following treatment with the MC3 LNP-formulated mRNA encoding TAL01-MQ1, TAL02-MQ1, or TAL03-MQ1 in AML12 cells (FIG. 3B) and Hepa1-6 cells (FIG. 3C) as measured by RT-qPCR. Control cells were untreated or treated with MC3 LNP-formulated mRNA encoding GFP.

FIGS. 3D-3E provide graphs depicting the level of mPCSK9 protein following treatment with the LNP-formulated mRNA encoding TAL01-MQ1, TAL02-MQ1, or TAL03-MQ1 in AML12 cells (FIG. 3D) and Hepa1-6 cells (FIG. 3E), as determined by enzyme-linked immunosorbent assay (ELISA). Control cells were untreated or treated with LNP-formulated mRNA encoding GFP.

FIGS. 4A-4D provide plots depicting percent methylation of an approximately 450 bp region containing the CpG island near the mPCSK9 promoter as measured in liver cell lysate obtained from mice one week following intravenous administration of a dose of LNP-formulated mRNA encoding TAL02-MQ1. DNA methylation was quantified by enzymatic methyl-seq (Em-Seq) following administration of PBS (FIG. 4A) or LNP-formulated mRNA at a dose of 0.3 mg/kg (FIG. 4B), 1 mg/kg (FIG. 4C), or 3 mg/kg (FIG. 4D). CpG methylation was determined by the ratio of methylated read coverage to total read coverage for each CpG. Dot plot figures show the percent methylation versus relative position of each CpG across the amplicon. Within the dot plots, dot size corresponds to the read depth for that CpG, and color represents technical replicates and biological replicates for biological replicate and group plots, respectively.

FIGS. 5A-5H provides graphs depicting mPCSK9 mRNA level measured in the liver and mPCSK9 protein expression, low density lipoprotein (LDL), and LDL-cholesterol (LDL-c) measured in serum obtained from mice one week following intravenous administration of a 0.3 mg/kg, 1 mg/kg, or 3 mg/kg dose of LNP-formulated mRNA encoding TAL02-MQ1. Control mice received an intravenous injection of PBS. mPCSK9 mRNA levels, normalized to HPRT1 (housekeeper) levels as measured by RT-qPCR and averaged across subjects, are shown in FIG. 5A and normalized mPCSK9 mRNA levels for individual subjects are shown in FIG. 5B. mPCSK9 serum levels (pg/ml) measured by ELISA and averaged across subjects are shown in FIG. 5C and mPCSK9 serum levels (pg/ml) for individual subjects is shown in FIG. 5D. LDL-c levels (mmol/L) measured by ELISA and averaged across subjects are shown in FIG. 5E and LDL-c levels (mmol/L) for individual subjects are shown in FIG. 5F. LDL levels (ng/ml) measured by ELISA and averaged across subjects are shown in FIG. 5G and LDL levels (ng/ml) for individual subjects are shown in FIG. 5H.

FIG. 6 provides a schematic showing a treatment schedule for mice receiving a single intravenous dose of LNP-formulated mRNA encoding TAL02-MQ1 (day 0) and subsequent time points for collection of livers (days 14, 28, 63, 90, 120, 152, and 180 post-administration) and serum (days 14, 28, 42, 63, 77, 90, 104, 120, 134, 152, 166, and 180 post-administration). Control mice received an intravenous injection of PBS.

FIGS. 7A-7F provide graphs depicting mPCSK9 mRNA level measured in the liver and mPCSK9 protein expression, LDL, and LDL-c measured in serum obtained at the indicated time points from mice intravenously administered PBS or a dose of LNP-formulated mRNA encoding TAL02-MQ1 according to the treatment schedule shown in FIG. 6. mPCSK9 mRNA levels normalized to HPRT (housekeeper) as measured by RT-qPCR and averaged across subjects are shown in FIG. 7A; normalized mPCSK9 mRNA level for individual subjects is shown in FIG. 7B. mPCSK9 serum levels (pg/ml) measured by ELISA and averaged across subjects are shown in FIG. 7C; mPCSK9 serum levels (pg/ml) for individual subjects is shown in FIG. 7D. LDL-c levels (mmol/L) measured by ELISA and averaged across subjects is shown in FIG. 7E; LDL-c levels (mmol/L) for individual subjects is shown in FIG. 7F.

FIGS. 8A-8E provide graphs depicting percent methylation of an approximately 450 bp region containing the CpG island in the mPCSK9 promoter methylation as measured in liver cell lysate obtained at the indicated time points from mice intravenously administered a single dose of LNP-formulated mRNA encoding TAL02-MQ1 (TA-1) according to the treatment schedule shown in FIG. 6. Control mice received an intravenous injection of PBS. DNA methylation was quantified by Em-Seq following administration of PBS or TA-1 14-days post-dose (FIG. 8A and FIG. 8B, respectively) and 28-days post-dose (FIG. 8C and FIG. 8D, respectively). FIG. 8E depicts promoter methylation at 14-days and 28-days post dose, as the average methylation/mouse for the tested conditions. FIGS. 8F-8I depict promoter methylation at 63 days, 90 days, 120 days, and 152 days post-dose respectively as the average methylation/mouse for the tested conditions. For FIGS. 8E-8I, each column represents an individual animal, and each plot shows the average overall methylation content of the CpG islands in the amplicon.

FIG. 9A provides a graph depicting the average methylation changes in the mice of FIG. 6 of four differential methylated regions (DMRs) in the mPCSK9 promoter.

FIG. 9B provides a graph depicting the normalized mRNA expression levels of mPCSK9 as measured in liver cell lysate obtained from the mice of FIG. 6.

### DETAILED DESCRIPTION

The present disclosure is based, at least in part, on the discovery of a region of the genome comprising one or more transcriptional control elements for regulating expression of PCSK9 (e.g., a region of the genome comprising a PCSK9 promoter), wherein an expression repressor of the disclosure comprising (i) a DNA targeting moiety (e.g., a ZF, TALE, or dCas9) that binds to a target sequence in the region; and (ii) an effector domain capable of epigenetic modification (e.g., DNA methylation) functions to decrease PCSK9 expression (e.g., by transcriptional repression) when introduced to a cell (e.g., in vitro or in vivo). As used herein, the term “expression repressor” refers to an agent or entity with one or more functionalities that decreases expression of a target gene in a cell and that specifically binds to a DNA sequence (e.g., a DNA sequence associated with a target gene or a transcription control element operably linked to a target gene).

As demonstrated herein, introducing an mRNA encoding an exemplary expression repressor of the disclosure to a cell, wherein the exemplary expression repressor comprised (i) a DNA targeting moiety that binds a target sequence in a region of the genome comprising a PCSK9 transcriptional control element (e.g., a PCSK9 promoter); and (ii) an effector domain comprising a DNA methyltransferase, resulted in methylation of the genome within the region, thereby decreasing expression of PCSK9. Furthermore, it was shown that in vivo administration of a single dose of an mRNA encoding the expression repressor resulted in reduced levels of PCSK9 (e.g., in the serum or liver) and LDLc (e.g., in the serum) for a prolonged period following administration. Without being bound by theory, administering a dose (e.g., a single dose) of an expression repressor of the disclosure engineered to target a region of the genome comprising a PCSK9 transcriptional control element (e.g., a PCSK9 promoter), or a nucleic acid encoding the expression repressor, results in reduction of PCSK9 levels, thereby increasing clearance of circulating LDLc for treatment, alleviation, and/or prevention of cholesterol-related diseases or disorders.

Accordingly, in some aspects, the present disclosure provides an expression repressor comprising (i) a DNA targeting moiety that binds a target sequence in a region of the genome comprising a PCSK9 transcriptional control element (e.g., a PCSK9 promoter); and (ii) an effector domain. In some embodiments, the target sequence is a span of nucleotides (e.g., 10-50, 10-40, 10-30, 15-30, 15-25, or 15-20 nucleotides) in or near an insulated genomic domain (IGD) comprising PCSK9 (“PCSK9 IGD”). As further described herein and understood by one of ordinary skill in the art, IGDs are units of genomic space with boundaries defined by factors that mechanistically drive functional insulation between gene transcription activities. Thus, IGDs are physical units that serve to parse chromosomes into discrete functional segments. For example, in some embodiments, an IGD comprises a DNA loop formed by interactions between two DNA sites bound by homodimerized CTCF and cohesin (see Dowen, et al (2014) Cell 159:374-87). In such an IGD, occupation of each of the DNA sites bound by CTCF and cohesin inhibits DNA-bound components on one chromosomal side of the DNA site from interacting with DNA-bound components on the opposite chromosomal side. Consequently, the DNA sites occupied by CTCF and cohesin in such DNA loops act as boundaries for the IGD. In some embodiments, the formation of such DNA loops facilitates (i) enhancer-promoter interactions in which both the enhancer and promoter are within the loop, (ii) inhibition of enhancer-promoter interactions in which one of those elements is within the loop and the other is outside the loop, or (iii) both (i) and (ii).

In some embodiments, the region of the genome comprising a PCSK9 transcriptional control element (e.g., a PCSK9 promoter) spans position 55,037,859 to position 55,041,755 of chromosome 1 (chr1), according to the hg38 reference genome for chr1. In some embodiments, the target sequence is about 10 to about 50 nucleotides in the region (e.g., 10-40, 10-30, 15-30, 15-25, or 15-20 nucleotides in the region). In some embodiments, the DNA targeting moiety comprises a polypeptide that binds the target sequence. In some embodiments, the DNA targeting moiety comprises a zinc finger (ZF) domain that binds the target sequence. In some embodiments, the DNA targeting moiety comprises a transcription activator-like effector (TALE) domain that binds the target sequence. In some embodiments, the DNA targeting moiety comprises a catalytically inactive site-directed nuclease (e.g., a catalytically inactive Cas nuclease) and a guide sequence, wherein the guide sequence is complementary, or substantially complementary, to the target sequence.

In some embodiments, the effector domain comprises a polypeptide for suppressing gene transcription, e.g., by inducing one or more epigenetic changes. In some embodiments, the effector domain comprises a transcriptional repressor moiety. In some embodiments, the transcriptional repressor moiety recruits components of the endogenous transcriptional machinery to decrease expression of the target gene. In some embodiments, the transcriptional repressor moiety is a polypeptide, that upon binding to a transcriptional control element, recruits one or more corepressor proteins and/or transcription factors to inactivate, or substantially inactivate, gene transcription. In some embodiments, the transcriptional repressor moiety inhibits recruitment of transcription factors, thereby decreasing expression of the target gene. In some embodiments, the transcriptional repressor moiety comprises an epigenetic modifying moiety (e.g., a moiety for introducing an epigenetic modification in or near the target gene). In some embodiments, the transcriptional repressor moiety is an enzyme, that upon binding to a transcriptional control element, catalyzes one or more modifications of a genomic region comprising the transcriptional control element, wherein the one or more modifications inactivates, or substantially inactivates, gene transcription. In some embodiments, the one or more modifications are selected from a DNA modification and a histone modification.

In some aspects, the disclosure provides a nucleic acid encoding an expression repressor described herein. In some embodiments, the nucleic acid is an mRNA. In some aspects, the disclosure provides a recombinant expression vector comprising the nucleic acid. In some embodiments, the expression repressor, the nucleic acid (e.g., mRNA), or the recombinant expression vector is formulated in a lipid nanoparticle (LNP).

In some aspects, the disclosure provides a system comprising two or more expression repressors described herein. In some embodiments, the system comprises 2, 3, 4, 5, 6, 7, 8, 9, or 10 expression repressors described herein. In some embodiments, the system comprises two or more nucleic acids, wherein each nucleic acid encodes an expression repressor described herein. In some embodiments, the two or more nucleic acids are each mRNAs. In some embodiments, the system comprises two or more recombinant expression vectors, wherein each recombinant expression vector comprises a nucleic acid encoding an expression repressor described herein. In some embodiments, the two or more expression repressors, the two or more nucleic acids, or the two or more recombinant expression vectors are formulated in the same LNP or in different LNPs.

In some aspects, the disclosure provides a nucleic acid encoding two or more expression repressors (e.g., 2, 3, 4, 5, 6, 7, 8, 9, or 10 expression repressors) described herein. In some embodiments, the nucleic acid is an mRNA. In some embodiments, the disclosure provides a recombinant expression vector comprising the nucleic acid. In some embodiments, the nucleic acid or the recombinant expression vector is formulated in an LNP.

In some aspects, the disclosure provides a pharmaceutical composition comprising an expression repressor described herein, a nucleic acid described herein, a recombinant expression vector described herein, an LNP described herein, or a system described herein, and a pharmaceutically acceptable carrier.

In some aspects, the disclosure provides a method of altering (e.g., decreasing) expression of PCSK9 in a cell, comprising contacting the cell with an expression repressor described herein, a nucleic acid described herein, a recombinant expression vector described herein, an LNP described herein, a system described herein, or a pharmaceutical composition described herein. In some embodiments, expression of PCSK9 is decreased compared to a control cell not contacted with the expression repressor, nucleic acid, recombinant expression vector, LNP, system, or pharmaceutical composition.

In some aspects, the disclosure provides a method of introducing one or more epigenetic modifications to a region comprising a transcriptional control element of PCSK9 in a cell, the method comprising contacting the cell with an expression repressor described herein, a nucleic acid described herein, a recombinant expression vector described herein, an LNP described herein, a system described herein, or a pharmaceutical composition described herein. In some embodiments, the transcriptional control element comprises a promoter of PCSK9. In some embodiments, the one or more epigenetic modifications comprises DNA methylation and/or histone modification.

In some aspects, the disclosure provides a method of treating a condition associated with PCSK9 in a subject in need thereof, comprising administering to the subject an effective amount of an expression repressor described herein, a nucleic acid described herein, a recombinant expression vector described herein, an LNP described herein, a system described herein, or a pharmaceutical composition described herein. In some embodiments, the method increases LDL-receptor mediated clearance of LDL cholesterol in the subject as compared to prior to the administration. In some embodiments, the method decreases a circulating cholesterol level in the subject as compared to prior to the administration.

### PCSK9 Expression Repressors

In some embodiments, the disclosure provides an expression repressor for altering (e.g., decreasing) expression of PCSK9 (e.g., human PCSK9). In some embodiments, the disclosure provides an expression repressor for decreasing expression of human PCSK9. As used herein, the term “human PCSK9” refers to a gene on human chromosome 1 encoding the enzyme proprotein convertase subtilisin/kexin type 9 (PCSK9). In some embodiments, human PCSK9 has the genomic coordinates 55,039,447 to 55,064,852, according to human reference genome Hg38 of chr1. The human PCSK9 gene encodes a 692 amino acid protein. See also, e.g., Ensembl ENSG00000169174 providing human PCSK9; Ensembl ENST00000302118.5 and NCBI Ref. Seq NM_174936.4 providing the human PCSK9 mRNA sequence; and UniProt Q8NBP7 and NCBI Reference Sequence NP_777596.2 providing the corresponding human PCSK9 polypeptide.

In some embodiments, an expression repressor of the disclosure has a targeting function and an effector function. In some embodiments, the targeting function localizes the effector function of the expression repressor to a region of the genome. In some embodiments, the region of the genome comprises the PCSK9 IGD. In some embodiments, the region of the genome is in the PCSK9 IGD. In some embodiments, the effector function comprises introducing one or more epigenetic modifications to the region of the genome.

In some embodiments, the expression repressor comprises a DNA targeting moiety and an effector domain. In some embodiments, the targeting function of the expression repressor is mediated by the DNA targeting moiety. In some embodiments, the targeting function is mediated by the DNA targeting moiety binding to a target sequence in the region of the genome.

In some embodiments, the effector domain is a transcriptional repressor moiety described herein. In some embodiments, the DNA targeting moiety binds to a target sequence in the PCSK9 gene, whereupon the effector domain of the expression repressor functions to introduce one or more epigenetic modifications to a region in the PCSK9 gene. In some embodiments, the DNA targeting moiety binds to a target sequence in a genomic region comprising the PCSK9 IGD (e.g., the human PCSK9 IGD), whereupon the effector domain of the expression repressor functions to introduce one or more epigenetic modifications to a region in or near the PCSK9 IGD (e.g., the human PCSK9 IGD). In some embodiments, the DNA targeting moiety binds to a target sequence in the PCSK9 IGD (e.g., the human PCSK9 IGD), whereupon the effector domain of the expression repressor functions to introduce one or more epigenetic modifications to a region in the PCSK9 IGD (e.g., the human PCSK9 IGD). In some embodiments, one or more epigenetic modifications is introduced to a transcriptional control element (e.g., promoter or enhancer) of PCSK9 (e.g., human PCSK9), or a portion thereof. In some embodiments, the one or more epigenetic modifications results in decreased expression of PCSK9 (e.g., human PCSK9), e.g., as compared to a control cell not contacted with the expression repressor.

### Target Sequences

In some embodiments, the DNA targeting moiety binds to a target sequence in the PCSK9 gene. In some embodiments, the DNA targeting moiety binds to a target sequence in the human PCSK9 gene. In some embodiments, the DNA targeting moiety binds to a target sequence in a genomic region comprising the PCSK9 IGD. In some embodiments, the DNA targeting moiety binds to a target sequence in the PCSK9 IGD. In some embodiments, the DNA targeting moiety binds to a target sequence in the human PCSK9 IGD. In some embodiments, the human PCSK9 IGD comprises the genomic coordinates 55,020,760-55,285,867, according to human reference genome Hg38 of chr1.

In some embodiments, the DNA targeting moiety comprises a ZF that binds the target sequence in a genomic region comprising the PCSK9 IGD. In some embodiments, the DNA targeting moiety comprises a ZF that binds the target sequence in the PCSK9 IGD. In some embodiments, the DNA targeting moiety comprises a TALE that binds the target sequence in a genomic region comprising the PCSK9 IGD. In some embodiments, the DNA targeting moiety comprises a TALE that binds the target sequence in the PCSK9 IGD. In some embodiments, the DNA targeting moiety comprises a site-directed nuclease (e.g., a catalytically inactive site-directed nuclease) that binds the target sequence in a genomic region comprising the PCSK9 IGD. In some embodiments, the DNA targeting moiety comprises a site-directed nuclease (e.g., a catalytically inactive site-directed nuclease) that binds the target sequence in the PCSK9 IGD. In some embodiments, the site-directed nuclease comprises a Cas nuclease described herein (e.g., a catalytically inactive Cas nuclease) and a gRNA comprising a spacer sequence corresponding to the target sequence. The spacer sequence is a sequence that defines the target sequence in the PCSK9 IGD. The target sequence is present in a double-stranded genomic DNA having one strand comprising the target sequence comprising a protospacer sequence adjacent to a PAM sequence that is referred to as the “PAM strand,” and a second strand that is referred to as the “non-PAM strand” and is complementary to the PAM strand. Both the gRNA spacer sequence and the target sequence are complementary to the non-PAM strand of the genomic DNA molecule. As used herein, a spacer sequence “corresponding to” a target sequence refers to a guide sequence that binds to the non-PAM strand of the target sequence by Watson-Crick base-pairing, wherein the spacer sequence has sufficient complementarity to the non-PAM strand as to enable targeting of the Cas nuclease to the target sequence in the genomic DNA molecule. In some embodiments, the spacer sequence has up to 1, 2, or 3 mismatches relative to the target sequence in the genomic DNA molecule, wherein the spacer sequence has sufficient complementarity to the non-PAM strand as to enable targeting of the Cas nuclease to the target sequence in the genomic DNA molecule.

In some embodiments, the DNA targeting moiety binds to a target sequence in a genomic region comprising the human PCSK9 IGD, wherein the target sequence is upstream of or in a 5′boundary of the human PCSK9 IGD. In some embodiments, the target sequence is between a 5′ and 3′boundary of the human PCSK9 IGD. In some embodiments, the target sequence is downstream of or in the 3′boundary of the human PCSK9 IGD. In some embodiments, the DNA targeting moiety binds to a target sequence in the human PCSK9 IGD, wherein the target sequence is in a region (e.g., a 0.5-2 kb region) comprising a transcriptional control element (e.g., a promoter or enhancer). In some embodiments, the region comprises a promoter. In some embodiments, the target sequence is in a promoter. In some embodiments, the region comprises an enhancer. In some embodiments, the target sequence is in an enhancer. In some embodiments, the target sequence is in or near a CpG island in the human PCSK9 IGD. In some embodiments, the target is in a region of about 100 bases, about 200 bases, about 300 bases, about 400 bases, about 500 bases, about 600 bases, about 700 bases, about 800 bases, about 900 bases, about 1,000 bases, about 1,100 bases, about 1,200 bases, about 1,300 bases, about 1,400 bases, about 1,500 bases, about 1,600 bases, about 1,700 bases, about 1,800 bases, about 1,900 bases, or about 2,000 bases comprising the CpG island. In some embodiments, the target is in a region of about 100 bases, about 200 bases, about 300 bases, about 400 bases, about 500 bases, about 600 bases, about 700 bases, about 800 bases, about 900 bases, or about 1,000 bases comprising the CpG island. In some embodiments, the target sequence is not more than about 300 bases, about 400 bases, or about 500 bases upstream or downstream the CpG island. In some embodiments, the target sequence is in the CpG island.

In some embodiments, the DNA targeting moiety binds to a target sequence in the human PCSK9 IGD, wherein the target sequence is in a region (e.g., a 0.5-2 kb region) comprising a transcriptional control element (e.g., a promoter) of human PCSK9. In some embodiments, the target sequence is in a region comprising a human PCSK9 promoter. As used herein, “a human PCSK9 promoter” refers to a genomic region upstream of a transcriptional start sequence (TSS) of a PCSK9 transcript. The promoter may include 50 bp, 100 bp, 150 bp, 200 bp, 250 bp, 300 bp, 400 bp, 500 bp, 600 bp, 700 bp, 800 bp, 900 bp, or 1000 bp upstream of a TSS. The promoter may comprise or lie within hg38 chr1: 55039499-55039558, hg38 chr1: 55038637-55041230, hg38 chr1: 55039100-55039999, hg38 chr1: 55038600-55039999, hg38 chr1: 55040181-55040295, or hg38 chr1: 55039681-55040295. Human PCSK9 has multiple TSSs, and any TSS recognized in the art may be used to define a promoter sequence. For example, and without limitation, the TSS may comprise hg38 chr1:55039548 or hg38 chr1:55040295. In some embodiments, the target sequence is in a region comprising an enhancer of human PCSK9. In some embodiments, the target sequence is in a coding region of human PCSK9.

The length of the target sequence depends on the DNA targeting moiety used. In some embodiments, the DNA targeting moiety comprises a ZF and the target sequence is about 10 to about 50 nucleotides, about 10 to about 40 nucleotides, about 10 to about 30 nucleotides, about 10 to about 20 nucleotides, or about 15 to about 20 nucleotides. In some embodiments, the DNA targeting moiety comprises a ZF and the target sequence is about 10, about 11, about 12, about 13, about 14, about 15, about 16, about 17, about 18, about 19, or about 20 nucleotides. In some embodiments, the DNA targeting moiety comprises a ZF and the target sequence is 15 nucleotides. In some embodiments, the DNA targeting moiety comprises a ZF and the target sequence is 16 nucleotides. In some embodiments, the DNA targeting moiety comprises a ZF and the target sequence is 17 nucleotides. In some embodiments, the DNA targeting moiety comprises a ZF and the target sequence is 18 nucleotides. In some embodiments, the DNA targeting moiety comprises a ZF and the target sequence is 19 nucleotides. In some embodiments, the DNA targeting moiety comprises a ZF and the target sequence is 20 nucleotides.

In some embodiments, the DNA targeting moiety comprises a TALE and the target sequence is about 10 to about 50 nucleotides, about 10 to about 40 nucleotides, about 10 to about 30 nucleotides, about 10 to about 20 nucleotides, or about 15 to about 20 nucleotides. In some embodiments, the DNA targeting moiety comprises a TALE and the target sequence is about 10, about 11, about 12, about 13, about 14, about 15, about 16, about 17, about 18, about 19, or about 20 nucleotides. In some embodiments, the DNA targeting moiety comprises a TALE and the target sequence is 15 nucleotides. In some embodiments, the DNA targeting moiety comprises a TALE and the target sequence is 16 nucleotides. In some embodiments, the DNA targeting moiety comprises a TALE and the target sequence is 17 nucleotides. In some embodiments, the DNA targeting moiety comprises a TALE and the target sequence is 18 nucleotides. In some embodiments, the DNA targeting moiety comprises a TALE and the target sequence is 19 nucleotides. In some embodiments, the DNA targeting moiety comprises a TALE and the target sequence is 20 nucleotides.

In some embodiments, the DNA targeting moiety comprises a site-directed nuclease (e.g., a catalytically inactive site-directed nuclease) and the target sequence is about 10 to about 50 nucleotides, about 10 to about 40 nucleotides, about 10 to about 30 nucleotides, about 10 to about 20 nucleotides, or about 15 to about 20 nucleotides. In some embodiments, the DNA targeting moiety comprises a site-directed nuclease (e.g., a catalytically inactive site-directed nuclease) and the target sequence is about 10, about 11, about 12, about 13, about 14, about 15, about 16, about 17, about 18, about 19, or about 20 nucleotides. In some embodiments, the DNA targeting moiety comprises a site-directed nuclease (e.g., a catalytically inactive site-directed nuclease) and the target sequence is 15 nucleotides. In some embodiments, the DNA targeting moiety comprises a site-directed nuclease (e.g., a catalytically inactive site-directed nuclease) and the target sequence is 16 nucleotides. In some embodiments, the DNA targeting moiety comprises a site-directed nuclease (e.g., a catalytically inactive site-directed nuclease) and the target sequence is 17 nucleotides. In some embodiments, the DNA targeting moiety comprises a site-directed nuclease (e.g., a catalytically inactive site-directed nuclease) and the target sequence is 18 nucleotides. In some embodiments, the DNA targeting moiety comprises a site-directed nuclease (e.g., a catalytically inactive site-directed nuclease) and the target sequence is 19 nucleotides. In some embodiments, the DNA targeting moiety comprises a site-directed nuclease (e.g., a catalytically inactive site-directed nuclease) and the target sequence is 20 nucleotides.

In some embodiments, the target sequence is 10-50 nucleotides (e.g., 10-40, 10-30, 15-30, 15-25, or 15-20 nucleotides) in a genomic region comprising the PCSK9 IGD (e.g., the human PCSK9 IGD). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a genomic region comprising the PCSK9 IGD (e.g., the human PCSK9 IGD). In some embodiments, the target sequence is about 10, about 11, about 12, about 13, about 14, about 15, about 16, about 17, about 18, about 19, or about 20 nucleotides in a genomic region comprising the PCSK9 IGD (e.g., the human PCSK9 IGD).

In some embodiments, the target sequence is 10-50 nucleotides (e.g., 10-40, 10-30, 15-30, 15-25, or 15-20 nucleotides) in the PCSK9 IGD (e.g., the human PCSK9 IGD). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in the PCSK9 IGD (e.g., the human PCSK9 IGD). In some embodiments, the target sequence is about 10, about 11, about 12, about 13, about 14, about 15, about 16, about 17, about 18, about 19, or about 20 nucleotides in the PCSK9 IGD (e.g., the human PCSK9 IGD).

In some embodiments, the target sequence is 10-50 nucleotides (e.g., 10-40, 10-30, 15-30, 15-25, or 15-20 nucleotides) in a region (e.g., a 0.1-2 kb region) of the PCSK9 IGD (e.g., the human PCSK9 IGD), wherein the region comprises a transcriptional control element (e.g., a promoter or enhancer). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of the PCSK9 IGD (e.g., the human PCSK9 IGD), wherein the region comprises a transcriptional control element (e.g., a promoter or enhancer). In some embodiments, the target sequence is about 10, about 11, about 12, about 13, about 14, about 15, about 16, about 17, about 18, about 19, or about 20 nucleotides in a region (e.g., a 0.1-2 kb region) of the PCSK9 IGD (e.g., the human PCSK9 IGD), wherein the region comprises a transcriptional control element (e.g., a promoter or enhancer).

In some embodiments, the target sequence is 10-50 nucleotides (e.g., 10-40, 10-30, 15-30, 15-25, or 15-20 nucleotides) in a transcriptional control element (e.g., a promoter or enhancer) in the PCSK9 IGD (e.g., human PCSK9 IGD). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a transcriptional control element (e.g., a promoter or enhancer) in the PCSK9 IGD (e.g., human PCSK9 IGD). In some embodiments, the target sequence is about 10, about 11, about 12, about 13, about 14, about 15, about 16, about 17, about 18, about 19, or about 20 nucleotides in a transcriptional control element (e.g., a promoter or enhancer) in the PCSK9 IGD (e.g., human PCSK9 IGD).

In some embodiments, the target sequence is 10-50 nucleotides (e.g., 10-40, 10-30, 15-30, 15-25, or 15-20 nucleotides) in a region (e.g., a 0.1-2 kb region) of the PCSK9 IGD (e.g., the human PCSK9 IGD), wherein the region comprises a promoter. In some embodiments, the target sequence is within or overlapping the promoter. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of the PCSK9 IGD (e.g., the human PCSK9 IGD), wherein the region comprises a promoter. In some embodiments, the target sequence is about 10, about 11, about 12, about 13, about 14, about 15, about 16, about 17, about 18, about 19, or about 20 nucleotides in a region (e.g., a 0.1-2 kb region) of the PCSK9 IGD (e.g., the human PCSK9 IGD), wherein the region comprises a promoter.

In some embodiments, the target sequence is 10-50 nucleotides (e.g., 10-40, 10-30, 15-30, 15-25, or 15-20 nucleotides) in a promoter in the PCSK9 IGD (e.g., human PCSK9 IGD). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a promoter in the PCSK9 IGD (e.g., human PCSK9 IGD). In some embodiments, the target sequence is about 10, about 11, about 12, about 13, about 14, about 15, about 16, about 17, about 18, about 19, or about 20 nucleotides in a promoter in the PCSK9 IGD (e.g., human PCSK9 IGD).

In some embodiments, the target sequence is 10-50 nucleotides (e.g., 10-40, 10-30, 15-30, 15-25, or 15-20 nucleotides) in a region (e.g., a 0.1-2 kb region) of the PCSK9 IGD (e.g., the human PCSK9 IGD), wherein the region comprises an enhancer. In some embodiments, the target sequence is within or overlapping the enhancer. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of the PCSK9 IGD (e.g., the human PCSK9 IGD), wherein the region comprises an enhancer. In some embodiments, the target sequence is about 10, about 11, about 12, about 13, about 14, about 15, about 16, about 17, about 18, about 19, or about 20 nucleotides in a region (e.g., a 0.1-2 kb region) of the PCSK9 IGD (e.g., the human PCSK9 IGD), wherein the region comprises an enhancer.

In some embodiments, the target sequence is 10-50 nucleotides (e.g., 10-40, 10-30, 15-30, 15-25, or 15-20 nucleotides) in an enhancer in the PCSK9 IGD (e.g., human PCSK9 IGD). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in an enhancer in the PCSK9 IGD (e.g., human PCSK9 IGD). In some embodiments, the target sequence is about 10, about 11, about 12, about 13, about 14, about 15, about 16, about 17, about 18, about 19, or about 20 nucleotides in an enhancer in the PCSK9 IGD (e.g., human PCSK9 IGD).

In some embodiments, the target sequence is 10-50 nucleotides (e.g., 10-40, 10-30, 15-30, 15-25, or 15-20 nucleotides) in a region (e.g., a 0.1-2 kb region) of the PCSK9 IGD (e.g., the human PCSK9 IGD), wherein the region comprises a CTCF binding site (e.g., a CTCF binding site at a boundary of the PCSK9 IGD or a CTCF binding site in the PCSK9 IGD). In some embodiments, the target sequence is within or overlapping the CTCF binding site. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of the PCSK9 IGD (e.g., the human PCSK9 IGD), wherein the region comprises a CTCF binding site (e.g., a CTCF binding site at a boundary of the PCSK9 IGD or a CTCF binding site in the PCSK9 IGD). In some embodiments, the target sequence is about 10, about 11, about 12, about 13, about 14, about 15, about 16, about 17, about 18, about 19, or about 20 nucleotides in a region (e.g., a 0.1-2 kb region) of the PCSK9 IGD (e.g., the human PCSK9 IGD), wherein the region comprises a CTCF binding site (e.g., a CTCF binding site at a boundary of the PCSK9 IGD or a CTCF binding site in the PCSK9 IGD).

In some embodiments, the target sequence is 10-50 nucleotides (e.g., 10-40, 10-30, 15-30, 15-25, or 15-20 nucleotides) in a region (e.g., a 0.1-2 kb region) of the PCSK9 IGD (e.g., the human PCSK9 IGD), wherein the region comprises a PCSK9 enhancer (e.g., a human PCSK9 enhancer). In some embodiments, the target sequence is within or overlapping the PCSK9 enhancer. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of the PCSK9 IGD (e.g., the human PCSK9 IGD), wherein the region comprises a PCSK9 enhancer (e.g., a human PCSK9 enhancer). In some embodiments, the target sequence is about 10, about 11, about 12, about 13, about 14, about 15, about 16, about 17, about 18, about 19, or about 20 nucleotides in a region (e.g., a 0.1-2 kb region) of the PCSK9 IGD (e.g., the human PCSK9 IGD), wherein the region comprises a PCSK9 enhancer (e.g., a human PCSK9 enhancer).

In some embodiments, the target sequence is 10-50 nucleotides (e.g., 10-40, 10-30, 15-30, 15-25, or 15-20 nucleotides) in a region (e.g., a 0.1-2 kb region) of the PCSK9 IGD (e.g., the human PCSK9 IGD), wherein the region comprises a CpG island. In some embodiments, the target sequence is within or overlapping the CpG island. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of the of the PCSK9 IGD (e.g., the human PCSK9 IGD), wherein the region comprises a CpG island. In some embodiments, the target sequence is about 10, about 11, about 12, about 13, about 14, about 15, about 16, about 17, about 18, about 19, or about 20 nucleotides in a region (e.g., a 0.1-2 kb region) of the of the PCSK9 IGD (e.g., the human PCSK9 IGD), wherein the region comprises a CpG island.

In some embodiments, the target sequence is 10-50 nucleotides (e.g., 10-40, 10-30, 15-30, 15-25, or 15-20 nucleotides) in a PCSK9 enhancer (e.g., a human PCSK9 enhancer). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a PCSK9 enhancer (e.g., a human PCSK9 enhancer). In some embodiments, the target sequence is about 10, about 11, about 12, about 13, about 14, about 15, about 16, about 17, about 18, about 19, or about 20 nucleotides in a PCSK9 enhancer (e.g., a human PCSK9 enhancer).

In some embodiments, the target sequence is 10-50 nucleotides (e.g., 10-40, 10-30, 15-30, 15-25, or 15-20 nucleotides) in a region (e.g., a 0.1-2 kb region) of the PCSK9 IGD (e.g., the human PCSK9 IGD), wherein the region comprises a PCSK9 promoter (e.g., a human PCSK9 promoter). In some embodiments, the target sequence is within or overlapping the PCSK9 promoter. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of the PCSK9 IGD (e.g., the human PCSK9 IGD), wherein the region comprises a PCSK9 promoter (e.g., a human PCSK9 promoter). In some embodiments, the target sequence is about 10, about 11, about 12, about 13, about 14, about 15, about 16, about 17, about 18, about 19, or about 20 nucleotides in a region (e.g., a 0.1-2 kb region) of the PCSK9 IGD (e.g., the human PCSK9 IGD), wherein the region comprises a PCSK9 promoter (e.g., a human PCSK9 promoter).

In some embodiments, the target sequence is 10-50 nucleotides (e.g., 10-40, 10-30, 15-30, 15-25, or 15-20 nucleotides) in a PCSK9 promoter (e.g., a human PCSK9 promoter). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a PCSK9 promoter (e.g., a human PCSK9 promoter). In some embodiments, the target sequence is about 10, about 11, about 12, about 13, about 14, about 15, about 16, about 17, about 18, about 19, or about 20 nucleotides in a PCSK9 promoter (e.g., a human PCSK9 promoter).

In some embodiments, the target sequence is 10-50 nucleotides (e.g., 10-40, 10-30, 15-30, 15-25, or 15-20 nucleotides) in a region (e.g., a 0.1-2 kb region) of PCSK9 (e.g., human PCSK9). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region (e.g., a 0.1-2 kb region) of PCSK9 (e.g., human PCSK9). In some embodiments, the target sequence is about 10, about 11, about 12, about 13, about 14, about 15, about 16, about 17, about 18, about 19, or about 20 nucleotides in a region (e.g., a 0.1-2 kb region) of PCSK9 (e.g., human PCSK9).

In some embodiments, the target sequence is 10-50 nucleotides (e.g., 10-40, 10-30, 15-30, 15-25, or 15-20 nucleotides) in a region spanning position 55,037,859 to position 55,041,755, according to the hg38 reference genome for chromosome 1 (chr1). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region spanning position 55,037,859 to position 55,041,755, according to the hg38 reference genome for chr1. In some embodiments, the target sequence is about 10, about 11, about 12, about 13, about 14, about 15, about 16, about 17, about 18, about 19, or about 20 nucleotides in a region spanning position 55,037,859 to position 55,041,755, according to the hg38 reference genome for chr1. In some embodiments, the target sequence is about 15 nucleotides in a region spanning position 55,037,859 to position 55,041,755, according to the hg38 reference genome for chr1. In some embodiments, the target sequence is about 16 nucleotides in a region spanning position 55,037,859 to position 55,041,755, according to the hg38 reference genome for chr1. In some embodiments, the target sequence is about 17 nucleotides in a region spanning position 55,037,859 to position 55,041,755, according to the hg38 reference genome for chr1. In some embodiments, the target sequence is about 18 nucleotides in a region spanning position 55,037,859 to position 55,041,755, according to the hg38 reference genome for chr1. In some embodiments, the target sequence is about 19 nucleotides in a region spanning position 55,037,859 to position 55,041,755, according to the hg38 reference genome for chr1. In some embodiments, the target sequence is about 20 nucleotides in a region spanning position 55,037,859 to position 55,041,755, according to the hg38 reference genome for chr1.

In some embodiments, the target sequence is 10-50 nucleotides (e.g., 10-40, 10-30, 15-30, 15-25, or 15-20 nucleotides) in a region spanning position 55,038,000 to position 55,040,500, according to the hg38 reference genome for chromosome 1 (chr1). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region spanning position 55,038,000 to position 55,040,500, according to the hg38 reference genome for chr1. In some embodiments, the target sequence is about 10, about 11, about 12, about 13, about 14, about 15, about 16, about 17, about 18, about 19, or about 20 nucleotides in a region spanning position 55,038,000 to position 55,040,500, according to the hg38 reference genome for chr1. In some embodiments, the target sequence is about 15 nucleotides in a region spanning position 55,038,000 to position 55,040,500, according to the hg38 reference genome for chr1. In some embodiments, the target sequence is about 16 nucleotides in a region spanning position 55,038,000 to position 55,040,500, according to the hg38 reference genome for chr1. In some embodiments, the target sequence is about 17 nucleotides in a region spanning position 55,038,000 to position 55,040,500, according to the hg38 reference genome for chr1. In some embodiments, the target sequence is about 18 nucleotides in a region spanning position 55,038,000 to position 55,040,500, according to the hg38 reference genome for chr1. In some embodiments, the target sequence is about 19 nucleotides in a region spanning position 55,038,000 to position 55,040,500, according to the hg38 reference genome for chr1. In some embodiments, the target sequence is about 20 nucleotides in a region spanning position 55,038,000 to position 55,040,500, according to the hg38 reference genome for chr1.

In some embodiments, the target sequence is 10-50 nucleotides (e.g., 10-40, 10-30, 15-30, 15-25, or 15-20 nucleotides) in a region spanning position 55,039,100 to position 55,039,600, according to the hg38 reference genome for chromosome 1 (chr1). In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region spanning position 55,039,100 to position 55,039,600, according to the hg38 reference genome for chr1. In some embodiments, the target sequence is about 10, about 11, about 12, about 13, about 14, about 15, about 16, about 17, about 18, about 19, or about 20 nucleotides in a region spanning position 55,039,100 to position 55,039,600, according to the hg38 reference genome for chr1. In some embodiments, the target sequence is about 15 nucleotides in a region spanning position 55,039,100 to position 55,039,600, according to the hg38 reference genome for chr1. In some embodiments, the target sequence is about 16 nucleotides in a region spanning position 55,039,100 to position 55,039,600, according to the hg38 reference genome for chr1. In some embodiments, the target sequence is about 17 nucleotides in a region spanning position 55,039,100 to position 55,039,600, according to the hg38 reference genome for chr1. In some embodiments, the target sequence is about 18 nucleotides in a region spanning position 55,039,100 to position 55,039,600, according to the hg38 reference genome for chr1. In some embodiments, the target sequence is about 19 nucleotides in a region spanning position 55,039,100 to position 55,039,600, according to the hg38 reference genome for chr1. In some embodiments, the target sequence is about 20 nucleotides in a region spanning position 55,039,100 to position 55,039,600, according to the hg38 reference genome for chr1.

In some embodiments, the region spans position 55,039,100 to position 55,039,200, according to the hg38 reference genome for chr1. In some embodiments, the region spans position 55,039,150 to position 55,039,250, according to the hg38 reference genome for chr1. In some embodiments, the region spans position 55,039,200 to position 55,039,300, according to the hg38 reference genome for chr1. In some embodiments, the region spans position 55,039,250 to position 55,039,350, according to the hg38 reference genome for chr1. In some embodiments, the region spans position 55,039,300 to position 55,039,400, according to the hg38 reference genome for chr1. In some embodiments, the region spans position 55,039,350 to position 55,039,450, according to the hg38 reference genome for chr1. In some embodiments, the region spans position 55,039,400 to position 55,039,500, according to the hg38 reference genome for chr1. In some embodiments, the region spans position 55,039,450 to position 55,039,550, according to the hg38 reference genome for chr1. In some embodiments, the region spans position 55,039,500 to position 55,039,600, according to the hg38 reference genome for chr1.

In some embodiments, the target sequence is 10-50 nucleotides (e.g., 10-40, 10-30, 15-30, 15-25, or 15-20 nucleotides) in a region spanning position 55,503,531 to position 55,507,429, according to the hg19 reference genome for chr1. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region spanning position 55,503,531 to position 55,507,429, according to the hg19 reference genome for chr1. In some embodiments, the target sequence is about 10, about 11, about 12, about 13, about 14, about 15, about 16, about 17, about 18, about 19, or about 20 nucleotides in a region spanning position 55,503,531 to position 55,507,429, according to the hg19 reference genome for chr1. In some embodiments, the target sequence is about 15 nucleotides in a region spanning position 55,503,531 to position 55,507,429, according to the hg19 reference genome for chr1. In some embodiments, the target sequence is about 16 nucleotides in a region spanning position 55,503,531 to position 55,507,429, according to the hg19 reference genome for chr1. In some embodiments, the target sequence is about 17 nucleotides in a region spanning position 55,503,531 to position 55,507,429, according to the hg19 reference genome for chr1. In some embodiments, the target sequence is about 18 nucleotides in a region spanning position 55,503,531 to position 55,507,429, according to the hg19 reference genome for chr1. In some embodiments, the target sequence is about 19 nucleotides in a region spanning position 55,503,531 to position 55,507,429, according to the hg19 reference genome for chr1. In some embodiments, the target sequence is about 20 nucleotides in a region spanning position 55,503,531 to position 55,507,429, according to the hg19 reference genome for chr1.

In some embodiments, the target sequence is 10-50 nucleotides (e.g., 10-40, 10-30, 15-30, 15-25, or 15-20 nucleotides) in a region spanning position 55,503,700 to position 55,505,700, according to the hg19 reference genome for chr1. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region spanning position 55,503,700 to position 55,505,700, according to the hg19 reference genome for chr1. In some embodiments, the target sequence is about 10, about 11, about 12, about 13, about 14, about 15, about 16, about 17, about 18, about 19, or about 20 nucleotides in a region spanning position 55,503,700 to position 55,505,700, according to the hg19 reference genome for chr1. In some embodiments, the target sequence is about 15 nucleotides in a region spanning position 55,503,700 to position 55,505,700, according to the hg19 reference genome for chr1. In some embodiments, the target sequence is about 16 nucleotides in a region spanning position 55,503,700 to position 55,505,700, according to the hg19 reference genome for chr1. In some embodiments, the target sequence is about 17 nucleotides in a region spanning position 55,503,700 to position 55,505,700, according to the hg19 reference genome for chr1. In some embodiments, the target sequence is about 18 nucleotides in a region spanning position 55,503,700 to position 55,505,700, according to the hg19 reference genome for chr1. In some embodiments, the target sequence is about 19 nucleotides in a region spanning position 55,503,700 to position 55,505,700, according to the hg19 reference genome for chr1. In some embodiments, the target sequence is about 20 nucleotides in a region spanning position 55,503,700 to position 55,505,700, according to the hg19 reference genome for chr1.

In some embodiments, the target sequence is 10-50 nucleotides (e.g., 10-40, 10-30, 15-30, 15-25, or 15-20 nucleotides) in a region spanning position 55,504,600 to position 55,505,300, according to the hg19 reference genome for chr1. In some embodiments, the target sequence is about 10 to about 50 nucleotides, about 10 to about 40 nucleotides, about 10 to about 30 nucleotides, about 10 to about 20 nucleotides, or about 15 to about 20 nucleotides in a region spanning position 55,504,600 to position 55,505,300, according to the hg19 reference genome for chr1. In some embodiments, the target sequence is about 10, about 11, about 12, about 13, about 14, about 15, about 16, about 17, about 18, about 19, or about 20 nucleotides in a region spanning position 55,504,600 to position 55,505,300, according to the hg19 reference genome for chr1. In some embodiments, the target sequence is about 15 nucleotides in a region spanning position 55,504,600 to position 55,505,300, according to the hg19 reference genome for chr1. In some embodiments, the target sequence is about 16 nucleotides in a region spanning position 55,504,600 to position 55,505,300, according to the hg19 reference genome for chr1. In some embodiments, the target sequence is about 17 nucleotides in a region spanning position 55,504,600 to position 55,505,300, according to the hg19 reference genome for chr1. In some embodiments, the target sequence is about 18 nucleotides in a region spanning position 55,504,600 to position 55,505,300, according to the hg19 reference genome for chr1. In some embodiments, the target sequence is about 19 nucleotides in a region spanning position 55,504,600 to position 55,505,300, according to the hg19 reference genome for chr1. In some embodiments, the target sequence is about 20 nucleotides in a region spanning position 55,504,600 to position 55,505,300, according to the hg19 reference genome for chr1.

In some embodiments, the region spans position 55,504,600 to position 55,504,700, according to the hg19 reference genome for chr1. In some embodiments, the region spans position 55,504,650 to position 55,504,750, according to the hg19 reference genome for chr1. In some embodiments, the region spans position 55,504,700 to position 55,504,800, according to the hg19 reference genome for chr1. In some embodiments, the region spans position 55,504,750 to position 55,504,850, according to the hg19 reference genome for chr1. In some embodiments, the region spans position 55,504,800 to position 55,504,900, according to the hg19 reference genome for chr1. In some embodiments, the region spans position 55,504,850 to position 55,504,950, according to the hg19 reference genome for chr1. In some embodiments, the region spans position 55,504,900 to position 55,505,000, according to the hg19 reference genome for chr1. In some embodiments, the region spans position 55,504,950 to position 55,505,050, according to the hg19 reference genome for chr1. In some embodiments, the region spans position 55,505,000 to position 55,505,100, according to the hg19 reference genome for chr1. In some embodiments, the region spans position 55,505,050 to position 55,505,150, according to the hg19 reference genome for chr1. In some embodiments, the region spans position 55,505,100 to position 55,505,200, according to the hg19 reference genome for chr1. In some embodiments, the region spans position 55,505,150 to position 55,505,250, according to the hg19 reference genome for chr1. In some embodiments, the region spans position 55,505,200 to position 55,505,300, according to the hg19 reference genome for chr1.

In some embodiments, the target sequence is 10-50 nucleotides (e.g., 10-40, 10-30, 15-30, 15-25, or 15-20 nucleotides) comprising at least 10 contiguous nucleotides of a sequence selected from SEQ ID NOs: 67-80. In some embodiments, the target sequence is about 10 to about 50 nucleotides, about 10 to about 40 nucleotides, about 10 to about 30 nucleotides, about 10 to about 20 nucleotides, or about 15 to about 20 nucleotides comprising at least 10 contiguous nucleotides of a sequence selected from SEQ ID NOs: 67-80. In some embodiments, the target sequence is about 10, about 11, about 12, about 13, about 14, about 15, about 16, about 17, about 18, about 19, or about 20 nucleotides comprising at least 10 contiguous nucleotides of a sequence selected from SEQ ID NOs: 67-80. In some embodiments, the target sequence is (i) 15 nucleotides and comprises 10-15 contiguous nucleotides of the sequence; (ii) 16 nucleotides and comprises 10-16 contiguous nucleotides of the sequence; (iii) 17 nucleotides and comprises 10-17 contiguous nucleotides of the sequence; (iv) 18 nucleotides and comprises 10-18 contiguous nucleotides of the sequence; (v) 19 nucleotides and comprises 10-19 contiguous nucleotides of the sequence; or (vi) 20 nucleotides and comprises 10-20 contiguous nucleotides of the sequence.

In some embodiments, the target sequence is 10-50 nucleotides (e.g., 10-40, 10-30, 15-30, 15-25, or 15-20 nucleotides) comprising at least 10 contiguous nucleotides of SEQ ID NO: 67. In some embodiments, the target sequence is 10-50 nucleotides (e.g., 10-40, 10-30, 15-30, 15-25, or 15-20 nucleotides) comprising at least 10 contiguous nucleotides of SEQ ID NO: 68. In some embodiments, the target sequence is 10-50 nucleotides (e.g., 10-40, 10-30, 15-30, 15-25, or 15-20 nucleotides) comprising at least 10 contiguous nucleotides of SEQ ID NO: 69. In some embodiments, the target sequence is 10-50 nucleotides (e.g., 10-40, 10-30, 15-30, 15-25, or 15-20 nucleotides) comprising at least 10 contiguous nucleotides of SEQ ID NO: 70. In some embodiments, the target sequence is 10-50 nucleotides (e.g., 10-40, 10-30, 15-30, 15-25, or 15-20 nucleotides) comprising at least 10 contiguous nucleotides of SEQ ID NO: 71. In some embodiments, the target sequence is 10-50 nucleotides (e.g., 10-40, 10-30, 15-30, 15-25, or 15-20 nucleotides) comprising at least 10 contiguous nucleotides of SEQ ID NO: 72. In some embodiments, the target sequence is 10-50 nucleotides (e.g., 10-40, 10-30, 15-30, 15-25, or 15-20 nucleotides) comprising at least 10 contiguous nucleotides of SEQ ID NO: 73. In some embodiments, the target sequence is 10-50 nucleotides (e.g., 10-40, 10-30, 15-30, 15-25, or 15-20 nucleotides) comprising at least 10 contiguous nucleotides of SEQ ID NO: 74. In some embodiments, the target sequence is 10-50 nucleotides (e.g., 10-40, 10-30, 15-30, 15-25, or 15-20 nucleotides) comprising at least 10 contiguous nucleotides of SEQ ID NO: 75. In some embodiments, the target sequence is 10-50 nucleotides (e.g., 10-40, 10-30, 15-30, 15-25, or 15-20 nucleotides) comprising at least 10 contiguous nucleotides of SEQ ID NO: 76. In some embodiments, the target sequence is 10-50 nucleotides (e.g., 10-40, 10-30, 15-30, 15-25, or 15-20 nucleotides) comprising at least 10 contiguous nucleotides of SEQ ID NO: 77. In some embodiments, the target sequence is 10-50 nucleotides (e.g., 10-40, 10-30, 15-30, 15-25, or 15-20 nucleotides) comprising at least 10 contiguous nucleotides of SEQ ID NO: 78. In some embodiments, the target sequence is 10-50 nucleotides (e.g., 10-40, 10-30, 15-30, 15-25, or 15-20 nucleotides) comprising at least 10 contiguous nucleotides of SEQ ID NO: 79. In some embodiments, the target sequence is 10-50 nucleotides (e.g., 10-40, 10-30, 15-30, 15-25, or 15-20 nucleotides) comprising at least 10 contiguous nucleotides of SEQ ID NO: 80.

In some embodiments, the target sequence is 18-50 nucleotides (e.g., 18-40, 18-35, 18-30, 18-25, or 18-20 nucleotides) comprising a sequence selected from SEQ ID NOs: 67-80. In some embodiments, the target sequence is about 18 to about 50 nucleotides, about 18 to about 40 nucleotides, about 18 to about 30 nucleotides, or about 18 to about 20 nucleotides comprising a sequence selected from SEQ ID NOs: 67-80. In some embodiments, the target sequence is about 18, about 19, or about 20 nucleotides comprising a sequence selected from SEQ ID NOs: 67-80.

In some embodiments, the target sequence is 18-50 nucleotides (e.g., 18-40, 18-35, 18-30, 18-25, or 18-20 nucleotides) comprising SEQ ID NO: 67. In some embodiments, the target sequence is 18-50 nucleotides (e.g., 18-40, 18-35, 18-30, 18-25, or 18-20 nucleotides) comprising SEQ ID NO: 68. In some embodiments, the target sequence is 18-50 nucleotides (e.g., 18-40, 18-35, 18-30, 18-25, or 18-20 nucleotides) comprising SEQ ID NO: 69. In some embodiments, the target sequence is 18-50 nucleotides (e.g., 18-40, 18-35, 18-30, 18-25, or 18-20 nucleotides) comprising SEQ ID NO: 70. In some embodiments, the target sequence is 18-50 nucleotides (e.g., 18-40, 18-35, 18-30, 18-25, or 18-20 nucleotides) comprising SEQ ID NO: 71. In some embodiments, the target sequence is 18-50 nucleotides (e.g., 18-40, 18-35, 18-30, 18-25, or 18-20 nucleotides) comprising SEQ ID NO: 72. In some embodiments, the target sequence is 18-50 nucleotides (e.g., 18-40, 18-35, 18-30, 18-25, or 18-20 nucleotides) comprising SEQ ID NO: 73. In some embodiments, the target sequence is 18-50 nucleotides (e.g., 18-40, 18-35, 18-30, 18-25, or 18-20 nucleotides) comprising SEQ ID NO: 74. In some embodiments, the target sequence is 18-50 nucleotides (e.g., 18-40, 18-35, 18-30, 18-25, or 18-20 nucleotides) comprising SEQ ID NO: 75. In some embodiments, the target sequence is 18-50 nucleotides (e.g., 18-40, 18-35, 18-30, 18-25, or 18-20 nucleotides) comprising SEQ ID NO: 76. In some embodiments, the target sequence is 18-50 nucleotides (e.g., 18-40, 18-35, 18-30, 18-25, or 18-20 nucleotides) comprising SEQ ID NO: 77. In some embodiments, the target sequence is 18-50 nucleotides (e.g., 18-40, 18-35, 18-30, 18-25, or 18-20 nucleotides) comprising SEQ ID NO: 78. In some embodiments, the target sequence is 18-50 nucleotides (e.g., 18-40, 18-35, 18-30, 18-25, or 18-20 nucleotides) comprising SEQ ID NO: 79. In some embodiments, the target sequence is 18-50 nucleotides (e.g., 18-40, 18-35, 18-30, 18-25, or 18-20 nucleotides) comprising SEQ ID NO: 80.

In some embodiments, the target sequence is about 10-50 nucleotides (e.g., 10-40, 10-30, 15-30, 15-25, or 15-20 nucleotides) within a region of about 200 or fewer nucleotides, wherein the region comprises at least 10 contiguous nucleotides of a sequence selected from SEQ ID NOs: 67-80, and wherein the region is located within position 55,037,859 to position 55,041,755, according to the hg38 reference genome for chr1. In some embodiments, the target sequence is about 10 to about 50 nucleotides, about 10 to about 40 nucleotides, about 10 to about 30 nucleotides, about 10 to about 20 nucleotides, or about 15 to about 20 nucleotides within a region of about 200 or fewer nucleotides, wherein the region comprises at least 10 contiguous nucleotides of a sequence selected from SEQ ID NOs: 67-80, and wherein the region is located within position 55,037,859 to position 55,041,755, according to the hg38 reference genome for chr1. In some embodiments, the target sequence is about 10, about 11, about 12, about 13, about 14, about 15, about 16, about 17, about 18, about 19, or about 20 nucleotides within a region of about 200 or fewer nucleotides, wherein the region comprises at least 10 contiguous nucleotides of a sequence selected from SEQ ID NOs: 67-80, and wherein the region is located within position 55,037,859 to position 55,041,755, according to the hg38 reference genome for chr1. In some embodiments, the region comprises 10, 11, 12, 13, 14, 15, 16, 17, or 18 contiguous nucleotides of the sequence. In some embodiments, the region is about 175, about 150, about 125, about 100, about 90, about 80, about 70, about 60, about 50, about 45, about 40, about 35, about 30, about 25, or about 20 nucleotides. In some embodiments, the target sequence is 15, 16, 17, 18, 19, or 20 nucleotides.

Exemplary target sequence of the disclosure in the human PCSK9 IGD are set forth in Table 1.

TABLE 1
Exemplary target sequences of the disclosure

| | SEQ ID | Chromosomal | Positive (+) or |
|---|---|---|---|
| Name/Identifier | NO | coordinates* | negative (−) strand |
| Target 1 | 67 | 55039201-55039218 | − |
| Target 2 | 68 | 55039211-55039228 | + |
| Target 3 | 69 | 55039239-55039256 | + |
| Target 4 | 70 | 55039237-55039254 | + |
| Target 5 | 71 | 55039423-55039440 | − |
| Target 6 | 72 | 55039458-55039475 | + |
| Target 7 | 73 | 55039452-55039469 | − |
| Target 8 | 74 | 55039526-55039543 | − |
| Target 9 | 75 | 55039525-55039542 | − |
| Target 10 | 76 | 55039523-55039540 | + |
| Target 11 | 77 | 55039193-55039210 | − |
| Target 12 | 78 | 55039458-55039475 | − |
| Target 13 | 79 | 55039527-55039544 | + |
| Target 14 | 80 | 55039527-55039544 | − |

*According to human reference genome hg38 of chromosome 1

### DNA Targeting Moiety

The present disclosure provides, e.g., expression repressors comprising a DNA targeting moiety that specifically targets, e.g., binds, a genomic sequence element (e.g., a promoter, a TSS, or an anchor sequence) in, proximal to, and/or operably linked to a target gene. In some embodiments, the DNA targeting moiety specifically binds to a DNA sequence, e.g., a DNA sequence associated with a target gene, e.g., PCSK9. Any molecule or compound that specifically binds a DNA sequence may be used as a DNA targeting moiety.

In some embodiments, the DNA targeting moiety targets, e.g., binds, a component of a genomic complex. In some embodiments, the DNA targeting moiety targets, e.g., binds, a transcriptional control sequence (e.g., a promoter or enhancer) operably linked to the target gene (e.g., PCSK9). In some embodiments, the DNA targeting moiety targets, e.g., binds, a target gene or a part of a target gene (e.g., PCSK9). The target of a DNA targeting moiety may be referred to as its targeted component. A targeted component may be any genomic sequence element operably linked to a target gene, or the target gene itself, including but not limited to a promoter, enhancer, anchor sequence, exon, intron, UTR encoding sequence, a splice site, or a transcription start site. In some embodiments, the DNA targeting moiety binds specifically to one or more target anchor sequences (e.g., within a cell) and not to non-targeted anchor sequences (e.g., within the same cell).

In some embodiments, the DNA targeting moiety comprises a CRISPR/Cas domain (e.g., a catalytically inactive CRISPR/Cas domain), a TAL effector domain, a Zn finger domain, a peptide nucleic acid (PNA), or a nucleic acid molecule.

In some embodiments, an expression repressor of the disclosure comprises one DNA targeting moiety. In some embodiments, the expression repressor comprises a plurality of DNA targeting moieties, wherein each DNA targeting moiety does not detectably bind, e.g., does not bind, to another DNA targeting moiety.

In some embodiments, the DNA targeting moiety binds to its target sequence with a KD of less than or equal to 500, 450, 400, 350, 300, 250, 200, 150, 100, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM (and optionally, a KD of at least 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM). In some embodiments, the DNA targeting moiety binds to its target sequence with a KD of 0.001 nM to 500 nM, e.g., 0.1 nM to 5 nM, e.g., about 0.5 nM. In some embodiments, a DNA targeting moiety binds to a non-target sequence with a KD of at least 500, 600, 700, 800, 900, 1000, 2000, 5000, 10,000, or 100,000 nM (and optionally, does not appreciably bind to a non-target sequence). In some embodiments, the DNA targeting moiety does not substantially bind to a non-target sequence.

### CRISPR/Cas Domains

In some embodiments, the DNA targeting moiety comprises a CRISPR/Cas domain. A CRISPR/Cas protein can comprise a CRISPR/Cas effector and optionally one or more other domains. A CRISPR/Cas domain typically has structural and/or functional similarity to a protein involved in the clustered regulatory interspaced short palindromic repeat (CRISPR) system, e.g., a Cas protein. The CRISPR/Cas domain optionally comprises a guide RNA, e.g., single guide RNA (sgRNA). In some embodiments, the gRNA comprised by the CRISPR/Cas domain is noncovalently bound by the CRISPR/Cas domain.

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. For example, in a typical CRISPR/Cas system, an endonuclease is directed to a target nucleotide sequence (e. g., a site in the genome that is to be sequence-edited) by sequence specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by Rnase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas1, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-targeting moiety includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram-positive bacteria or a gram-negative bacteria. In certain embodiments, a Cas protein may be from a Streptococcus (e.g., an S. pyogenes, or an S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 2. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises DI 135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions.

TABLE 2
Exemplary Cas Proteins of the Disclosure

| | | | # of | | Mutations | Mutations to |
|---|---|---|---|---|---|---|
| | | | amino | | to alter | make |
| | | | acid | | PAM | catalytically |
| Name | Enzyme | Species | residues | PAM | recognition | dead |
| FnCas9 | Cas9 | Francisella | 1629 | 5′-NGG-3′ | Wt | D11A/H969A/ |
| | | novicida | | | | N995A |
| FnCas9 | Cas9 | Francisella | 1629 | 5′-YG-3′ | E1369R/ | D11A/H969A/ |
| RHA | | novicida | | | E1449H/ | N995A |
| | | | | | R1556A | |
| SaCas9 | Cas9 | Staphylococcus | 1053 | 5′- | Wt | D10A/H557A |
| | | aureus | | NNGRRT- | | |
| | | | | 3′ | | |
| SaCas9 | Cas9 | Staphylococcus | 1053 | 5′- | E782K/ | D10A/H557A |
| KKH | | aureus | | NNNRRT- | N968K/ | |
| | | | | 3′ | R1015H | |
| SpCas9 | Cas9 | Streptococcus | 1368 | 5′-NGG-3′ | Wt | D10A/D839A/ |
| | | pyogenes | | | | H840A/N863A |
| SpCas9 | Cas9 | Streptococcus | 1368 | 5′-NGA-3′ | D1135V/ | D10A/D839A/ |
| VQR | | pyogenes | | | R1335Q/ | H840A/N863A |
| | | | | | T1337R | |
| AsCpf1 | Cpf1 | Acidaminococcus | 1307 | 5′-TYCV- | S542R/ | E993A |
| RR | | sp. BV3L6 | | 3′ | K607R | |
| AsCpf1 | Cpf1 | Acidaminococcus | 1307 | 5′-TATV- | S542R/ | E993A |
| RVR | | sp. BV3L6 | | 3′ | K548V/ | |
| | | | | | N552R | |
| FnCpf1 | Cpf1 | Francisella | 1300 | 5′-NTTN- | Wt | D917A/ |
| | | novicida | | 3′ | | E1006A/ |
| | | | | | | D1255A |
| NmCas9 | Cas9 | Neisseria | 1082 | 5′- | Wt | D16A/D587A/ |
| | | meningitidis | | NNNGAT | | H588A/N611A |
| | | | | T-3′ | | |

In some embodiments, the Cas protein is modified to deactivate the nuclease, e.g., nuclease-deficient Cas. In some embodiments, the Cas protein is a Cas9 protein. Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas binding to a DNA sequence may interfere with transcription at that site by steric hindrance. In some embodiments, a DNA-targeting moiety is or comprises a catalytically inactive Cas, e.g., dCas. Many catalytically inactive Cas proteins are known in the art. In some embodiments, dCas9 comprises mutations in each endonuclease domain of the Cas protein, e.g., D10A and H840A mutations.

In some embodiments, a catalytically inactive Cas9 protein, e.g., dCas9, comprises a D11A mutation or an analogous substitution to the amino acid corresponding to said position. In some embodiments, a catalytically inactive Cas9 protein, e.g., dCas9, comprises a H969A mutation or an analogous substitution to the amino acid corresponding to said position. In some embodiments, a catalytically inactive Cas9 protein, e.g., dCas9, comprises a N995A mutation or an analogous substitution to the amino acid corresponding to said position. In some embodiments, a catalytically inactive Cas9 protein, e.g., dCas9, comprises D11A, H969A, and N995A mutations or analogous substitutions to the amino acids corresponding to said positions.

In some embodiments, a catalytically inactive Cas9 protein, e.g., dCas9, comprises a D10A mutation or an analogous substitution to the amino acid corresponding to said position. In some embodiments, a catalytically inactive Cas9 protein, e.g., dCas9, comprises a H557A mutation or an analogous substitution to the amino acid corresponding to said position. In some embodiments, a catalytically inactive Cas9 protein, e.g., dCas9, comprises D10A and H557A mutations or analogous substitutions to the amino acids corresponding to said positions.

In some embodiments, a catalytically inactive Cas9 protein, e.g., dCas9, comprises a D839A mutation or an analogous substitution to the amino acid corresponding to said position. In some embodiments, a catalytically inactive Cas9 protein, e.g., dCas9, comprises a H840A mutation or an analogous substitution to the amino acid corresponding to said position. In some embodiments, a catalytically inactive Cas9 protein, e.g., dCas9, comprises a N863A mutation or an analogous substitution to the amino acid corresponding to said position. In some embodiments, a catalytically inactive Cas9 protein, e.g., dCas9, comprises D10A, D839A, H840A, and N863A mutations or analogous substitutions to the amino acids corresponding to said positions.

In some embodiments, a catalytically inactive Cas9 protein, e.g., dCas9, comprises a E993A mutation or an analogous substitution to the amino acid corresponding to said position.

In some embodiments, a catalytically inactive Cas9 protein, e.g., dCas9, comprises a D917A mutation or an analogous substitution to the amino acid corresponding to said position. In some embodiments, a catalytically inactive Cas9 protein, e.g., dCas9, comprises a E1006A mutation or an analogous substitution to the amino acid corresponding to said position. In some embodiments, a catalytically inactive Cas9 protein, e.g., dCas9, comprises a D1255A mutation or an analogous substitution to the amino acid corresponding to said position. In some embodiments, a catalytically inactive Cas9 protein, e.g., dCas9, comprises D917A, E1006A, and D1255A mutations or analogous substitutions to the amino acids corresponding to said positions.

In some embodiments, a catalytically inactive Cas9 protein, e.g., dCas9, comprises a D16A mutation or an analogous substitution to the amino acid corresponding to said position. In some embodiments, a catalytically inactive Cas9 protein, e.g., dCas9, comprises a D587A mutation or an analogous substitution to the amino acid corresponding to said position. In some embodiments, a catalytically inactive Cas9 protein, e.g., dCas9, comprises a H588A mutation or an analogous substitution to the amino acid corresponding to said position. In some embodiments, a catalytically inactive Cas9 protein, e.g., dCas9, comprises an N611A mutation or an analogous substitution to the amino acid corresponding to said position. In some embodiments, a catalytically inactive Cas9 protein, e.g., dCas9, comprises D16A, D587A, H588A, and N611A mutations or analogous substitutions to the amino acids corresponding to said positions.

In another aspect, the disclosure is directed to an expression repressor or a polypeptide comprising one or more (e.g., one) DNA targeting moiety and one or more effector domain, wherein the one or more DNA targeting moiety is or comprises a CRISPR/Cas domain comprising a Cas protein, e.g., catalytically inactive Cas9 protein, e.g., dCas9, or a functional variant or fragment thereof. In some embodiments, dCas9 comprises an amino acid sequence of SEQ ID NO: 95.

In some embodiments, the dCas9 is encoded by a nucleic acid sequence of SEQ ID NO: 96.

In some embodiments, a DNA targeting moiety comprises a Cas domain comprising or linked (e.g., covalently linked) to a gRNA. A gRNA is a short synthetic RNA composed of a “scaffold” sequence necessary for Cas-protein binding and a user-defined about 20 nucleotide targeting sequence for a genomic target. In practice, guide RNA sequences are generally designed to have a length of between 17-24 nucleotides (e.g., 19, 20, or 21 nucleotides) and be complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective for use with Cas proteins; see, for example, Hendel et al. (2015) Nature Biotechnol, 985-991.

In some embodiments, a gRNA comprises a nucleic acid sequence that is complementary to a target sequence described herein. In some embodiments, a gRNA comprises a nucleic acid sequence that is at least 90, 95, 99, or 100% complementary to a target sequence described herein. In some embodiments, a gRNA for use with a DNA-targeting moiety that comprises a Cas molecule is an sgRNA.

### TAL Domains

In some embodiments, a DNA-targeting moiety is or comprises a TAL effector (also sometimes referred to herein as a “TALE”) domain. A TAL effector domain, e.g., a TAL effector domain that specifically binds a DNA sequence, comprises a plurality of TAL effector repeats or fragments thereof, and optionally one or more additional portions of naturally occurring TAL effector repeats (e.g., N- and/or C-terminal of the plurality of TAL effector domains) wherein each TAL effector repeat recognizes a nucleotide. In some embodiments, a TAL effector protein can comprise a TAL effector domain and optionally one or more other domains. Many TAL effector domains are known to those of skill in the art and are commercially available, e.g., from Thermo Fisher Scientific.

TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector features a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 3 listing exemplary repeat variable di-residues (RVD) and their correspondence to nucleic acid base targets.

TABLE 3
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, in some embodiments, the TAL effector repeat of the TAL effector domain of the present disclosure may be derived from a TAL effector from any bacterial species (e.g., Xanthomonas species such as the African strain of Xanthomonas oryzae pv. Oryzae (Yu et al. 2011), Xanthomonas campestris pv. raphani strain strain 756C and Xanthomonas oryzae pv. Oryzicolastrain BLS256 (Bogdanove et al. 2011). As used herein, the TAL effector domain in accordance with the present disclosure comprises an RVD domain as well as flanking sequence(s) (sequences on the N-terminal and/or C-terminal side of the RVD domain) also from the naturally occurring TAL effector. In some embodiments, it may comprise more or fewer repeats than the RVD of the naturally occurring TAL effector domain. The TAL effector domain of the present disclosure is designed to target a given DNA sequence based on the above code and others known in the art. The number of TAL effector repeats (e.g., monomers or modules) and their specific sequence(s) are selected based on the desired DNA target sequence. For example, TAL effector repeats may be removed or added in order to suit a specific target sequence. In an embodiment, the TAL effector domain of the present disclosure comprises between 6.5 and 33.5 TAL effector repeats. In an embodiment, TAL effector domain of the present disclosure comprises between 8 and 33.5 TAL effector repeats, e.g., between 10 and 25 TAL effector repeats, e.g., between 10 and 14 TAL effector repeats.

In some embodiments, the TAL effector domain comprises TAL effector repeats that correspond to a perfect match to the DNA target sequence. In some embodiments, a mismatch between a repeat and a target base-pair on the DNA target sequence is permitted as along as it allows for the function of the expression repression system, e.g., the expression repressor comprising the TAL effector domain. In general, TALE binding is inversely correlated with the number of mismatches. In some embodiments, the TAL effector domain of an expression repressor of the present disclosure comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector repeats in the TAL effector domain, the smaller the number of mismatches will be tolerated while still allowing for the function of the expression repressor or expression repressor system, e.g., the expression repressor comprising the TAL effector domain. The binding affinity is thought to depend on the sum of matching repeat-DNA combinations. For example, TAL effector domains having 25 TAL effector repeats or more may be able to tolerate up to 7 mismatches.

In addition to the TAL effector repeats, in some embodiments, the TAL effector domain of the present disclosure may comprise additional sequences derived from a naturally occurring TAL effector. The length of the C-terminal and/or N-terminal sequence(s) included on each side of the TAL effector repeat portion of the TAL effector domain can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of the N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector repeats of the naturally occurring TAL effector are included in the TAL effector domain of an expression repressor of the present disclosure. Accordingly, in an embodiment, a TAL effector domain of the present disclosure comprises 1) one or more TAL effector repeats derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector repeats; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector repeats.

In some embodiments, a modulating agent comprises a DNA targeting moiety comprising an engineered DNA binding domain (DBD), e.g., a TAL effector comprising a TAL effector repeat that binds to a target sequence, e.g., a promoter or transcription start site (TSS)) sequence operably linked to a target gene (e.g., PCSK9), e.g., a sequence proximal to the transcription regulatory element, e.g., an anchor sequence of an anchor sequence mediated conjunction (ASMC) comprising a target gene (e.g., PCSK9), e.g., a sequence proximal to the anchor sequence. In some embodiments, the TAL effector binds to a target sequence described herein. In some embodiments, the TAL effector domain can be engineered to carry epigenetic effector domains to target sites.

The amino acid sequences of exemplary DNA targeting moieties disclosed herein are listed in Table 4. In some embodiments, an expression repressor or system described herein comprises a DNA targeting moiety comprising a sequence set forth in Table 4, or a sequence with at least 70%, 75%, 80%, 85%, 90%, 95%, 98%, or 99% identity thereto.

TABLE 4
Protein Sequences for Exemplary TAL Effector
Domains of the Disclosure and Corresponding
Target Sequence in the human PCSK9 IGD

| | DNA | | | |
|---|---|---|---|---|
| | Targeting | | | Target |
| | Moiety | | | sequence |
| | amino acid | Target | | positive (+) |
| | sequence | sequence | Target sequence | or |
| Name/ | (SEQ ID | (SEQ ID | Chromosomal | negative (−) |
| Identifier | NO) | NO) | coordinates* | strand |
| DBD473 | 81 | 67 | 55039201-55039218 | − |
| DBD474 | 82 | 68 | 55039211-55039228 | + |
| DBD475 | 83 | 69 | 55039239-55039256 | + |
| DBD476 | 84 | 70 | 55039237-55039254 | + |
| DBD477 | 85 | 71 | 55039423-55039440 | − |
| DBD478 | 86 | 72 | 55039458-55039475 | + |
| DBD479 | 87 | 73 | 55039452-55039469 | − |
| DBD480 | 88 | 74 | 55039526-55039543 | − |
| DBD481 | 89 | 75 | 55039525-55039542 | − |
| DBD482 | 90 | 76 | 55039523-55039540 | + |
| DBD483 | 91 | 77 | 55039193-55039210 | − |
| DBD484 | 92 | 78 | 55039458-55039475 | − |
| DBD485 | 93 | 79 | 55039527-55039544 | + |
| DBD486 | 94 | 80 | 55039527-55039544 | − |

*According to human reference genome hg38 of chromosome 1.

In some embodiments, an expression repressor or system described herein comprises a DNA targeting moiety comprising a sequence set forth in SEQ ID NO:81. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:81. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 81.

In some embodiments, an expression repressor or system described herein comprises a DNA targeting moiety comprising a sequence set forth in SEQ ID NO:82. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:82. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 82.

In some embodiments, an expression repressor or system described herein comprises a DNA targeting moiety comprising a sequence set forth in SEQ ID NO:83. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:83. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 83.

In some embodiments, an expression repressor or system described herein comprises a DNA targeting moiety comprising a sequence set forth in SEQ ID NO:84. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:84. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 84.

In some embodiments, an expression repressor or system described herein comprises a DNA targeting moiety comprising a sequence set forth in SEQ ID NO:85. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:85. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 85.

In some embodiments, an expression repressor or system described herein comprises a DNA targeting moiety comprising a sequence set forth in SEQ ID NO:86. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:86. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 86.

In some embodiments, an expression repressor or system described herein comprises a DNA targeting moiety comprising a sequence set forth in SEQ ID NO:87. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:87. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 87.

In some embodiments, an expression repressor or system described herein comprises a DNA targeting moiety comprising a sequence set forth in SEQ ID NO:88. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:88. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 88.

In some embodiments, an expression repressor or system described herein comprises a DNA targeting moiety comprising a sequence set forth in SEQ ID NO:89. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:89. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 89.

In some embodiments, an expression repressor or system described herein comprises a DNA targeting moiety comprising a sequence set forth in SEQ ID NO:90. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:90. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 90.

In some embodiments, an expression repressor or system described herein comprises a DNA targeting moiety comprising a sequence set forth in SEQ ID NO:91. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:91. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 91.

In some embodiments, an expression repressor or system described herein comprises a DNA targeting moiety comprising a sequence set forth in SEQ ID NO:92. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:92. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 92.

In some embodiments, an expression repressor or system described herein comprises a DNA targeting moiety comprising a sequence set forth in SEQ ID NO:93. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:93. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 93.

In some embodiments, an expression repressor or system described herein comprises a DNA targeting moiety comprising a sequence set forth in SEQ ID NO:94. In some embodiments, an expression repressor or system described herein comprises a DNA targeting moiety comprising a sequence with at least 70%, 75%, 80%, 85%, 90%, 95%, 98%, or 99% identity to the sequence set forth in SEQ ID NO:94. In some embodiments, an expression repressor or system described herein comprises a DNA targeting moiety comprising a sequence with at least 90% (e.g., at least 91%, at least 92%, at least 93%, at least 94%, at least 95%, at least 96%, at least 97%, at least 98%, at least 99%, or 100%) identity to the sequence set forth in SEQ ID NO: 94.

### Zn Finger Domains

In some embodiments, a DNA-targeting moiety is or comprises a Zn finger domain. A Zn finger domain comprises a Zn finger, e.g., a naturally occurring Zn finger or engineered Zn finger, or fragment thereof. Many Zn fingers are known to those of skill in the art and are commercially available, e.g., from Sigma-Aldrich. Generally, a Zn finger domain comprises a plurality of Zn fingers, wherein each Zn finger recognizes three nucleotides. A Zn finger protein can comprise a Zn finger domain and optionally one or more other domains.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger may have a novel binding specificity, compared to a naturally-occurring Zn finger. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc fingers and/or multi-fingered zinc finger domains may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in International Patent Publication No. WO 02/077227.

Zn fingers and methods for design and construction of expression repressors (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In certain embodiments, the DNA-targeting moiety comprises a Zn finger domain comprising an engineered zinc finger that binds (in a sequence-specific manner) to a target DNA sequence. In some embodiments, the Zn finger domain comprises one Zn finger or fragment thereof. In some embodiments, the Zn finger domain comprises a plurality of Zn fingers (or fragments thereof), e.g., 2, 3, 4, 5, 6 or more Zn fingers (and optionally no more than 12, 11, 10, 9, 8, 7, 6, 5, 4, 3, or 2 Zn fingers). In some embodiments, the Zn finger domain comprises at least three Zn fingers. In some embodiments, the Zn finger domain comprises four, five or six Zn fingers. In some embodiments, the Zn finger domain comprises 8, 9, 10, 11 or 12 Zn fingers. In some embodiments, a Zn finger domain comprising three Zn fingers recognizes a target DNA sequence comprising 9 or 10 nucleotides. In some embodiments, a Zn finger domain comprising four Zn fingers recognizes a target DNA sequence comprising 12 to 14 nucleotides. In some embodiments, a Zn finger domain comprising six Zn fingers recognizes a target DNA sequence comprising 18 to 21 nucleotides.

In some embodiments, a DNA targeting domain comprises a two-handed Zn finger protein. Two handed zinc finger proteins are those proteins in which two clusters of zinc fingers are separated by intervening amino acids so that the two zinc finger domains bind to two discontinuous target DNA sequences. An example of a two-handed type of zinc finger binding protein is SIP1, where a cluster of four zinc fingers is located at the amino terminus of the protein and a cluster of three Zn fingers is located at the carboxyl terminus (see Remade, et al. (1999) EMBO Journal 18(18): 5073-5084). Each cluster of zinc fingers in these domains is able to bind to a unique target sequence and the spacing between the two target sequences can comprise many nucleotides.

In some embodiments, an expression repressor comprises a DNA targeting moiety comprising an engineered DNA binding domain (DBD), e.g., a Zn finger domain comprising a Zn finger (ZFN) that binds to a target sequence, e.g., a promoter or transcription start site (TSS)) sequence operably linked to a target gene (e.g., PCSK9), e.g., a sequence proximal to the transcription regulatory element, e.g., an anchor sequence of an anchor sequence mediated conjunction (ASMC) comprising a target gene (e.g., PCSK9), e.g., a sequence proximal to the anchor sequence. In some embodiments, the ZFN binds to a target sequence described herein. In some embodiments, the ZFN can be engineered to carry epigenetic effector molecules to target sites.

### Effector Domain

In some embodiments, expression repressors of the present disclosure comprise one or more effector domains. In some embodiments, an effector domain, when used as part of an expressor repressor or an expression repression system described herein, decreases expression of a target gene in a cell.

In some embodiments, the effector domain has functionality unrelated to the binding of the DNA targeting moiety. For example, effector domains may target, e.g., bind, a genomic sequence element or genomic complex component proximal to the genomic sequence element targeted by the DNA targeting moiety or recruit a transcription factor. As a further example, an effector domain may comprise an enzymatic activity, e.g., a genetic modification functionality.

In some embodiments, the effector domain is any one described in Int Pub No. WO2022/132195; Int Pub No WO2022/067033; or U.S. Pat. No. 11,312,955 (herein incorporated by reference).

In some embodiments, an effector domain comprises a transcriptional repressor moiety. In some embodiments, an effector domain comprises a DNA modifying functionality, e.g., a DNA methyltransferase. In some embodiments, the effector domain comprises a polypeptide that induces DNA methylation. In some embodiments, the effector domain comprises a polypeptide that induces DNA methylation of a CpG island (i.e., a region of the genome comprising a high concentration of CpG residues). In some embodiments, the effector domain comprises a DNA methyltransferase enzyme (DNMT). In some embodiments, the effector domain comprises a polypeptide that induces histone modification. In some embodiments, the effector domain comprises a histone modifying enzyme. In some embodiments, the histone modifying enzyme is selected from a histone acetyltransferase, a histone deacetylase (HDAC), a histone lysine methyl-transferase, and a histone lysine demethylase. In some embodiments, the effector domain comprises a polypeptide that forms a complex for epigenetic modification. In some embodiments, the polypeptide forms a complex that induces DNA modification and/or histone modification. In some embodiments, the effector domain comprises a Krüppel-associated box (KRAB) domain.

In some embodiments, an effector domain is or comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.

In some embodiments, an effector domain comprises a transcription repressor that stimulates or promotes transcription, e.g., of the target gene. In some embodiments, the transcription repressor recruits a factor that inhibits transcription, e.g., of the target gene. In some embodiments, an effector domain, e.g., transcription repressor, is or comprises a protein chosen from KRAB, MeCP2, HP1, RBBP4, REST, FOG1, SUZ12, or a functional variant or fragment of any thereof.

In some embodiments an effector domain promotes epigenetic modification, e.g., directly or indirectly. For example, an effector domain can indirectly promote epigenetic modification by recruiting an endogenous protein that epigenetically modifies the chromatin. An effector domain can directly promote epigenetic modification by catalyzing epigenetic modification, wherein the effector domain comprises enzymatic activity and directly places an epigenetic mark on the chromatin.

In some embodiments, an effector domain comprises a histone modifying functionality, e.g., a histone methyltransferase, histone demethylase, or histone deacetylase activity. In some embodiments, an effector domain is or comprises a protein chosen from SETDB1, SETDB2, EHMT2, EHMT1, SUV39H1, EZH2, EZH1, SUV39H2, SETD8, SUV420H1, SUV420H2, or a functional variant or fragment thereof. In some embodiments, an effector domain is or comprises a protein chosen from KDM1A (i.e., LSD1), KDM1B (i.e., LSD2), KDM2A, KDM2B, KDM5A, KDM5B, KDM5C, KDM5D, KDM4B, NO66, or a functional variant or fragment of any thereof. In some embodiments, an effector domain is or comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

In some embodiments, an effector domain comprises a protein having a functionality described herein. In some embodiments, an effector domain is or comprises a protein selected from: KRAB (e.g., as according to NP_056209.2 or the protein encoded by NM_015394.5); a SET domain (e.g., the SET domain of: SETDB1 (e.g., as according to NP 001353347.1 or the protein encoded by NM_001366418.1); EZH2 (e.g., as according to NP-004447.2 or the protein encoded by NM_004456.5); G9A (e.g., as according to NP_001350618.1 or the protein encoded by NM_001363689.1); or SUV39H1 (e.g., as according to NP_003164.1 or the protein encoded by NM_003173.4)); histone demethylase LSD1 (e.g., as according to NP 055828.2 or the protein encoded by NM 015013.4); FOG1 (e.g., the N-terminal residues of FOG1) (e.g., as according to NP_722520.2 or the protein encoded by NM_153813.3); or KAP1 (e.g., as according to NP_005753.1 or the protein encoded by NM_005762.3); a functional fragment or variant of any thereof, or a polypeptide with a sequence that has at least 80, 85, 90, 91, 92, 93, 94, 95, 96, 97, 98, or 99% identity to any of the above-referenced sequences.

In some embodiments, an effector domain is or comprises a protein selected from: DNMT3A (e.g., human DNMT3A) (e.g., as according to NP_072046.2 or the protein encoded by NM_022552.4); DNMT3B (e.g., as according to NP 008823.1 or the protein encoded by NM_006892.4); DNMT3L (e.g., as according to NP_787063.1 or the protein encoded by NM_175867.3); DNMT3A/3L complex, bacterial MQ1 (e.g., as according to CAA35058.1 or P15840.3); a functional fragment of any thereof, or a polypeptide with a sequence that has at least 80, 85, 90, 91, 92, 93, 94, 95, 96, 97, 98, or 99% identity to any of the above-referenced sequences.

In another aspect, the disclosure is directed to an expression repressor or a polypeptide comprising one or more (e.g., one) DNA targeting moiety and one or more effector domains, wherein the one or more effector domains are or comprise Krueppel-associated box (KRAB) e.g., as according to NP_056209.2 or the protein encoded by NM_015394.5 or a functional variant or fragment thereof. In some embodiments, KRAB is a synthetic KRAB construct. In some embodiments, KRAB comprises an amino acid sequence of SEQ ID NO: 101.

In some embodiments, the KRAB effector domain comprises the amino acid sequence of SEQ ID NO: 101. In some embodiments, the KRAB effector domain is encoded by a nucleotide sequence of SEQ ID NO: 102. In some embodiments, a nucleotide sequence described herein comprises a sequence of SEQ ID NO: 102 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, KRAB for use in a polypeptide or an expression repressor described herein is a variant, e.g., comprising one or more mutations, relative to the KRAB sequence of SEQ ID NO: 101. In some embodiments, a KRAB variant comprises one or more amino acid substitutions, deletions, or insertions relative to SEQ ID NO: 101. In some embodiments, the effector domain comprises an amino acid sequence having at least about 70%, about 75%, about 80%, about 85%, or about 90% identity to SEQ ID NO: 101. In some embodiments, the effector domain comprises an amino acid sequence having at least about 90%, about 95%, about 98%, or about 99% identity to SEQ ID NO: 101.

In some embodiments, the polypeptide or the expression repressor is a fusion protein comprising an effector domain that is or comprises KRAB and a DNA-targeting moiety. In some embodiments, the DNA targeting moiety is or comprises a zinc finger domain, TAL domain, or CRISPR/Cas domain, e.g., comprising a CRISPR/Cas protein, e.g., a dCas9 protein. In some embodiments, the polypeptide or the expression repressor comprises an additional moiety described herein. In some embodiments, the polypeptide or the expression repressor decreases expression of a target gene, e.g., PCSK9. In some embodiments, the polypeptide or the expression repressor may be used in methods of modulating, e.g., decreasing, gene expression, methods of treating a condition, or methods of epigenetically modifying a target gene, e.g., PCSK9 or transcription control element described herein, e.g., in place of an expression repressor system. In some embodiments, an expression repressor system comprises two or more (e.g., two, three, or four) expression repressors, wherein the first expression repressor comprises an effector domain comprising the KRAB sequence of SEQ ID NO: 101, or a functional variant or fragment thereof.

In another aspect, the disclosure is directed to an expression repressor or a polypeptide comprising one or more (e.g., one) DNA targeting moiety and one or more effector domains, wherein the one or more effector domains are or comprise MQ1, e.g., bacterial MQ1, or a functional variant or fragment thereof. In some embodiments, MQ1 is Mollicutes spiroplasma MQ1. In some embodiments, MQ1 is Spiroplasma monobiae MQ1. In some embodiments, MQ1 is MQ1 derived from strain ATCC 33825 and/or corresponding to Uniprot ID P15840. In some embodiments, MQ1 comprises an amino acid sequence of SEQ ID NO: 47. In some embodiments, MQ1 comprises an amino acid sequence of SEQ ID NO: 99. In some embodiments, an effector domain described herein comprises SEQ ID NO: 47 or 99, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, MQ1 is encoded by a nucleotide sequence of SEQ ID NO: 98 or 100. In some embodiments, a nucleic acid described herein comprises a sequence of SEQ ID NO: 98, 100 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, MQ1 for use in a polypeptide or an expression repressor described herein is a variant, e.g., comprising one or more mutations, relative to a wildtype MQ1 (e.g., SEQ ID NO: 47). In some embodiments, an MQ1 variant comprises one or more amino acid substitutions, deletions, or insertions relative to a wildtype MQ1, e.g., the MQ1 of SEQ ID NO: 47. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1).

In some embodiments, the polypeptide or the expression repressor is a fusion protein comprising an effector domain that is or comprises MQ1 and a DNA targeting moiety is or comprises a zinc finger domain, TAL domain, or CRISPR/Cas domain, a dCas9 domain. In some embodiments, the polypeptide or the expression repressor comprises an additional moiety described herein. In some embodiments, the polypeptide or the expression repressor decreases expression of a target gene, e.g., PCSK9. In some embodiments, the polypeptide or the expression repressor may be used in methods of modulating, e.g., decreasing, gene expression, methods of treating a condition, or methods of epigenetically modifying a target gene, e.g., PCSK9 or transcription control element described herein, e.g., in place of an expression repressor system. In some embodiments, an expression repressor system comprises two or more (e.g., two, three, or four) expression repressors, wherein the first expression repressor comprises an effector domain comprising MQ1, e.g., bacterial MQ1, or a functional variant or fragment thereof.

In another aspect, the disclosure is directed to an expression repressor or a polypeptide comprising one or more (e.g., one) DNA targeting moiety and one or more effector domains, wherein the one or more effector domains are or comprise DNMT1, e.g., human DNMT1, or a functional variant or fragment thereof. In some embodiments, DNMT1 is human DNMT1, e.g., corresponding to Gene ID 1786, e.g., corresponding to UniProt ID P26358.2. In some embodiments, DNMT1 comprises an amino acid sequence of SEQ ID NO: 103. In some embodiments, an effector domain described herein comprises a sequence according to SEQ ID NO: 103 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, DNMT1 is encoded by a nucleotide sequence of SEQ ID NO: 104. In some embodiments, a nucleic acid described herein comprises a sequence of SEQ ID NO: 104 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, DNMT1 for use in a polypeptide or an expression repressor described herein is a variant, e.g., comprising one or more mutations, relative to a DNMT sequence of SEQ ID NO: 103. In some embodiments, the effector domain comprises one or more amino acid substitutions, deletions, or insertions relative to wild type DNMT1. In some embodiments, the polypeptide is a fusion protein comprising a repressor domain that is or comprises DNMT1 and a DNA targeting moiety. In some embodiments, the DNA targeting moiety is or comprises a zinc finger domain, TAL domain, or CRISPR/Cas domain, e.g., a dCas9 domain. In some embodiments, an expression repression system comprises two or more (e.g., two, three, or four) expression repressors, wherein the first expression repressor comprises an effector domain comprising DNMT1, or a functional variant or fragment thereof.

In another aspect, the disclosure is directed to an expression repressor or a polypeptide comprising one or more (e.g., one) DNA targeting moiety and one or more effector domains, wherein the one or more effector domains are or comprise DNMT3a/3Lcomplex, or a functional variant or fragment thereof. In some embodiments, the one or more effector domains are or comprise a DNMT3a/3L complex fusion construct. In some embodiments, the DNMT3a/3L complex comprises DNMT3A (e.g., human DNMT3A) (e.g., as according to NP_072046.2 or the protein encoded by NM_022552.4). In some embodiments, the DNMT3a/3L complex comprises DNMT3L (e.g., as according to NP_787063.1 or the protein encoded by NM_175867.3). In some embodiments, DNMT3a/3L comprises an amino acid sequence of SEQ ID NO: 38 or SEQ ID NO: 39. In some embodiments, an effector domain described herein comprises SEQ ID NO: 38 or SEQ ID NO: 39, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, DNMT3a/3L is encoded by a nucleotide sequence of SEQ ID NO: 40. In some embodiments, a nucleic acid described herein comprises a sequence of SEQ ID NO: 40 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, DNMT3a/3L for use in a polypeptide or an expression repressor described herein is a variant, e.g., comprising one or more mutations, relative to the DNMT3a/3L of SEQ ID NO: 38 or SEQ ID NO: 39. In some embodiments, a DNMT3a/3L variant comprises one or more amino acid substitutions, deletions, or insertions relative to SEQ ID NO: 38 or SEQ ID NO: 39. In some embodiments, the polypeptide or the expression repressor is a fusion protein comprising an effector domain that is or comprises DNMT3a/3L and a DNA targeting moiety. In some embodiments, the DNA targeting moiety is or comprises a zinc finger domain, TAL domain, or CRISPR/Cas domain e.g., a dCas9 domain. In some embodiments, an expression repressor system comprises two or more (e.g., two, three, or four) expression repressors, wherein the first expression repressor comprises an effector domain comprising DNMT3a/3L, or a functional variant or fragment thereof.

In some embodiments, an effector domain is or comprises a polypeptide. In some embodiments, an effector domain is or comprises a nucleic acid. In some embodiments, an effector domain is a chemical, e.g., a chemical that modulates a cytosine I or an adenine (A) (e.g., Na bisulfite, ammonium bisulfite). In some embodiments, an effector domain has enzymatic activity (e.g., methyl transferase, demethylase, nuclease (e.g., Cas9), or deaminase activity). An effector domain may be or comprise one or more of a small molecule, a peptide, a nucleic acid, a nanoparticle, an aptamer, or a pharmaco-agent with poor PK/PD.

In some embodiments, an effector domain may comprise a peptide ligand, a full-length protein, a protein fragment, an antibody, an antibody fragment, and/or a targeting aptamer. In some embodiments, the protein may bind a receptor such as an extracellular receptor, neuropeptide, hormone peptide, peptide drug, toxic peptide, viral or microbial peptide, synthetic peptide, or agonist or antagonist peptide.

In some embodiments, an effector domain may comprise antigens, antibodies, antibody fragments such as, e.g. single domain antibodies, ligands, or receptors such as, e.g., glucagon-like peptide-1 (GLP-1), GLP-2 receptor 2, cholecystokinin B (CCKB), or somatostatin receptor, peptide therapeutics such as, e.g., those that bind to specific cell surface receptors such as G protein-coupled receptors (GPCRs) or ion channels, synthetic or analog peptides from naturally-bioactive peptides, anti-microbial peptides, poreforming peptides, tumor targeting or cytotoxic peptides, or degradation or self-destruction peptides such as an apoptosis-inducing peptide signal or photosensitizer peptide.

Peptide or protein moieties for use in effector domains as described herein may also include small antigen-binding peptides, e.g., antigen binding antibody or antibody-like fragments, such as, e.g., single chain antibodies, nanobodies (see, e.g., Steeland et al. 2016. Nanobodies as therapeutics: big opportunities for small antibodies. Drug Discov Today: 21(7): 1076-1 13). Such small antigen binding peptides may bind, e.g., a cytosolic antigen, a nuclear antigen, an intra-organellar antigen.

In some embodiments, an effector domain comprises a dominant negative component (e.g., dominant negative moiety), e.g., a protein that recognizes and binds a sequence (e.g., an anchor sequence, e.g., a CTCF binding motif), but with an inactive (e.g., mutated) dimerization domain, e.g., a dimerization domain that is unable to form a functional anchor sequence-mediated conjunction), or binds to a component of a genomic complex (e.g., a transcription factor subunit, etc.) preventing formation of a functional transcription factor, etc. For example, the Zinc Finger domain of CTCF can be altered so that it binds a specific anchor sequence (by adding zinc fingers that recognize flanking nucleic acids), while the homo-dimerization domain is altered to prevent the interaction between engineered CTCF and endogenous forms of CTCF. In some embodiments, a dominant negative component comprises a synthetic nucleating polypeptide with a selected binding affinity for an anchor sequence within a target anchor sequence-mediated conjunction. In some embodiments, binding affinity may be at least 10%, 15%, 20%, 25%, 30%, 35%, 40%, 45%, 50%, 55%, 60%, 65%, 70%, 75%, 80%, 85%, 90%, 95%, or higher or lower than binding affinity of an endogenous nucleating polypeptide (e.g., CTCF) that associates with a target anchor sequence. In some embodiments, A synthetic nucleating polypeptide may have between 30-90%, 30-85%, 30-80%, 30-70%, 50-80%, 50-90% amino acid sequence identity to a corresponding endogenous nucleating polypeptide. A nucleating polypeptide may modulate (e.g., disrupt), such as through competitive binding, e.g., competing with binding of an endogenous nucleating polypeptide to its anchor sequence.

In some embodiments, an effector domain comprises an antibody or antigen-binding fragment thereof. In some embodiments, target gene (e.g., PCSK9) expression is altered via use of effector domains that are or comprise one or more antibodies or antigen-binding fragments thereof. In some embodiments, gene expression is altered via use of effector domains that are or comprise one or more antibodies (or antigen-binding fragments thereof) and dCas9.

In some embodiments, an antibody or antigen-binding fragment thereof for use in an effector domain may be monoclonal. An antibody may be a fusion, a chimeric antibody, a non-humanized antibody, a partially or fully humanized antibody, a single chain antibody, Fab fragment, Fv fragment, F(ab′)2 fragment, scFv fragment, etc. As will be understood by one of skill in the art, format of antibody(ies) used may be the same or different depending on a given target.

In some embodiments, an effector domain comprises one or more RNAs (e.g., gRNA) and dCas9. In some embodiments, one or more RNAs is/are targeted to a genomic sequence element via dCas9 and target-specific guide RNA. As will be understood by one of skill in the art, RNAs used for targeting may be the same or different depending on a given target. An effector domain may comprise an aptamer, such as an oligonucleotide aptamer or a peptide aptamer. Aptamer moieties are oligonucleotide or peptide aptamers.

An effector domain may comprise an oligonucleotide aptamer. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues, and organisms. Aptamers provide discriminate molecular recognition and can be produced by chemical synthesis. In addition, aptamers possess desirable storage properties, and elicit little or no immunogenicity in therapeutic applications.

Both DNA and RNA aptamers show robust binding affinities for various targets. For example, DNA and RNA aptamers have been selected for t lysozyme, thrombin, human immunodeficiency virus trans-acting responsive element (HIV TAR), hemin, interferon y, vascular endothelial growth factor (VEGF), prostate specific antigen (PSA), dopamine, and the non-classical oncogene, heat shock factor 1 (HSF1).

An effector domain may comprise a peptide aptamer moiety. Peptide aptamers have one (or more) short variable peptide domains, including peptides having low molecular weight, 12-14 Da. Peptide aptamers may be designed to specifically bind to and interfere with protein-protein interactions inside cells.

Peptide aptamers are artificial proteins selected or engineered to bind specific target molecules. These proteins include one or more peptide complexes of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer complex attached to a transcription factor binding domain is screened against a target protein attached to a transcription factor activating domain. In vivo binding of a peptide aptamer to its target via this selection strategy is detected as expression of a downstream yeast marker gene. Such experiments identify particular proteins bound by aptamers, and protein interactions that aptamers disrupt, to cause a given phenotype. In addition, peptide aptamers derivatized with appropriate functional moieties can cause specific post-translational modification of their target proteins or change subcellular localization of the targets. Peptide aptamers can also recognize targets in vitro. They have found use in lieu of antibodies in biosensors and may be used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most commonly used is currently a yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. Peptides panned from combinatorial peptide libraries have been stored in a special database with named MimoDB.

An exemplary effector domain may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3A, DNMT3B, DNMT3a/3L, MQ1), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), specific domains from proteins, such as a KRAB domain, and fusions of one or more thereof (e.g., dCas9-DNMT, dCas9-MQ1, dCas9-KRAB).

In some embodiments, a candidate effector domain may be determined to be suitable for use as an effector domain by methods known to those of skill in the art. For example, a candidate effector domain may be tested by assaying whether, when the candidate effector domain is present in the nucleus of a cell and appropriately localized (e.g., to a target gene or transcription control element operably linked to said target gene, e.g., via a DNA targeting moiety), the candidate effector domain decreases expression of the target gene in the cell, e.g., decreases the level of RNA transcript encoded by the target gene (e.g., as measured by RNASeq or Northern blot) or decreases the level of protein encoded by the target gene (e.g., as measured by ELISA).

In some embodiments, an expression repressor comprises a plurality of effector domains, wherein each effector domain does not detectably bind, e.g., does not bind, to another effector domain. In some embodiments, an expression repressor system comprises a first expression repressor comprising a first effector domain and a second expression repressor comprising a second effector domain, wherein the first effector domain does not detectably bind, e.g., does not bind, to the second effector domain.

In some embodiments, an expression repressor system comprises a plurality of expression repressors, wherein each member of the plurality of expression repressors comprises an effector domain, wherein each effector domain does not detectably bind, e.g., does not bind, to another effector domain. In some embodiments, an expression repressor system comprises a first expression repressor comprising a first effector domain and a second expression repressor comprising a second effector domain, wherein the first effector domain does not detectably bind, e.g., does not bind, to the second effector domain. In some embodiments, an expression repressor system comprises a first expression repressor comprising a first effector domain and a second expression repressor comprising a second effector domain, wherein the first effector domain does not detectably bind, e.g., does not bind, to another first effector domain, and the second effector domain does not detectably bind, e.g., does not bind, to another second effector domain.

In some embodiments, an effector domain for use in the compositions and methods described herein is functional in a monomeric, e.g., non-dimeric, state.

In some embodiments, an effector domain is or comprises a transcriptional repressor moiety. In some embodiments, the transcriptional repressor moiety e.g. modulates the two-dimensional structure of chromatin (i.e., modulates structure of chromatin in a way that would alter its two-dimensional representation).

Transcriptional repressor moieties useful in methods and compositions of the present disclosure include agents that affect epigenetic markers, e.g., DNA methylation, histone methylation, histone acetylation, histone sumoylation, histone phosphorylation, and RNA-associated silencing. Exemplary epigenetic enzymes that can be targeted to a genomic sequence element as described herein include DNA methylases (e.g., DNMT3a, DNMT3b, DNMT3a/3L, MQ1), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdbl), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012): 1-18.

In some embodiments, an expression repressor, e.g., comprising an epigenetic modifying moiety, useful herein comprises or is a construct described in Koferle et al. Genome Medicine 7.59 (2015): 1-3 incorporated herein by reference. For example, in some embodiments, an expression repressor comprises or is a construct found in Table 1 of Koferle et al., e.g., histone deacetylase, histone methyltransferase, DNA demethylation, or H3K4 and/or H3K9 histone demethylase described in Table 1 (e.g., dCas9-p300, TALE-TET1, ZF-DNMT3A, or TALE-LSD1).

In some embodiments, an effector domain comprises a component of a gene editing system, e.g, a CRISPR/Cas domain, e.g., a Zn Finger domain, e.g., a TAL effector domain. In some embodiments, a transcriptional repressor moiety may comprise a polypeptide (e.g., peptide or protein moiety) linked to a gRNA and a targeted nuclease, e.g., a Cas9, e.g., a wild type Cas9, a nickase Cas9 (e.g., Cas9 D10A), a catalytically inactive Cas9 (dCas9), eSpCas9, Cpf1, C2C1, or C2C3, or a nucleic acid encoding such a nuclease.

In some embodiments, an effector domain comprises a biologically active fragment of the effector domain. As used herein, a “biologically active fragment of an effector domain” is a portion that maintains function (e.g., completely, partially, minimally) of an effector domain (e.g., a “minimal” or “core” domain). In some embodiments, fusion of a dCas9 with all or a portion of one or more effector domains of an epigenetic modifying agent (such as a DNA methylase or enzyme with a role in DNA demethylation, e.g., DNMT3a, DNMT3b, DNMT3L, a DNMT inhibitor, combinations thereof, TET family enzymes, protein acetyl transferase or deacetylase, dCas9-DNMT3a/3L, dCas9-DNMT3a/3L/KRAB, dCas9/VP64) creates a chimeric protein that is linked to the polypeptide and useful in the methods described herein. An effector domain comprising such a chimeric protein is referred to as either a genetic modifying moiety (because of its use of a gene editing system component, Cas9) or a transcriptional repressor moiety (because of its use of an effector domain of a transcriptional repressor agent).

In some embodiments, provided technologies are described as comprising a gRNA that specifically targets a target gene. In some embodiments, the target gene is PCSK9.

### Additional Moieties

An expression repressor may further comprise one or more additional moieties (e.g., in addition to one or more targeting moieties and one or more effector domains). In some embodiments, an additional moiety is selected from a tagging or monitoring moiety, a cleavable moiety (e.g., a cleavable moiety positioned between a DNA-targeting moiety and an effector domain or at the N- or C-terminal end of a polypeptide), a small molecule, a membrane translocating polypeptide, or a pharmaco-agent moiety.

### Linkers

An expression repressor or an expression repressor system as disclosed herein may comprise one or more linkers. A linker may connect a targeting moiety to an effector moiety, an effector moiety to another effector moiety, or a targeting moiety to another targeting moiety. A linker may be a chemical bond, e.g., one or more covalent bonds or non-covalent bonds. In some embodiments, a linker is covalent. In some embodiments, a linker is non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first domain or moiety from a second domain or moiety, e.g., a DNA-targeting moiety from an effector moiety. In some embodiments, for example, a linker can be positioned between a DNA-targeting moiety and an effector moiety, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g., polypeptide) from a modulating agent, but sufficiently stable to prevent premature cleavage.

In some embodiments, one or more moieties and/or domains of an expression repressor described herein are linked with one or more linkers. In some embodiments, an expression repression may comprise a linker situated between the targeting moiety and the effector moiety. In some embodiments, the linker may have a sequence of ASGSGGGSGGARD (SEQ ID NO: 55), or ASGSGGGSGG (SEQ ID NO: 62). In some embodiments, a system comprising a first and second repressor may comprise a first linker situated between the first targeting moiety and the first effector moiety, and a second linker situated between the second targeting moiety and the second effector moiety. In some embodiments, the first and the second linker may be identical. In some embodiments, the first and the second linker may be different. In some embodiments, the first linker may comprise an amino acid sequence according to SEQ ID NO: 55 or a sequence with at least 80, 85, 90, 95, 99, or 100% identity thereto and the second linker may comprise an amino acid sequence according to SEQ ID NO: 62 or a sequence with at least 80, 85, 90, 95, 99, or 100% identity thereto.

As will be known by one of skill in the art, commonly used flexible linkers have sequences consisting primarily of stretches of Gly and Ser residues (“GS” linker). Flexible linkers may be useful for joining domains/moieties that require a certain degree of movement or interaction and may include small, non-polar (e.g., Gly) or polar (e.g., Ser or Thr) amino acids. Incorporation of Ser or Thr can also maintain the stability of a linker in aqueous solutions by forming hydrogen bonds with water molecules, and therefore reduce unfavorable interactions between a linker and moieties/domains. In some embodiments, the linker is a GS linker or a variant thereof e.g., G4S (SEQ ID NO: 63).

Rigid linkers are useful to keep a fixed distance between domains/moieties and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP) n, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments. In some embodiment, the cleavable linker may be a self-cleaving linker, e.g., a T2A peptide linker. In some embodiments, the linker may comprise a “ribosome skipping” sequence, e.g., a tPT2A linker.

Examples of molecules suitable for use in linkers described herein include a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof; noncarbon linkers; carbohydrate linkers; phosphodiester linkers, or other molecule capable of covalently linking two or more components of an expression repressor. Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine, or other hydrophobic residues. Components of an expression repressor may be linked using charge-based chemistry, such that a positively charged component of an expression repressor is linked to a negative charge of another component.

### Exemplary PCSK9 Expression Repressors

In some embodiments, the disclosure provides an expression repressor comprising a TAL effector domain that binds a target sequence described herein operably linked to an MQ1 effector domain, or a functional variant or fragment thereof. In some embodiments, the disclosure provides an expression repressor comprising a ZFN effector domain that binds a target sequence described herein operably linked to an MQ1 effector domain, or a functional variant or fragment thereof. In some embodiments, the disclosure provides an expression repressor comprising a dCas9 effector domain comprising a guide sequence that binds a target sequence described herein operably linked to an MQ1 effector domain, or a functional variant or fragment thereof.

In some embodiments, the disclosure provides an expression repressor comprising a TAL effector domain that binds a target sequence described herein operably linked to a KRAB domain, or a functional variant or fragment thereof. In some embodiments, the disclosure provides an expression repressor comprising a ZFN effector domain that binds a target sequence described herein operably linked to an KRAB domain, or a functional variant or fragment thereof. In some embodiments, the disclosure provides an expression repressor comprising a dCas9 effector domain comprising a guide sequence that binds a target sequence described herein operably linked to an KRAB domain, or a functional variant or fragment thereof.

In some embodiments, the disclosure provides an expression repressor comprising a TAL effector domain operably linked to an MQ1 effector domain, or a functional variant or fragment thereof, wherein the TAL effector domain binds to a target sequence of about 15-20 nucleotides in a region of the genome spanning positions 55,037,859 to position 55,041,755, according to the hg38 reference genome for chr1. In some embodiments, the disclosure provides an expression repressor comprising a ZFN effector domain operably linked to an MQ1 effector domain, or a functional variant or fragment thereof, wherein the ZFN effector domain binds to a target sequence of about 15-20 nucleotides in a region of the genome spanning positions 55,037,859 to position 55,041,755, according to the hg38 reference genome for chr1. In some embodiments, the disclosure provides an expression repressor comprising a dCas9 effector domain described herein operably linked to an MQ1 effector domain, or a functional variant or fragment thereof, wherein the effector domain comprises a guide sequence that binds to a target sequence of about 15-20 nucleotides in a region of the genome spanning positions 55,037,859 to position 55,041,755, according to the hg38 reference genome for chr1.

In some embodiments, the disclosure provides an expression repressor comprising a TAL effector domain operably linked to a KRAB domain, or a functional variant or fragment thereof, wherein the TAL effector domain binds to a target sequence of about 15-20 nucleotides in a region of the genome spanning positions 55,037,859 to position 55,041,755, according to the hg38 reference genome for chr1. In some embodiments, the disclosure provides an expression repressor comprising a ZFN effector domain that binds a target sequence described herein operably linked to an KRAB domain, or a functional variant or fragment thereof, wherein the ZFN effector domain binds to a target sequence of about 15-20 nucleotides in a region of the genome spanning positions 55,037,859 to position 55,041,755, according to the hg38 reference genome for chr1. In some embodiments, the disclosure provides an expression repressor comprising a dCas9 effector domain comprising a guide sequence that binds a target sequence described herein operably linked to an KRAB domain, or a functional variant or fragment thereof, wherein the effector domain comprises a guide sequence that binds to a target sequence of about 15-20 nucleotides in a region of the genome spanning positions 55,037,859 to position 55,041,755, according to the hg38 reference genome for chr1.

In some embodiments, the disclosure provides an expression repressor comprising a TAL effector domain operably linked to an MQ1 effector domain, or a functional variant or fragment thereof, wherein the TAL effector domain binds to a target sequence of about 15-20 nucleotides in a region of the genome spanning position 55,038,000 to position 55,040,500, according to the hg38 reference genome for chr1. In some embodiments, the disclosure provides an expression repressor comprising a ZFN effector domain operably linked to an MQ1 effector domain, or a functional variant or fragment thereof, wherein the ZFN effector domain binds to a target sequence of about 15-20 nucleotides in a region of the genome spanning position 55,038,000 to position 55,040,500, according to the hg38 reference genome for chr1. In some embodiments, the disclosure provides an expression repressor comprising a dCas9 effector domain described herein operably linked to an MQ1 effector domain, or a functional variant or fragment thereof, wherein the effector domain comprises a guide sequence that binds to a target sequence of about 15-20 nucleotides in a region of the genome spanning position 55,038,000 to position 55,040,500, according to the hg38 reference genome for chr1.

In some embodiments, the disclosure provides an expression repressor comprising a TAL effector domain operably linked to a KRAB domain, or a functional variant or fragment thereof, wherein the TAL effector domain binds to a target sequence of about 15-20 nucleotides in a region of the genome spanning position 55,038,000 to position 55,040,500, according to the hg38 reference genome for chr1. In some embodiments, the disclosure provides an expression repressor comprising a ZFN effector domain that binds a target sequence described herein operably linked to an KRAB domain, or a functional variant or fragment thereof, wherein the ZEN effector domain binds to a target sequence of about 15-20 nucleotides in a region of the genome spanning position 55,038,000 to position 55,040,500, according to the hg38 reference genome for chr1. In some embodiments, the disclosure provides an expression repressor comprising a dCas9 effector domain comprising a guide sequence that binds a target sequence described herein operably linked to an KRAB domain, or a functional variant or fragment thereof, wherein the effector domain comprises a guide sequence that binds to a target sequence of about 15-20 nucleotides in a region of the genome spanning position 55,038,000 to position 55,040,500, according to the hg38 reference genome for chr1.

In some embodiments, the disclosure provides an expression repressor comprising a TAL effector domain operably linked to an MQ1 effector domain, or a functional variant or fragment thereof, wherein the TAL effector domain binds to a target sequence of about 15-20 nucleotides in a region of the genome spanning position 55,039,100 to position 55,039,600, according to the hg38 reference genome for chr1. In some embodiments, the disclosure provides an expression repressor comprising a ZFN effector domain operably linked to an MQ1 effector domain, or a functional variant or fragment thereof, wherein the ZFN effector domain binds to a target sequence of about 15-20 nucleotides in a region of the genome spanning position 55,039,100 to position 55,039,600, according to the hg38 reference genome for chr1. In some embodiments, the disclosure provides an expression repressor comprising a dCas9 effector domain described herein operably linked to an MQ1 effector domain, or a functional variant or fragment thereof, wherein the effector domain comprises a guide sequence that binds to a target sequence of about 15-20 nucleotides in a region of the genome spanning position 55,039,100 to position 55,039,600, according to the hg38 reference genome for chr1.

In some embodiments, the disclosure provides an expression repressor comprising a TAL effector domain operably linked to a KRAB domain, or a functional variant or fragment thereof, wherein the TAL effector domain binds to a target sequence of about 15-20 nucleotides in a region of the genome spanning position 55,039,100 to position 55,039,600, according to the hg38 reference genome for chr1. In some embodiments, the disclosure provides an expression repressor comprising a ZFN effector domain that binds a target sequence described herein operably linked to an KRAB domain, or a functional variant or fragment thereof, wherein the ZEN effector domain binds to a target sequence of about 15-20 nucleotides in a region of the genome spanning position 55,039,100 to position 55,039,600, according to the hg38 reference genome for chr1. In some embodiments, the disclosure provides an expression repressor comprising a dCas9 effector domain comprising a guide sequence that binds a target sequence described herein operably linked to an KRAB domain, or a functional variant or fragment thereof, wherein the effector domain comprises a guide sequence that binds to a target sequence of about 15-20 nucleotides in a region of the genome spanning position 55,039,100 to position 55,039,600, according to the hg38 reference genome for chr1.

In some embodiments, the disclosure provides an expression repressor comprising a TAL effector domain operably linked to an MQ1 effector domain, or a functional variant or fragment thereof, wherein the TAL effector domain binds to a target sequence having at least 90% identity to a sequence selected from SEQ ID NOs: 67-80. In some embodiments, the disclosure provides an expression repressor comprising a ZFN effector domain operably linked to an MQ1 effector domain, or a functional variant or fragment thereof, wherein the ZFN effector domain binds to a target sequence having at least 90% identity to a sequence selected from SEQ ID NOs: 67-80. In some embodiments, the disclosure provides an expression repressor comprising a dCas9 effector domain described herein operably linked to an MQ1 effector domain, or a functional variant or fragment thereof, wherein the effector domain comprises a guide sequence that binds to a target sequence having at least 90% identity to a sequence selected from SEQ ID NOs: 67-80.

In some embodiments, the disclosure provides an expression repressor comprising a TAL effector domain operably linked to a KRAB domain, or a functional variant or fragment thereof, wherein the TAL effector domain binds to a target sequence having at least 90% identity to a sequence selected from SEQ ID NOs: 67-80. In some embodiments, the disclosure provides an expression repressor comprising a ZFN effector domain that binds a target sequence described herein operably linked to an KRAB domain, or a functional variant or fragment thereof, wherein the ZFN effector domain binds to a target sequence having at least 90% identity to a sequence selected from SEQ ID NOs: 67-80. In some embodiments, the disclosure provides an expression repressor comprising a dCas9 effector domain comprising a guide sequence that binds a target sequence described herein operably linked to an KRAB domain, or a functional variant or fragment thereof, wherein the effector domain comprises a guide sequence that binds to a target sequence having at least 90% identity to a sequence selected from SEQ ID NOs: 67-80.

In some embodiments, the disclosure provides an expression repressor comprising a TAL effector domain operably linked to an MQ1 effector domain, or a functional variant or fragment thereof, wherein the TAL effector domain binds to a target sequence selected from SEQ ID NOs: 67-80. In some embodiments, the disclosure provides an expression repressor comprising a ZFN effector domain operably linked to an MQ1 effector domain, or a functional variant or fragment thereof, wherein the ZFN effector domain binds to a target sequence selected from SEQ ID NOs: 67-80. In some embodiments, the disclosure provides an expression repressor comprising a dCas9 effector domain described herein operably linked to an MQ1 effector domain, or a functional variant or fragment thereof, wherein the effector domain comprises a guide sequence that binds to a target sequence selected from SEQ ID NOs: 67-80.

In some embodiments, the disclosure provides an expression repressor comprising a TAL effector domain operably linked to a KRAB domain, or a functional variant or fragment thereof, wherein the TAL effector domain binds to a target sequence selected from SEQ ID NOs: 67-80. In some embodiments, the disclosure provides an expression repressor comprising a ZFN effector domain that binds a target sequence described herein operably linked to an KRAB domain, or a functional variant or fragment thereof, wherein the ZFN effector domain binds to a target sequence selected from SEQ ID NOs: 67-80. In some embodiments, the disclosure provides an expression repressor comprising a dCas9 effector domain comprising a guide sequence that binds a target sequence described herein operably linked to a KRAB domain, or a functional variant or fragment thereof, wherein the effector domain comprises a guide sequence that binds to a target sequence selected from SEQ ID NOs: 67-80.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 67. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:81. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 68. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:82. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 69. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:83. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 70. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:84. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 71. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:85. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 72. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:86. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 73. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:87. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 74. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:88. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 75. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:89. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 76. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:90. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 77. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:91. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 78. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:92. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 79. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:93. In some embodiments, the fusion protein comprises a linker sequence between the MQ1 effector domain and the TAL effector domain. In some embodiments, the linker sequence comprises an amino acid sequence selected from SEQ ID NOs: 55 and 62.

In some embodiments, an expression repressor of the present disclosure comprises a fusion protein comprising an MQ1 effector domain and a TAL effector domain, wherein the TAL effector domain targets a nucleotide sequence in the human PCSK9 gene set forth as SEQ ID NO: 80. In some embodiments, the MQ1 effector domain comprises an mRNA sequence as set forth in SEQ ID NO:46. In some embodiments, the MQ1 effector domain comprises an amino acid sequence as set forth in SEQ ID NO:47. In some embodiments, the TAL effector domain comprises an amino acid sequence as set forth in SEQ ID NO:94. In some embodiments, the fusion protein comprises a linker sequence between the MQ1 effector domain and the TAL effector domain. In some embodiments, the linker sequence comprises an amino acid sequence selected from SEQ ID NOs: 55 and 62.

### Expression Repressor Systems

In some embodiments, the disclosure provides an expression repression system comprising two or more expression repressors described herein. In some embodiments, an expression repression system comprises 2, 3, 4, 5, 6, 7, 8, 9, 10, 11, 12, or more expression repressors (and optionally no more than 15, 14, 13, 12, 11, 10, 9, 8, 7, 6, 5, 4, 3, or 2).

In some embodiments, the expression repressor system comprises a first expression repressor comprising (i) a first DNA targeting moiety that binds a first target sequence described herein, and (ii) a first effector domain; and at least one additional expression repressor comprising (i) a second DNA targeting moiety that binds a second target sequence described herein, and (ii) a second effector domain. In some embodiments, the first target sequence is different from the second target sequence.

In some embodiments, the expression repressor system comprises a first expression repressor comprising (i) a first DNA targeting moiety that binds a first target sequence described herein, and (ii) a first effector domain; and at least one additional expression repressor comprising (i) a second DNA targeting moiety that binds a second target sequence described herein, and (ii) a second effector domain, wherein the first target sequence is different from the second target sequence. In some embodiments, the first effector domain is the same as the second effector domain. In some embodiments, the first effector domain is different from the second effector domain.

In some embodiments, the expression repressor system comprises at least 1, 2, 3, 4, 5, 6, 7, 8, 9, or 10 additional expression repressors. In some embodiments, the expression repressor system comprises 1, 2, 3, 4, 5, 6, 7, 8, 9, or 10 additional expression repressors

In some embodiments, the expression repressor system comprises a first expression repressor comprising (i) a first DNA targeting moiety that binds a first target sequence described herein, and (ii) a first effector domain; and at least 1, 2, 3, 4, 5, 6, 7, 8, 9, or 10 additional expression repressors, wherein each of the additional expression repressors comprises (i) a DNA targeting moiety that binds a target sequence described herein; and (ii) an effector domain, wherein the target sequence of each of the additional expression repressors is different from one another and from the first target sequence. In some embodiments, the first effector domain and the effector domain of each of the additional expression repressors are the same or different.

In some embodiments, each of the expression repressors of the expression repressor system binds to a different target sequence described herein.

In some embodiments, each of the expression repressors of the expression repressor system are formulated in the same composition. In some embodiments, each of the expression repressors of the expression repressor system are formulated in different compositions.

In some embodiments, the expression repressors of an expression repressor system each comprise a different DNA targeting moiety (e.g., the first, second, third, or further expression repressors each comprise different targeting moieties from one another). For example, an expression repression system may comprise a first expression repressor and a second expression repressor wherein the first expression repressor comprises a first targeting moiety (e.g., a Cas9 domain, TAL effector domain, or Zn Finger domain), and the second expression repressor comprises a second targeting moiety (e.g., a Cas9 domain, TAL effector domain, or Zn Finger domain) different from the first targeting moiety. In some embodiments, different is comprising distinct types of targeting moiety, e.g., the first targeting moiety comprises a Cas9 domain, and the second DNA-targeting moiety comprises a Zn finger domain. In some embodiments, different is comprising distinct variants of the same type of targeting moiety, e.g., the first targeting moiety comprises a first Cas9 domain (e.g., from a first species) and the second targeting moiety comprises a second Cas9 domain (e.g., from a second species). In an embodiment, when an expression repressor system comprises two or more targeting moieties of the same type, e.g., two or more Cas9 or ZF domains, the targeting moieties specifically bind two or more different target sequences. For example, in an expression repressor system comprising two or more Cas9 domains, the two or more Cas9 domains may be chosen or altered such that they only appreciably bind the gRNA corresponding to their target sequence (e.g., and do not appreciably bind the gRNA corresponding to the target of another Cas9 domain). In a further example, in an expression repressor system comprising two or more effector moieties, the two or more effector moieties may be chosen or altered such that they only appreciably bind to their target sequence (e.g., and do not appreciably bind the target sequence of another effector moiety).

In some embodiments, an expression repressor system comprises three or more expression repressors and two or more expression repressors comprise the same DNA targeting moiety. For example, an expression repressor system may comprise three expression repressors, wherein the first and second expression repressors both comprise a first DNA targeting moiety and the third expression repressor comprises a second different DNA targeting moiety. For a further example, an expression repressor system may comprise four expression repressors, wherein the first and second expression repressors both comprise a first DNA targeting moiety and the third and fourth expression repressors comprises a second different DNA targeting moiety. For a further example, an expression repressor system may comprise five expression repressors, wherein the first and second expression repressors both comprise a first DNA targeting moiety, the third and fourth expression repressors both comprise a second different DNA targeting moiety, and the fifth expression repressor comprises a third different DNA targeting moiety. As described above, different can mean comprising different types of DNA-targeting moieties or comprising distinct variants of the same type of targeting moiety.

In some embodiments, the expression repressors of an expression repressor system each bind to a different target sequence described herein (e.g., the first, second, third, or further expression repressors each bind DNA sequences that are different from one another). For example, an expression repression system may comprise a first expression repressor and a second expression repressor wherein the first expression repressor binds to a first target sequence described herein, and the second expression repressor binds to a second target sequence described herein. In some embodiments, different can mean that: there is at least one position that is not identical between the target sequence bound by one expression repressor and the target sequence bound by another expression repressor, or that there is at least one position present in the target sequence bound by one expression repressor that is not present in the target sequence bound by another expression repressor.

In some embodiments, the expression repressors of an expression repressor system each comprise a different effector domain (e.g., the first, second, third, or further expression repressors each comprise a different effector domain from one another). For example, an expression repression system may comprise a first expression repressor and a second expression repressor wherein the first expression repressor comprises a first effector moiety (e.g., comprising a DNA methyltransferase or functional fragment thereof), and the second expression repressor comprises a second effector moiety (e.g., comprising a transcription repressor (e.g., KRAB) or functional fragment thereof) different from the first effector moiety. In some embodiments, different can mean comprising distinct types of effector moiety. In other embodiments, different can mean comprising distinct variants of the same type of effector moiety, e.g., the first effector moiety comprises a first DNA methyltransferase (e.g., having a first site specificity or amino acid sequence) and the second effector moiety comprises a second DNA methyltransferase (e.g., having a second site specificity or amino acid sequence).

In some embodiments, an expression repressor system comprises a first expression repressor comprising a first effector moiety and a second expression repressor comprising a second effector moiety, wherein the first effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB, MeCP2, HP1, RBBP4, REST, FOG1, SUZ12 or a functional variant or fragment thereof, and the second effector moiety comprises a different protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB, MeCP2, HP1, RBBP4, REST, FOG1, SUZ12, or a functional variant or fragment thereof.

In some embodiments, the first or second effector moiety comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof, and the other effector moiety comprises a transcription repressor activity (e.g., KRAB, MeCP2, HP1, RBBP4, REST, FOG1, SUZ12, or a functional variant or fragment of any thereof), the first or second effector moiety comprises a histone methyltransferase activity and the other effector moiety comprises a histone deacetylase activity (e.g., HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, the first or second effector moiety comprises a histone methyltransferase activity and the other effector moiety comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof). In some embodiments, the first or second effector moiety comprises a DNA methyltransferase activity and the other effector moiety comprises a transcription repressor activity. In some embodiments the first or second effector moiety comprises a histone methyltransferase activity and the other effector moiety comprises a transcription repressor activity (e.g., KRAB, MeCP2, HP1, RBBP4, REST, FOG1, SUZ12, or a functional variant or fragment of any thereof). In some embodiments, the first or second effector moiety comprises a transcription repressor activity and the other effector moiety comprises a different transcription repressor activity. In some embodiments, the first or second effector moiety comprises a DNA methyltransferase activity and the other effector moiety comprises the same DNA methyltransferase activity. In some embodiments, the first or second effector moiety comprises a DNA methyltransferase activity and the other effector moiety comprises a histone deacetylase activity. In some embodiments, the first or second effector moiety comprises a histone demethylase activity and the other effector moiety comprises a DNA methyltransferase activity.

In some embodiments, the first or second effector moiety comprises a histone methyltransferase activity and the other effector moiety comprises a DNA demethylase activity. In some embodiments, the first or second effector moiety comprises a histone demethylase activity and the other effector moiety comprises a transcription repressor activity. In some embodiments, the first or second effector moiety comprises a histone demethylase activity and the other effector moiety comprises a different histone demethylase activity. In some embodiments, the first or second effector moiety comprises a histone demethylase activity and the other effector moiety comprises the same histone demethylase activity. In some embodiments, the first or second effector moiety comprises a histone deacetylase activity and the other effector moiety comprises a DNA methyltransferase activity. In some embodiments, the first or second effector moiety comprises a histone deacetylase activity and the other effector moiety comprises a DNA demethylase activity. In some embodiments, the first or second effector moiety comprises a histone deacetylase activity and the other effector moiety comprises a transcription repressor activity. In some embodiments, the first or second effector moiety comprises a histone deacetylase activity and the other effector moiety comprises a different histone deacetylase activity. In some embodiments, the first or second effector moiety comprises a histone deacetylase activity and the other effector moiety comprises the same histone deacetylase activity. In some embodiments, the first or second effector moiety comprises a DNA methyltransferase activity and the other effector moiety comprises a DNA demethylase activity. In some embodiments, the first or second effector moiety comprises a DNA demethylase activity and the other effector moiety comprises a transcription repressor activity. In some embodiments, the first or second effector moiety comprises a DNA methyltransferase activity and the other effector moiety comprises a different DNA methyltransferase activity. In some embodiments, the first or second effector moiety comprises a DNA methyltransferase activity and the other effector moiety comprises the same DNA methyltransferase activity. In some embodiments, the first or second effector moiety comprises a DNA demethylase activity and the other effector moiety comprises a transcription repressor activity. In some embodiments, the first or second effector moiety comprises a DNA demethylase activity and the other effector moiety comprises a different DNA demethylase activity. In some embodiments, the first or second effector moiety comprises a DNA demethylase activity and the other effector moiety comprises the same DNA demethylase activity. In some embodiments, the first or second effector moiety comprises a transcription repressor activity and the other effector moiety comprises a different transcription repressor activity. In some embodiments, the first or second effector moiety comprises a transcription repressor activity and the other effector moiety comprises the same transcription repressor activity.

In some embodiments, an expression repressor system comprises three or more expression repressors and two or more expression repressors comprise the same DNA-targeting moiety. For example, an expression repressor system may comprise three expression repressors, wherein the first and second expression repressors both comprise a first effector moiety and the third expression repressor comprises a second different effector moiety. For a further example, an expression repressor system may comprise four expression repressors, wherein the first and second expression repressors both comprise a first effector moiety and the third and fourth expression repressors comprises a second different effector moiety. For a further example, an expression repressor system may comprise five expression repressors, wherein the first and second expression repressors both comprise a first effector moiety, the third and fourth expression repressors both comprise a second different effector moiety, and the fifth expression repressor comprises a third different effector moiety. As described above, different can mean comprising different types of effector moiety or comprising distinct variants of the same type of effector moiety.

In some embodiments, two or more (e.g., all) expression repressors of an expression repressor system are not covalently associated with each other, e.g., each expression repressor is not covalently associated with any other expression repressor. In another embodiment, two or more expression repressors of an expression repressor system are covalently associated with one another. In an embodiment, an expression repression system comprises a first expression repressor and a second expression repressor disposed on the same polypeptide, e.g., as a fusion molecule, e.g., connected by a peptide bond and optionally a linker. In some embodiments, the peptide is a self-cleaving peptide, e.g., a T2A self-cleaving peptide. In an embodiment, an expression repression system comprises a first expression repressor and a second expression repressor that are connected by a non-peptide bond, e.g., are conjugated to one another.

### Methods of Making Expression Repressors

In some embodiments, a protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007. For longer peptides, recombinant methods may be used. Methods of making a recombinant therapeutic polypeptide are routine in the art. See, e.g., Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing an expression repressor or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In some embodiments, large amounts of the expression repressor or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include, without limitation, CHO cells, COS cells, HeLA and BHK cell lines. Processes of host cell culture for production of protein therapeutics are described, for example, in Zhou and Kantardjieff (Eds.), Mammalian Cell Cultures for Biologies Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein. Compositions described herein may include a lipid nanoparticle encapsulating a vector, such as a viral vector, e.g., a lentiviral vector, encoding a recombinant protein. In some embodiments, a lipid nanoparticle encapsulating a vector, e.g., a viral vector, may comprise a nucleic acid encoding a recombinant protein.

Purification of protein therapeutics is described, for example, in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Proteins comprise one or more amino acids. Amino acids include any compound and/or substance that can be incorporated into a polypeptide chain, e.g., through formation of one or more peptide bonds. In some embodiments, an amino acid has the general structure H2N—C(H)I—COOH. In some embodiments, an amino acid is a naturally occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

### Nucleic Acids of the Disclosure

In another aspect, provided herein are nucleic acids encoding an expression repressor or an expression repressor system of the present disclosure. In some embodiments, an expression repressor may be provided via a composition comprising a nucleic acid encoding the expression repressor, wherein the nucleic acid is associated with sufficient other sequences to achieve expression in a system of interest (e.g., in a particular cell, tissue, organism, etc.).

In some embodiments, the present disclosure provides compositions of nucleic acids that encode an expression repressor or fragment thereof. In some embodiments, nucleic acids may be or may include DNA, RNA, or any other nucleic acid moiety or entity as described herein and may be prepared by any technology described herein or otherwise available in the art (e.g., synthesis, cloning, amplification, in vitro or in vivo transcription, etc.). In some embodiments, provided nucleic acids that encode an expression repressor or fragment thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, a composition for delivering an expression repressor or an expression repressor system described herein is or comprises a vector, e.g., a viral vector, comprising one or more nucleic acids encoding an expression repressor or one or more components of an expression repressor as described herein.

In some embodiments, the present disclosure provides compositions of nucleic acids that encode an expression repressor, one or more expression repressors, or fragments thereof. In some embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc.). The nucleic acid sequence may include, for example and without limitation, DNA, RNA, modified oligonucleotides (e.g., chemical modifications, such as modifications that alter the backbone linkages, sugar molecules, and/or nucleic acid bases), and artificial nucleic acids. In some embodiments, the nucleic acid sequence includes, for example and without limitation, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules. In some embodiments, provided nucleic acids encoding an expression repressor, one or more expression repressors, or polypeptide fragments thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, the nucleic acid sequence has a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

In some embodiments, a composition for delivering an expression repressor or an expression repressor system described herein is or comprises RNA, e.g., mRNA, comprising one or more nucleic acids encoding an expression repressor as described herein. In some embodiments, a composition for delivering an expression repressor or an expression repressor system described herein is or comprises RNA, e.g., mRNA, comprising one or more components of an expression repressor, as described herein.

In some embodiments, a nucleic acid of the disclosure comprises nucleosides, e.g., purines or pyrimidines, e.g., adenine, cytosine, guanine, thymine, and uracil. In some embodiments, the nucleic acid sequence includes one or more nucleoside analogs. The nucleoside analog includes, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain. Additional modifications are known and described, e.g., in WO 2012/019168; WO 2015/038892; WO 2015/038892; WO 2015/089511; WO 2015/196130; WO 2015/196118, and WO 2015/196128.

mRNA

In one aspect, provided herein is an RNA, e.g., an mRNA, encoding an expression repressor or an expression repressor system as described herein. In some embodiments, an mRNA comprises an open reading frame (ORF), e.g., a sequence of codons that is translatable into a peptide or protein, e.g., into an expression repressor or an expression repressor system.

### Open Reading Frames (ORFs)

An open reading frame includes a start codon at its 5′-end and a subsequent nucleotide region which usually exhibits a length which is a multiple of 3 nucleotides. In some embodiments, an ORF is terminated by a stop-codon (e.g., TAA, TAG, or TGA). In certain embodiments, the ORF may be isolated, or it may be incorporated in a longer nucleic acid sequence, e.g., in a vector or an mRNA. An ORF may also be known in the art as a protein coding region.

In some embodiments, an mRNA of the disclosure comprises an ORF, e.g., encoding a DNA targeting moiety and/or an effector domain of an expression repressor or an expression repressor system described herein. In certain embodiments, an ORF comprises a sequence that has been sequence optimized. Sequence-optimized nucleotide sequences disclosed herein are distinct from the corresponding wild-type nucleotide acid sequences and from other known sequence-optimized nucleotide sequences, e.g., these sequence-optimized nucleic acids have unique compositional characteristics.

In some embodiments, the mRNA comprises a bicistronic RNA. As used herein, a bicistronic RNA is typically an RNA, preferably an mRNA, comprising two ORFs. In some embodiments, the mRNA comprises a multicistronic RNA. As used herein, a multicistronic RNA is typically an RNA, preferably an mRNA, comprising more than two ORFs.

In some embodiments, the nucleic acid encoding the expression repressor system is a multicistronic sequence. In some embodiments, the multicistronic sequence is a bicistronic sequence. In some embodiments, the multicistronic sequence comprises a sequence encoding the first expression repressor and a sequence encoding the second expression repressor. In some embodiments, the multicistronic sequence encodes a self-cleavable peptide sequence, e.g., a 2A peptide sequence, e.g., a T2A peptide sequence or a P2A sequence. In some embodiments, the multicistronic sequence encodes a T2A peptide sequence and a P2A peptide sequence.

In some embodiments, a bicistronic construct further comprises a polyA tail. In some embodiments, upon transcription of a bicistronic gene construct, a single mRNA transcript encoding the first expression repressor, and the second expression repressor are produced, which upon translation gets cleaved, e.g., after the glycine residue within the 2A peptide, to yield the first expression repressor and the second expression repressor as two separate proteins. In some embodiments, the first and the second expression repressor are separated by “ribosome-skipping.” In some embodiments, the first expression repressor and/or the second expression repressor retains a fragment of the 2A peptide after ribosome skipping. In some embodiments, the expression level of the first and second expression repressor are equal. In some embodiments, the expression level of the first and the second expression repressor are different. In some embodiments, the protein level of the first expression repressor is within about 1%, 2%, 5%, or 10% of (greater than or less than) the protein level of the second expression repressor.

In some embodiments, a system encoded by a bicistronic nucleic acid decreases expression of a target gene (e.g., PCSK9) at least 1%, at least 2%, at least 3%, at least 4%, at least 5%, at least 10%, at least 20%, at least 30%, at least 40%, or at least 50%, in a cell, as compared to an otherwise similar system wherein the first and second expression repressor are encoded by monocistronic nucleic acids.

### Untranslated Regions (UTRs)

In certain embodiments, a polynucleotide (e.g., mRNA) encoding an expression repressor or an expression repressor system of the present disclosure further comprises a 5′ UTR and/or a translation initiation sequence. Natural 5′UTRs bear features which function in initiation of protein translation. They harbor signatures, e.g., Kozak sequences, which are commonly involved in ribosomal initiation of translation of many genes. 5′UTRs also may form secondary structures that function in elongation factor binding to further facilitate translation. The skilled person would recognize that engineering these features may enhance the stability and protein production of the polynucleotides of the disclosure. Untranslated regions useful in the design and manufacture of polynucleotides include, for example and without limitation, those disclosed in International Patent Publication No. WO 2014/164253 (see also US 2016/0022840).

Other non-UTR sequences may be used as regions or subregions within the polynucleotides. For example and without limitation, introns or fragments of introns sequences can be incorporated into regions of the polynucleotides. In some embodiments, incorporation of one or more intronic sequences may increase protein production and/or polynucleotide levels.

Combinations of features can be included in flanking regions and can be contained within other features. For example, an ORF can be flanked by a 5′ UTR which can contain a strong Kozak translational initiation signal and/or a 3′ UTR which can include an oligo (dT) sequence for templated addition of a poly-A tail. 5′UTR can comprise a first polynucleotide fragment and a second polynucleotide fragment from the same and/or different genes such as the 5′UTRs described in U.S. Patent Application Publication No. 2010/0293625.

A UTR, or a fragment thereof, can be placed in the same orientation as in the transcript from which it was selected, or can be altered in orientation and/or location. For example, a 5′ or 3′ UTR can be inverted, shortened, lengthened, or made with one or more other 5′ UTRs or 3′ UTRs. In some embodiments, a UTR sequence can be changed in some way relative to a reference sequence, e.g., an endogenous UTR. For example, a 3′ or 5′ UTR can be altered relative to a wild-type or native UTR by a change in orientation or location, by inclusion of additional nucleotides, deletion of nucleotides, or swapping or transposition of nucleotides.

In some embodiments, two copies of the same UTR are encoded either in series or substantially in series. In some embodiments, more than two copies of the same UTR are encoded either in series or substantially in series.

In some embodiments, flanking regions, e.g., flanking an ORF, can be heterologous. In some embodiments, a 5′ untranslated region can be derived from a different species than a 3′ untranslated region. The untranslated region can also include translation enhancer elements (TEE). For example and without limitation, TEEs are described in U.S. Patent Application Publication No. 2009/0226470.

In certain embodiments, a polynucleotide (e.g., an mRNA) encoding an expression repressor or an expression repressor system further comprises a 3′ UTR. A 3′-UTR is the section of mRNA immediately following the translation termination codon. In some embodiments, a 3′-UTR includes regulatory regions that post-transcriptionally influence gene expression. Such regulatory regions within a 3′-UTR can influence polyadenylation, translation efficiency, localization, and/or stability of the mRNA. In some embodiments, a 3′-UTR comprises a binding site for regulatory proteins and/or microRNAs. In some embodiments, the 3′-UTR has a silencer region, which binds to repressor proteins and inhibits the expression of the mRNA. In other embodiments, a 3′-UTR comprises an AU-rich element (ARE). Proteins may bind AREs to affect the stability and/or decay rate of mRNA. In some embodiments, a 3′-UTR comprises a sequence SEQ ID NO:54 that directs addition of adenine residues in a poly(A) tail to the end of the mRNA transcript.

### Terminal Modifications

In some embodiments, an mRNA described herein comprises one or more terminal modifications, e.g., a 5′Cap structure and/or a poly-A tail (e.g., between 100-200 nucleotides in length). The 5′ cap structure may be selected from the group consisting of CapO, Capl, ARCA, inosine, Nl-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contains a 5′ UTR comprising at least one Kozak sequence, and a 3′ UTR. Such modifications are known and are described, e.g., in WO 2012/135805 and WO 2013/052523. Additional terminal modifications are described, e.g., in WO 2014/164253, WO 2016/011306, WO 2012/045075, and WO 2014/093924.

The polynucleotide comprising an mRNA encoding an expression repressor or an expression repressor system of the present disclosure can further comprise a 5′ cap. The 5′ cap can bind the mRNA Cap Binding Protein (CBP), thereby increasing mRNA stability. The cap can further assist the removal of 5′ proximal introns removal during mRNA splicing.

In some embodiments, a polynucleotide comprising an mRNA encoding an expression repressor or an expression repressor system of the present disclosure comprises a non-hydrolyzable cap structure preventing decapping. In some embodiments, a non-hydrolyzable cap structure increases mRNA half-life. Cap structure hydrolysis requires cleavage of 5′-ppp-5′ phosphorodiester linkages; thus, modified nucleotides can be used during the capping reaction. Modified guanosine nucleotides may also be suitable for use in the present disclosure, e.g., a-thio-guanosine, α-methyl-phosphonate, and seleno-phosphate nucleotides.

In certain embodiments, a 5′ cap comprises 2′-0-methylation of the ribose sugars at 5 ‘-terminal and/or 5’-anteterminal nucleotides at the 2′-hydroxyl group of the sugar ring. In some embodiments, a cap may include cap analogs, i.e., synthetic cap analogs, chemical caps, chemical cap analogs, or structural/functional cap analogs differing from naturally occurring (i.e., endogenous, wild-type, or physiological) 5′-caps in chemical structure. Cap analogs may be chemically (i.e., non-enzymatically) or enzymatically synthesized and/or linked to the polynucleotides of the disclosure.

In certain embodiments, an mRNA encoding an expression repressor or an expression repressor system of the present disclosure can be capped after manufacture (e.g., IVT or chemical synthesis), using enzymes, to generate 5 ‘-cap structures.

In certain embodiments, 5’ terminal caps can include endogenous caps or cap analogs. In certain embodiments, a 5′ terminal cap can comprise a guanine analog. Suitable guanine analogs include, for example and without limitation, inosine, N1-methyl-guanosine, 2′fluoro-guanosine, 7-deaza-guanosine, 8-oxo-guanosine, 2-amino-guanosine, LNA-guanosine, and 2-azido-guanosine.

In some embodiments, an mRNA encoding an expression repressor or an expression repressor system of the present disclosure further comprises a poly A tail. In some embodiments, one or more terminal groups on the poly-A tail can be incorporated for stabilization. Such poly-A tails can also include structural moieties or 2′-0-methyl modifications, for example, as taught by Li et al. (2005) Current Biology 15:1501-1507.

In some embodiments, a poly-A tail when present is greater than 30 nucleotides in length. In some embodiments, a poly-A tail is greater than 35 nucleotides in length (e.g., at least about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides)

In some embodiments, a poly-A tail is designed relative to the length of the overall polynucleotide or the length of a particular region of the polynucleotide. For example, this can be based on the length of a coding region, the length of a particular feature or region, or based on the length of the product expressed from the polynucleotide. Accordingly, in some embodiments, a poly-A tail can be 10, 20, 30, 40, 50, 60, 70, 80, 90, or 100% greater in length than the polynucleotide or fragment thereof.

In some embodiments, one or more polynucleotides may be linked together by a Poly-A binding protein (PABP) by the 3′-end of the PABP, using modified nucleotides at the 3′-terminus of a poly-A tail.

In some embodiments, an mRNA encoding an expression repressor or an expression repressor of the present disclosure comprises, consists essentially of, or consists of a 5′ terminal cap, a 5′ UTR, an open reading frame (ORF), a 3′ UTR, and a polyA tail.

In some embodiments, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

### Recombinant Expression Vectors

Nucleic acids as described herein or nucleic acids encoding an expression repressor or an expression repressor system described herein may be incorporated into a vector. Vectors, including those derived from retroviruses such as lentivirus, are suitable tools to achieve long-term gene transfer since they allow long-term, stable integration of a transgene, and its propagation in daughter cells. Examples of suitable vectors include expression vectors, replication vectors, probe generation vectors, and sequencing vectors. In some embodiments, an expression vector may be provided to a cell in the form of a viral vector. Viral vector technology is well known in the art and described in a variety of virology and molecular biology manuals. Viruses that are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter and incorporating the construct into an expression vector. Vectors can be suitable for replication and integration in eukaryotes. Typical cloning vectors contain transcription and translation terminators, initiation sequences, and promoters useful for expression of the desired nucleic acid sequence. Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well.

In some embodiments, an expression repressor or an expression repressor system described herein acts at an enhancing sequence. In some embodiments, the enhancing sequence is an enhancer, a stretch enhancer, a shadow enhancer, a locus control region (LCR), or a super enhancer. In some embodiments, the super enhancer comprises a cluster of enhancers and other regulatory elements. In some embodiments, these sequences are located in a region 0.2-2 Mb upstream or downstream of a transcription start site. In some embodiments, the region is a non-coding region. In some embodiments, the region is associated with long-range regulation of a target gene, e.g., PCSK9. In some embodiments, the regions are cell-type specific. In some embodiments, a super-enhancer modifies (e.g., increases or decreases) target gene expression, e.g., PCSK9 expression, by recruiting the target gene promoter, e.g., PCSK9 promoter. In some embodiments, the super enhancer interacts with a target gene promoter, e.g., PCSK9 promoter, through an enhancer docking site. In some embodiments, the enhancer docking site is an anchor sequence. In some embodiments, the enhancer docking site is located at least 100 bp, 200 bp, 500 bp, 1000 bp, 1500 bp, 2000 bp, or 3000 bp away from the target gene promoter, e.g., PCSK9 promoter. In some embodiments, a super enhancer region is at least 100 bp, at least 200 bp, at least 300 bp, at least 500 bp, at least 1 kb, at least 2 kb, at least 3 kb, at least 5 kb, at least 10 kb, at least 15 kb, at least 20 kb, or at least 25 kb long.

Spacing between promoter elements frequently is flexible, so that promoter function is preserved when elements are inverted or moved relative to one another. For example, in a thymidine kinase (tk) promoter, spacing between promoter elements can be increased to about 50 bp apart before activity begins to decline. Without wishing to be bound by theory, it is hypothesized that depending upon the promoter, individual elements can function either cooperatively or independently to activate transcription.

One example of a suitable promoter for use in the present disclosure is the immediate early cytomegalovirus (CMV) promoter sequence. This promoter sequence is a strong constitutive promoter sequence capable of driving high levels of expression of any polynucleotide sequence operatively linked thereto. In some embodiments, a suitable promoter is Elongation Growth Factor-1a (EF-1a). Alternatively, other constitutive promoter sequences may also be used, including, but not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters including, but not limited to, an actin promoter, a myosin promoter, a hemoglobin promoter, and a creatine kinase promoter.

The present disclosure should not be interpreted to be limited to use of any particular promoter or category of promoters (e.g., constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to, a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neomycin, etc.

In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

### Cells

The present disclosure is further directed, in part, to cells comprising an expression repressor or expression repressor system described herein. Any cell, e.g., cell line, e.g., a cell line suitable for expression of a recombinant polypeptide, known to one of skill in the art is suitable to comprise an expression repressor or an expression repressor system described herein. In some embodiments, a cell, e.g., cell line, may be used to express an expression repressor or an expression repressor system, e.g., expression repressor(s), described herein. In some embodiments, a cell, e.g., cell line, may be used to express or amplify a nucleic acid, e.g., a vector, encoding an expression repressor or an expression repressor system, e.g., expression repressor(s), described herein. In some embodiments, a cell comprises a nucleic acid encoding an expression repressor or an expression repressor system, e.g., expression repressor(s), described herein.

In some embodiments, a cell comprises a first nucleic acid encoding a first component of an expression repressor system, e.g., a first expression repressor, and a second nucleic acid encoding a second component of the expression repressor system, e.g., a second expression repressor. In some embodiments, wherein a cell comprises nucleic acid encoding an expression repressor system comprising two or more expression repressors, the sequences encoding each expression repressor are disposed on separate nucleic acid molecules, e.g., on different vectors, e.g., a first vector encoding a first expression repressor and a second vector encoding a second expression repressor. In some embodiments, the sequences encoding each expression repressor are disposed on the same nucleic acid molecule, e.g., on the same vector. In some embodiments, some or all of the nucleic acid encoding the expression repressor system is integrated into the genomic DNA of the cell. In some embodiments, the nucleic acid encoding a first expression repressor of an expression repressor system is integrated into the genomic DNA of a cell, and the nucleic acid encoding a second expression repressor of an expression repressor system is not integrated into the genomic DNA of a cell (e.g., is situated on a vector). In some embodiments, the nucleic acid(s) encoding a first and a second expression repressor of an expression repressor system are integrated into the genomic DNA of a cell, e.g., at the same (e.g., adjacent or colocalized) or different sites in the genomic DNA.

Examples of cells that may comprise and/or express an expression repressor system or an expression repressor described herein include, but are not limited to, hepatocytes, neuronal cells, endothelial cells, myocytes, and lymphocytes.

### Methods of Making RNA

Methods of making and purifying modified RNAs are known and disclosed in the art. For example and without limitation, modified RNAs are made using in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667, and WO 2013/151736. Methods of purification include purifying an RNA transcript comprising a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified RMNA sample to DNAse treatment (WO 2014/152030).

Modified RNAs encoding proteins in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; and Tables 6, 185 and 186 of International Publication No WO 2013/151667. Any of the foregoing may be synthesized as an IVT polynucleotide, chimeric polynucleotide or a circular polynucleotide and linked to the polypeptide described herein, and each may comprise one or more modified nucleotides or terminal modifications.

In some embodiments, an expression repressor comprises or consists of a protein and may thus be produced by methods of making proteins as known in the art, for example, as provided in the present disclosure. In some embodiments, an expression repressor system, e.g., the expression repressor(s) of an expression repressor system, comprise one or more proteins and may thus be produced by methods of making proteins. As will be appreciated by one of skill in the art, methods of making proteins or polypeptides (which may be included in modulating agents as described herein) are routine in the art. See, e.g., Smales & James (Eds.), Therapeutic Proteins: Methods and Protocols (Methods in Molecular Biology), Humana Press (2005); see also Crommelin, Sindelar & Meibohm (Eds.), Pharmaceutical Biotechnology: Fundamentals and Applications, Springer (2013).

### Delivery

### Lipid Particles

Expression repressors or expression repressor systems as described herein can be delivered using any biological delivery system/formulation including a particle, for example, a nanoparticle delivery system. Nanoparticles include particles with a dimension (e.g. diameter) between about 1 and about 1000 nanometers, between about 1 and about 500 nanometers in size, between about 1 and about 100 nm, between about 30 nm and about 200 nm, between about 50 nm and about 300 nm, between about 75 nm and about 200 nm, between about 100 nm and about 200 nm, and any range therebetween. A nanoparticle has a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In some embodiments, nanoparticles have a greatest dimension ranging between 25 nm and 200 nm. Nanoparticles as described herein comprise delivery systems that may be provided in any form, including but not limited to solid, semi-solid, emulsion, or colloidal nanoparticles. A nanoparticle delivery system may include but is not limited to lipid-based systems, liposomes, micelles, micro-vesicles, exosomes, or gene gun. In one embodiment, the nanoparticle is a lipid nanoparticle (LNP). In some embodiments, the LNP is a particle that comprises a plurality of lipid molecules physically associated with each other by intermolecular forces.

In some embodiments, an LNP may comprise multiple components, e.g., 3-4 components. In one embodiment, the expression repressor or a pharmaceutical composition comprising said expression repressor (or a nucleic acid encoding the same, or pharmaceutical composition comprising said expression repressor nucleic acid) is encapsulated in an LNP. In one embodiment, the expression repressor system or a pharmaceutical composition comprising said expression repressor system (or a nucleic acid encoding the same, or pharmaceutical composition comprising said expression repressor system nucleic acid) is encapsulated in an LNP. In some embodiments, the nucleic acid encoding the first expression repressor and the nucleic acid encoding the second expression repressor are present in the same LNP. In some embodiments, the nucleic acid encoding the first expression repressor and the nucleic acid encoding the second expression repressor are present in different LNPs. Preparation of LNPs and the modulating agent encapsulation may be used/and or adapted from Rosin et al, Molecular Therapy, vol. 19, no. 12, pages 1286-2200, December 2011. In some embodiments, lipid nanoparticle compositions disclosed herein are useful for expression of a protein encoded by mRNA. In some embodiments, nucleic acids, when present in the lipid nanoparticles, are resistant in aqueous solution to degradation with a nuclease.

In some embodiments, the LNP formulations may include a CCD lipid, a neutral lipid, and/or a helper lipid. In some embodiments, the LNP formulation comprises an ionizable lipid. In some embodiments, an ionizable lipid may be a cationic lipid, an ionizable cationic lipid, or an amine-containing lipid that can be readily protonated. In some embodiments, the lipid is a cationic lipid that can exist in a positively charged or neutral form depending on pH. In some embodiments, the cationic lipid is a lipid capable of being positively charged, e.g., under physiological conditions. In some embodiments, the lipid particle comprises a cationic lipid in formulation with one or more of neutral lipids, ionizable amine-containing lipids, biodegradable alkyl lipids, steroids, phospholipids including polyunsaturated lipids, structural lipids (e.g., sterols), PEG, cholesterol, and polymer conjugated lipids.

In some embodiments, LNP formulation (e.g., MC3 and/or SSOP) includes cholesterol, PEG, and/or a helper lipid. The LNPs may be, e.g., microspheres (including unilamellar and multilamellar vesicles, lamellar phase lipid bilayers that, in some embodiments, are substantially spherical).

In some embodiments, the LNP can comprise an aqueous core, e.g., comprising a nucleic acid encoding an expression repressor or a system as disclosed herein and referred to herein as “cargo.” In some embodiments of the present disclosure, the cargo for the LNP formulation includes at least one guide RNA. In some embodiments, the cargo, e.g., a nucleic acid encoding an expression repressor, or a system as disclosed herein, may be adsorbed to the surface of an LNP, e.g., an LNP comprising a cationic lipid. In some embodiments, the cargo, e.g., a nucleic acid encoding an expression repressor, or a system as disclosed herein, may be associated with the LNP. In some embodiments, the cargo, e.g., a nucleic acid encoding an expression repressor, or a system as disclosed herein, may be encapsulated, e.g., fully encapsulated and/or partially encapsulated in an LNP.

In some embodiments, an LNP comprising a cargo may be administered for systemic delivery, e.g., delivery of a therapeutically effective dose of cargo that can result in a broad exposure of an active agent within an organism. Systemic delivery of lipid nanoparticles can be by any means known in the art including, for example and without limitation, intravenous, intraarterial, subcutaneous, and intraperitoneal delivery. In some embodiments, systemic delivery of lipid nanoparticles is by intravenous delivery. In some embodiments, an LNP comprising a cargo may be administered for local delivery, e.g., delivery of an active agent directly to a target site within an organism.

In some embodiments, an LNP may be locally delivered into a disease site, e.g., a tumor, or other target site, e.g., a site of inflammation, or to a target organ, e.g., the liver, lung, stomach, colon, pancreas, uterus, breast, lymph nodes, and the like. In some embodiments, an LNP as disclosed herein may be locally delivered to a specific cell, e.g., hepatocytes, stellate cells, Kupffer cells, endothelial, alveolar, and/or epithelial cells. In some embodiments, an LNP as disclosed herein may be locally delivered to a specific site, e.g., a tumor site, e.g., by subcutaneous or orthotopic administration. The LNPs may be formulated as a dispersed phase in an emulsion, micelles, or an internal phase in a suspension. In some embodiments, the LNPs are biodegradable. In some embodiments, the LNPs do not accumulate to cytotoxic levels or cause toxicity in vivo at a therapeutically effective dose. In some embodiments, the LNPs do not accumulate to cytotoxic levels or cause toxicity in vivo after repeat administrations at a therapeutically effective dose. In some embodiments, the LNPs do not cause an innate immune response that leads to a substantially adverse effect at a therapeutically effective dose.

In some embodiments, the LNP used comprises the formula (6Z,9Z,28Z,31Z)-heptatriacont-6,9,28,31-tetraene-19-yl 4-(dimethylamino)butanoate or ssPalm0-phenyl-P4C2 (ssPalmO-Phe, SS-OP). In some embodiments, the LNP formulation comprises the formula, (6Z,9Z,28Z,31Z)-heptatriacont-6,9,28,31-tetraene-19-yl4-(dimethylamino)butanoate (MC3), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., MC3 LNP or ssPalmO-phenyl-P4C2 (ssPalmO-Phe, SS-OP), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., SSOP-LNP.

Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011).

Vesicles can be made from several different types of lipids; however, phospholipids are most used to generate liposomes as drug carriers. Vesicles may comprise, for example and without limitation, DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see, for example, U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011). Extruded lipids can be prepared by extruding through filters of decreasing size, as described in Templeton et al, Nature Biotech, 15:647-652, 1997, the teachings of which relate to extruded lipid preparation are incorporated herein by reference.

### Viral Vectors

In some embodiments, viral vector systems which can be utilized with the methods and compositions described herein. Suitable viral vector systems for use include, for example and without limitation, (a) adenovirus vectors (e.g., an Ad5/F35 vector); (b) retrovirus vectors, including but not limited to lentiviral vectors (including integration competent or integration-defective lentiviral vectors), moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g., EPV and EBV vectors. See, e.g., U.S. Pat. Nos. 6,534,261; 6,607,882; 6,824,978; 6,933,113; 6,979,539; 7,013,219; and 7,163,824, the entire contents of each of which is incorporated by reference herein. Vectors, including those derived from retroviruses such as lentivirus, are suitable tools to achieve long-term gene transfer since they allow long-term, stable integration of a transgene and its propagation in daughter cells.

Examples of vectors include expression vectors, replication vectors, probe generation vectors, and sequencing vectors. In certain embodiments, an expression vector may be provided to a cell in the form of a viral vector. Viral vector technology is known in the art and described in a variety of virology and molecular biology manuals.

In some embodiments, a suitable viral vector for use in the present invention is an adeno-associated viral vector, such as a recombinant adeno-associated viral vector. Recombinant adeno-associated virus vectors (rAAV) are gene delivery systems based on the defective and nonpathogenic parvovirus adeno-associated type 2 virus. In some embodiments, the vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:91171702-3 (1998), Kearns et al., Gene Ther. 9:748-55 (1996)). AAV serotypes, including AAV1, AAV2, AAV3, AAV4, AAV5, AAV6, AAV7, AAV8 and AAV9, can be used in accordance with the present invention. Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently, the replication defective vector is propagated in a suitable cell system, e.g., HEK293 and variants thereof, that supply deleted gene function in trans.

Ad vectors can transduce multiple types of tissues in vivo, including nondividing, differentiated cells, such as those found in liver, kidney, and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:15-10 (1996); Sterman et al., Hum. Gene Ther. 9:71083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); and Sterman et al., Hum. Gene Ther. 7:1083-1089 (1998).

Packaging cells are used to form virus particles that are capable of infecting a host cell. Such cells include, for example and without limitation, HEK293 cells, and variants thereof, ψ2 cells, and PA317 cells. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. In some embodiments, viral DNA is packaged in a cell line, which contains a helper plasmid encoding the other AAV genes, namely rep and cap, but lacking ITR sequences. In certain embodiments, the cell line is also infected with adenovirus as a helper. The helper virus promotes replication of the AAV vector and expression of AAV genes from the helper plasmid. In certain embodiments, the helper plasmid is not packaged in significant amounts due to a lack of ITR sequences. In certain embodiments, contamination with adenovirus can be reduced by, e.g., heat treatment to which adenovirus is more sensitive than AAV.

### Methods of Use

### Epigenetic Modification

In some aspects, the present disclosure provides a method for introducing one or more epigenetic modifications (e.g., DNA methylation and/or histone modification) to a site in a region of the genome comprising PCSK9 (e.g., human PCSK9) in a cell or a population of cells, comprising contacting the cell or the population of cells with a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) an effector domain described herein (e.g., a DNA methyltransferase; a histone modifying enzyme).

In some aspects, the present disclosure provides a method for introducing one or more epigenetic modifications (e.g., DNA methylation and/or histone modification) to a site in a region of the genome comprising PCSK9 (e.g., human PCSK9) in a cell or a population of cells, comprising contacting the cell or the population of cells with a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) an effector domain described herein (e.g., a DNA methyltransferase; a histone modifying enzyme).

In some aspects, the present disclosure provides a method for introducing one or more epigenetic modifications to a site in a region of the genome comprising PCSK9 (e.g., human PCSK9) in a subject, comprising administering to the subject a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) an effector domain described herein.

In some aspects, the present disclosure provides a method of introducing one or more epigenetic modifications to a site in a region of the genome comprising PCSK9 (e.g., human PCSK9) in a subject, comprising administering to the subject a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) an effector domain described herein.

In some embodiments, epigenetic modification at the site is increased as compared to prior to the contacting or as compared to a control cell or control population of cells not contacted with the expression repressor or expression repressor system.

In some aspects, the present disclosure provides a method of increasing DNA methylation at a site in a region of the genome comprising PCSK9 (e.g., human PCSK9) in a cell or a population of cells, comprising contacting the cell or the population of cells with a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) a DNA methyltransferase.

In some aspects, the present disclosure provides a method of increasing DNA methylation at a site in a region of the genome comprising PCSK9 (e.g., human PCSK9) in a cell or a population of cells, comprising contacting the cell or the population of cells with a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) a DNA methyltransferase.

In some embodiments, increasing DNA methylation comprises increasing a percentage of methylated CpG dinucleotides in a region of the genome comprising a target gene.

In some aspects, the present disclosure provides a method of increasing DNA methylation at a site in a region of the genome comprising PCSK9 (e.g., human PCSK9) in a cell or a population of cells, comprising contacting the cell or the population of cells with a dose of an expression repressor described herein or a nucleic acid encoding the expression repressor (e.g., an LNP-formulated expression repressor or an LNP-formulated nucleic acid encoding the expression repressor), wherein a percentage of methylated CpG dinucleotides is maintained at the site, and wherein the expression repressor comprises (i) a DNA targeting moiety (e.g., a TALE, a ZFN, a dCas9/gRNA) that binds a target sequence described herein, and (ii) a DNA methyltransferase.

In some aspects, the present disclosure provides a method of increasing DNA methylation at a site in a region of the genome comprising PCSK9 (e.g., human PCSK9) in a cell or a population of cells, comprising contacting the cell or the population of cells with a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein a percentage of methylated CpG dinucleotides is maintained at the site, and wherein the at least one expression repressor comprises (i) a DNA targeting moiety (e.g., a TALE, a ZFN, a dCas9/gRNA) that binds a target sequence described herein, and (ii) a DNA methyltransferase.

Methods to measure DNA methylation are known in the art, including, but not limited to, mass spectrometry, methylation-specific PCR, sequencing based-assay such as bisulfite sequencing, the Hpall tiny fragment Enrichment by Ligation-mediated PCR (HELP) assay, GLAD-PCR assay, ChIP-on-chip assay, restriction landmark genomic scanning, methylated DNA immuneprecipitation, methyl sensitive southern blotting, high resolution Melt analysis, and methylation sensitive single nucleotide primer extension assay. In some embodiments, the method to measure DNA methylation of a target gene (e.g., PCSK9) comprises use of a DNA methylation microarray (e.g., an Illumina Methylation Array). Approaches for methylation analysis by microarray are described in Deatherage, et al (2009) Methods Mol Biol 556:117-139; Schumacher, et al (2006) Nucleic Acids Res 34:528-42; and Willhelm-Benartzi, et al (2013) Br J Cancer 109:1394-1402. In some embodiments, the method comprises a sequencing-based assay, wherein genomic DNA is treated with an agent prior to sequencing that converts cytosine residues to uracil (or another base having distinct hybridization properties from cytosine), but does not affect 5-methylcytosine residues. Exemplary agents are known in the art and include bisulfite, hydrogen sulfite, disulfite, and combinations thereof. Therefore, DNA treated with bisulfite retains the methylated cytosines, but not unmethylated cytosines. The treated DNA is then subjected to sequencing analysis (see, e.g., Campan et al (2009) Methods Mol Biol 507:325-37; Adusumalli, et al (2015) Brief Bioinform 16:369-79). Exemplary methods for sequencing analysis are known in the art and include use of next generation sequencing platforms based on sequencing-by-synthesis or sequencing-by-ligation as employed by Illumina, Life Technologies, and Roche; or based on nanopore sequencing or electronic-detection as employed by Ion Torrent technology. In some embodiments, the method to measure DNA methylation comprises enzymatic methyl-seq (EM-seq) (see, e.g., Vaisvila et al (2021) Genome Res 31:1280). In EM-seq, enzymatic reactions (e.g., performed using TET2 and T4-BGT) are used to convert 5-methylcytosine (5mC) and 5-hydroxymethylcytosine (i.e., the oxidation product of 5mC; also referred to as 5hmC) into products resistant to an enzymatic reaction that deaminates unmodified cytosines by converting them to uracils (e.g., performed using APOBEC3A). The enzymatically processed DNA is then amplified by PCR using EM-seq adaptor primers and subjected to sequencing analysis, e.g., using Illumina sequencing.

In some embodiments, DNA methylation at the site is increased as compared to prior to the contacting or as compared to a control cell or control population of cells not contacted with the expression repressor or expression repressor system.

In some embodiments, DNA methylation at the site is increased for an extended duration following the contacting. In some embodiments, DNA methylation at the site is increased for an extended duration following the contacting and prior to contacting the cell or the population of cells with a subsequent dose of the expression repressor or the expression repressor system.

In some embodiments, DNA methylation at the site is increased for an extended duration following the contacting and prior to contacting the cell or the population of cells with a subsequent dose of the expression repressor or the expression repressor system, wherein the extended duration is at least about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days. In some embodiments, the extended duration is at least about 21 days. In some embodiments, the extended duration is at least about 28 days.

In some embodiments, DNA methylation at the site is increased for an extended duration following the contacting and prior to contacting the cell or the population of cells with a subsequent dose of the expression repressor or the expression repressor system, wherein the extended duration is about 10 to about 100 days, about 20 days to about 90 days, about 20 days to about 80 days, about 20 days to about 70 days, about 20 days to about 60 days, about 25 days to about 75 days, about 25 days to about 65 days, about 30 days to about 100 days, about 30 days to about 90 days, about 30 days to about 80 days, or about 30 days to about 70 days. In some embodiments, the extended duration is about 21 days to about 100 days. In some embodiments, the extended duration is about 21 days to about 200 days. In some embodiments, the extended duration is about 28 days to about 100 days. In some embodiments, the extended duration is about 28 days to about 200 days.

In some embodiments, DNA methylation at the site is increased for an extended duration following the contacting and prior to contacting the cell or the population of cells with a subsequent dose of the expression repressor or the expression repressor system, wherein the extended duration is at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In some embodiments, the extended duration is at least about 3 weeks. In some embodiments, the extended duration is at least about 4 weeks.

In some embodiments, DNA methylation at the site is increased for an extended duration following the contacting and prior to contacting the cell or the population of cells with a subsequent dose of the expression repressor or the expression repressor system, wherein the extended duration is about 1 week to about 48 weeks, about 1 week to about 36 weeks, about 1 week to about 24 weeks, about 1 week to about 12 weeks, about 2 weeks to about 48 weeks, about 2 weeks to about 36 weeks, about 2 weeks to about 24 weeks, about 2 weeks to about 12 weeks, about 3 weeks to about 48 weeks, about 3 weeks to about 36 weeks, about 3 weeks to about 24 weeks, about 3 weeks to about 12 weeks, about 4 weeks to about 48 weeks, about 4 weeks to about 36 weeks, about 4 weeks to about 24 weeks, or about 4 weeks to about 12 weeks. In some embodiments, the extended duration is at least about 3 weeks to about 12 weeks. In some embodiments, the extended duration is at least about 3 weeks to about 24 weeks. In some embodiments, the extended duration is at least about 3 weeks to about 48 weeks. In some embodiments, the extended duration is at least about 4 weeks to about 12 weeks. In some embodiments, the extended duration is at least about 4 weeks to about 24 weeks. In some embodiments, the extended duration is at least about 4 weeks to about 48 weeks.

In some embodiments, DNA methylation at the site is increased for an extended duration following the contacting and prior to contacting the cell or the population of cells with a subsequent dose of the expression repressor or the expression repressor system, wherein the extended duration is at least 21 days.

In some embodiments, increased DNA methylation is maintained at the site for an extended duration following the contacting. In some embodiments, increased DNA methylation is maintained at the site for an extended duration following the contacting and prior to contacting the cell or the population of cells with a subsequent dose of the expression repressor or the expression repressor system.

In some embodiments, increased DNA methylation is maintained at the site for an extended duration following the contacting and prior to contacting the cell or the population of cells with a subsequent dose of the expression repressor or the expression repressor system, wherein the extended duration is at least about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days. In some embodiments, the extended duration is about 10 to about 100 days, about 20 days to about 90 days, about 20 days to about 80 days, about 20 days to about 70 days, about 20 days to about 60 days, about 25 days to about 75 days, about 25 days to about 65 days, about 30 days to about 100 days, about 30 days to about 90 days, about 30 days to about 80 days, or about 30 days to about 70 days.

In some embodiments, increased DNA methylation is maintained at the site for an extended duration following the contacting and prior to contacting the cell or the population of cells with a subsequent dose of the expression repressor or the expression repressor system, wherein the extended duration is at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks.

In some embodiments, increased DNA methylation is maintained at the site for an extended duration following the contacting and prior to contacting the cell or the population of cells with a subsequent dose of the expression repressor or the expression repressor system, wherein the extended duration is about 1 week to about 48 weeks, about 1 week to about 36 weeks, about 1 week to about 24 weeks, about 1 week to about 12 weeks, about 2 weeks to about 48 weeks, about 2 weeks to about 36 weeks, about 2 weeks to about 24 weeks, about 2 weeks to about 12 weeks, about 3 weeks to about 48 weeks, about 3 weeks to about 36 weeks, about 3 weeks to about 24 weeks, about 3 weeks to about 12 weeks, about 4 weeks to about 48 weeks, about 4 weeks to about 36 weeks, about 4 weeks to about 24 weeks, or about 4 weeks to about 12 weeks.

In some aspects, the disclosure provides a method of increasing DNA methylation at a site in a region of the genome comprising PCSK9 (e.g., human PCSK9) in a subject, comprising administering to the subject a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) a DNA methyltransferase.

In some aspects, the disclosure provides a method of increasing DNA methylation at a site in a region of the genome comprising PCSK9 (e.g., human PCSK9) in a subject, comprising administering to the subject a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) a DNA methyltransferase.

In some embodiments, DNA methylation at the site is increased as compared to prior to the administering or as compared to a control subject.

In some embodiments, DNA methylation at the site is increased for an extended duration following the administering. In some embodiments, DNA methylation at the site is increased for an extended duration following the administering and prior to administering to the subject a subsequent dose of the expression repressor or the expression repressor system.

In some embodiments, DNA methylation at the site is increased for an extended duration following the administering and prior to administering to the subject a subsequent dose of the expression repressor or the expression repressor system, wherein the extended duration is at least about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days. In some embodiments, the extended duration is about 10 to about 100 days, about 20 days to about 90 days, about 20 days to about 80 days, about 20 days to about 70 days, about 20 days to about 60 days, about 25 days to about 75 days, about 25 days to about 65 days, about 30 days to about 100 days, about 30 days to about 90 days, about 30 days to about 80 days, or about 30 days to about 70 days.

In some embodiments, DNA methylation at the site is increased for an extended duration following the administering and prior to administering to the subject a subsequent dose of the expression repressor or the expression repressor system, wherein the extended duration is at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks.

In some embodiments, DNA methylation at the site is increased for an extended duration following the administering and prior to administering to the subject a subsequent dose of the expression repressor or the expression repressor system, wherein the extended duration is about 1 week to about 48 weeks, about 1 week to about 36 weeks, about 1 week to about 24 weeks, about 1 week to about 12 weeks, about 2 weeks to about 48 weeks, about 2 weeks to about 36 weeks, about 2 weeks to about 24 weeks, about 2 weeks to about 12 weeks, about 3 weeks to about 48 weeks, about 3 weeks to about 36 weeks, about 3 weeks to about 24 weeks, about 3 weeks to about 12 weeks, about 4 weeks to about 48 weeks, about 4 weeks to about 36 weeks, about 4 weeks to about 24 weeks, or about 4 weeks to about 12 weeks.

In some embodiments, DNA methylation at the site is increased for an extended duration following the administering and prior to administering to the subject a subsequent dose of the expression repressor or the expression repressor system, wherein the extended duration is at least 21 days.

In some embodiments, the method increases DNA methylation at a site in a PCSK9 IGD (e.g., a human PCSK9 IGD). In some embodiments, the method increases DNA methylation at a site in the PCSK9 IGD (e.g., a human PCSK9 IGD), wherein the site is a span of at least about 300 bases, about 400 bases, about 500 bases, about 600 bases, about 700 bases, about 800 bases, about 900 bases, about 1,000 bases, about 1,200 bases, about 1,400 bases, about 1600 bases, about 1,800 bases, or about 2,000 bases. In some embodiments, the method increases DNA methylation at a site in a PCSK9 IGD (e.g., a human PCSK9 IGD), wherein the site is a span of at least about 300 bases, about 400 bases, about 500 bases, about 600 bases, about 700 bases, about 800 bases, about 900 bases, about 1,000 bases, about 1,200 bases, about 1,400 bases, about 1600 bases, about 1,800 bases, or about 2,000 bases, and wherein the site comprises a plurality of CpG sequences. In some embodiments, the method increases DNA methylation at a site in a PCSK9 IGD (e.g., a human PCSK9 IGD), wherein the site is a span of at least about 300 bases, about 400 bases, about 500 bases, about 600 bases, about 700 bases, about 800 bases, about 900 bases, about 1,000 bases, about 1,200 bases, about 1,400 bases, about 1600 bases, about 1,800 bases, or about 2,000 bases, and wherein the site comprises a frequency of CpG sequences that is higher than the average frequency of CpG sequences in the full genome or in a control region of the genome. In some embodiments, the method increases DNA methylation at a site in a PCSK9 IGD (e.g., a human PCSK9 IGD), wherein the site is a span of at least about 300 bases, about 400 bases, about 500 bases, about 600 bases, about 700 bases, about 800 bases, about 900 bases, about 1,000 bases, about 1,200 bases, about 1,400 bases, about 1600 bases, about 1,800 bases, or about 2,000 bases, and wherein the site comprises a CpG island.

In some aspects, the disclosure provides a method of increasing DNA methylation at a site in a region of the genome comprising PCKS9 (e.g., human PCSK9) in a cell or a population of cells, comprising contacting the cell or the population with a dose of an expression repressor described herein, or a nucleic acid encoding the expression repressor, wherein the expression repressor comprises (i) a DNA targeting moiety that binds a target sequence described herein, and (ii) a DNA methyltransferase, wherein the site is a span of at least about 300 bases, about 400 bases, about 500 bases, about 600 bases, about 700 bases, about 800 bases, about 900 bases, about 1,000 bases, about 1,100 bases, about 1,200 bases, about 1,300 bases, about 1,400 bases, about 1,500 bases, about 1,600 bases, about 1,700 bases, about 1,800 bases, about 1,900 bases, or about 2,000 bases comprising a CpG island, and wherein the average percentage of methylated CpG sequences in the CpG island is increased as compared to prior to the contacting or as compared to control cell or cell population. In some embodiments, the location (i.e., genomic coordinates relative to a reference genome) of the CpG island is identified using UCSC Genome Browser. In some embodiments, the target sequence is in or proximal to the CpG island (e.g., not more than about 500 to about 1,000 bases upstream or downstream the CpG island). In some embodiments, the average percentage of methylated CpG sequences in the CpG island is measured using EM-seq in the test cell or population of cells (i.e., the cell or the population contacted with the expression repressor or nucleic acid) as compared to a control cell or population of cells (e.g., a cell or population not contacted with the expression repressor or nucleic acid). In some embodiments, performing the EM-seq comprises amplifying an about 300-500 base region comprising the CpG island or a portion thereof, e.g., using PCR. In some embodiments, the amplified region is sequenced using next-generation sequencing, e.g., by Illumina, and the percentage of methylated CpG sequences in the amplified region is determined as an average across sequence reads. In some embodiments, the average percentage of methylated CpG sequences in the amplified region obtained from the test cell or population of cells is compared to that of the control cell or population of cells. In some embodiments, the increase in DNA methylation is presented as a fold-increase in average percentage of methylated CpG sequences in the amplified region between the test cell or population of cells and the control cell or population of cells.

In some embodiments, the method increases DNA methylation of CpG sequences at the site as compared to prior to the contacting or administering. In some embodiments, the method results in DNA methylation of at least about 20%, about 30%, about 40%, about 50%, about 60%, about 70%, about 80%, about 90%, or about 100% of CpG sequences at the site. In some embodiments, the method results in DNA methylation of about 20% of CpG sequences at the site. In some embodiments, the method results in DNA methylation of about 30% of CpG sequences at the site. In some embodiments, the method results in DNA methylation of about 40% of CpG sequences at the site. In some embodiments, the method results in DNA methylation of about 50% of CpG sequences at the site. In some embodiments, the method results in a frequency of methylated CpG sequences at the site that is at least about 5-fold, about 10-fold, about 15-fold, about 20-fold, about 25-fold, about 30-fold, about 35-fold, about 35-fold, about 40-fold, about 50-fold, about 60-fold, about 70-fold, about 80-fold, about 90-fold, or about 100-fold higher than prior to the contacting or the administering. In some embodiments, the method results in a frequency of methylated CpG sequences at the site that is about 10-fold to about 50-fold higher than prior to the contacting or the administering.

In some embodiments, the method increases DNA methylation at a site in a PCSK9 IGD (e.g., a human PCSK9 IGD), wherein the site is a span of at least about 300 bases, about 400 bases, about 500 bases, about 600 bases, about 700 bases, about 800 bases, about 900 bases, about 1,000 bases, about 1,200 bases, about 1,400 bases, about 1600 bases, about 1,800 bases, or about 2,000 bases, wherein the span comprises a CpG island, and wherein a plurality of the CpG sequences in the CpG island are methylated.

In some embodiments, the method increases DNA methylation at a site in a PCSK9 IGD (e.g., a human PCSK9 IGD), wherein the site is a span of at least about 500 bases, wherein the site comprises a CpG island, and wherein at least about 20%, about 30%, about 40%, or about 50% of the CpG sequences in the CpG island are methylated. In some embodiments, the method increases DNA methylation at a site in a PCSK9 IGD (e.g., a human PCSK9 IGD), wherein the site is a span of at least about 600 bases, wherein the site comprises a CpG island, and wherein at least about 20%, about 30%, about 40%, or about 50% of the CpG sequences in the CpG island are methylated. In some embodiments, the method increases DNA methylation at a site in a PCSK9 IGD (e.g., a human PCSK9 IGD), wherein the site is a span of at least about 700 bases, wherein the site comprises a CpG island, and wherein at least about 20%, about 30%, about 40%, or about 50% of the CpG sequences in the CpG island are methylated. In some embodiments, the method increases DNA methylation at a site in a PCSK9 IGD (e.g., a human PCSK9 IGD), wherein the site is a span of at least about 800 bases, wherein the site comprises a CpG island, and wherein at least about 20%, about 30%, about 40%, or about 50% of the CpG sequences in the CpG island are methylated. In some embodiments, the method increases DNA methylation at a site in a PCSK9 IGD (e.g., a human PCSK9 IGD), wherein the site is a span of at least about 900 bases, wherein the site comprises a CpG island, and wherein at least about 20%, about 30%, about 40%, or about 50% of the CpG sequences in the CpG island are methylated. In some embodiments, the method increases DNA methylation at a site in a PCSK9 IGD (e.g., a human PCSK9 IGD), wherein the site is a span of at least about 1,000 bases, wherein the site comprises a CpG island, and wherein at least about 20%, about 30%, about 40%, or about 50% of the CpG sequences in the CpG island are methylated.

In some embodiments, the site is in or near a promoter of PCSK9 (e.g., human PCSK9). In some embodiments, the site is in or near an enhancer of PCSK9 (e.g., human PCSK9). In some embodiments, the site is in PCSK9 (e.g., human PCSK9). In some embodiments, the site is in a non-coding region of PCSK9 (e.g., human PCSK9). In some embodiments, the site is in a coding region of PCSK9 (e.g., human PCSK9).

### Modulating Gene Expression

In another aspect, the present disclosure provides a method of modulating, e.g., decreasing, expression of a target gene, e.g., PCSK9. In some embodiments, the method comprises providing an expression repressor (or a nucleic acid encoding the same, or a pharmaceutical composition comprising said expression repressor nucleic acid) or an expression repressor system described herein (or a nucleic acid encoding the same, or pharmaceutical composition comprising said expression repressor system or nucleic acid), and contacting the target gene, e.g., PCSK9, and/or operably linked transcription control element(s) with the expression repressor or the expression repressor system. In some embodiments, modulating, e.g., decreasing expression of a target gene, e.g., PCSK9, comprises modulation of transcription of a target gene, e.g., PCSK9, as compared with a reference value, e.g., transcription of a target gene, e.g., PCSK9 in absence of the expression repressor or the expression repressor system. In some embodiments, the method of modulating, e.g., decreasing, expression of a target gene, e.g., PCSK9 are used ex vivo, e.g., on a cell from a subject, e.g., a mammalian subject, e.g., a human subject. In some embodiments, the methods of modulating, e.g., decreasing, expression of a target gene, e.g., PCSK9, are used in vivo, e.g., on a mammalian subject, e.g., a human subject. In some embodiments, the method of modulating, e.g., decreasing, expression of a target gene, e.g., PCSK9 are used in vitro, e.g., on a cell or cell line as described herein.

In some aspects, the present disclosure provides a method of modulating (e.g., decreasing) expression of PCSK9 (e.g., human PCSK9) in a cell or a population of cells, comprising contacting the cell or the population of cells with a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) an effector domain described herein (e.g., a DNA methyltransferase; a histone modifying enzyme).

In some aspects, the present disclosure provides a method of modulating (e.g., decreasing) expression of PCSK9 (e.g., human PCSK9) in a cell or a population of cells, comprising contacting the cell or the population of cells with a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) an effector domain described herein (e.g., a DNA methyltransferase; a histone modifying enzyme).

In some embodiments, expression of PCSK9 (e.g., human PCSK9) is decreased as compared to prior to the contacting or as compared to a control cell or control population of cells not contacted with the expression repressor or the expression repressor system.

In some aspects, the present disclosure provides a method of modulating (e.g., decreasing) expression of PCSK9 (e.g., human PCSK9) in a subject, comprising administering to the subject a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) an effector domain described herein (e.g., a DNA methyltransferase; a histone modifying enzyme).

In some aspects, the present disclosure provides a method of modulating (e.g., decreasing) expression of PCSK9 (e.g., human PCSK9) in a subject, comprising administering to the subject a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) an effector domain described herein (e.g., a DNA methyltransferase; a histone modifying enzyme).

In some embodiments, expression of PCSK9 (e.g., human PCSK9) is decreased in the subject (e.g., as measured in a tissue sample or serum obtained from the subject) as compared to prior to the administering or as compared to a control subject who has not received a dose of the expression repressor or the expression repressor system. In some embodiments, a level of a transcriptional or translation product of PCSK9 (e.g., human PCSK9) is decreased in the subject (e.g., as measured in a tissue sample or serum obtained from the subject) as compared to prior to the administering or as compared to a control subject who has not received a dose of the expression repressor or the expression repressor system. In some embodiments, expression of PCSK9 or the level of a transcriptional or translation product thereof is measured in a tissue sample obtained from the subject following administering of the dose of the expression repressor or the expression repressor system. in some embodiments, the tissue sample is a fresh, frozen, and/or preserved organ, biopsy, and/or aspirate obtained from the subject. In some embodiments, the tissue sample is blood or any blood constituent (e.g., plasma) collected from the subject. In some embodiments, expression of PCSK9 or the level of a transcriptional or translation product thereof as measured in the tissue sample is compared to expression or a level in a reference tissue sample obtained from the subject prior to the administering or from a control subject. Methods to measure expression of PCSK9 or the level of a transcriptional or translation product thereof are known in the art and include assays for measuring genomic DNA, mRNA, or cDNA (e.g., RNAseq, RT-PCR, real-time RT-PCR, competitive RT-PCR, northern blotting, nucleic acid microarray) and assays for measuring protein expression (e.g., quantitative immunofluorescence, flow cytometry, western blotting, ELISA, tissue immunostaining, immunoprecipitation, mass spectrometry, immunohistochemistry).

In some aspects, the disclosure provides a method of decreasing expression of PCSK9 (e.g., human PCSK9) in a cell or a population of cells, comprising contacting the cell or the population with a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) a DNA methyltransferase.

In some aspects, the disclosure provides a method of decreasing expression of PCSK9 (e.g., human PCSK9) in a cell or a population of cells, comprising contacting the cell or the population of cells with a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) a DNA methyltransferase.

In some embodiments, expression of PCSK9 (e.g., human PCSK9) is decreased as compared to prior to the contacting or as compared to a control cell or control population of cells not contacted with the expression repressor or the expression repressor system.

In some aspects, the disclosure provides a method of decreasing expression of PCSK9 (e.g., human PCSK9) in a cell or a population of cells, comprising contacting the cell or the population with a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) a DNA methyltransferase, and wherein expression of PCSK9 (e.g., human PCSK9) is decreased for an extended duration following the contacting and prior to contacting the cell or the population of cells with a subsequent dose of the expression repressor.

In some aspects, the disclosure provides a method of decreasing expression of PCSK9 (e.g., human PCSK9) in a cell or a population of cells, comprising contacting the cell or the population of cells with a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) a DNA methyltransferase, and wherein expression of PCSK9 (e.g., human PCSK9) is decreased for an extended duration following the contacting and prior to contacting the cell or the population of cells with a subsequent dose of the expression repressor system.

In some aspects, the disclosure provides a method of decreasing expression of PCSK9 (e.g., human PCSK9) in a subject, comprising administering to the subject a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) a DNA methyltransferase.

In some aspects, the disclosure provides a method of decreasing expression PCSK9 (e.g., human PCSK9) in a subject, comprising administering to the subject a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) a DNA methyltransferase.

In some aspects, the disclosure provides a method of decreasing expression of PCSK9 (e.g., human PCSK9) in a subject, comprising administering to the subject a dose of an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) a DNA methyltransferase, and wherein expression of PCSK9 (e.g., human PCSK9) or the level of a transcriptional or translation product thereof (e.g., as measured in a tissue sample obtained from the subject) is decreased for an extended duration following the administering and prior to administering to the subject a subsequent dose of the expression repressor (e.g., as compared to prior to the administering or as compared to expression of PCSK9 (e.g., human PCSK9) or the level of a transcriptional or translation product thereof as measured in a control subject).

In some aspects, the disclosure provides a method of decreasing expression PCSK9 (e.g., human PCSK9) in a subject, comprising administering to the subject a dose of an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) a DNA methyltransferase, and wherein expression of PCSK9 (e.g., human PCSK9) or the level of a transcriptional or translation product thereof (e.g., as measured in a tissue sample obtained from the subject) is decreased for an extended duration following the administering and prior to administering to the subject a subsequent dose of the expression repressor (e.g., as compared to prior to the administering or as compared to expression of PCSK9 (e.g., human PCSK9) or the level of a transcriptional or translation product thereof as measured in a control subject).

In some embodiments, the extended duration is at least about 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days. In some embodiments, the extended duration is at least about 21 days. In some embodiments, the extended duration is at least about 28 days. In some embodiments, the extended duration is about 10 to about 100 days, about 20 days to about 90 days, about 20 days to about 80 days, about 20 days to about 70 days, about 20 days to about 60 days, about 25 days to about 75 days, about 25 days to about 65 days, about 30 days to about 100 days, about 30 days to about 90 days, about 30 days to about 80 days, or about 30 days to about 70 days. In some embodiments, the extended duration is about 21 days to about 100 days. In some embodiments, the extended duration is about 21 days to about 200 days. In some embodiments, the extended duration is about 28 days to about 100 days. In some embodiments, the extended duration is about 28 days to about 200 days. In some embodiments, the extended duration is at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In some embodiments, the extended duration is at least about 3 weeks. In some embodiments, the extended duration is at least about 4 weeks. In some embodiments, the extended duration is about 1 week to about 48 weeks, about 1 week to about 36 weeks, about 1 week to about 24 weeks, about 1 week to about 12 weeks, about 2 weeks to about 48 weeks, about 2 weeks to about 36 weeks, about 2 weeks to about 24 weeks, about 2 weeks to about 12 weeks, about 3 weeks to about 48 weeks, about 3 weeks to about 36 weeks, about 3 weeks to about 24 weeks, about 3 weeks to about 12 weeks, about 4 weeks to about 48 weeks, about 4 weeks to about 36 weeks, about 4 weeks to about 24 weeks, or about 4 weeks to about 12 weeks. In some embodiments, the extended duration is at least about 3 weeks to about 12 weeks. In some embodiments, the extended duration is at least about 3 weeks to about 24 weeks. In some embodiments, the extended duration is at least about 3 weeks to about 48 weeks. In some embodiments, the extended duration is at least about 4 weeks to about 12 weeks. In some embodiments, the extended duration is at least about 4 weeks to about 24 weeks. In some embodiments, the extended duration is at least about 4 weeks to about 48 weeks.

The present disclosure is further directed, in another aspect, to a cell made by a method or process described herein. In some embodiments, the disclosure provides a cell produced by: providing an expression repressor or an expression repressor system described herein, providing the cell, and contacting the cell with the expression repressor (or a nucleic acid encoding the expression repressor, or a composition comprising said expression repressor or nucleic acid) or the expression repressor system (or a nucleic acid encoding the expression repressor system, or a composition comprising said expression repressor system or nucleic acid). In some embodiments, contacting a cell with an expression repressor comprises contacting the cell with a nucleic acid encoding the expression repressor under conditions that allow the cell to produce the expression repressor. In some embodiments, contacting a cell with an expression repressor comprises contacting an organism that comprises the cell with the expression repressor or a nucleic acid encoding the expression repressor under conditions that allow the cell to produce the expression repressor.

Without wishing to be bound by theory, it is hypothesized that a cell contacted with an expression repressor or an expression repressor system described herein may exhibit: a decrease in expression of a target gene (e.g., PCSK9) and/or a modification of epigenetic markers associated with the target gene, e.g., PCSK9, a transcription control element operably linked to the target gene, e: g., PCSK9, or an anchor sequence proximal to the target gene or associated with an anchor sequence-mediated conjunction operably linked to the target gene, e.g., PCSK9 compared to a similar cell that has not been contacted by the expression repressor or the expression repressor system. The decrease in expression and/or modification of epigenetic markers may persist, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after contact with the expression repressor or the expression repressor system. In certain embodiments, the epigenetic modification comprises methylation, e.g., DNA methylation or histone methylation.

In some embodiments, a cell previously contacted by an expression repressor or expression repressor system retains the decrease in expression and/or modification of epigenetic markers after the expression repressor or the expression repressor system is no longer present in the cell, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after the expression repressor or the expression repressor system is no longer present in the cell.

Methods and compositions as provided herein may treat a condition associated with overexpression or mis-regulation of a target gene, e.g., PCSK9 by stably or transiently altering (e.g., decreasing) transcription of a target gene, e.g., PCSK9. In some embodiments, such a modulation persists for at least about 1 hour to about 30 days, or at least about 2 hours, 6 hours, 12 hours, 18 hours, 24 hours, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or at least 5 years (e.g., permanently or indefinitely). Optionally, such a modulation persists for no more than 10, 9, 8, 7, 6, 5, 4, 3, 2, or 1 years.

In some embodiments, a method or composition provided herein may decrease expression of a target gene, e.g., PCSK9, in a cell by at least 10, 20, 30, 40, 50, 60, 70, 80, 90, or 100% (and optionally up to 100%) relative to expression of the target gene in a cell not contacted by the composition or treated with the method.

In some embodiments, a method provided herein is used to modulate, e.g., decrease, expression of a target gene, e.g., PCSK9, by disrupting a genomic complex, e.g., an anchor sequence-mediated conjunction, associated with said target gene. In some embodiments, modulating expression of a gene, e.g., PCSK9, comprises altering accessibility of a transcriptional control sequence to a gene, e.g., PCSK9. A transcriptional control sequence, whether internal or external to an anchor sequence-mediated conjunction, can be an enhancing sequence or a silencing (or repressive) sequence.

In some embodiments, such provided technologies may be used to treat a gene mis-regulation disorder, e.g., a PCSK9 gene mis-regulation disorder, e.g., a symptom associated with a PCSK9 gene mis-regulation in a subject, e.g., a patient, in need thereof. In some embodiments, such provided technologies may be used to treat a PCSK9 gene mis-regulation disorder or a symptom associated with a PCSK9 gene mis-regulation disorder in a subject, e.g., a patient, in need thereof. In some embodiments, the disorder is associated with PCSK9 mis-regulation, e.g., PCSK9 overexpression. In some embodiments, such provided technologies may be used to methylate the promoter of a target gene, e.g., PCSK9, to treat a gene mis-regulation disorder, e.g., PCSK9 gene mis-regulation disorder, e.g., a symptom associated with a PCSK9 gene mis-regulation in a subject, e.g., a patient, in need thereof. In some embodiments, such provided technologies may selectively affect the viability of a cell which aberrantly expresses a polypeptide encoded by a target gene, e.g., PCSK9.

In some embodiments, the disclosure provides a method of modulating (e.g., decreasing) expression of PCSK9 in a subject having a disorder associated with dysregulation (e.g., overexpression) of PCSK9 in a subject, comprising administering to the subject an expression repressor described herein or a nucleic acid encoding the expression repressor (e.g., an LNP-formulated expression repressor or an LNP-formulated nucleic acid encoding the expression repressor), wherein the expression repressor comprises (i) a DNA targeting moiety (e.g., a TALE, a ZFN, a dCas9/gRNA) that binds a target sequence described herein, and (ii) an effector domain described herein. In some embodiments, the disclosure provides a method of modulating (e.g., decreasing) expression of PCSK9 in a subject having a disorder associated with dysregulation (e.g., overexpression) of PCSK9 in a subject an expression repressor system described herein comprising at least one expression repressor (e.g., 1, 2, 3, 4, 5 or more expression repressors described herein) or a nucleic acid encoding the at least one expression repressor (e.g., an expression repressor system comprising at least one LNP-formulated expression repressor or an LNP-formulated nucleic acid encoding the at least one expression repressor), wherein the at least one expression repressor comprises (i) a DNA targeting moiety (e.g., a TALE, a ZFN, a dCas9/gRNA) that binds a target sequence described herein, and (ii) an effector domain described herein.

In some embodiments, the disorder associated with dysregulation (e.g., overexpression) of PCSK9 is a metabolic disorder. In some embodiments, the disorder is a hepatic disorder. In some embodiments, the disorder comprises alcohol misuse. In some embodiments, the disorder is a neurological disorder (e.g., Alzheimer's Disease). In some embodiments, the disorder is an inflammatory disorder. In some embodiments, the disorder comprises a viral infection. In some embodiments, the disorder comprises a pulmonary disorder. In some embodiments, the disorder comprises a neoplasia.

In some embodiments, such provided technologies may be used to treat a hepatic disorder or a disorder, e.g., a symptom associated with a hepatic disorder in a subject, e.g., a patient, in need thereof. In some embodiments, such provided technologies may be used to treat a pulmonary disorder or a disorder, e.g., a symptom associated with a hepatic disorder in a subject, e.g., a patient, in need thereof. In some embodiments, such provided technologies may be used to treat a neoplasia, e.g., a disorder or, a symptom associated with a neoplasia in a subject, e.g., a patient, in need thereof. In some embodiments, such provided technologies may be used to treat a viral infection related disorder, e.g., a disorder or a symptom associated with viral infection related disorder in a subject, e.g., a patient, in need thereof. In some embodiments, such provided technologies may be used to treat an alcohol misuse related disorder, e.g., a disorder or a symptom associated with an alcohol misuse related disorder in a subject, e.g., a patient, in need thereof. In some embodiments, such provided technologies may be used to treat a neurological disease or disorder, e.g., Alzheimer's Disease, in a subject, e.g., a patient, in need thereof. In some embodiments, such provided technologies may be used to treat a neoplasia disorder associated with a viral infection or alcohol misuse, e.g., a disorder or a symptom associated with a neoplasia disorder that is associated with a viral infection or alcohol misuse in a subject, e.g., a patient, in need thereof.

In some embodiments, the condition to be treated is a hepatic disease or disorder. In some embodiments the condition treated is a hepatocellular disease or disorder. In some embodiments the condition treated is associated with PCSK9 mis-regulation, e.g., PCSK9 overexpression. In some embodiments the condition treated is a chronic disease. In some embodiments the condition treated is a chronic liver disease. In some embodiments the condition treated is a viral infection. In some embodiments, the condition treated is an alcohol misuse associated disorder. In some embodiments the condition treated is a pulmonary disease. In some embodiments the condition treated is associated with PCSK9 mis-regulation, e.g., PCSK9 overexpression. In some embodiments the condition treated is a chronic disease. In some embodiments the condition treated is a chronic pulmonary disease. In some embodiments, such provided technologies may be used to treat or reduce liver cancer growth, metastasis, drug resistance, and/or cancer stem cell (CSC) maintenance. In some embodiments, the condition treated is a carcinoma, e.g., non-small cell lung cancer (NSCLC). In some embodiments, the chronic pulmonary disease is associated with tobacco misuse.

In some embodiments, the condition to be treated is a hepatocarcinoma (HCC). In certain embodiments, the cancer hepatocarcinoma is subtype SI (HCC SI), hepatocarcinoma subtype S2 (HCC S2), or hepatocarcinoma subtype S3 (HCC S2). In some embodiments, the HCC subtype is associated with PCSK9 overexpression. In some embodiments, the cancer is HCC SI or HCC S2. In some embodiments, the cancer subtype is associated with an aggressive tumor and poor clinical outcome.

In some embodiments, the disclosure provides a treatment regimen that may be devised for the subject on the basis of the HCC subtype in the subject, e.g., a personalized approach to tailor the aggressiveness of treatment based on HCC subtype on a subject. In some embodiments, the disclosure provides a method of treatment using the expression repressor or expression repressor systems disclosed herein, the method comprising, identifying the HCC subtype in a patient and determine a dosage and administration schedule of said expression repressor and/or expression repressor systems based on the HCC subtype identification.

Methods are described herein to deliver agents, or a composition as disclosed herein, to a subject for treatment of a disorder such that the subject suffers minimal side effects or systemic toxicity in comparison to an alternative, e.g., standard of care, treatment. In some embodiments, the subject does not experience any significant side effects typically associated with standard of care, when treated with the agents and/or compositions described herein. In some embodiments, the subject does not experience a significant side effect including but not limited to alopecia, nausea, vomiting, poor appetite, soreness, neutropenia, anemia, thrombocytopenia, dizziness, fatigue, constipation, oral ulcers, itchy skin, peeling, nerve and muscle damage, auditory changes, weight loss, diarrhea, immunosuppression, bruising, heart damage, bleeding, liver damage, kidney damage, edema, mouth and throat sores, infertility, fibrosis, epilation, moist desquamation, mucosal dryness, vertigo and encephalopathy when treated with the agents and/or compositions described herein. In some embodiments, the subject does not show a significant loss of body weight when treated with the agents and/or compositions described herein. The agents and compositions described herein can be administered to a subject, e.g., a mammal, e.g., in vivo, to treat or prevent a variety of disorders as described herein. This includes disorders involving cells characterized by altered expression patterns of PCSK9.

### Therapeutic Methods

In some aspects, provided herein is a method of treating a disease or disorder associated with PCSK9 expression.

In some aspects, the disclosure provides a method of treating a disease or disorder associated with PCSK9 expression in a subject, comprising administering to the subject an expression repressor described herein or a nucleic acid encoding the expression repressor (e.g., an LNP-formulated expression repressor, an LNP-formulated nucleic acid encoding the expression repressor, or a pharmaceutical composition comprising the expression repressor, the LNP-formulated expression repressor, the nucleic acid, or the LNP-formulated nucleic acid), wherein the expression repressor comprises (i) a DNA targeting moiety (e.g., a TALE, a ZFN, a dCas9/gRNA) that binds a target sequence described herein, and (ii) an effector domain described herein.

In some aspects, the disclosure provides a method of treating a disease or disorder associated with PCSK9 expression in a subject, comprising administering to the subject an expression repressor system described herein comprising at least one expression repressor (e.g., 1, 2, 3, 4, 5 or more expression repressors described herein) or a nucleic acid encoding the at least one expression repressor (e.g., an expression repressor system comprising a pharmaceutical composition comprising the at least one expression repressor or the nucleic acid, e.g., an expression repressor system comprising at least one LNP-formulated expression repressor or an LNP-formulated nucleic acid encoding the at least one expression repressor, or pharmaceutical compositions thereof), wherein the at least one expression repressor comprises (i) a DNA targeting moiety (e.g., a TALE, a ZFN, a dCas9/gRNA) that binds a target sequence described herein, and (ii) an effector domain described herein.

In some aspects, the disclosure provides a method of treating a disease or disorder associated with dysregulated PCSK9 expression (e.g., PCSK9 overexpression) in a subject, comprising administering to the subject an expression repressor described herein or a nucleic acid encoding the expression repressor (e.g., an LNP-formulated expression repressor, an LNP-formulated nucleic acid encoding the expression repressor, or a pharmaceutical composition comprising the expression repressor, the LNP-formulated expression repressor, the nucleic acid, or the LNP-formulated nucleic acid), wherein the expression repressor comprises (i) a DNA targeting moiety (e.g., a TALE, a ZFN, a dCas9/gRNA) that binds a target sequence described herein, and (ii) an effector domain described herein. In some aspects, the disclosure provides a method of treating a disease or disorder associated with PCSK9 expression (e.g., PCSK9 overexpression) in a subject, comprising administering to the subject an expression repressor system described herein comprising at least one expression repressor (e.g., 1, 2, 3, 4, 5 or more expression repressors described herein) or a nucleic acid encoding the at least one expression repressor (e.g., an expression repressor system comprising a pharmaceutical composition comprising the at least one expression repressor or the nucleic acid, e.g., an expression repressor system comprising at least one LNP-formulated expression repressor or an LNP-formulated nucleic acid encoding the at least one expression repressor, or pharmaceutical compositions thereof), wherein the at least one expression repressor comprises (i) a DNA targeting moiety (e.g., a TALE, a ZFN, a dCas9/gRNA) that binds a target sequence described herein, and (ii) an effector domain described herein. In some embodiments, the disease or disorder is due to a genetic mutation in PCSK9.

In some embodiments, the disease or disorder is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and combinations thereof. Other conditions associated with PCSK9 are known in the art.

In another aspect, the present disclosure provides a method of treating a condition associated with mis-regulation, e.g., over-expression of a target gene, e.g., PCSK9, in a subject, comprising administering to the subject an expression repressor (or a nucleic acid encoding the same, or a pharmaceutical composition comprising said expression repressor nucleic acid) or an expression repressor system described herein (or a nucleic acid encoding the same, or pharmaceutical composition comprising said expression repressor system or nucleic acid). Conditions associated with overexpression of particular genes are known to those of skill in the art. Such conditions include, but are not limited to, metabolic disorders, cancer (e.g., solid tumors), and hepatitis.

In some embodiments, the disease or disorder to be treated is a liver disease. In some embodiments, the disease or disorder to be treated is a disease associated with a blood or serum ratio of high density lipoprotein (HDL)-cholesterol to low density lipoprotein (LDL)-cholesterol (HDL/LDL) of <0.3. The ratio of HDL-cholesterol and LDL-cholesterol can be determined by any appropriate lipid panel or assay known in the art. Such panels and assays are generally known to one of skill in the art. In certain embodiments, the disease is selected from the group consisting of non-alcoholic fatty liver disease (NAFLD), non-alcoholic steatohepatitis (NASH), alcoholic liver disease (ALD), and a high LDL-cholesterol associated disease. Ratios of HDL-cholesterol to LDL-cholesterol are determined after measuring both HDL and LDLs cholesterol and comparing the levels of HLD to LDL. An HDL/LDL cholesterol ratio of greater than 0.3 is generally considered a healthy ratio. An HDL/LDL cholesterol ratio of less than or equal to (<) 0.3 is generally considered an unhealthy ratio. In some embodiments, the compound that modulates PCSK9 or ANGPTL3 expression is administered to a subject with an HDL/LDL ratio of less than 0.3, less than 0.25, less than 0.2, less than 0.15, less than 0.10, less than 0.5, less than 0.1, less than 0.5, and less than 0.01. In some embodiments, the compound that modulates PCSK9 expression is administered to a subject with an HDL/LDL ratio of between about 0.01-0.3, between about 0.01-0.5, between about 0.5-0.1, between about 0.1-0.15, between about 0.15-0.2, between about 0.2-0.25, and between about 0.25-0.3.

In some embodiments, the low-density lipoprotein (LDL) cholesterol disease to be treated is a liver disease such as non-alcoholic steatohepatitis (NASH), non-alcoholic fatty liver disease (NAFLD), and/or alcoholic liver disease (ALD).

In some embodiments, the high LDL-cholesterol associated disease to be treated occurs in a subject having a PCSK9-activating (GOF) mutation, a marked elevation of low-density lipoprotein particles in the plasma, primary hypercholesterolemia, or a heterozygous Familial Hypercholesterolemia (heFH).

PCSK9 mutations, such as those resulting in PCSK9 gain of function and loss of function mutations, are described in, for example, “Loss- and Gain-of-function PCSK9 Variants”, Benjannet S, et al., J Biol Chem. 2012 Sep. 28; 287(40): 33745-33755 and “Mutations and polymorphisms in the proprotein convertase subtilisin kexin 9 (PCSK9) gene in cholesterol metabolism and disease” Abifadel M, et al, Hum Mutat. 2009 April; 30(4): 520-9. doi: 10.1002/humu.20882, both of which are hereby incorporated by reference in their entirety. PCSK9 gain of function mutations include, but are not limited to, L108R, D374Y, D374H, D374W, D374M, D374F, D374E, D374K, and D374L.

Hypercholesterolemia is characterized by high levels of cholesterol in the blood. Subjects with high levels of cholesterol can develop heart disease, e.g., coronary artery disease. When excess cholesterol in the blood is deposited on the walls of blood vessels, the abnormal buildup of cholesterol forms plaques that narrow and harden the blood vessels and arteries. This build-up causes chest pain and increases a person's risk of having a heart attack. Hypercholesterolemia, includes, for example and without limitation, heterozygous Familial Hypercholesterolemia (heFH), homozygous Familial Hypercholesterolemia (hoFH), Autosomal Dominant Hypercholesterolemia (ADH, e.g., ADH associated with one or more gain-of-function mutations in the PCSK9 gene), as well as incidences of hypercholesterolemia that are distinct from Familial Hypercholesterolemia (nonFH). Familial Hypercholesterolemia (FH) is an inherited genetic disorder that results in high cholesterol levels and heart disease, heart attacks, or strokes. Patients with FH have elevated serum low-density lipoprotein (LDL) cholesterol levels. Heterozygous HF (heHF) is more common than homozygous HF (HoHF). Exemplary genetics and diagnosis are discussed in “Familial hypercholesterolemia: A review,” Varghese M J et al, Ann Pediatr Cardiol. 2014 May-August; 7(2): 107-117, hereby incorporated by reference in its entirety.

In some embodiments, a patient who is treatable by a method disclosed herein has a hyperlipidemia, including hypercholesterolemia (sometimes referred to herein as “a hypercholesterolemic patient”). “Hypercholesterolemia,” as used herein, includes a serum LDL-C concentration of greater than or equal to 70 mg/dL, or a serum LDL-C concentration greater than or equal to 100 mg/dL, depending on the patient's cardiovascular risk (“CV risk”). For example, in some embodiments, the patient is regarded as having hypercholesterolemia if the patient's serum LDL-C concentration is greater than or equal to about 70 mg/dL. For patients with moderate or high CV risk, the patient is regarded as having hypercholesterolemia if the patient's serum LDL-C concentration is greater than or equal to about 100 mg/dL.

In some embodiments, a method is provided for reducing serum LDL-C levels in a patient in need thereof, e.g., increasing LDL receptor mediated clearance of LDL cholesterol. In some embodiments, a method as disclosed herein decreases circulating cholesterol. The patient may be a hypercholesterolemic, e.g., statin intolerant patient, or any other patient for whom a reduction in serum LDL-C is deemed beneficial or desirable. In some embodiments, the present disclosure includes methods for reducing serum LDL-C levels in a patient without inducing skeletal muscle pain, discomfort, weakness, or cramping. As used in this context, “reducing serum LDL-C levels” means causing the patient's serum LDL-C level to decrease by at least 10% (e.g., at least 15%, 20%, 25%, 30%, 35%, 40%, 45%, 50%, 55%, 60%, 65%, 70%, 75% or more).

In some embodiments, a method as provided herein decreases PCSK9 mRNA levels in the subject treated with an expression repressor or an expression repressor system of the present disclosure. In some embodiments, a method as provided herein decreases PCSK9 mRNA levels in vitro following treatment with an expression repressor or an expression repressor system of the present disclosure. In certain embodiments, PCSK9 mRNA is decreased at least 10%, at least 20%, at least 30%, at least 40%, at least 50%, at least 60%, at least 70%, at least 80%, or more than 90% as compared to a control, e.g., untreated, sample. In certain embodiments, the decreased PCSK9 expression is maintained at least 21 days. Methods of quantifying mRNA are known in the art and include, for example and without limitation, RT-qPCR and Northern blot.

In some embodiments, a method as provided herein increases PCSK9 promoter methylation levels in the subject treated with an expression repressor or an expression repressor system of the present disclosure. In some embodiments, a method as provided herein increases PCSK9 promoter methylation levels in vitro following treatment with an expression repressor or an expression repressor system of the present disclosure. In certain embodiments, PCSK9 methylation is increased at least 10%, at least 20%, at least 30%, at least 40%, at least 50%, at least 60%, at least 70%, at least 80%, or more than 90% as compared to a control, e.g., untreated, sample. In certain embodiments, the increase in PCSK9 promoter methylation is maintained at least 21 days. Methods of quantifying mRNA are known in the art and include, for example and without limitation, Em-Seq.

In some embodiments, a method as provided herein decreases PCSK9 protein levels in the subject treated with an expression repressor or an expression repressor system of the present disclosure. In some embodiments, a method as provided herein decreases PCSK9 protein levels in vitro following treatment with an expression repressor or an expression repressor system of the present disclosure. In certain embodiments, PCSK9 protein is decreased at least 10%, at least 20%, at least 30%, at least 40%, at least 50%, at least 60%, at least 70%, at least 80%, or more than 90% as compared to a control, e.g., untreated, sample. In certain embodiments, the decreased PCSK9 expression is maintained at least 21 days. Methods of quantifying protein are known in the art and include, for example and without limitation, ELISA and Western blot.

In some embodiments, a method as provided herein decreases circulating serum PCSK9 levels in the subject treated with an expression repressor or an expression repressor system of the present disclosure. In certain embodiments, the decrease in serum PCSK9 is at least 10%, at least 20%, at least 30%, at least 40%, at least 50%, at least 60%, at least 70%, at least 80%, or more than 90% that of the initial PCSK9 serum level. In certain embodiments, the decrease in serum PCSK9 is maintained at least 21 days.

In some embodiments, a method as provided herein improves the serum levels of one or more lipid components. In certain embodiments, the method reduces the patient's low density lipoprotein cholesterol (LDL-C) by at least 10%, at least 20%, at least 30%, at least 40%, at least 50%, at least 60%, at least 70%, at least 80%, or more than 90% as compared to a control, e.g., untreated, sample. In certain embodiments, the method reduces the low density lipoprotein by at least 10%, at least 20%, at least 30%, at least 40%, at least 50%, at least 60%, at least 70%, at least 80%, or more than 90% as compared to a control, e.g., untreated, sample.

In some embodiments, administering an expression repressor or an expression repressor system as provided herein decreases PCSK9 gene expression. PCSK9 gene expression can be measured by any RNA, mRNA, or protein quantitative assay as known in the art, including, for example and without limitation, RNA-sequencing, quantitative reverse transcription PCR (qRT-PCR), RNA microarrays, fluorescent in situ hybridization (FISH), PCSK9 antibody binding, Western blotting, or ELISA.

### Pharmaceutical Compositions

The present disclosure is further directed, in part, to pharmaceutical compositions comprising an expression repressor or an expression repressor system, e.g., expression repressor(s), described herein, to pharmaceutical compositions comprising nucleic acids encoding the expression repressor or expression repressor system, e.g., expression repressor(s), described herein, and/or to and/or compositions that deliver an expression repressor or an expression repressor system, e.g., expression repressor(s), described herein to a cell, tissue, organ, and/or subject.

As used herein, the term “pharmaceutical composition” refers to an active agent (e.g., an expression repressor or nucleic acids of the expression repressor, e.g., an expression repressor system, e.g., expression repressor(s) of an expression repressor system, or a nucleic acid encoding the same), formulated together with one or more pharmaceutically acceptable carriers (e.g., pharmaceutically acceptable carriers known to those of skill in the art). In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition comprising an expression repressor of the present disclosure comprises an expression repressor or nucleic acid(s) encoding the same. In some embodiments, a pharmaceutical composition comprising an expression repressor system of the present disclosure comprises each of the expression repressors of the expression repressor system or nucleic acid(s) encoding the same (e.g., if an expression repressor system comprises a first expression repressor and a second expression repressor, the pharmaceutical composition comprises the first and second expression repressor). In some embodiments, a pharmaceutical composition comprises less than all of the expression repressors of an expression repressor system comprising a plurality of expression repressors. For example, an expression repressor system may comprise a first expression repressor and a second expression repressor, and a first pharmaceutical composition may comprise the first expression repressor or nucleic acid encoding the same and a second pharmaceutical composition may comprise the second expression repressor or nucleic acid encoding the same. In some embodiments, a pharmaceutical composition may comprise coformulation of one or more expression repressors, or nucleic acid(s) encoding the same.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977).

In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid, or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate, and aryl sulfonate. In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing, and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, pharmaceutical compositions may be formulated for delivery to a cell and/or to a subject via any route of administration. Modes of administration to a subject may include injection, infusion, inhalation, intranasal, intraocular, topical delivery, inter-cannular delivery, or ingestion. Injection includes, without limitation, intravenous, intramuscular, intra-arterial, intrathecal, intraventricular, intracapsular, intra-orbital, intracardiac, intradermal, intraperitoneal, transtracheal, subcutaneous, subcuticular, intraarticular, sub capsular, subarachnoid, intraspinal, intra-cerebrospinal, and intra-stemal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, or intravitreal injection). In some embodiments, one or more compositions is administered systemically. In some embodiments, administration is non-parenteral and a therapeutic is a parenteral therapeutic. In some embodiments, administration may be bronchial (e.g., by bronchial instillation), buccal, dermal (which may be or comprise, for example, one or more of topical to the dermis, intradermal, inter-dermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, six, eight, ten, 12, 15 or 20 or more administrations may be given to the subject during one treatment or over a period of time as a treatment regimen.

In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

### Dosage

The dosage of the administered agent or composition can vary based on, e.g., the condition being treated, the severity of the disease, the subject's individual parameters, including age, physiological condition, size and weight, duration of treatment, the type of treatment to be performed (if any), the particular route of administration and similar factors. Thus, the dose administered of the agents described herein can depend on such various parameters. The dosage of an administered composition may also vary depending upon other factors as the subject's sex, general medical condition, and severity of the disorder to be treated. It may be desirable to provide the subject with a dosage of a modulatory agent or combination of modulatory agents disclosed herein that is in the range of from about 1 mg/kg to 6 mg/kg as a single intravenous infusion, although a lower or higher dosage also may be administered as circumstances dictate.

The dosage may be repeated as needed, for example, once every day (e.g., for 1-30 days), once every 3 days (e.g., for 1-30 days) once every 5 days (e.g., for 1-30 days), once per week (e.g., for 1-6 weeks or for 2-5 weeks). In some embodiments, dosages may include, but are not limited to, 1.0 mg/kg-6 mg/kg, 1.0 mg/kg-5 mg/kg, 1.0 mg/kg-4 mg/kg, 1.0 mg/kg-3.0 mg/kg, 1.5 mg/kg-3.0 mg/kg, 1.0 mg/kg-1.5 mg/kg, 1.5 mg/kg-3 mg/kg, 3 mg/kg-4 mg/kg, 4 mg/kg-5 mg/kg, or 5 mg/kg-6 mg/kg. The dosage may be administered multiple times, e.g., once, or twice a week, once every 1, or once every 2 weeks. In some embodiments, the subject is provided with a dosage of a modulatory agent or combination of modulatory agents disclosed herein that is in the range of from about 1 mg/kg to 6 mg/kg as multiple intravenous infusions although a lower or higher dosage also may be administered as circumstances dictate.

A modulatory agent or a combination of modulatory agents as disclosed herein may be administered as one dosage every 3-5 days, repeated for a total of at least 3 dosages. Alternatively, a modulatory agent or a combination of modulatory agents as disclosed herein may be administered at 3 mg/kg every 5 days for 25 days. Alternatively, a modulatory agent or a combination of modulatory agents as disclosed herein may be administered at 1.0-5.0 mg/kg every 3-5 days for 1-10 doses. Alternatively, a modulatory agent or a combination of modulatory agents as disclosed herein may be administered at 1.0-3.0 mg/kg every 5 days for 3 doses then every 3 days for 3 doses. Alternatively, a modulatory agent or a combination of modulatory agents as disclosed herein may be administered at 1.0-3.0 mg/kg every 5 days for 4 doses then every 3 days for 3 doses. Alternatively, a modulatory agent or a combination of modulatory agents as disclosed herein may be administered at 6 mg/kg every 5 days for 1-10 doses. Alternatively, a modulatory agent or a combination of modulatory agents as disclosed herein may be administered at 3 mg/kg every 5 days for 1-10 doses. Alternatively, a modulatory agent or a combination of modulatory agents as disclosed herein may be administered at 1.5 mg/kg every 5 days for 2 doses, 3 mg/kg every 5 days for 3 doses, 3 mg/kg every 3 days for 1 dose. Alternatively, a modulatory agent or a combination of modulatory agents as disclosed herein may be administered at 6 mg/kg at every 5 days or at 1.5 mg/kg once a day for 5 days with 2 days off. The dosing schedule can optionally be repeated at other intervals and dosage may be given through various parenteral routes, with appropriate adjustment of the dose and schedule. In some embodiments, the dosing of modulatory agents or a combination of modulatory agents may include a dosage of between 1.0 mg/kg to 6.0 mg/kg, optionally given either weekly, twice per week, or every other week. The person of ordinary skill will realize that a variety of factors, such as age, sex, weight, severity of disorder to be treated may be considered in selecting a dosage of a modulatory agent or a combination of modulatory agents as disclosed herein, and that the dosage and/or frequency of administration may be increased or decreased during the course of therapy. The dosage may be repeated as needed, with evidence of reduction of tumor volume observed after as few as 2 to 8 doses. The dosages and schedules of administration disclosed herein show minimal effect on overall weight of the subject compared to cisplatin, sorafenib, or a small molecule comparator. The subject methods may include use of CT and/or PET/CT, or MRI, to measure tumor response at regular intervals. Blood levels of tumor markers may also be monitored. Dosages and/or administration schedules may be adjusted as needed, according to the results of imaging and/or marker blood levels.

In some embodiments, an expression repressor or expression repressor system is administered to the patient in combination with a non-statin lipid modifying therapy. In some embodiments, the non-statin lipid modifying therapy comprises a therapeutic agent selected from the group consisting of ezetimibe, a fibrate, niacin, an omega-3 fatty acid, and a bile acid resin.

In some embodiments, in a method of treating a subject with a cancer, the compositions disclosed herein may be administered in combination with one or more therapeutic agents or methods chosen from surgical resection, tyrosine kinase inhibitors (TKIs), e.g., sorafenib, bromodomain inhibitors, e.g., BET inhibitors, e.g., JQ1, e.g., BET672, e.g., birabresib, MEK inhibitors (e.g., Trametinib), orthotopic liver transplantation, radiofrequency ablation, immunotherapy, immune checkpoint plus anti-vascular-endothelial-growth-factor combination therapy, photodynamic therapy (PDT), laser therapy, brachytherapy, radiation therapy, trans-catheter arterial chemo- or radio-embolization, stereotactic radiation therapy, chemotherapy, and/or systemic chemotherapy to treat a disease or disorder.

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

### Administration

In some aspects, the present disclosure provides methods of delivering a therapeutic comprising administering a composition as described herein to a subject, wherein a modulating agent is a therapeutic and/or wherein delivery of a therapeutic causes changes in gene expression relative to gene expression in absence of a therapeutic.

Methods as provided in various embodiments herein may be utilized in any some aspects further delineated herein. In some embodiments, one or more compositions, e.g., comprising an expression repressor or an expression repressor system described herein, is/are targeted to specific cells, or one or more specific tissues.

For example, in some embodiments one or more compositions, e.g., comprising an expression repressor or an expression repressor system described herein, is/are targeted to hepatic, epithelial, connective, muscular, reproductive, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof. In certain embodiments, an expression repressor or an expression repressor system described herein is targeted to the liver or liver cells.

In some embodiments, a composition of the present disclosure crosses a blood-brain-barrier, a placental membrane, or a blood-testis barrier. In some embodiments, a pharmaceutical composition as provided herein is administered systemically. In some embodiments, administration is non-parenteral and a therapeutic is a parenteral therapeutic.

Methods and compositions provided herein, e.g., comprising an expression repressor or an expression repressor system described herein, may comprise a pharmaceutical composition administered by a regimen sufficient to alleviate a symptom of a disease, disorder, and/or condition. In some aspects, the present disclosure provides methods of delivering a therapeutic by administering compositions as described herein.

Pharmaceutical uses of the present disclosure may include compositions (e.g., modulating agents, e.g., disrupting agents) as described herein.

In some embodiments, a pharmaceutical composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to an active agent alone. In some embodiments, a pharmaceutical composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced) as compared to an active agent alone. In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to an active agent alone. Further, in certain embodiments, the present disclosure provides methods for preventing at least one symptom in a subject that would benefit from a modulation of PCSK9 expression, such as a subject having an PCSK9-associated disease, by administering to the subject an agent or composition of the invention in a prophylactically effective amount.

When the subject to be treated is a mammal such as a human, the composition can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection.

In some embodiments, administration of the agents or compositions according to the methods of the invention may result in a reduction of the severity, signs, symptoms, or markers of an PCSK9-associated disease or disorder in a patient with an PCSK9-associated disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

### Kits

The present disclosure further provides a kit comprising an expression repressor or an expression repressor system, e.g., expression repressor(s), described herein. In some embodiments, a kit comprises an expression repressor or an expression repressor system (e.g., the expression repressor(s) of the expression repressor system) and instructions for the use of said an expression repressor or an expression repressor system. In some embodiments, a kit comprises a nucleic acid encoding the expression repressor or a nucleic acid encoding the expression repressor system or a component thereof (e.g., the expression repressor(s) of the expression repressor system) and instructions for the use of said expression repressor (and/or said nucleic acid) and/or said expression repressor system (and/or said nucleic acid). In some embodiments, a kit comprises a cell comprising a nucleic acid encoding the expression repressor or a nucleic acid encoding the expression repressor system or a component thereof (e.g., the expression repressor(s) of the expression repressor system) and instructions for the use of said cell, nucleic acid, and/or said expression repressor or expression repressor system.

In some aspects, the kit comprises a container comprising a composition comprising a system comprising two expression repressors, comprising a first expression repressor comprising a first DNA targeting moiety and optionally a first effector domain, wherein the first expression repressor binds to a transcription regulatory element (e.g., a promoter or transcription start site (TSS)) operably linked to target gene, e.g., PCSK9, or to a sequence proximal to the transcription regulatory element and an expression repressor comprising a second DNA targeting moiety and optionally a second effector domain, wherein the second expression repressor binds to an anchor sequence of an anchor sequence mediated conjunction (ASMC) comprising the target gene, e.g., PCSK9, or to a sequence proximal to the anchor sequence.

In some aspects, the kit comprises a container comprising a composition comprising a system comprising two expression repressors, comprising a first expression repressor comprising a first DNA targeting moiety and optionally a first effector domain, wherein the first expression repressor binds to a transcription regulatory element (e.g., a promoter or transcription start site (TSS)) operably linked to target gene, e.g., PCSK9, or to a sequence proximal to the transcription regulatory element and an expression repressor comprising a second DNA targeting moiety and optionally a second effector domain, wherein the second expression repressor binds to a genomic locus located in a super enhancer region of a target gene, e.g., PCSK9.

In some embodiments the kit further comprises a set of instructions comprising at least one method for treating a disease or modulating, e.g., decreasing the expression of target gene, e.g., PCSK9, within a cell with said composition. In some embodiments, the kits can optionally include a delivery vehicle for said composition (e.g., a lipid nanoparticle). The reagents may be provided suspended in the excipient and/or delivery vehicle or may be provided as a separate component which can be later combined with the excipient and/or delivery vehicle. In some embodiments, the kits may optionally contain additional therapeutics to be co-administered with the compositions to affect the desired target gene expression, e.g., PCSK9, gene expression modulation. While the instructional materials typically comprise written or printed materials, they are not limited to such. Any medium capable of storing such instructions and communicating them to an end user is contemplated. Such media include but are not limited to electronic storage media (e.g., magnetic discs, tapes, cartridges, chips), optical media (e.g., CD ROM), and the like. Such media may include addresses to internet sites that provide such instructional materials.

In some embodiments, a kit comprises a unit dosage of an expression repressor or an expression repressor system, e.g., expression repressor(s), described herein, or a unit dosage of a nucleic acid, e.g., a vector, encoding an expression repressor system, e.g., expression repressor(s), described herein.

### Definitions

As used herein, the singular forms “a,” “an” and “the” include plural referents unless the context clearly dictates otherwise.

As used herein, the term “agent”, may be used to refer to a compound or entity of any chemical class including, for example, a polypeptide, nucleic acid, saccharide, lipid, small molecule, metal, or combination or complex thereof. As will be clear from context to those skilled in the art, in some embodiments, the term may be utilized to refer to an entity that is or comprises a cell or organism, or a fraction, extract, or component thereof. Alternatively, or additionally, as those skilled in the art will understand in light of context, in some embodiments, the term may be used to refer to a natural product in that it is found in and/or is obtained from nature. In some embodiments, again as will be understood by those skilled in the art in light of context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some embodiments, the term “agent” may refer to a compound or entity that is or comprises a polymer; in some embodiments, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.

The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequence.

Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic or transcription complex when the nucleic acid is at least partially within the target genomic or transcription complex, and expression of a gene in the DNA sequence is affected by formation or disruption of the target genomic or transcription complex.

As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

As used herein, the term “CpG sequence,” also called “CpG site” or “CpG dyad,” are regions of DNA having 5′ to 3′ a cytosine nucleoside linked to a guanine nucleoside by a phosphate group (i.e., 5′-C-phosphate linkage-G-3′). A CpG sequence is also referred to as a “CpG dinucleotide.”

As used herein, the term “CpG islands,” also called “CG islands,” are regions of the genome comprising a high frequency of CpG sequences. GpG islands and criteria for identifying CpG islands are known in the art and described in, for example, Bird et al, (1985) Cell 40:91-99). One definition of a CpG island is a region of (1) at least 200 bp in length, (2) a GC percentage greater than 50%, and (3) an observed-to-expected CpG ratio greater than 60%. The observed-to-expected CpG ratio may be calculated in multiple ways. Two methods of calculating the observed-to-expected CpG ratio are as follows:
(a) (number of C*number of G)/length of sequence
(b) ((number of C+number of G)/length of sequence)²

See, e.g., Gardiner-Garden M, Frommer M (1987). “CpG islands in vertebrate genomes”. Journal of Molecular Biology. 196(2): 261-282. doi: 10.1016/0022-2836(87) 90689-9. PMID 3656447; Saxonov S, Berg P, Brutlag DL (2006). “A genome-wide analysis of CpG dinucleotides in the human genome distinguishes two distinct classes of promoters”. Proc Natl Acad Sci USA. 103 (5): 1412-1417. Bibcode:2006 PNAS . . . 103.1412S. doi: 10.1073/pnas.0510310103. PMC 1345710. PMID 16432200. Sources for identification of CpG islands in mammalian genomes (e.g., the hg19 (GRCh37) or hg38 (GRch38) human reference genomes) are known in the art, and include, for example, the UCSC Genome Browser (world wide web: genome.ucsc.edu/cgi-bin/hgTrackUi?g=cpgIslandExt). CpG islands often occur near transcription start sites and promote regions. Indeed, many gene promoters reside within or near CpG islands (see, e.g., Saxonov et al (2006) PNAS 103:1412-17).

As used herein, the term “DNA targeting moiety” refers to an agent or entity that specifically targets, e.g., binds, a target sequence in genomic DNA (e.g., an transcriptional control element or an anchor sequence).

As used herein, the term “effector domain” refers to a domain capable of altering the expression of a target gene when localized to an appropriate site in the nucleus of a cell.

As used herein, the term “expression repressor” refers to an agent or entity with one or more functionalities that decreases expression of a target gene in a cell and that specifically binds to a DNA sequence (e.g., a DNA sequence associated with a target gene or a transcription control element operably linked to a target gene). An expression repressor comprises at least one targeting moiety and optionally one effector domain.

As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that it does not adopt when the complex is not formed.

As used herein, the term “moiety” refers to a defined chemical group or entity with a particular structure and/or or activity, as described herein.

As used herein, the term “modulating agent” refers to an agent comprising one or more targeting moieties and one or more effector moieties that is capable of altering (e.g., increasing or decreasing) expression of a target gene, e.g., PCSK9.

As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

As used herein, the term “nucleating polypeptide” or “conjunction nucleating polypeptide” as used herein, refers to a protein that associates with an anchor sequence directly or indirectly and may interact with one or more conjunction nucleating polypeptides (that may interact with an anchor sequence or other nucleic acids) to form a dimer (or higher order structure) comprised of two or more such conjunction nucleating polypeptides, which may or may not be identical to one another. When conjunction nucleating polypeptides associated with different anchor sequences associate with each other so that the different anchor sequences are maintained in physical proximity with one another, the structure generated thereby is an anchor-sequence-mediated conjunction. That is, the close physical proximity of a nucleating polypeptide-anchor sequence interacting with another nucleating polypeptide-anchor sequence generates an anchor sequence-mediated conjunction (e.g., in some cases, a DNA loop), that begins and ends at the anchor sequence. As those skilled in the art, reading the present specification will immediately appreciate, terms such as “nucleating polypeptide”, “nucleating molecule”, “nucleating protein”, “conjunction nucleating protein”, may sometimes be used to refer to a conjunction nucleating polypeptide. As will similarly be immediately appreciated by those skilled in the art reading the present specification, an assembles collection of two or more conjunction nucleating polypeptides (which may, in some embodiments, include multiple copies of the same agent and/or in some embodiments one or more of each of a plurality of different agents) may be referred to as a “complex”, a “dimer” a “multimer”, etc.

As used herein, the phrase “operably linked” refers to a juxtaposition wherein the components described are in a relationship permitting them to function in their intended manner. A transcription control element “operably linked” to a functional element, e.g., gene, is associated in such a way that expression and/or activity of the functional element, e.g., gene, is achieved under conditions compatible with the transcription control element. In some embodiments, “operably linked” transcription control elements are contiguous (e.g., covalently linked) with coding elements, e.g., genes, of interest; in some embodiments, operably linked transcription control elements act in trans to or otherwise at a distance from the functional element, e.g., gene, of interest. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1,000, 500, 100, 50, or 10 base pairs of each other or directly adjacent to each other.

As used herein, the terms “PCSK9 locus” refer to the portion of the human genome that encodes a PCSK9 polypeptide (e.g., the polypeptide disclosed in NCBI Accession Number NP-777596, or a mutant or variant thereof), the promoter operably linked to PCSK9 (“PCSK9 promoter”), and the anchor sequences that form an ASMC comprising the PCSK9 gene. In some embodiments, the PCSK9 locus encodes a nucleic acid having NCBI Accession Number NM-174936. In certain instances, a PCSK9 gene is found on chromosome 1, at 1p32.3. PCSK9 may also be known in the art as FH3, PC9, FHCL3, NARC1, LDLCQ1, NARC-1, and HCHOLA3.

As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.

As used herein, the term “proximal” refers to the location of a first site and a second site in the genome that occur sufficiently close (e.g., occurring within a span of bases of up to 2,000 bases) for a function directed to the first site results in a desired functional outcome at the second site or vice versa. For example, in some embodiments, the first site is a target sequence described herein and the second site is a site for epigenetic modulation (e.g., a CpG island), wherein the first site and the second site are sufficiently close that an expression repressor targeting the first site via its DNA targeting moiety results in a desired epigenetic modulation at the second site via its effector domain. In some embodiments, the first site is a site for epigenetic modulation (e.g., a CpG island) and the second site is a transcriptional control element (e.g., a promoter) operably linked to a target gene, wherein the first site and the second site are sufficiently close that an expression repressor that introduces an epigenetic modulation at the first site via its effector domain results in altered transcriptional regulation at the second site (e.g., transcriptional regulation resulting in decreased expression of the target gene). In some embodiments, the location of the first site and the location of the second site occur within or overlapping a span of about 300 bases to about 2,000 bases. In some embodiments, the location of the first site and the location of the second site occur within or overlapping a span of about 500 bases to about 1,500 bases. In some embodiments, the location of the first site and the location of the second site occur within or overlapping a span of about 500 bases to about 1,000 bases.

As used herein, the term “pharmaceutical composition” refers to an active agent (e.g., a modulating agent, e.g., an expression repressor or expression repressor system of the present disclosure), formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces.

As used herein, “proximal” refers to a closeness of two sites, e.g., nucleic acid sites, such that binding of an expression repressor at the first site and/or modification of the first site by an expression repressor will produce the same or substantially the same effect as binding and/or modification of the other site. For example, a targeting moiety may bind to a first site that is proximal to an enhancer (the second site), and the effector moiety associated with said targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, or proximal to an anchor sequence is less than 5,000, 4,000, 3,000, 2,000, 1,000, 900, 800, 700, 600, 500, 400, 300, 200, 100, 50, or 25 base pairs from the target gene (e.g., an exon, intron, or splice site within the target gene), transcription control element, or anchor sequence (and optionally at least 20, 25, 50, 100, 200, or 300 base pairs from the target gene (e.g., an exon, intron, or splice site within the target gene), transcription control element, or anchor sequence).

As used herein, the term “specific”, referring to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In some embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments, specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

As used herein, the term “specific binding” refers to an ability to discriminate between possible binding partners in the environment in which binding is to occur. In some embodiments, a binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex. In some embodiments, specific binding is assessed by detecting or determining ability of the binding agent to compete with an alternative interaction between its partner and another entity. In some embodiments, specific binding is assessed by performing such detections or determinations across a range of concentrations.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence.

As used herein, the term “target gene” means a gene that is targeted for modulation, e.g., of expression. In some embodiments, a target gene is part of a targeted genomic complex (e.g. a gene that has at least part of its genomic sequence as part of a target genomic complex, e.g. inside an anchor sequence-mediated conjunction), which genomic complex is targeted by one or more modulating agents as described herein. In some embodiments, modulation comprises inhibition of expression of the target gene. In some embodiments, a target gene is modulated by contacting the target gene or a transcription control element operably linked to the target gene with an expression repression system, e.g., expression repressor(s), described herein. In some embodiments, a target gene is aberrantly expressed (e.g., over-expressed) in a cell, e.g., a cell in a subject (e.g., patient).

As used herein, the term “targeting moiety” means an agent or entity that specifically targets, e.g., binds, a genomic sequence element (e.g., an expression control sequence or anchor sequence). In some embodiments, the genomic sequence element is proximal to and/or operably linked to a target gene (e.g., PCSK9).

As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent comprises an expression repression system, e.g., an expression repressor, described herein. In some embodiments, a therapeutic agent comprises a nucleic acid encoding an expression repression system, e.g., an expression repressor, described herein. In some embodiments, a therapeutic agent comprises a pharmaceutical composition described herein.

As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition.

As used herein, the term “transcriptional repressor moiety” refers to a domain capable of decreasing expression of a target gene when localized to an appropriate site in the genome of a cell (e.g., in or near a transcriptional control element of the target gene).

### OTHER EMBODIMENTS

Embodiment 1. An expression repressor targeting a gene encoding proprotein convertase subtilisin/kexin type 9 (PCSK9) comprising
(i) a DNA targeting moiety that binds to a target sequence, wherein the target sequence is about 15-20 nucleotides of a region spanning position 55,037,859 to position 55,041,755, according to the hg38 reference genome for chromosome 1 (chr1); and
(ii) an effector domain.

Embodiment 2. The expression repressor of embodiment 1, wherein the region spans position 55,038,000 to position 55,040,500, according to the hg38 reference genome for chr1.

Embodiment 3. The expression repressor of embodiment 1 or 2, wherein the region spans position 55,039,100 to position 55,039,600, according to the hg38 reference genome for chr1.

Embodiment 4. The expression repressor of any one of embodiments 1-3, wherein the region spans position 55,039,100 to position 55,039,200; position 55,039,150 to position 55,039,250; position 55,039,200 to position 55,039,300; position 55,039,250 to position 55,039,350; position 55,039,300 to position 55,039,400; position 55,039,350 to position 55,039,450; position 55,039,400 to position 55,039,500; position 55,039,450 to position 55,039,550; position 55,039,500 to position 55,039,600, according to the hg38 reference genome for chr1.

Embodiment 5. The expression repressor of any one of embodiments 1-4, wherein the PCSK9 target sequence is 15, 16, 17, 18, 19, or 20 nucleotides in length.

Embodiment 6. An expression repressor targeting PCSK9 comprising
(i) a DNA targeting moiety that binds to a target sequence, wherein the target sequence comprises a sequence having at least 90% identity to a sequence selected from SEQ ID NOs: 67-80; and
(ii) an effector domain.

Embodiment 7. The expression repressor of embodiment 6, wherein the target sequence comprises a sequence selected from SEQ ID NOs: 67-80.

Embodiment 8. The expression repressor of embodiment 6, wherein the target sequence consists of a sequence selected from SEQ ID NOs: 67-80

Embodiment 9. The expression repressor of one of embodiments 1-8, wherein the DNA-targeting moiety comprises a zinc finger (ZF) domain.

Embodiment 10. The expression repressor of one of embodiments 1-8, wherein the DNA-targeting moiety comprises a transcription activator-like effector (TALE) domain.

Embodiment 11. The expression repressor of one of embodiments 1-8, wherein the DNA-targeting moiety comprises a nuclease inactive Cas polypeptide (dCas9) and a gRNA comprising a sequence complementary to the target sequence.

Embodiment 12. An expression repressor targeting PCSK9 comprising
(i) a DNA targeting moiety that binds to a target sequence comprising an amino acid sequence having at least 90% identity to a sequence selected from 81-94; and
(ii) an effector domain.

Embodiment 13. The expression repressor of any one of embodiments 1-12, wherein the effector domain comprises a transcriptional repressor moiety.

Embodiment 14. The expression repressor of embodiment 13, wherein the transcriptional repressor moiety comprises a Kruppel associated box (KRAB) domain or a functional variant or fragment thereof.

Embodiment 15. The expression repressor of embodiment 13, wherein the transcriptional repressor moiety comprises a histone modifying enzyme selected from a histone methyltransferase, a histone deacetylase, and a histone demethylase.

Embodiment 16. The expression repressor of embodiment 15, wherein the histone modifying enzyme is a histone deacetylase.

Embodiment 17. The expression repressor of embodiment 16, wherein the histone deacetylase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment thereof.

Embodiment 18. The expression repressor of embodiment 15, wherein the histone modifying enzyme is a histone methyltransferase.

Embodiment 19. The expression repressor of embodiment 18, wherein the histone methyltransferase is SETDB1, SETDB2, EHMT2, EHMT1, SUV39H1, EZH2, EZH1, SUV39H2, SETD8, SUV420H1, SUV420H2, or a functional variant or fragment thereof.

Embodiment 20. The expression repressor of embodiment 13, wherein the transcriptional repressor moiety comprises a DNA methyltransferase.

Embodiment 21. The expression repressor of embodiment 20, wherein the DNA methyltransferase is MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment thereof.

Embodiment 22. The expression repressor of embodiment 20, wherein the DNA methyltransferase is MQ1, or a functional variant or fragment thereof.

Embodiment 23. A nucleic acid comprising a nucleotide sequence encoding the expression repressor of any one of embodiments 1-22.

Embodiment 24. A recombinant expression vector comprising the nucleic acid of embodiment 23.

Embodiment 25. A messenger RNA (mRNA) encoding the expression repressor of any one of embodiments 1-22.

Embodiment 26. A lipid nanoparticle (LNP) comprising the expression repressor of any one of embodiments 1-22, the nucleic acid of embodiment 23, the recombinant expression vector of embodiment 24, or the mRNA of embodiment 25.

Embodiment 27. A pharmaceutical composition comprising the expression repressor of any one of embodiments 1-22, the nucleic acid of embodiment 23, the recombinant expression vector of embodiment 24, the mRNA of embodiment 25, or the LNP of embodiment 26, and a pharmaceutically acceptable carrier.

Embodiment 28. A system for modulating expression of human PCSK9 comprising
(i) the expression repressor according to any one of embodiments 1-22, and
(ii) a second expression repressor comprising:
   a second DNA targeting moiety that binds to a second target sequence that is different from the target sequence, and
   a second effector domain,
      wherein the second effector domain is the same as or different from the effector domain.

Embodiment 29. The system of embodiment 28, wherein the expression repressor and the second expression repressor are in the same composition.

Embodiment 30. The system of embodiment 28, wherein the expression repressor and the second expression repressor are in different compositions.

Embodiment 31. The system of embodiment 28, comprising a first nucleic acid encoding the expression repressor and a second nucleic acid encoding the second expression repressor.

Embodiment 32. The system of embodiment 31, wherein the first nucleic acid and the second nucleic acid are in the same composition.

Embodiment 33. The system of embodiment 31, wherein the first nucleic acid and the second nucleic acid are in different compositions.

Embodiment 34. The system of embodiment 31, wherein the first nucleic acid and the second nucleic acid are formulated in the same LNP.

Embodiment 35. The system of embodiment 31, wherein the first nucleic acid and the second nucleic acid are formulated in different LNPs.

Embodiment 36. The system of embodiment 31, comprising a first recombinant expression vector comprising the first nucleic acid and a second recombinant expression vector comprising the second nucleic acid.

Embodiment 37. The system of embodiment 36, wherein the first recombinant expression vector and the second recombinant expression vector are formulated in the same LNP.

Embodiment 38. The system of embodiment 36, wherein the first recombinant expression vector and the second recombinant expression vector are formulated in different LNPs.

Embodiment 39. The system of embodiment 31, comprising a recombinant expression vector comprising the first nucleic acid and the second nucleic acid.

Embodiment 40. The system of embodiment 39, wherein the recombinant expression vector is formulated in an LNP.

Embodiment 41. A nucleic acid comprising a first nucleotide sequence encoding the expression repressor according to any one of embodiments 1-22, and a second nucleotide sequence encoding a second expression repressor comprising
a second DNA targeting moiety that binds to a second target sequence that is different from the target sequence, and
a second effector domain,
wherein the second effector domain is the same as or different from the effector domain.

Embodiment 42. A recombinant expression vector comprising the nucleic acid of embodiment 41.

Embodiment 43. An mRNA that encodes:
the expression repressor according to any one of embodiments 1-22; and
a second expression repressor comprising:
   a second DNA targeting moiety that binds to a second target sequence that is different from the target sequence, and
   a second effector domain,
wherein the second effector domain is the same as or different from the effector domain.

Embodiment 44. The mRNA of embodiment 43, wherein the ORF comprises a ribosome skipping sequence between the first nucleotide sequence and the second nucleotide sequence.

Embodiment 45. An LNP comprising the nucleic acid of embodiment 41, the recombinant expression vector of embodiment 42, or the mRNA of embodiment 43 or 44.

Embodiment 46. The system, nucleic acid, recombinant expression vector, mRNA, or LNP of any one of embodiments 28-45, wherein the second target sequence is about 15-20 nucleotides in an insulated genomic domain (IGD) comprising human PCSK9.

Embodiment 47. The system, nucleic acid, recombinant expression vector, mRNA, or LNP of embodiment 46, wherein the second target sequence is in a region spanning position 55,037,859 to position 55,041,755, according to the hg38 reference genome for chromosome 1 (chr1).

Embodiment 48. The system, nucleic acid, recombinant expression vector, mRNA, or LNP of embodiment 47, wherein the second target sequence is in a region spanning position 55,038,000 to position 55,040,500, according to the hg38 reference genome for chr1.

Embodiment 49. The system, nucleic acid, recombinant expression vector, mRNA, or LNP of embodiment 47 or 53, wherein the second target sequence is in a region spanning position 55,039,100 to position 55,039,600, according to the hg38 reference genome for chr1.

Embodiment 50. The system, nucleic acid, recombinant expression vector, mRNA, or LNP of embodiment 49, wherein the second target sequence is in a region spans position 55,039,100 to position 55,039,200; position 55,039,150 to position 55,039,250; position 55,039,200 to position 55,039,300; position 55,039,250 to position 55,039,350; position 55,039,300 to position 55,039,400; position 55,039,350 to position 55,039,450; position 55,039,400 to position 55,039,500; position 55,039,450 to position 55,039,550; position 55,039,500 to position 55,039,600, according to the hg38 reference genome for chr1.

Embodiment 51. The system, nucleic acid, recombinant expression vector, mRNA, or LNP of embodiment 47, wherein the second target sequence comprises a sequence having at least 90% identity to a sequence selected from SEQ ID NOs: 67-80.

Embodiment 52. The system, nucleic acid, recombinant expression vector, mRNA, or LNP of embodiment 51, wherein the second target sequence comprises a sequence selected from SEQ ID NOs: 67-80.

Embodiment 53. The system, nucleic acid, recombinant expression vector, mRNA, or LNP embodiment 51, wherein the second target sequence consists of a sequence selected from SEQ ID NOs: 67-80

Embodiment 54. The system, nucleic acid, recombinant expression vector, mRNA, or LNP of any one of embodiments 28-53, wherein the DNA-targeting moiety of the second fusion protein comprises a zinc finger (ZF) domain.

Embodiment 55. The system, nucleic acid, recombinant expression vector, mRNA, or LNP of any one of embodiments 28-53, wherein the DNA-targeting moiety of the second fusion protein comprises a TALE domain.

Embodiment 56. The system, nucleic acid, recombinant expression vector, mRNA, or LNP of embodiment 55, wherein the DNA targeting moiety comprises an amino acid sequence having at least 90% identity to a sequence selected from 81-94.

Embodiment 57. The system, nucleic acid, recombinant expression vector, mRNA, or LNP of embodiment 55 or 56, wherein the DNA targeting moiety comprises an amino acid sequence selected from 81-94.

Embodiment 58. The system, nucleic acid, recombinant expression vector, mRNA, or LNP of any one of embodiments 28-53, wherein the DNA-targeting moiety of the second fusion protein comprises a dCas9 and a gRNA comprising a sequence complementary to the second target sequence.

Embodiment 59. The system, nucleic acid, recombinant expression vector, mRNA, or LNP of any one of embodiments 28-58, wherein the second effector domain comprises a second transcriptional repressor moiety.

Embodiment 60. The system, nucleic acid, recombinant expression vector, mRNA, or LNP of embodiment 59, wherein the second transcriptional repressor moiety comprises a Kruppel associated box (KRAB) domain or a functional variant or fragment thereof.

Embodiment 61. The system, nucleic acid, recombinant expression vector, mRNA, or LNP of embodiment 59, wherein the second transcriptional repressor moiety comprises a histone modifying enzyme selected from a histone methyltransferase, a histone deacetylase, and a histone demethylase.

Embodiment 62. The system, nucleic acid, recombinant expression vector, mRNA, or LNP of embodiment 61, wherein the histone modifying enzyme is a histone deacetylase.

Embodiment 63. The system, nucleic acid, recombinant expression vector, mRNA, or LNP of embodiment 62, wherein the histone deacetylase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment thereof.

Embodiment 64. The system, nucleic acid, recombinant expression vector, mRNA, or LNP of embodiment 61, wherein the histone modifying enzyme is a histone methyltransferase.

Embodiment 65. The system, nucleic acid, recombinant expression vector, mRNA, or LNP of embodiment 64, wherein the histone methyltransferase is SETDB1, SETDB2, EHMT2, EHMT1, SUV39H1, EZH2, EZH1, SUV39H2, SETD8, SUV420H1, SUV420H2, or a functional variant or fragment thereof.

Embodiment 66. The system, nucleic acid, recombinant expression vector, mRNA, or LNP of embodiment 59, wherein the second transcriptional repressor moiety comprises a DNA methyltransferase.

Embodiment 67. The system, nucleic acid, recombinant expression vector, mRNA, or LNP of embodiment 66, wherein the DNA methyltransferase is MQ1, DNMT1, DNMT3A1, DNMT3A1, DNMT3B1, DNMT3B2, DNMT3B3, DNMT3B4, DNMT3B5, DNMT3B6, DNMT3L, or a functional variant or fragment thereof.

Embodiment 68. The system, nucleic acid, recombinant expression vector, mRNA, or LNP of embodiment 66, wherein the DNA methyltransferase is MQ1, or a functional variant or fragment thereof.

Embodiment 69. A pharmaceutical composition comprising the system of any one of embodiments 28-40 and 46-68, the nucleic acid of any one of embodiments 41 and 46-68, the recombinant expression vector of any one of embodiments 42 and 46-68, the mRNA of any one of embodiments 43-44 and 46-68, or the LNP of any one of embodiments 45-68, and a pharmaceutically acceptable carrier.

Embodiment 70. A cell comprising the expression repressor of any one of embodiments 1-22, the system of any one of embodiments 28-40 and 46-68, the nucleic acid of any one of embodiments 23, 41, and 46-68, the recombinant expression vector of any one of embodiments 24, 42, and 46-68, the mRNA of any one of embodiments 25, 43-44, and 46-68, the LNP of any one of embodiments 26 and 45-68, or the pharmaceutical composition of embodiment 27 or 69.

Embodiment 71. A method of altering expression of PCSK9 in a cell, comprising contacting the cell with the expression repressor of any one of embodiments 1-22, the system of any one of embodiments 28-40 and 46-68, the nucleic acid of any one of embodiments 23, 41, and 46-68, the recombinant expression vector of any one of embodiments 24, 42, and 46-68, the mRNA of any one of embodiments 25, 43-44, and 46-68, the LNP of any one of embodiments 26 and 45-68, or the pharmaceutical composition of embodiment 27 or 69.

Embodiment 72. The method of embodiment 71, wherein expression of PCSK9 is decreased.

Embodiment 73. A method of introducing one or more epigenetic modifications to PCSK9 in a cell, comprising contacting the cell with the expression repressor of any one of embodiments 1-22, the system of any one of embodiments 28-40 and 46-68, the nucleic acid of any one of embodiments 23, 41, and 46-68, the recombinant expression vector of any one of embodiments 24, 42, and 46-68, the mRNA of any one of embodiments 25, 43-44, and 46-68, the LNP of any one of embodiments 26 and 45-68, or the pharmaceutical composition of embodiment 27 or 69.

Embodiment 74. The method of embodiment 73, wherein the epigenetic modification is DNA methylation or histone methylation.

Embodiment 75. A method of treating a condition associated with PCSK9 expression in a subject, comprising administering to the subject the expression repressor of any one of embodiments 1-22, the system of any one of embodiments 28-40 and 46-68, the nucleic acid of any one of embodiments 23, 41, and 46-68, the recombinant expression vector of any one of embodiments 24, 42, and 46-68, the mRNA of any one of embodiments 25, 43-44, and 46-68, the LNP of any one of embodiments 26 and 45-68, or the pharmaceutical composition of embodiment 27 or 69.

Embodiment 76. The method of embodiment 75, wherein the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

Embodiment 77. A method of increasing LDL receptor-mediated clearance of LDL cholesterol in a subject, comprising administering to the subject the expression repressor of any one of embodiments 1-22, the system of any one of embodiments 28-40 and 46-68, the nucleic acid of any one of embodiments 23, 41, and 46-68, the recombinant expression vector of any one of embodiments 24, 42, and 46-68, the mRNA of any one of embodiments 25, 43-44, and 46-68, the LNP of any one of embodiments 26 and 45-68, or the pharmaceutical composition of embodiment 27 or 69.

Embodiment 78. A method of decreasing a circulating cholesterol level in a subject, comprising administering to the subject the expression repressor of any one of embodiments 1-22, the system of any one of embodiments 28-40 and 46-68, the nucleic acid of any one of embodiments 23, 41, and 46-68, the recombinant expression vector of any one of embodiments 24, 42, and 46-68, the mRNA of any one of embodiments 25, 43-44, and 46-68, the LNP of any one of embodiments 26 and 45-68, or the pharmaceutical composition of embodiment 27 or 69.

Embodiment 79. A kit comprising a container comprising a pharmaceutical composition comprising the expression repressor of any one of embodiments 1-22, the system of any one of embodiments 28-40 and 46-68, the nucleic acid of any one of embodiments 23, 41, and 46-68, the recombinant expression vector of any one of embodiments 24, 42, and 45-68, the mRNA of any one of embodiments 25, 43-44, and 46-68, or the LNP of any one of embodiments 26 and 46-68, and a pharmaceutically acceptable carrier, and instructions for use in treating a condition associated with PCSK9 expression in a subject.

Embodiment 80. The kit of embodiment 79, wherein the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

Embodiment 81. A kit comprising a container comprising a pharmaceutical composition comprising the expression repressor of any one of embodiments 1-22, the nucleic acid of any one of embodiments 23, 41, and 46-68, the recombinant expression vector of any one of embodiments 24, 42, and 46-68, the mRNA of any one of embodiments 25, 43-44, and 46-68, or the LNP of any one of embodiments 26 and 45-68, and a pharmaceutically acceptable carrier, and instructions for use in increasing LDL receptor-mediated clearance of LDL cholesterol and/or decreasing a circulating cholesterol level in a subject.

Embodiment 82. A method of treating a condition associated with PCSK9 expression in a subject, comprising administering to the subject (i) the expression repressor according to any one of embodiments 1-22, and (ii) a second expression repressor comprising:
a second DNA targeting moiety that binds to a second target sequence that is different from the target sequence, and
a second effector domain,
   wherein the second effector domain is the same as or different from the effector domain.

Embodiment 83. The method of embodiment 82, wherein the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

Embodiment 84. A method of increasing LDL receptor-mediated clearance of LDL cholesterol in a subject, comprising administering to the subject (i) the expression repressor according to any one of embodiments 1-22, and (ii) a second expression repressor comprising:
a second DNA targeting moiety that binds to a second target sequence that is different from the target sequence, and
a second effector domain,
   wherein the second effector domain is the same as or different from the effector domain.

Embodiment 85. A method of decreasing a circulating cholesterol level in a subject, comprising administering to the subject (i) the expression repressor according to any one of embodiments 1-22, and (ii) a second expression repressor comprising:
a second DNA targeting moiety that binds to a second target sequence that is different from the target sequence, and
a second effector domain,
   wherein the second effector domain is the same as or different from the effector domain.

Embodiment 86. The method of any one of embodiments 82-85, comprising administering the expression repressor and the second expression repressor in the same composition or in different compositions.

Embodiment 87. The method of any one of embodiments 82-85, comprising administering a first nucleic acid encoding the expression repressor and a second nucleic acid encoding the second repression repressor.

Embodiment 88. The method of embodiment 87, wherein the first nucleic acid is an mRNA encoding the expression repressor.

Embodiment 89. The method of embodiment 87 or 88, wherein the second nucleic acid is an mRNA encoding the second expression repressor.

Embodiment 90. The method of any one of embodiments 87-89, comprising administering the first nucleic acid and the second nucleic acid in the same composition or in different compositions.

Embodiment 91. The method of any one of embodiments 87-89, comprising administering the first nucleic acid and the second nucleic acid formulated in the same LNP or in separate LNPs.

Embodiment 92. The method of embodiment 87, comprising administering a first recombinant expression vector comprising the first nucleic acid and a second recombinant expression vector comprising the second nucleic acid.

Embodiment 93. The method of embodiment 92, wherein the first recombinant expression vector and the second recombinant expression vector are formulated in the same LNP or in separate LNPs.

Embodiment 94. The method of embodiment 87, comprising administering a recombinant expression vector comprising the first nucleic acid and the second nucleic acid.

Embodiment 95. The method of embodiment 94, wherein the recombinant expression vector is formulated in an LNP.

Embodiment 96. The method of any one of embodiments 82-95, wherein the second target sequence is about 15-20 nucleotides in an insulated genomic domain (IGD) comprising human PCSK9.

Embodiment 97. The method of embodiment 96, wherein the second target sequence is in a region spanning position 55,037,859 to position 55,041,755, according to the hg38 reference genome for chromosome 1 (chr1).

Embodiment 98. The method of embodiment 96, wherein the second target sequence is in a region spanning position 55,038,000 to position 55,040,500, according to the hg38 reference genome for chr1.

Embodiment 99. The method of embodiment 97 or 98, wherein the second target sequence is in a region spanning position 55,039,100 to position 55,039,600, according to the hg38 reference genome for chr1.

Embodiment 100. The method of any one of embodiments 97-99, wherein the second target sequence is in a region spanning position 55,039,100 to position 55,039,200; position 55,039,150 to position 55,039,250; position 55,039,200 to position 55,039,300; position 55,039,250 to position 55,039,350; position 55,039,300 to position 55,039,400; position 55,039,350 to position 55,039,450; position 55,039,400 to position 55,039,500; position 55,039,450 to position 55,039,550; or position 55,039,500 to position 55,039,600, each according to the hg38 reference genome for chr1.

Embodiment 101. The method of embodiment 96 or 97, wherein the second target sequence comprises a sequence having at least 90% identity to a sequence selected from SEQ ID NOs: 67-80.

Embodiment 102. The method of embodiment 96 or 97, wherein the second target sequence comprises a sequence selected from SEQ ID NOs: 67-80.

Embodiment 103. The method of embodiment 102, wherein the second target sequence consists of a sequence selected from SEQ ID NOs: 67-80.

Embodiment 104. The method of any one of embodiments 82-103, wherein the second DNA-targeting moiety comprises a zinc finger (ZF) domain.

Embodiment 105. The method of any one of embodiments 82-103, wherein the second DNA-targeting moiety comprises a TALE domain.

Embodiment 106. The method of embodiment 105, wherein the second DNA targeting moiety comprises an amino acid sequence having at least 90% identity to a sequence selected from 81-94.

Embodiment 107. The method of any one of embodiments 82-103, wherein the second DNA-targeting moiety comprises a dCas9 and a gRNA comprising a sequence complementary to the second target sequence.

Embodiment 108. The method of any one of embodiments 82-107, wherein the second effector domain comprises a second transcriptional repressor moiety.

Embodiment 109. The method of embodiment 108, wherein the second transcriptional repressor moiety comprises a Kruppel associated box (KRAB) domain or a functional variant or fragment thereof.

Embodiment 110. The method of embodiment 108, wherein the second transcriptional repressor moiety comprises a histone modifying enzyme selected from a histone methyltransferase, a histone deacetylase, and a histone demethylase.

Embodiment 111. The method of embodiment 110, wherein the histone modifying enzyme is a histone deacetylase.

Embodiment 112. The method of embodiment 111, wherein the histone deacetylase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment thereof.

Embodiment 113. The method of embodiment 110, wherein the histone modifying enzyme is a histone methyltransferase.

Embodiment 114. The method of embodiment 113, wherein the histone methyltransferase is SETDB1, SETDB2, EHMT2, EHMT1, SUV39H1, EZH2, EZH1, SUV39H2, SETD8, SUV420H1, SUV420H2, or a functional variant or fragment thereof.

Embodiment 115. The method of embodiment 108, wherein the second transcriptional repressor moiety comprises a DNA methyltransferase.

Embodiment 116. The method of embodiment 115, wherein the DNA methyltransferase is MQ1, DNMT1, DNMT3A1, DNMT3A1, DNMT3B1, DNMT3B2, DNMT3B3, DNMT3B4, DNMT3B5, DNMT3B6, DNMT3L, or a functional variant or fragment thereof.

Embodiment 117. The method of embodiment 115, wherein the DNA methyltransferase is MQ1, or a functional variant or fragment thereof.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Targeted Modification of the CTCF Motif Results in Downregulation of Mouse PCSK9 Protein Expression

This example describes nuclease-editing with a fusion of a catalytically inactive Cas9 (dCas9) to a MQ1 DNA methyltransferase (dCas9-MQ1) targeted by a single-guide RNA (sgRNA) to a target sequence within the mouse PCSK9 (“mPCSK9”) promoter to downregulate mPCSK9 expression.

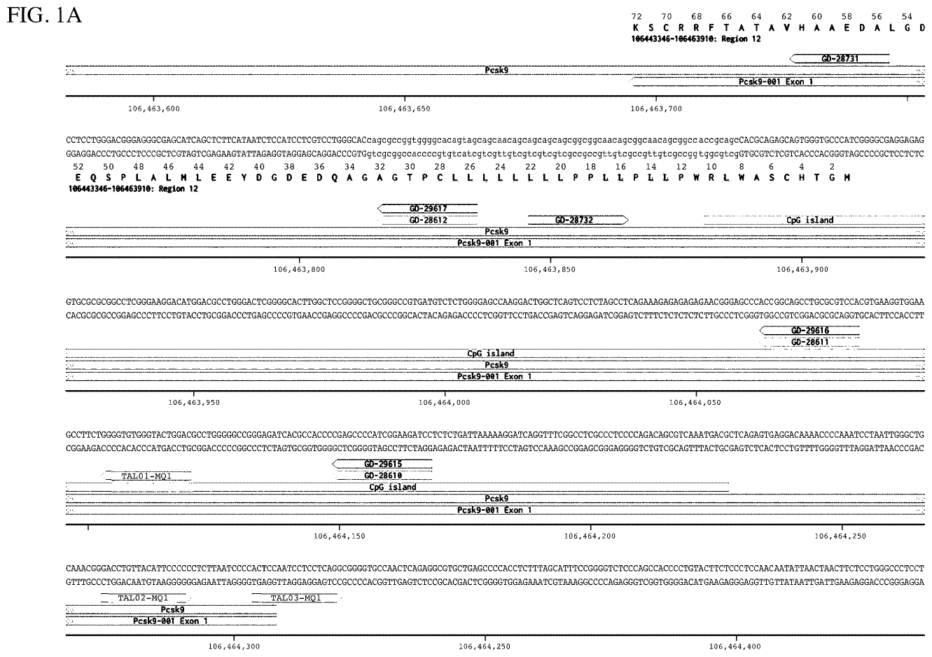

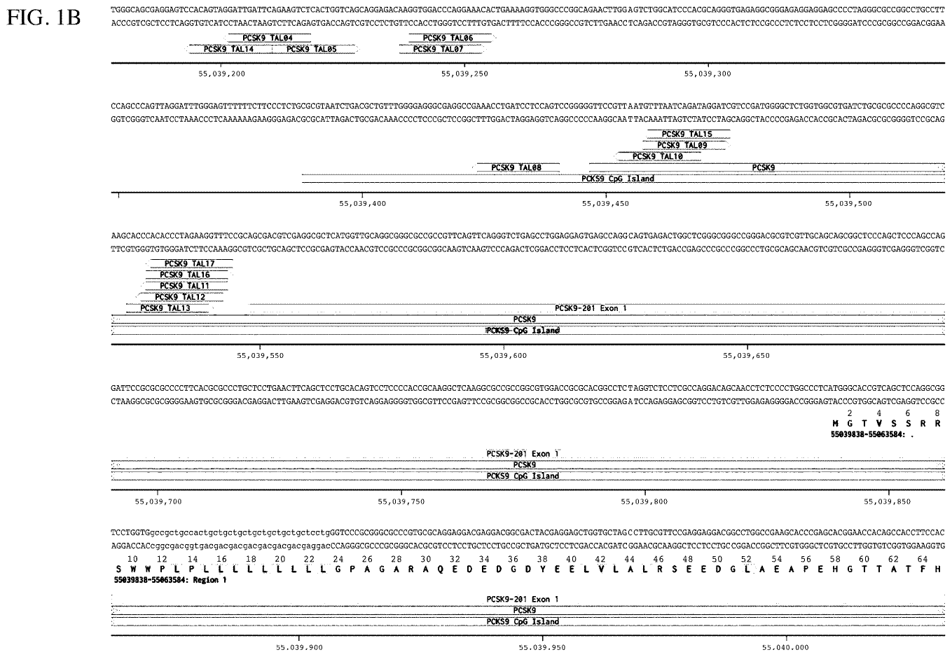

As indicated in FIG. 1A, the mPCSK9 gene contains CpG islands that can be methylated to decrease gene expression. The human PCSK9 gene is depicted in FIG. 1B. One such CpG island overlaps with exon 1. This region was evaluated bioinformatically for potential target sequences, e.g., for sgRNAs as used in this Example.

A set of sgRNAs having a spacer sequence complementary to a target sequence near the promoter region CpG-island of mouse mPCSK9 were tested for mPCSK9 downregulation (sequences are indicated in Table 5). CRISPR-dCas9 was modified by tethering it to MQ1 (a DNA methyltransferase from the bacteria Mollicutes spiroplasma) to generate a dCas9-MQ1 fusion that was further encoded by an mRNA having a nucleotide sequence as set forth in SEQ ID NO:46.

To assess the effect of dCas9-MQ1 on mPCSK9 expression in vivo, a study using a murine model was designed. Details of the study design are presented in Table 6 infra. RNA concentration was varied to determine which dose was suitable for further studies. For administration to mice, a test article (TA-1 or TA-2) was created by formulating mRNA in a lipid nanoparticle (LNP). TA-1 (co-formulation of dCas9-MQ1 [MR-28125-2] and GD-29615 at 6 mg/kg or TA-2 (co-formulation of dCas9-MQ1 [MR-28125-2] and GD-29615) were prepared; details of the compositions are presented in Table 7 infra. The N: P ratio (ionizable lipid nitrogen: oligonucleotide phosphate) describes the relationship between the nucleic acid and cationic lipid, and by extension, all lipids. TA-1 and TA-2 were dosed at different concentrations (1.2 mg RNA/mL and 0.6 mg RNA/mL); the lipids in TA-1 are twice the concentration of the lipids in TA-2. In Table 7, “mg/mL” RNA refers both to the vial concentration and dosing concentration of the TAs. The dosing concentration (mg/mL) is related to the dose (mg/kg) by the dosing volume (mL/kg). The dosing volume was set at a constant 5 mL/kg, which typically corresponds to 100-125 μL for a 20-25 gram mouse. This is a simple dosing volume for IV administration in mice, as it is large enough to minimize random error in volume measurements, but half the typically accepted maximum IV dosing volume for mice, which is 10 mL/kg. RNA concentration and encapsulation were quantified by RiboGreen fluorescence assay (Thermo Fisher Scientific) according to the manufacturer's instructions. Size, PDI (polydispersity index), and charge were simultaneously determined by dynamic light scattering/phase analysis light scattering (DLS/PALS) using a Mobius™ from Wyatt Technology according to the manufacturer's instructions.

TABLE 5
Guide Sequences in mPCSK9

| | Sequence | Sequence | | | |
|---|---|---|---|---|---|
| | SEQ ID | SEQ ID | | | |
| | NO: of | NO of | DNA | | |
| | DNA | guide | Strand | | Genomic |
| | Target | RNA | of Target | | Coordinates of |
| Guide ID | Sequence | sequence | Sequence | Genome | Target sequence |
| GD-29617* | 1 | 21 | − | GRCm38 | chr4: 106463813- |
| | | | | | 106463835 |
| GD-29616* | 2 | 22 | − | GRCm38 | chr4: 106464060- |
| | | | | | 106464082 |
| GD-29615* | 3 | 23 | − | GRCm38 | chr4: 106464146- |
| | | | | | 106464168 |
| GD-28733 | 4 | 24 | + | GRCm38 | chr4: 106463846- |
| | | | | | 106463868 |
| GD-28732 | 5 | 25 | + | GRCm38 | chr4: 106463846- |
| | | | | | 106463868 |
| GD-28731 | 6 | 26 | − | GRCm38 | chr4: 106463724- |
| | | | | | 106463746 |
| GD-28612 | 1 | 27 | − | GRCm38 | chr4: 106463813- |
| | | | | | 106463835 |
| GD-28611 | 2 | 28 | − | GRCm38 | chr4: 106464060- |
| | | | | | 106464082 |
| GD-28610 | 3 | 29 | − | GRCm38 | chr4: 106464146- |
| | | | | | 106464168 |
| GD-27661 | 10 | 30 | − | GRCm38 | chr4: 106474397- |
| | | | | | 106474419 |
| GD-27660 | 11 | 31 | − | GRCm38 | chr4: 106474384- |
| | | | | | 106474406 |
| GD-27659 | 12 | 32 | + | GRCm38 | chr4: 106474372- |
| | | | | | 106474394 |
| GD-27658 | 13 | 33 | + | GRCm38 | chr4: 106474360- |
| | | | | | 106474382 |
| GD-27657 | 14 | 34 | + | GRCm38 | chr4: 106472482- |
| | | | | | 106472504 |
| GD-27656 | 15 | 35 | − | GRCm38 | chr4: 106472487- |
| | | | | | 106472509 |
| GD-27655 | 16 | 36 | + | GRCm38 | chr4: 106472475- |
| | | | | | 106472497 |
| GD-27654 | 17 | 37 | − | GRCm38 | chr4: 106472475- |
| | | | | | 106472497 |
| GD-27653 | 18 | 7 | − | GRCm38 | chr4: 106436672- |
| | | | | | 106436694 |
| GD-27652 | 19 | 8 | − | GRCm38 | chr4: 106436669- |
| | | | | | 106436691 |
| GD-27651 | 20 | 9 | + | GRCm38 | chr4: 106436654- |
| | | | | | 106436676 |

*indicates sgRNA was used as an in vivo guide

TABLE 6
dCas9-MQ1 mPCSK9 Mouse Study Design

| | | | | | Terminal | Samples |
|---|---|---|---|---|---|---|
| Group | | Test | Dose | Dosing | Time | and |
| No. | Animals | Article | (mg/kg) | regimen | Points | Analytics |
| 1 | 24 | TA-1 | 6 | 1 IV | 6 animals | Liver |
| 2 | 24 | TA-2 | 3 | injection | from | (RT-qPCR) |
| 3 | 24 | PBS | n/a | at Day 0 | each | liver |
| | | | | | group at | function tests |
| | | | | | 72 h, 1, 2 | |
| | | | | | and 4 | |
| | | | | | weeks | |
| | | | | | post IV | |

TABLE 7
dCas9-MQ1 mPCSK9 Lipid Nanoparticle (LNP) Parameters

| LNP | | | | | | | | |
|---|---|---|---|---|---|---|---|---|
| (mRNA, | [RNA] | Vol. | Encapsul | | | Charge | | N:P |
| sgRNA) | mg/ml | (mL) | Effic. | Diameter | PDI | (mV) | Lipids | ratio |
| TA-1 | 1.2 | 5.0 | 93.1% | 80.6 ± 2.0 | 0.124 ± | −6.29 ± | 45% MC3/ | 3 |
| (MR- | | | | | 0.054 | 1.29 | 44% | |
| 28125, | | | | | | | cholesterol/ | |
| GD- | | | | | | | 9% DOPC/ | |
| 29615) | | | | | | | 2% | |
| TA-2 | 0.6 | 5.0 | 93.1% | 108.2 ± | 0.141 ± | −6.12 ± | PEG2K- | |
| (MR- | | | | 0.3 | 0.017 | 0.38 | DMG | |
| 28125, | | | | | | | | |
| GD- | | | | | | | | |
| 29615) | | | | | | | | |

C57 BL/6 mice were dosed intravenously (iv) with TA-1 (LNP coformulation of dCas9-MQ1 [MR-28125-2] and GD-29615) at 6 mg/kg or TA-2 (LNP coformulation of dCas9-MQ1 [MR-28125-2] and GD-29615) at 3 mg/kg at the indicated time points. PBS-only was used as a control.

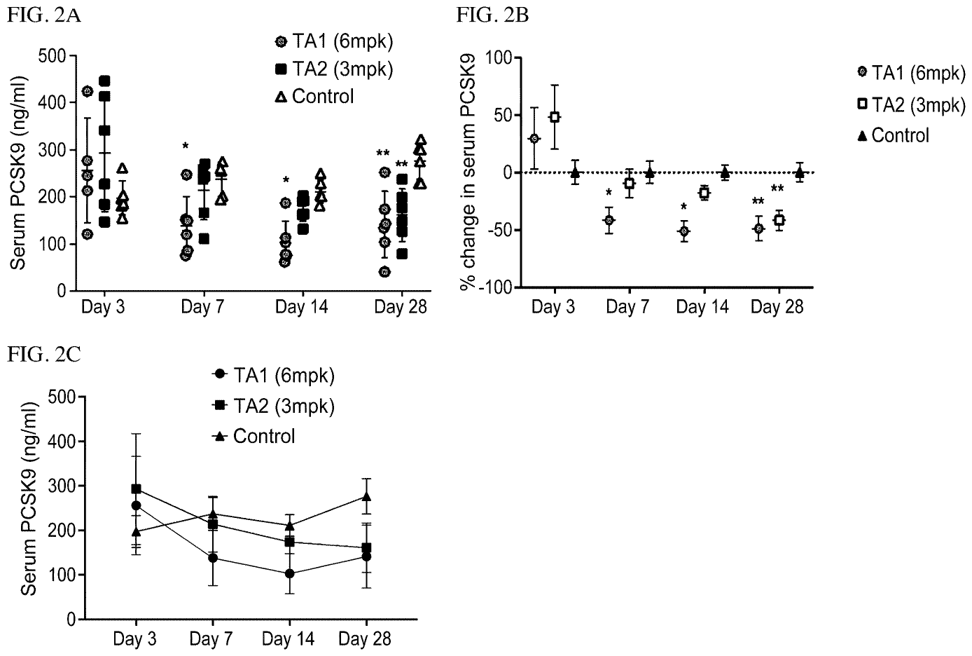

To determine levels of mPCSK9 protein in mouse serum following dosing of TA-1 and TA-2, serum was isolated from each mouse at 3, 7, 14, and 28-days post LNP administration. An ELISA assay was performed per the manufacturer's recommendations using R&D Systems® PCSK9 Quantikine® ELISA Kit (MPC900). Data were analyzed using two-way ANOVA with Sidak's multiple comparison tests. Administration of either TA-1 or TA-2 resulted in a decrease in serum mPCSK9 protein levels relative to control as early as Day 7 following treatment; a significant decrease was observed at Days 14 and 28, with strongest effect (p<0.0021) observed at Day 28. TA-1 treatment demonstrated a significant decrease in serum mPCSK9 at days 7, 14, and 28; TA-2 treatment demonstrated a significant decrease in serum mPCSK9 at day 28. mPCSK9 levels are shown in FIG. 2A, and percent change is shown in FIG. 2B. Change over time in serum mPCSK9 is indicated in the line graph shown in FIG. 2C.

Taken together, these data demonstrate that targeting the mPCSK9 promoter with dCas9-MQ1 results in decreased serum mPCSK9 in a mouse model.

### Example 2: Downregulation of mPCSK9 Expression by MQ1 Effectors Fused to TALEs

This example describes downregulating mPCSK9 expression by targeting MQ1 effector fused to TALE domains to the mPCSK9 promoter.

As described in Example 1 supra, sgRNAs were identified for targeting dCas9-MQ1 near the mPCSK9 CpG island at the promoter. This region was scanned for identification of target sequences for TALEs and a bioinformatics approach was taken to select target sequences for further validation based upon criteria that included likelihood of off-target binding.

A TALE-MQ1 fusion proteins were designed to regulate methylation of CpG dyads in proximity to the promoter of mPCSK9. The TALE portion was designed to bind to a specific DNA target sequence, and the MQ1 portion contains the MQ1 DNA methyltransferase from Mollicutes spiroplasma. Target sequences near the mPCSK9 promoter CpG island for three TALEs (encoding TAL01, TAL02, and TAL03) that flank the target sequence for GD-29615 sgRNA described in Example 1 and selected for evaluation are shown in FIG. 1 and identified in Table 8.

Upon identification of potential target sequences, expression repressors comprising fusion proteins were designed to target these sequences. Fusion proteins comprising TALEs directed to these target sequences were developed using methods as known in the art.

mRNA encoding the expression repressors comprised an ORF encoding 5′ to 3′: (i) a TALE as set forth in SEQ ID NO: 45 (TALE01), SEQ ID NO: 52 (TALE02), or SEQ ID NO: 60 (TALE03); (ii) a linker; and (iii) MQ1 as set forth in SEQ ID NO: 47. The mRNA further included a 5′UTR and 3′UTR. The sequences of the full-length mRNA and encoded TAL-MQ1 fusion proteins are identified in Table 8. The mRNA sequences were prepared by in vitro-transcription. Furthermore, the mRNAs were synthesized to have a polyA-tail, and a Cap 1 structure. For administration to mice, a test article (TA-1) was created by formulating mRNA in an LNP. The composition of TA-1 is provided in Table 9.

TABLE 8
TALE-Design Sequences
TAL01-MQ1

| | |
|---|---|
| mRNA ID | MR-33111 |
| Plasmid ID | PL-33089 |
| mRNA sequence | SEQ ID NO: 41 |
| DNA sequence | SEQ ID NO: 42 |
| Protein sequence | SEQ ID NO: 43 |
| DNA Binding | SEQ ID NO: 44 |
| Domain (DBD) | |
| mRNA sequence | |
| DBD protein | SEQ ID NO: 45 |
| sequence | |
| MQ1 Effector | SEQ ID NO: 46 |
| mRNA sequence | |
| MQ1 Effector | SEQ ID NO: 47 |
| protein sequence | |
| Target DNA | SEQ ID NO: 48 |
| sequence | |
| Target DNA | chr4: 106464103-106464120 |
| coordinates | |

TAL02-MQ1

| | |
|---|---|
| mRNA ID | MR-33112 |
| Plasmid ID | PL-33090 |
| mRNA sequence | SEQ ID NO: 49 |
| DNA sequence | SEQ ID NO: 50 |
| Protein sequence | SEQ ID NO: 51 |
| DBD mRNA | SEQ ID NO: 52 |
| sequence | |
| DBD protein | SEQ ID NO: 53 |
| sequence | |
| MQ1 Effector | SEQ ID NO: 46 |
| mRNA sequence | |
| MQ1 Effector | SEQ ID NO: 47 |
| protein sequence | |
| Target DNA | SEQ ID NO: 56 |
| sequence | |
| Target DNA | chr4: 106464295-106464312 |
| coordinates | |

TAL03-MQ1

| | |
|---|---|
| mRNA ID | MR-33113 |
| Plasmid ID | PL-33091 |
| mRNA sequence | SEQ ID NO: 57 |
| DNA sequence | SEQ ID NO: 58 |
| Protein sequence | SEQ ID NO: 59 |
| DBD mRNA | SEQ ID NO: 60 |
| sequence | |
| DBD protein | SEQ ID NO: 61 |
| sequence | |
| Effector mRNA | SEQ ID NO: 46 |
| sequence | |
| MQ1 Effector | SEQ ID NO: 47 |
| protein sequence | |
| Target DNA | SEQ ID NO: 64 |
| sequence | |
| Target DNA | chr4: 106464325-106464342 |
| coordinates | |

TABLE 9
TAL02-MQ1 mPCSK9 Test Article Parameters for mPCSK9 in vivo Study

| | | [RNA] | Vol. | Encapsul. | Diameter | | Charge | | N:P |
|---|---|---|---|---|---|---|---|---|---|
| LNP | mRNA | mg/ml | (mL) | Effic. | (nm) | PDI | (mV) | Lipids | ratio |
| TA- | MR- | 0.66 | 3.0 | 95.3 | 98 ± | 0.034 ± | −0.43 ± | 50% MC3/ | 3 |
| 1 | 33112- | | | | 0.4 | 0.029 | 1.74 | 38.5% | |
| | 1 | | | | | | | cholesterol/ | |
| | | | | | | | | 10% | |
| | | | | | | | | DSPC/ | |
| | | | | | | | | 1.5% | |
| | | | | | | | | PEG2K- | |
| | | | | | | | | DMG | |

### Assaying for Downregulation In Vitro

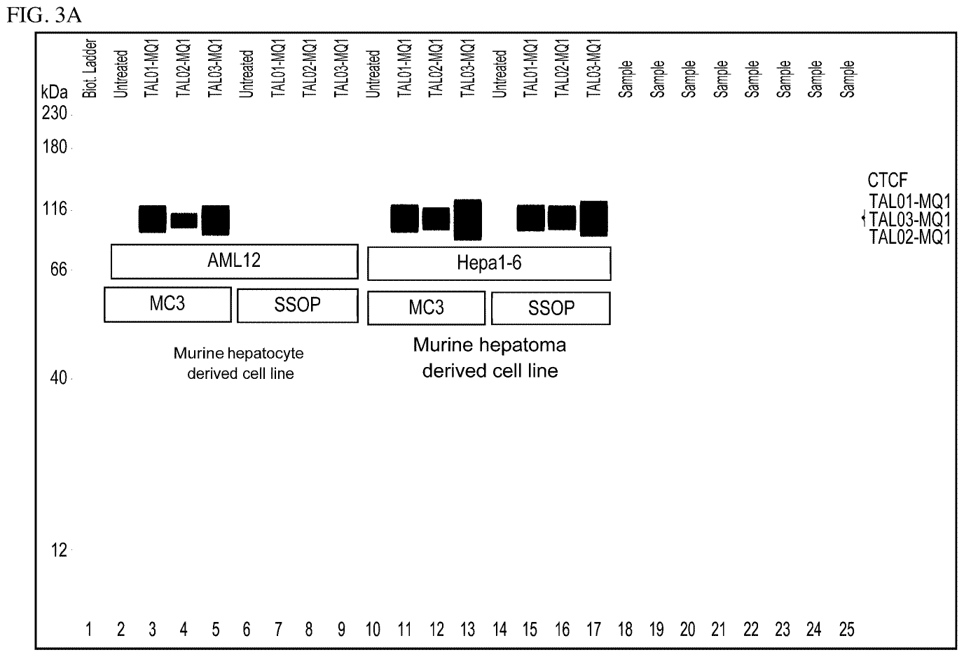

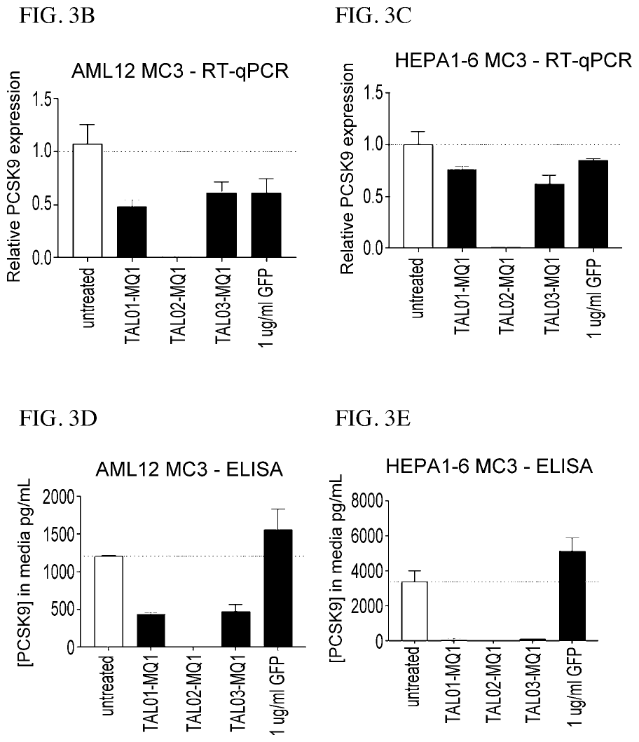

RNA encoding TAL01-MQ1 (MR-33111), TAL02-MQ1 (MR-33112), and TAL03-MQ1 (MR-33112) each were formulated into either MC3 or SSOP LNPs. Each LNP contained about: 1) 45% MC3 or 45% SS-OP; 2) 44% cholesterol; 3) 9% DOPC; and 4) 2% DMG-PEG2000. Similar LNP composition was used throughout the experiments, with minor modifications for the data presented infra to improve stability when frozen. Each TALE-MQ1 fusion was tagged with HA for this experiment. An mRNA encoding GFP and formulated in MC3 and SSOP LNP was used as a control. AML-12 murine hepatocyte cell line or HEPA1-6 murine hepatoma cells were treated with 1 μg/ml of the indicated TALE-MQ1 fusion in either lipid formulation. For protein expression analysis, cells were treated with 1 μg/ml of the indicated formulation in MC3 or SSOP lipids for 6 hours. At this time, the cells were lysed, and protein was harvested. TAL-MQ1 protein expression was detected by HA expression relative to the housekeeper control, CTCF, using the ProteinSimple® JESS system with antibodies targeting either the HA-tag or CTCF, per the manufacturer's instructions. TAL-MQ1 protein expression in both cell lines, with both LNP formulations, is shown in FIG. 3A. SSOP LNPs generally do not effectively transfect AML 12 cells. All other TAL-MQ1 transfections demonstrated effective protein expression.

For mPCSK9 gene expression and secretion analysis, 48 hours post-treatment, media was collected, cells were lysed, and RNA was isolated. RNA was then reverse-transcribed to cDNA and analyzed by multiplexed qPCR using TaqMan® primer probes specific to B2M (housekeeper control) and mPCSK9. Relative mPCSK9 mRNA expression was determined through the comparative ΔΔCt method. As shown in FIG. 3B and FIG. 3C, each TAL-MQ1 tested resulted in decreased mPCSK9 mRNA compared to untreated cells, with TAL02-MQ1 displaying the greatest effect and reproducible results across both cell lines.

Secreted mPCSK9 protein expression was determined using the Abcam® Mouse PCSK9 ELISA Kit (ab215538) from the media of the indicated cells and conditions according to the manufacturer's instructions. Each TAL-MQ1 effector displayed a significant decrease in mPCSK9 protein as determined by ELISA, with each resulting in undetectable levels in the HEPA1-6 cell line (FIG. 3D and FIG. 3E).

### Assaying for Downregulation In Vivo

To assess the effect of TAL02-MQ1 on mPCSK9 expression in vivo, a study using a C57 BL/6 murine model was designed. Details of the study design are presented in Table 10 infra, with dosing shown as total nucleic acid per kg animal body mass.

TABLE 10
mPCSK9 Mouse in vivo Study Design

| | | | | | Dosing | | Sample |
|---|---|---|---|---|---|---|---|
| | | Ani- | | Dose | Regimen | Terminal | collection |
| Group | Co- | mal | Test | (mg/ | (One IV | Time | and |
| No. | hort | No. | Article | kg) | injection) | Point | Analytics |
| 1 | A | 10 | TA-1 | 3.0 | Day 0 | 10 animals | Liver |
| | | | | | | at 1 w | punches |
| | | | | | | post TA-1 | (flash |
| | | | | | | injection | frozen for |
| 2 | A | 10 | TA-2 | 1.0 | Day 0 | 10 animals | RT-qPCR |
| | | | | | | at 1 w | and |
| | | | | | | post TA-2 | EM-seq) |
| | | | | | | injection | Serum |
| | | | | | | | (mPCSK9, |
| 3 | A | 10 | TA-3 | 0.3 | Day 0 | 10 animals | LDL-c) |
| | | | | | | at 1 w | Analytics: |
| | | | | | | post TA-3 | AST/ALT |
| | | | | | | injection | to evalu- |
| | | | | | | | ate liver |
| | | | | | | | health |
| 4 | A | 10 | PBS | N/A | Day 0 | 10 animals | |
| | | | | | | at 1 w | |

10 mice from each group (Group Nos. 1-4, supra) were treated with the indicated concentration of TA-1 (TAL02-MQ1 (MR-33112-1)) LNP, with characteristics as indicated in Table 10. Seven days post-LNP administration, serum and liver punches were isolated from each murine subject. Upon isolation, serum samples were frozen, and liver punches were flash frozen, until testing.

To determine if treatment with TAL02-MQ1 was altering methylation at the mPCSK9 promoter, enzymatic methyl-seq (EM-Seq) was performed. Briefly, genomic DNA was isolated from liver samples retrieved from animals that had been treated with either TAL02-MQ1 LNPs or PBS control. Genomic DNA was normalized to 200 ng in 100 μl low TE buffer and briefly sheared using the PIXUL® (Active Motif) Sonicator to obtain fragments less than 15 kb in size using the following parameters: (5 Pulse/1 kHz PRF/3 min/20 Hz Burst). Fragmented DNA then was purified using SPRI beads (1×SPRISelect, Beckman-Coulter® Cat #B23319) and subjected to EM-conversion using the NEB® EM-seq Conversion Kit (Cat #E7125) according to the manufacturer's instructions. Purified, converted DNA was PCR amplified for 40 cycles at the mPCSK9 locus using NEB® Q5U® MasterMix (Cat #M0597) according to the manufacturer's instructions. Primers (500 nM each in 20 μl reactions with a 64° C. annealing temperature) comprising SEQ ID NO:65 and SEQ ID NO:66 were used.

Following SPRI bead purification (1.8× SPRISelect®), the 457 bp amplicon was transposase-labeled with Illumina® sequencing adapters using Tagment DNA Enzyme 1 (Illumina® Cat #20034197) and following the manufacturer's instructions. Tagmentation was performed using 0.1 μl enzyme per 10 μl reaction containing approximately 30 ng of the amplicon for 5 minutes at 37° C., and the reaction was stopped with 0.04% SDS. Libraries were dual-indexed (combinatorial) via PCR using KAPA HiFi ReadyStart MasterMix (Roche® Cat, #KK2602) and i5/i7 primers derived from Mezger A, et al. Nature Comm. 2018 (PMID: 30194434). PCR reactions occurred in 40 μl volumes with 100 nM of each primer for 13 cycles.

Final libraries were purified using SPRI beads (1×SPRISelect), pooled at equimolar ratios, and sequenced on a MiSeq (Illumina®) using a v2 Nano 2×150 bp reagent kit (Cat #MS-103-1001) according to the manufacturer's instructions.

To analyze the EM-Seq data, EMseq.fastq files were assessed for alignment quality using FastQC, adapter-trimmed and quality filtered using TrimGalore, and aligned to the mm 10 reference genome using Bismark. Fragment-level methylation calls were made using the “Bismark Methylation Extractor” tool for each sample and aggregated into strand-specific CpG, CHG, and CHH context files. CpG context was the measure of interest while CHG and CHG files were used to assess conversion efficiency. Fragments were flagged if there was any sign of incomplete conversion in CHG and CHH contexts, and these fragment IDs were used to filter the CpG context files prior to quantifying methylation levels. CpG methylation was determined by the ratio of methylated read coverage to total read coverage for each CpG. Amplicon-wide and CpG-specific mean methylation values were calculated at the level of technical replicates, biological replicates, and experimental groups and summarized in box/dot plots.

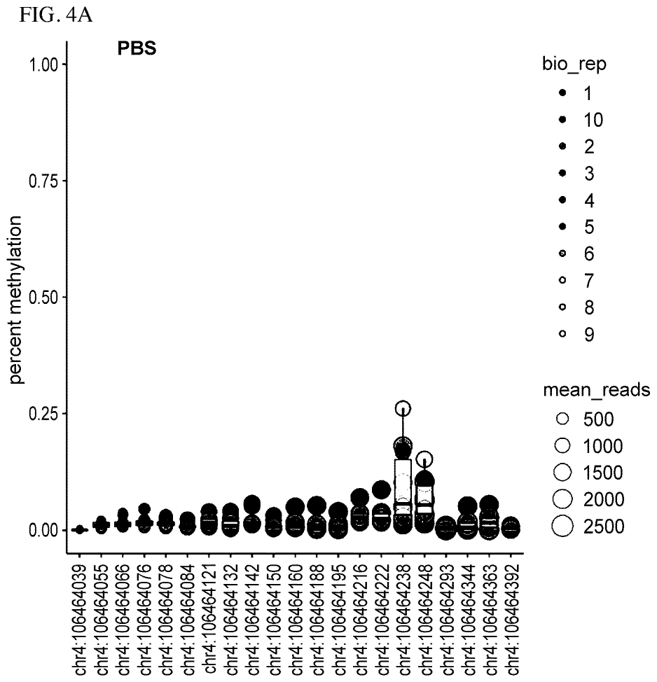

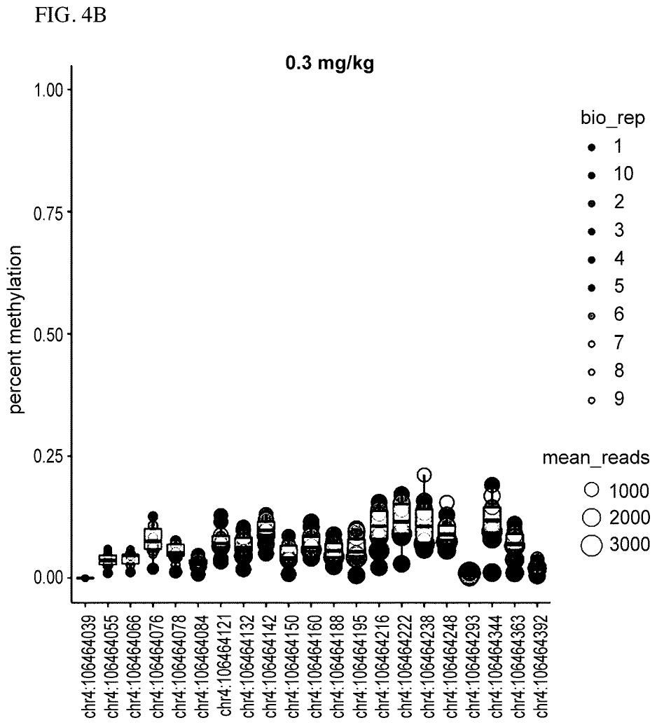

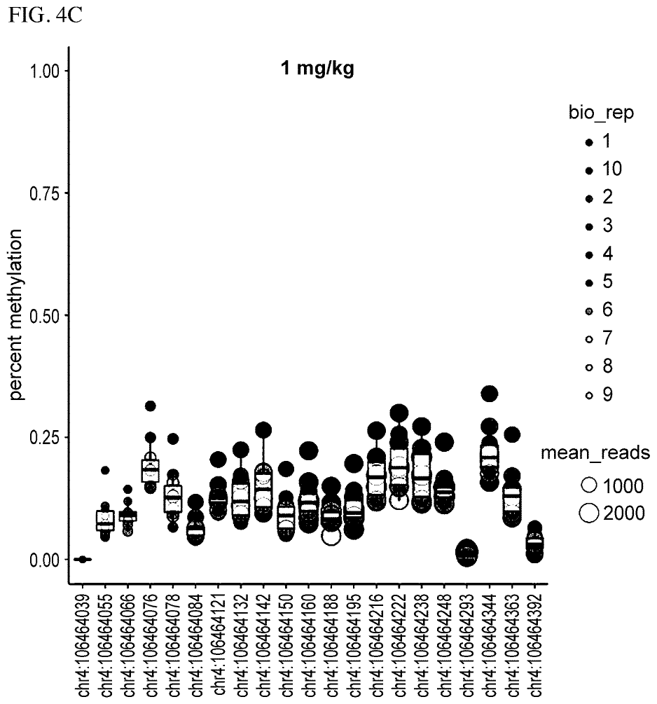

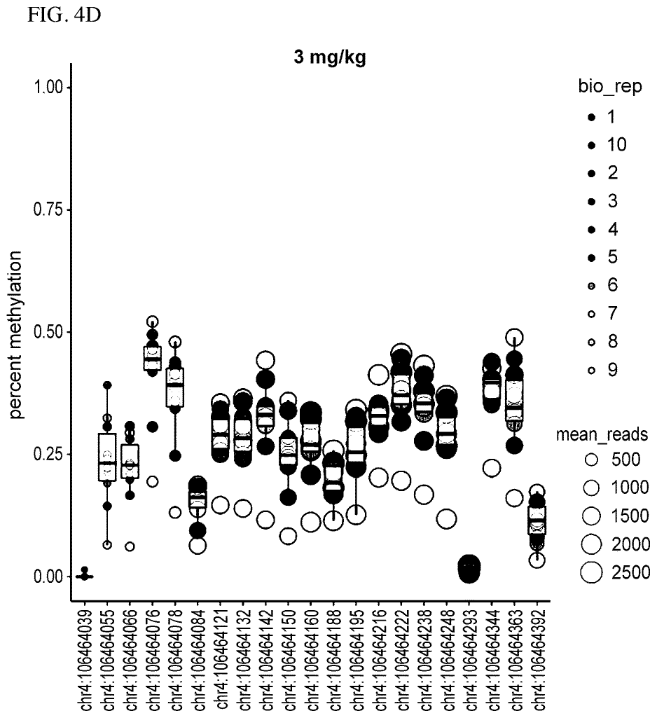

As shown in FIGS. 4A-4D, mice treated with TAL02-MQ1 showed a dose-dependent increase in mPCSK9 promoter methylation as determined by EM-Seq. Increased levels of DNA methylation were observed at doses 0.3 mg/kg, 1 mg/kg, and 3 mg/kg, respectively, with the increase in a dose-dependent fashion.

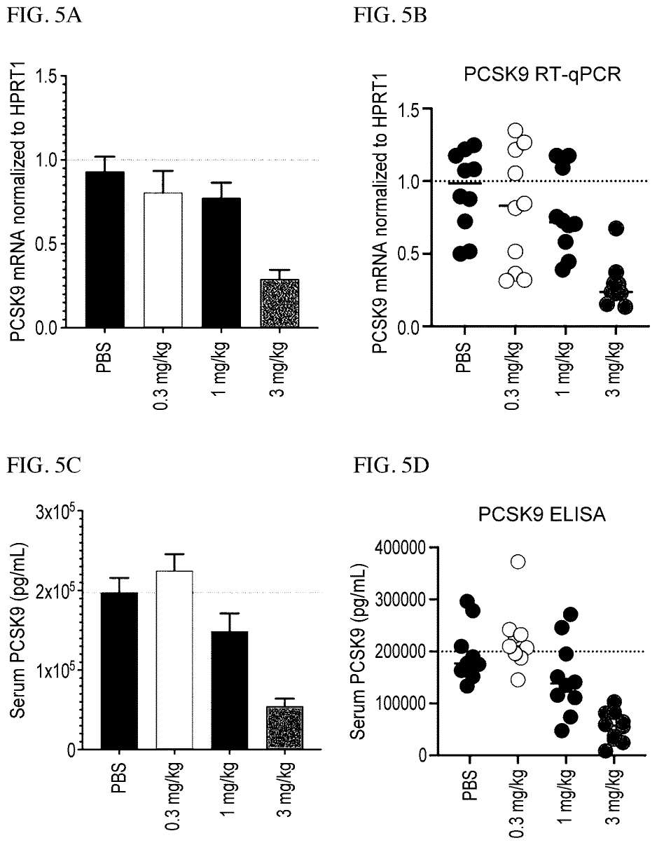

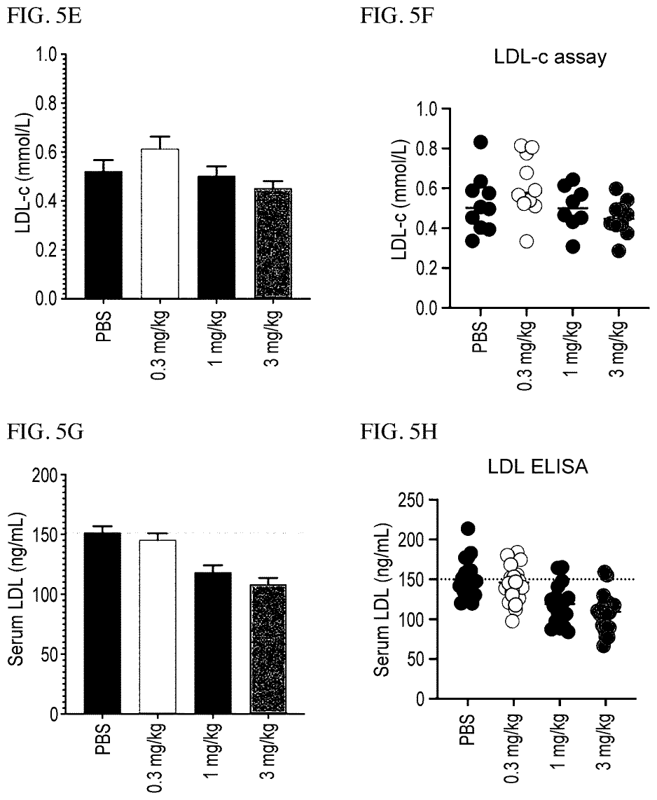

To assess the effect of LNP-formulated TAL02-MQ1 treatment on mPCSK9 mRNA levels, liver punches were homogenized, and RNA was isolated. RNA was then reverse-transcribed to cDNA. cDNA was analyzed by multiplexed qPCR using TaqMan® primer probes specific to HPRT1 (housekeeper control) and mPCSK9. Relative mPCSK9 mRNA expression was determined through the comparative ΔΔCt method. Average mPCSK9 normalized mRNA levels across subjects are shown in FIG. 5A and for individual subjects in FIG. 5B. Serum mPCSK9 levels were detected using the Abcam® Mouse PCSK9 ELISA Kit (ab215538) according to the manufacturer's instructions. Average mPCSK9 serum levels across subjects are shown in FIG. 5C and for individual subjects in FIG. 5D. Serum LDL-cholesterol was analyzed using the Mybiosource® mouse LDL-cholesterol kit (MBS2540573) according to the manufacturer's instructions. Average LDL-c across subjects is shown in FIG. 5E and for individual subjects in FIG. 5F. Serum LDL levels were detected using the Novus Biologicals® mouse LDL ELISA kit (NBP2-81135) according to the manufacturer's instructions. Average LDL across subjects is shown in FIG. 5G and for individual subjects in FIG. 5H.

Taken together, these data show that mice treated with LNP-formulated TAL02-MQ1 demonstrated decreased mPCSK9 mRNA, and decreased serum mPCSK9, LDL-cholesterol, and LDL, as compared to control mice 7-days after dosing.

### Example 3: Durability of In Vivo of Downregulation of mPCSK9 by MQ1 Effectors Fused to TALEs

This example describes the durability of mPCSK9 downregulation resulting from targeting the mPCSK9 mouse promoter with TALE-MQ1 effector fusions.

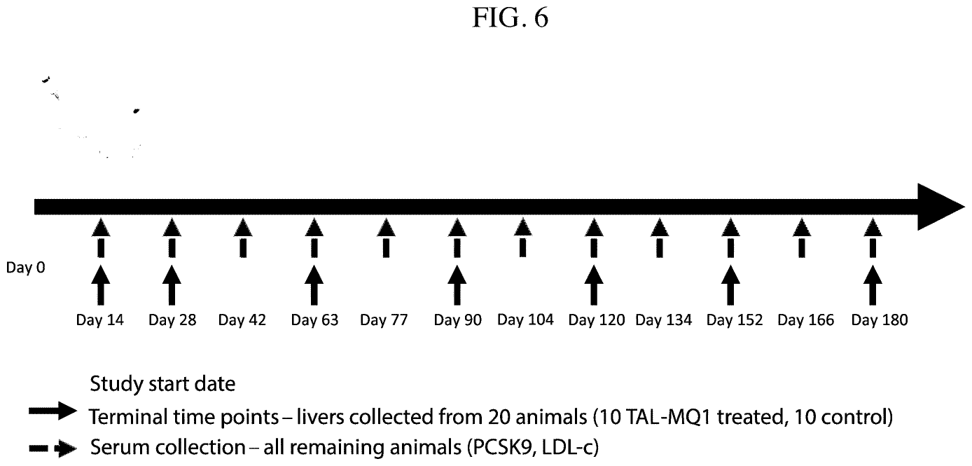

To determine the lasting effects of TAL-MQ1, a 6-month in vivo durability study was designed. A summary of the study design including dosing and time points for sample collection is schematically depicted in FIG. 6. Details of the study design are presented in Table 11 infra. An LNP formulation, TA-1 (TAL02-MQ1 (MR-33112-1)) was prepared; details of the LNPs are presented in Table 12 infra.

TABLE 11
Mouse mPCSK9 C57 BL/6 in vivo Durability Study Design

| | | | | | Dosing | | |
|---|---|---|---|---|---|---|---|
| | | | | | Regimen | Terminal | |
| Group | | Animal | Test | Dose | (1 IV | Time | |
| No. | Cohort | No. | Article | (mg/kg) | injection) | Point | Sample collection and Analytics |
| 1 | A | 70 | TA-1 | 3.0 | Day 0 | 10 | Liver punches (flash frozen) |
| 2 | A | 70 | PBS | NA | Day 0 | animals | RT-qPCR |
| | | | | | | at 7 | EM-seq |
| | | | | | | intervals | Serum (frozen) |
| | | | | | | up to 6 | mPCSK9 |
| | | | | | | months | LDL-c (or LDL) |

TABLE 12
TAL02-MQ1 Test Article mPCSK9 Lipid LNP Parameters for in vivo
Durability Study

| | LNP | | [RNA] | Vol. | Encapsul. | Diam. | | Charge | | N:P |
|---|---|---|---|---|---|---|---|---|---|---|
| LNP | Lot | mRNA | mg/ml | (mL) | Effic. | (nm) | PDI | (mV) | Lipids | ratio |
| TA-1 | FC189- | MR- | 0.70 | 20.4 | 96.9 | 100 ± | 0.057 ± | −3.17 ± | 50% | 3 |
| | 001 | 33112- | | | | 0.3 | 0.010 | 1.65 | MC3/ | |
| | | 1 | | | | | | | 38.5% | |
| | | | | | | | | | cholesterol/ | |
| | | | | | | | | | 10% | |
| | | | | | | | | | DSPC/ | |
| | | | | | | | | | 1.5% | |
| | | | | | | | | | PEG2 | |
| | | | | | | | | | K- | |
| | | | | | | | | | DMG | |

Briefly, the study comprised administering a single dose of LNP-formulated TAL02-MQ1. Selected mice were sacrificed at 2 weeks, 1 month, 2 months, 3 months, 4 months, 5 months, and 6 months, and their livers were harvested for analysis. Serum was collected for all animals every two weeks for mPCSK9 expression and LDL-c analysis.

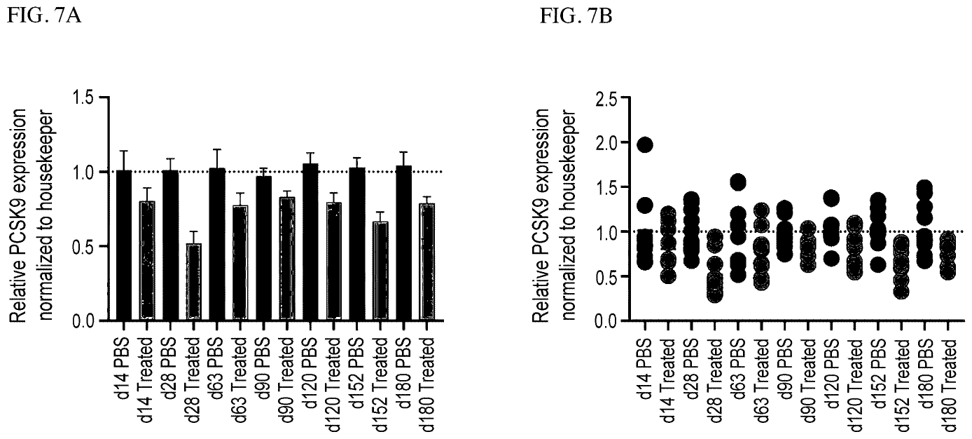

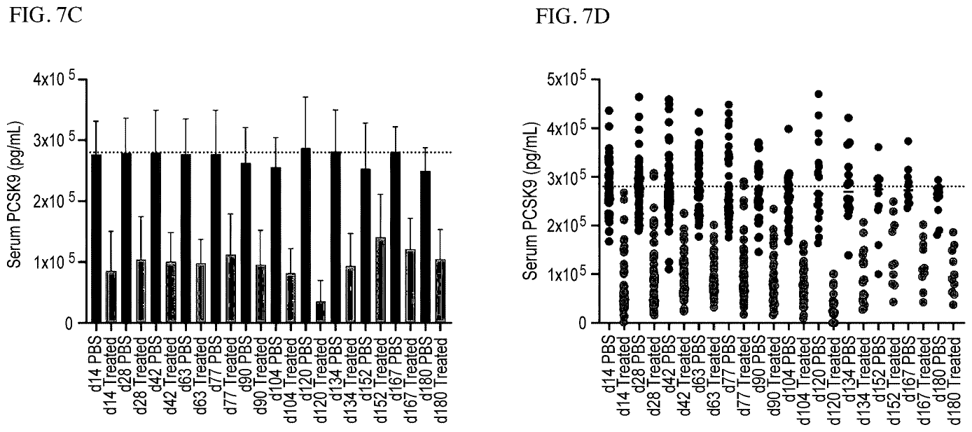

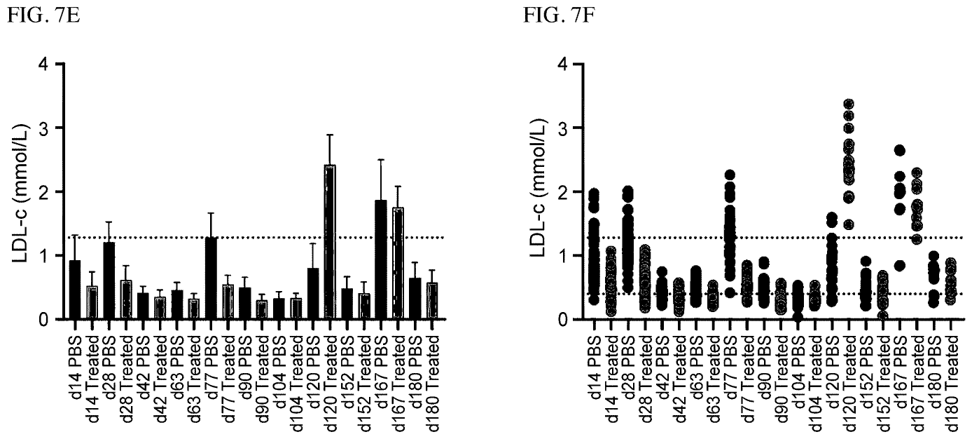

Liver mPCSK9 mRNA expression was quantified as described in Example 2 supra. RT-qPCR showed a trend in repression for mPCSK9 mRNA levels, with decreased mRNA levels observed 63-days post-dose. Average mPCSK9 normalized mRNA levels across subjects are shown in FIG. 7A and for individual subjects in FIG. 7B.

Serum levels of mPCSK9 were detected using the Abcam® Mouse PCSK9 ELISA Kit (ab215538) as described in Example 2 supra. Serum mPCSK9 was reduced about 70% following administration of LNP-formulated TAL02-MQ1, with continued reduction observed through 2.5 months. Average mPCSK9 serum levels across subjects are shown in FIG. 7C and for individual subjects in FIG. 7D.

Serum LDL-cholesterol (LDL-c) was analyzed using the Mybiosource® mouse LDL-cholesterol kit (MBS2540573) as described in Example 2 supra. Serum LDL-c was reduced about 55% following administration of TAL02-MQ1, with continued reduction through 2.5 months. Average LDL-c across subjects is shown in FIG. 7E and for individual subjects in FIG. 7F.

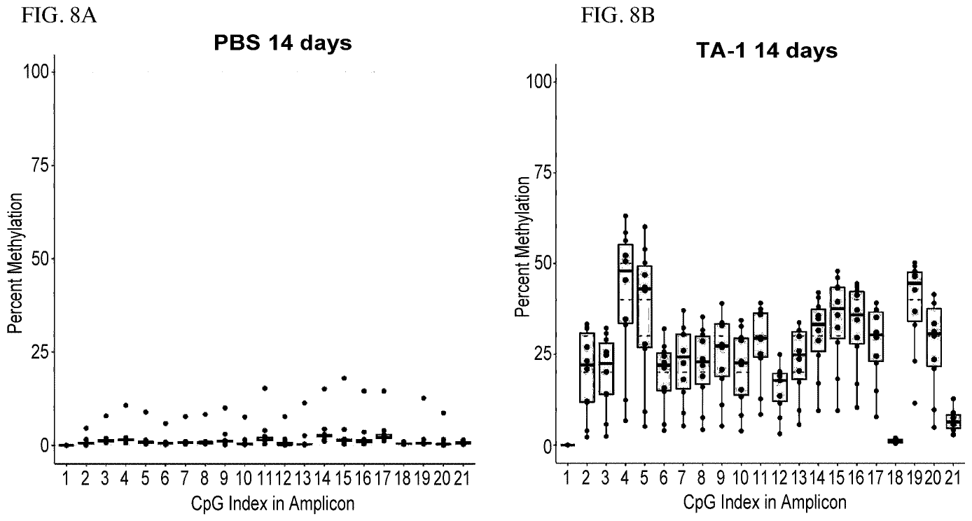

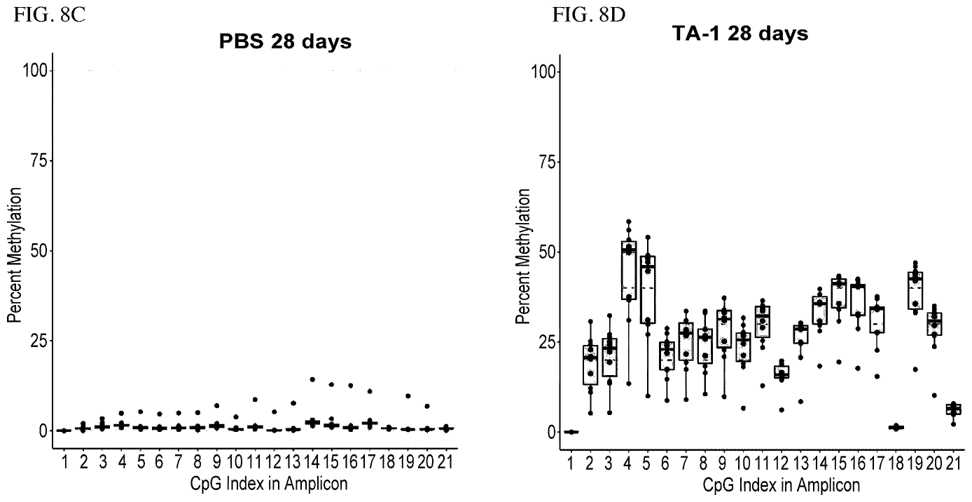

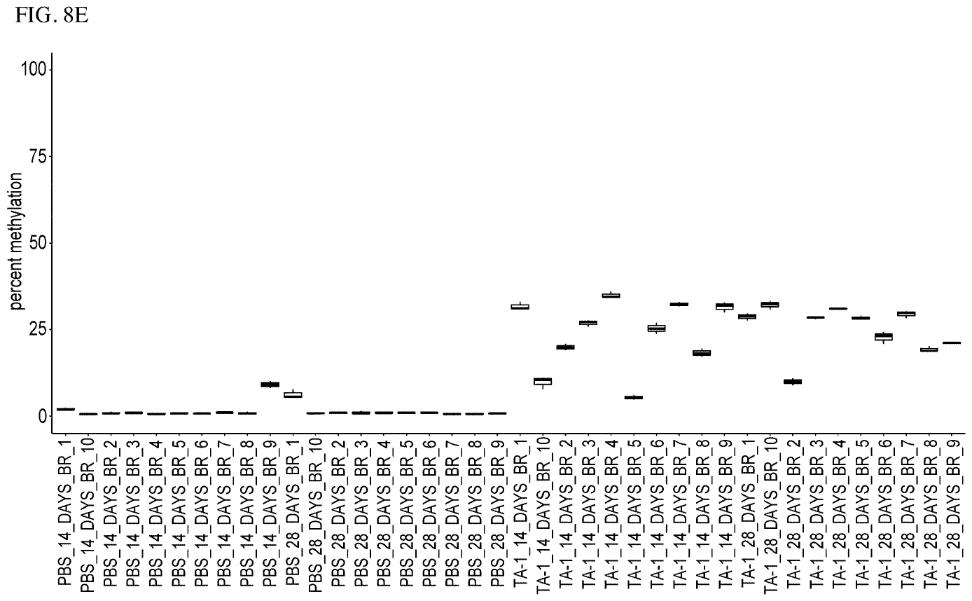

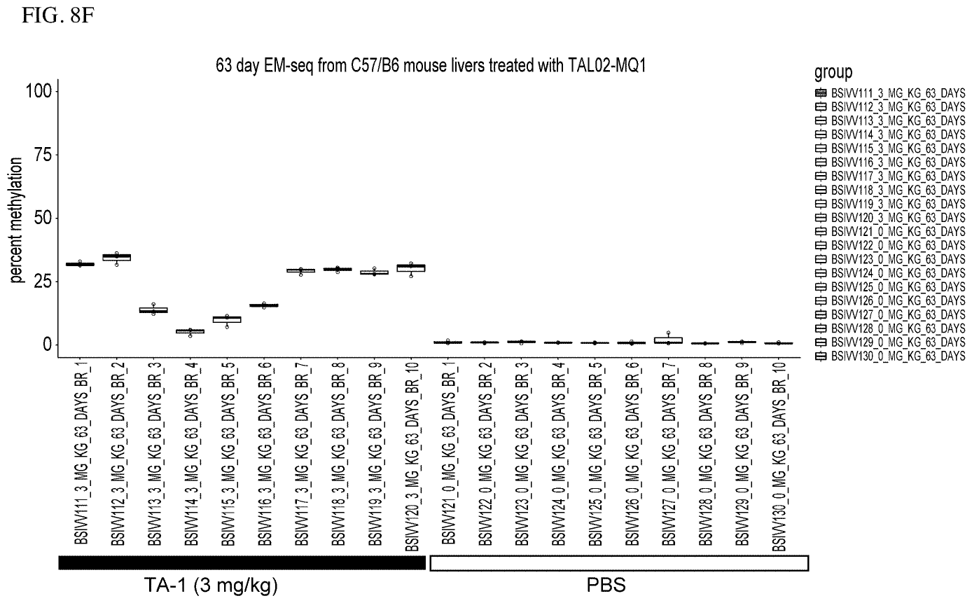

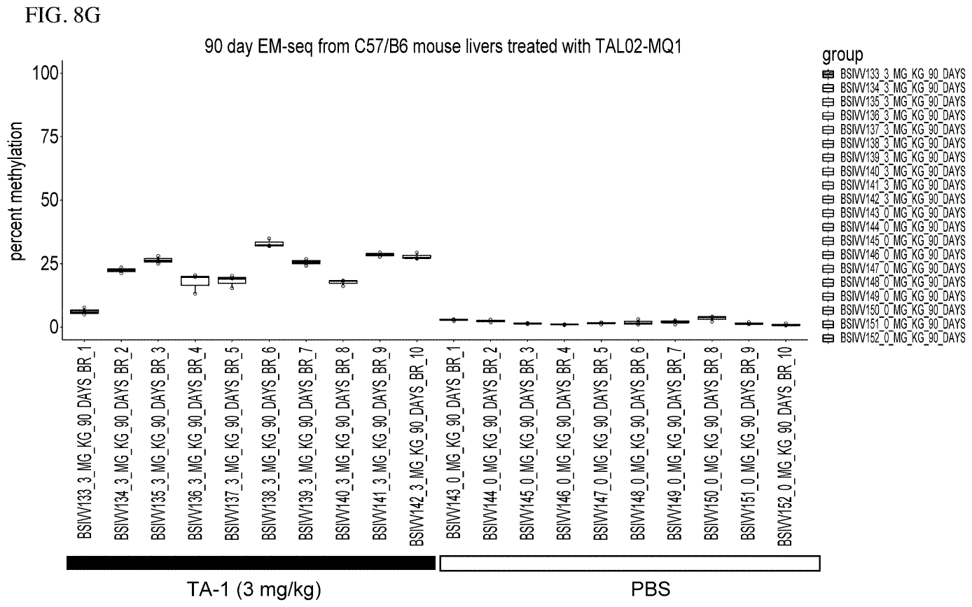

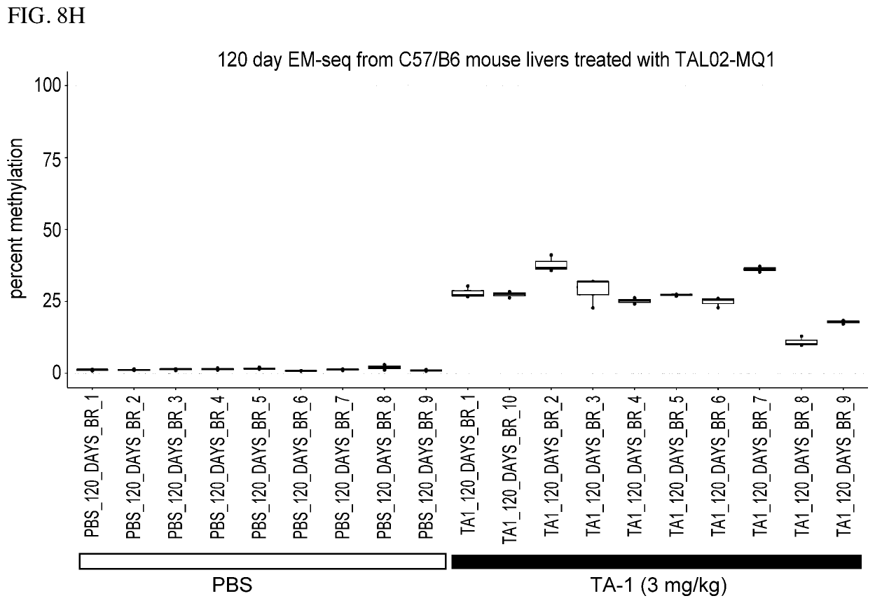

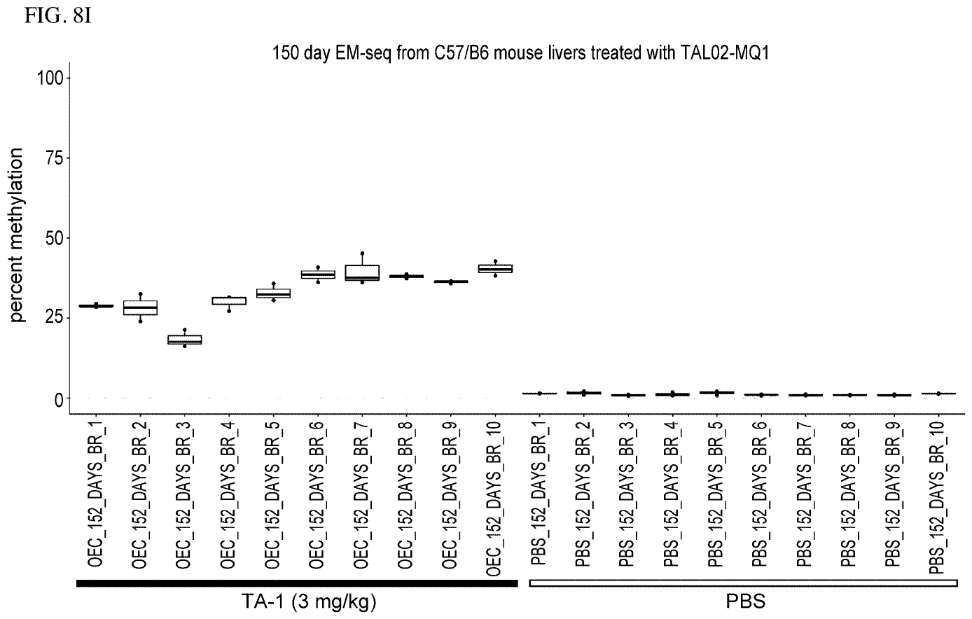

To determine if treatment with LNP-formulated TAL02-MQ1 was altering methylation near the mPCSK9 promoter, enzymatic methyl-seq (EM-Seq) was performed using the methods described in Example 2 supra. Mice treated with LNP-formulated TAL02-MQ1 showed increased in mPCSK9 promoter methylation at least up to 28-days post-treatment. A portion of the promoter was amplified in manner to preserve the methylation state, and methylation of 21 individual CpG dinucleotides (“CpG indexes”) within the amplicon were analyzed. FIGS. 8A-8D depict an increase in methylation of individual CpG indexes within the promoter of mPCSK9 in a murine model following intravenous administration of a dose of TAL02-MQ1 (TA-1). DNA methylation was quantified by Em-Seq following administration of PBS or TA-1 14-days post-dose (FIG. 8A and FIG. 8B, respectively) and 28-days post-dose (FIG. 8C and FIG. 8D, respectively). FIG. 8E depicts promoter methylation at 14-days and 28-days post dose, as the average methylation across the entire amplicon for each mouse for the tested conditions. FIGS. 8F-8I depict promoter methylation at 63 days, 90-days, 120-days, and 150-days post-dose respectively. Each hash mark on the X-axes of FIGS. 8E-8I represents an individual mouse in the study.

A single injection of LNP-formulated TAL02-MQ1 targeting the mPCSK9 promoter resulted in: lower serum mPCSK9 compared to PBS control for at least 180 days post treatment; lower serum LDL-cholesterol compared to PBS control for at least 180 days post treatment; and increased mPCSK9 promoter methylation for at least 150 days post treatment. Taken together, these data indicate a durable in vivo effect on transcription following administration mRNA encoding a fusion protein that increases methylation of the mPCSK9 promoter.

### Example 4: Specificity of In Vivo of Downregulation of mPCSK9 by MQ1 Effectors Fused to TALES

This example describes the specificity of mPCSK9 downregulation resulting from targeting the mPCSK9 mouse promoter with TALE-MQI effector fusions

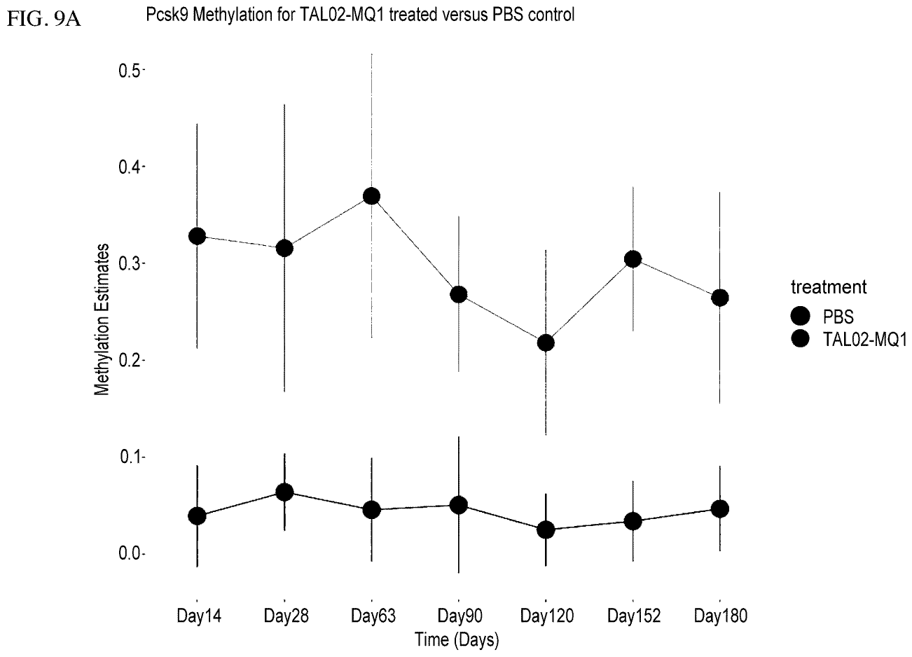

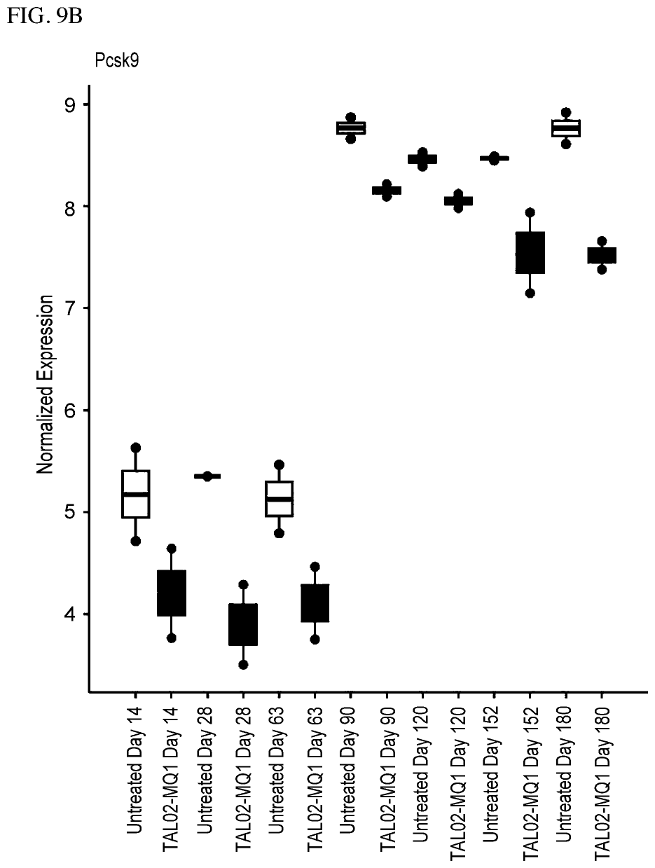

To understand the genomic specificity of EC effect in vivo and assess off-target effects, whole-genome methylation sequencing (WGMS) was performed on gDNA extracted from TALE-MQI effector fusions treated liver along with 3′ mRNA sequencing on the 6-month in vivo mouse study as detailed in Example 3. Briefly, whole genome methylation sequencing libraries were prepared using the NEB Enzymatic Methyl-Seq Kit (NEB Cat #E7120L) according to the manufacturer's instructions and sequenced on a NextSeq2000 using a 2×150 bp strategy. Bismark was used to align raw sequencing reads to a converted hg19 genome and estimate CpG methylation level. DMRSeq was used to identify differential methylated regions (DMR) between the treatment groups from the raw methylation estimate. The ‘adjustCovariate’ parameter was set to control time points. 4 DMRs were identified when comparing all Pcsk9-EC treated animals to PBS-treated controls adjusted for timepoints (adj. p<0.05, Table 13). These DMRs indicate loci that sustain methylation changes throughout the entirety of the 6-month treatment duration and include the Pcsk9 promoter as a top hit (FIG. 9A). Spock1, an extracellular matrix proteoglycan, and Slc20a2, a sodium-phosphate transporter, showed increased methylation at their promoter and intron, respectively (Table 13). Further, an intragenic region on chromosome 16 that showed a decrease of DNA methylation in treated animals compared to controls (Table 13), suggesting this was not a product of the TALE-MQI effector fusions.

To assess the effect of TALE-MQI effector fusions over 6 months, 3′ Digital Gene Expression (DGE) RNA-seq was performed using the Lexogen QuantSeq 3′ mRNA-seq V2 kit (Lexogen Cat #191.24) according to the manufacturer's instructions. Libraries were sequenced on a NextSeq 2000 using a 1×100 bp strategy. Raw sequence data was processed in accordance with the manufacturer's guidelines (QuantSeq 3′ mRNA-Seq Integrated Data Analysis Pipelines on Bluebee® Genomics Platform 015UG108V0201). Gene-level counts data were imported using tximport, and differential expression was calculated using DESeq2 with the ~timepoint+treatment for the experiment-wide analysis. Differential gene expression analysis using 3′ mRNA sequencing showed that only Pcsk9 (adj. p=7.36e-6, log 2FC=−1.08, Table 14, FIG. 9B) and Cd163, a macrophage scavenger receptor associated with anti-inflammation were significant (adj. p=1.16e-7, log 2FC=−1.39) after controlling for time. Neither Spock1 nor Slc20a2 showed significant differences in expression. Taken together, these data indicate high specificity for the TALE-MQI effector fusions in vivo over 6 months.

TABLE 13
Long-term significant differential methylated regions
in TALE-MQ1 effector treated mice after 6 months

| | | | DMR | |
|---|---|---|---|---|
| | | Gene | width | |
| Positions | Gene | Region | (bp) | β |
| chr4: 106463890-106463987 | Pcsk9 | PP | 98 | 0.822 |
| chr4: 106464483-106465223 | Pcsk9 | PP | 741 | 0.839 |
| chr4: 106464075-106464187 | Pcsk9 | PP | 113 | 0.607 |
| chr4: 106463708-106463803 | Pcsk9 | PP | 96 | 0.728 |
| chr13: 57908322-57908349 | Spock1 | PP | 28 | 0.547 |
| chr16: 10823600-10823995 | NA | Intergenic | 396 | −0.731 |
| chr8: 22508763-22508933 | Slc20a2 | Intronic | 171 | 0.770 |

PP: Promoter-proximal;
Intronic: non-regulatory region

TABLE 14
Time adjusted significant differential expression genes
in TALE-MQ1 effector treated mice after 6 months

| Gene | Ensembl ID | log2 Fold Change | Adj. P-value |
|---|---|---|---|
| Cd163 | ENSMUSG00000008845 | −1.39 | 1.16E−07 |
| Pcsk9 | ENSMUSG00000044254 | −1.08 | 7.36E−06 |

TABLE 15
Target Sequences

| | SEQ ID |
|---|---|
| Target Sequence | NO |
| CTACTGTGCCCCACCGGCGC | 1 |
| TGGACGCGCAGGCTGCCGGT | 2 |
| GGATCTTCCGATGGGGCTCG | 3 |
| GAGCAGCAGCGGCGGCAACAG | 4 |
| AGCAGCAGCGGCGGCAACAG | 5 |
| GGCTGATGAGGCCGCACATG | 6 |
| AGATTTCTCACTTGTTGTAC | 10 |
| GTTGTACTGGTGCCCTCTGC | 11 |
| GACTACAATGGACCAGCAGA | 12 |
| TCTAGGAGCAGTGACTACAA | 13 |
| TATGACCAGAAGATGGCGTC | 14 |
| GGTTGCCAGACGCCATCTTC | 15 |
| CCATAACTATGACCAGAAGA | 16 |
| CCATCTTCTGGTCATAGTTA | 17 |
| AACTACTCTTTACTGACCCC | 18 |
| TACTCTTTACTGACCCCTGG | 19 |
| TTTGTTGCTGAGGTGCCACC | 20 |
| AGACTTCTGAATCAATCC | 67 |
| AGAAGTCTCACTGGTCAG | 68 |
| GGTGGACCCAGGAAACAC | 69 |
| AAGGTGGACCCAGGAAAC | 70 |
| ACTGGAGGATCAGGTTTC | 71 |
| GTTTAATCAGATAGGATC | 72 |
| ATCTGATTAAACATTAAC | 73 |
| AAACCTTCTAGGGTGTGG | 74 |
| AACCTTCTAGGGTGTGGG | 75 |
| CACCCACACCCTAGAAGG | 76 |
| GAATCAATCCTACTGTGG | 77 |
| GATCCTATCTGATTAAAC | 78 |
| CACACCCTAGAAGGTTTC | 79 |
| GAAACCTTCTAGGGTGTG | 80 |

TABLE 16
gRNA sequences

| | SEQ |
|---|---|
| | ID |
| Sequence | NO |
| mCs;mUs;mAs;rC;rU;rG;rU;rG;rC;rC;rC;rC;rA;rC;rC;rG;rG;rC;rG;rC;rG;rU;rU;rU; | 21 |
| rU;rA;rG;mA;mG;mC;mU;mA;mG;mA;mA;mA;mU;mA;mG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;mA;mA;mC;mU;mU;mG;mA; | |
| mA;mA;mA;mA;mG;mU;mG;mG;mC;mA;mC;mC;mG;mA;mG;mU;mC;mG;mG;mU;mG;mC;mUs;mUs; | |
| mUs;mU | |
| mUs;mGs;mGs;rA;rC;rG;rC;rG;rC;rA;rG;rG;rC;rU;rG;rC;rC;rG;rG;rU;rG;rU;rU;rU; | 22 |
| rU;rA;rG;mA;mG;mC;mU;mA;mG;mA;mA;mA;mU;mA;mG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;mA;mA;mC;mU;mU;mG;mA; | |
| mA;mA;mA;mA;mG;mU;mG;mG;mC;mA;mC;mC;mG;mA;mG;mU;mC;mG;mG;mU;mG;mC;mUs;mUs; | |
| mUs;mU | |
| mGs;mGs;mAs;rU;rC;rU;rU;rC;rC;rG;rA;rU;rG;rG;rG;rG;rC;rU;rC;rG;rG;rU;rU;rU; | 23 |
| rU;rA;rG;mA;mG;mC;mU;mA;mG;mA;mA;mA;mU;mA;mG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;mA;mA;mC;mU;mU;mG;mA; | |
| mA;mA;mA;mA;mG;mU;mG;mG;mC;mA;mC;mC;mG;mA;mG;mU;mC;mG;mG;mU;mG;mC;mUs;mUs; | |
| mUs;mU | |
| mGs;mAs;mGs;rC;rA;rG;rC;rA;rG;rC;rG;rG;rC;rG;rG;rC;rA;rA;rC;rA;rG;rG;rU;rU; | 24 |
| rU;rU;rA;rG;mA;mG;mC;mU;mA;mG;mA;mA;mA;mU;mA;mG;rC;rA;rA;rG;rU;rU;rA;rA;rA; | |
| rA;rU;rA;rA;rG;rG;rC;rU;rA;rG;rU;rC;rC;rG;rU;rU;rA;rU;rC;mA;mA;mC;mU;mU;mG; | |
| mA;mA;mA;mA;mA;mG;mU;mG;mG;mC;mA;mC;mC;mG;mA;mG;mU;mC;mG;mG;mU;mG;mC;mUs; | |
| mUs;mUs;mU | |
| mAs;mGs;mCs;rA;rG;rC;rA;rG;rC;rG;rG;rC;rG;rG;rC;rA;rA;rC;rA;rG;rG;rU;rU;rU; | 25 |
| rU;rA;rG;mA;mG;mC;mU;mA;mG;mA;mA;mA;mU;mA;mG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;mA;mA;mC;mU;mU;mG;mA; | |
| mA;mA;mA;mA;mG;mU;mG;mG;mC;mA;mC;mC;mG;mA;mG;mU;mC;mG;mG;mU;mG;mC;mUs;mUs; | |
| mUs;mU | |
| mGs;mGs;mCs;rU;rG;rA;rU;rG;rA;rG;rG;rC;rC;rG;rC;rA;rC;rA;rU;rG;rG;rU;rU;rU; | 26 |
| rU;rA;rG;mA;mG;mC;mU;mA;mG;mA;mA;mA;mU;mA;mG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;mA;mA;mC;mU;mU;mG;mA; | |
| mA;mA;mA;mA;mG;mU;mG;mG;mC;mA;mC;mC;mG;mA;mG;mU;mC;mG;mG;mU;mG;mC;mUs;mUs; | |
| mUs;mU | |
| mCs;mUs;mAs;rC;rU;rG;rU;rG;rC;rC;rC;rC;rA;rC;rC;rG;rG;rC;rG;rC;rG;rU;rU;rU; | 27 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mUs;mGs;mGs;rA;rC;rG;rC;rG;rC;rA;rG;rG;rC;rU;rG;rC;rC;rG;rG;rU;rG;rU;rU;rU; | 28 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mGs;mGs;mAs;rU;rC;rU;rU;rC;rC;rG;rA;rU;rG;rG;rG;rG;rC;rU;rC;rG;rG;rU;rU;rU; | 29 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mAs;mGs;mAs;rU;rU;rU;rC;rU;rC;rA;rC;rU;rU;rG;rU;rU;rG;rU;rA;rC;rG;rU;rU;rU; | 30 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mGs;mUs;mUs;rG;rU;rA;rC;rU;rG;rG;rU;rG;rC;rC;rC;rU;rC;rU;rG;rC;rG;rU;rU;rU; | 31 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mGs;mAs;mCs;rU;rA;rC;rA;rA;rU;rG;rG;rA;rC;rC;rA;rG;rC;rA;rG;rA;rG;rU;rU;rU; | 32 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mUs;mCs;mUs;rA;rG;rG;rA;rG;rC;rA;rG;rU;rG;rA;rC;rU;rA;rC;rA;rA;rG;rU;rU;rU; | 33 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mUs;mAs;mUs;rG;rA;rC;rC;rA;rG;rA;rA;rG;rA;rU;rG;rG;rC;rG;rU;rC;rG;rU;rU;rU; | 34 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mGs;mGs;mUs;rU;rG;rC;rC;rA;rG;rA;rC;rG;rC;rC;rA;rU;rC;rU;rU;rC;rG;rU;rU;rU; | 35 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mCs;mCs;mAs;rU;rA;rA;rC;rU;rA;rU;rG;rA;rC;rC;rA;rG;rA;rA;rG;rA;rG;rU;rU;rU; | 36 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mCs;mCs;mAs;rU;rC;rU;rU;rC;rU;rG;rG;rU;rC;rA;rU;rA;rG;rU;rU;rA;rG;rU;rU;rU; | 37 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mAs;mAs;mCs;rU;rA;rC;rU;rC;rU;rU;rU;rA;rC;rU;rG;rA;rC;rC;rC;rC;rG;rU;rU;rU; | 7 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mUs;mAs;mCs;rU;rC;rU;rU;rU;rA;rC;rU;rG;rA;rC;rC;rC;rC;rU;rG;rG;rG;rU;rU;rU; | 8 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |
| mUs;mUs;mUs;rG;rU;rU;rG;rC;rU;rG;rA;rG;rG;rU;rG;rC;rC;rA;rC;rC;rG;rU;rU;rU; | 9 |
| rU;rA;rG;rA;rG;rC;rU;rA;rG;rA;rA;rA;rU;rA;rG;rC;rA;rA;rG;rU;rU;rA;rA;rA;rA; | |
| rU;rA;rA;rG;rG;rC;rU;rA;rG;rU;rC;rC;rG;rU;rU;rA;rU;rC;rA;rA;rC;rU;rU;rG;rA; | |
| rA;rA;rA;rA;rG;rU;rG;rG;rC;rA;rC;rC;rG;rA;rG;rU;rC;rG;rG;rU;rG;rC;rUs;mUs; | |
| mUs;mU | |

wherein
m is 2′-O-methyl,
r is standard ribose sugar, and
s is phosphorothioate linker

TABLE 17
Sequences

| | | SEQ |
|---|---|---|
| | | ID |
| Name | Sequence | NO |
| DNMT3a/3L | EWGPFDLVIGGSPCNDLSIVNPARKGLYEGTGRLFFEFYRLLHD | 38 |
| amino acid 1 | ARPKEGDDRPFFWLFENVVAMGVSDKRDISRFLESNPVMIDAK | |
| | EVSAAHRARYFWGNLPGMNRPLASTVNDKLELQECLEHGRIA | |
| | KFSKVRTITTRSNSIKQGKDQHFPVFMNEKEDILWCTEMERVFG | |
| | FPVHYTDVSNMSRLARQRLLGRSWSVPVIRHLFAPLKEYFACV | |
| | SSGNSNANSRGPSFSSGLVPLSLRGSHMNPLEMFETVPVWRRQP | |
| | VRVLSLFEDIKKELTSLGFLESGSDPGQLKHWDVTDTVRKDVE | |
| | EWGPFDLVYGATPPLGHTCDRPPSWYLFQFHRLLQYARPKPGS | |
| | PRPFFWMFVDNLVLNKEDLDVASRFLEMEPVTIPDVHGGSLQN | |
| | AVRVWSNIPAIRSRHWALVSEEELSLLAQNKQSSKLAAKWPTK | |
| | LVKNCFLPLREYFKYFSTELTSSL | |
| DNMT3a/3L | NHDQEFDPPKVYPPVPAEKRKPIRVLSLFDGIATGLLVLKDLGI | 39 |
| amino acid 2 | QVDRYIASEVCEDSITVGMVRHQGKIMYVGDVRSVTQKHIQE | |
| | WGPFDLVIGGSPCNDLSIVNPARKGLYEGTGRLFFEFYRLLHDA | |
| | RPKEGDDRPFFWLFENWAMGVSDKRDISRFLESNPVMIDAKEV | |
| | SAAHRARYFWGNLPGMNRPLASTVNDKLELQECLEHGRIAKFS | |
| | KVRTITTRSNSIKQGKDQHFPVFMNEKEDILWCTEMERVFGFPV | |
| | HYTDVSNMSRLARQRLLGRSWSVPVIRHLFAPLKEYFACVSSG | |
| | NSNANSRGPSFSSGLVPLSLRGSHMNPLEMFETVPVWRRQPVR | |
| | VLSLFEDIKKELTSLGFLESGSDPGQLKHWDVTDTVRKDVEEW | |
| | GPFDLVYGATPPLGHTCDRPPSWYLFQFHRLLQYARPKPGSPRP | |
| | FFWMFVDNLVLNKEDLDVASRFLEMEPVTIPDVHGGSLQNAV | |
| | RVWSNIPAIRSRHWALVSEEELSLLAQNKQSSKLAAKWPTKLV | |
| | KNCFLPLREYFKYFSTELTSSL | |
| DNMT3a/3L | AACCACGACCAGGAGTTCGACCCCCCCAAGGTGTACCCCCC | 40 |
| nucleotide | CGTGCCCGCCGAGAAGCGGAAGCCCATCCGGGTGCTGAGCC | |
| | TGTTCGACGGCATCGCCACCGGCCTGCTGGTGCTGAAGGACC | |
| | TGGGCATCCAGGTGGACCGGTACATCGCCAGCGAGGTGTGC | |
| | GAGGACAGCATCACCGTGGGCATGGTGCGGCACCAGGGCAA | |
| | GATCATGTACGTGGGCGACGTGCGGAGCGTGACCCAGAAGC | |
| | ACATCCAGGAGTGGGGCCCCTTCGACCTGGTGATCGGCGGC | |
| | AGCCCCTGCAACGACCTGAGCATCGTGAACCCCGCCCGGAA | |
| | GGGCCTGTACGAGGGCACCGGCCGGCTGTTCTTCGAGTTCTA | |
| | CCGGCTGCTGCACGACGCCCGGCCCAAGGAGGGCGACGACC | |
| | GGCCCTTCTTCTGGCTGTTCGAGAACGTGGTGGCCATGGGCG | |
| | TGAGCGACAAGCGGGACATCAGCCGGTTCCTGGAGAGCAAC | |
| | CCCGTGATGATCGACGCCAAGGAGGTGAGCGCCGCCCACCG | |
| | GGCCCGGTACTTCTGGGGCAACCTGCCCGGCATGAACCGGC | |
| | CCCTGGCCAGCACCGTGAACGACAAGCTGGAGCTGCAGGAG | |
| | TGCCTGGAGCACGGCCGGATCGCCAAGTTCAGCAAGGTGCG | |
| | GACCATCACCACCCGGAGCAACAGCATCAAGCAGGGCAAGG | |
| | ACCAGCACTTCCCCGTGTTCATGAACGAGAAGGAGGACATC | |
| | CTGTGGTGCACCGAGATGGAGCGGGTGTTCGGCTTCCCCGTG | |
| | CACTACACCGACGTGAGCAACATGAGCCGGCTGGCCCGGCA | |
| | GCGGCTGCTGGGCCGGAGCTGGAGCGTGCCCGTGATCCGGC | |
| | ACCTGTTCGCCCCCCTGAAGGAGTACTTCGCCTGCGTGAGCA | |
| | GCGGCAACAGCAACGCCAACAGCCGGGGCCCCAGCTTCAGC | |
| | AGCGGCCTGGTGCCCCTGAGCCTGCGGGGCAGCCACATGAA | |
| | TCCTCTGGAGATGTTCGAGACAGTGCCCGTGTGGAGAAGGC | |
| | AACCCGTGAGGGTGCTGAGCCTCTTCGAGGACATTAAGAAG | |
| | GAGCTGACCTCTCTGGGCTTTCTGGAATCCGGCAGCGACCCC | |
| | GGCCAGCTGAAACACGTGGTGGACGTGACCGACACAGTGAG | |
| | GAAGGACGTGGAAGAGTGGGGCCCCTTTGACCTCGTGTATG | |
| | GAGCCACACCTCCTCTCGGCCACACATGCGATAGGCCTCCCA | |
| | GCTGGTATCTCTTCCAGTTCCACAGACTGCTCCAGTACGCCA | |
| | GACCTAAGCCCGGCAGCCCCAGACCCTTCTTCTGGATGTTCG | |
| | TGGACAATCTGGTGCTGAACAAGGAGGATCTGGATGTGGCC | |
| | AGCAGATTTCTGGAGATGGAACCCGTGACAATCCCCGACGT | |
| | GCATGGCGGCTCTCTGCAGAACGCCGTGAGAGTGTGGTCCA | |
| | ACATCCCCGCCATTAGAAGCAGACACTGGGCTCTGGTGAGC | |
| | GAGGAGGAACTGTCTCTGCTGGCCCAGAATAAGCAGTCCTC | |
| | CAAGCTGGCCGCCAAGTGGCCCACCAAGCTGGTGAAGAACT | |
| | GCTTTCTGCCTCTGAGGGAGTATTTCAAGTATTTCAGCACCG | |
| | AACTGACCAGCAGCCTG | |
| TAL01-MQ1 | GGGAAAUAAGAGAGAAAAGAAGAGUAAGAAGAAAUAUAA | 41 |
| full-length | GAGCCACCAUGGCCCCAAAGAAGAAGCGGAAAGUCGGAGG | |
| mRNA | UUCAGGCGGCUCGCAUAUGGUGGAUCUGAGAACCCUGGGU | |
| sequence | UACAGCCAGCAGCAGCAGGAAAAGAUUAAGCCGAAGGUCC | |
| | GCUCAACCGUGGCCCAACACCACGAGGCCUUGGUCGGACA | |
| | UGGGUUCACUCACGCCCACAUCGUGGCGCUCUCGCAACACC | |
| | CGGCAGCCCUGGGAACCGUGGCUGUGAAGUACCAAGACAU | |
| | GAUCGCGGCCCUGCCUGAAGCAACGCAUGAGGCCAUUGUC | |
| | GGCGUGGGAAAGCGCGGAGCUGGAGCCAGAGCGCUGGAAG | |
| | CCCUGCUGACCGUCGCUGGGGAACUGCGGGGACCACCUCU | |
| | GCAACUGGACACUGGACAACUGCUCAAGAUCGCUAAGCGC | |
| | GGGGGAGUGACUGCCGUGGAGGCGGUGCAUGCCUGGAGGA | |
| | ACGCCCUGACUGGAGCACCUCUGAAUCUGACGCCCGAACA | |
| | GGUGGUGGCCAUUGCGUCCAACAACGGAGGUAAACAGGCC | |
| | CUUGAGACUGUGCAACGGUUGCUGCCAGUCCUGUGCCAGG | |
| | CCCAUGGCCUUACACCAGAACAAGUGGUCGCUAUUGCCAG | |
| | CAACGGAGGAGGCAAGCAGGCACUGGAGACUGUUCAACGC | |
| | CUCCUGCCGGUCCUGUGCCAGGCCCAUGGAUUGACUCCUG | |
| | AGCAAGUCGUGGCUAUCGCGUCGCACGAUGGAGGCAAACA | |
| | GGCUCUCGAAACGGUGCAAAGACUUCUGCCCGUCCUGUGU | |
| | CAAGCGCAUGGCCUCACCCCUGAACAAGUAGUGGCCAUAG | |
| | CCUCCCAUGACGGGGGAAAACAGGCUUUGGAGACUGUGCA | |
| | GAGGCUGCUGCCUGUCCUGUGCCAGGCCCACGGUUUGACC | |
| | CCGGAACAGGUUGUGGCCAUUGCCUCGAAUAUCGGAGGGA | |
| | AACAAGCGCUAGAAACCGUGCAGAGACUUCUUCCUGUCCU | |
| | CUGUCAGGCUCACGGUCUGACUCCUGAACAGGUGGUGGCU | |
| | AUUGCUUCUAACAACGGUGGGAAGCAGGCCCUAGAAACUG | |
| | UGCAAAGGCUGCUUCCCGUGCUAUGCCAGGCUCAUGGACU | |
| | CACCCCGGAGCAAGUCGUGGCCAUUGCAUCCAAUGGAGGG | |
| | GGGAAGCAGGCCCUGGAAACUGUGCAGCGCUUGCUGCCUG | |
| | UGCUGUGCCAAGCUCACGGCCUAACCCCCGAACAGGUGGU | |
| | GGCCAUCGCCUCCAACAUCGGCGGGAAGCAGGCCCUCGAA | |
| | ACAGUCCAGAGAUUGCUCCCGGUGCUGUGUCAGGCCCACG | |
| | GACUCACGCCGGAACAAGUGGUGGCGAUUGCCUCACACGA | |
| | UGGUGGCAAACAAGCACUGGAAACGGUCCAGAGACUGCUC | |
| | CCGGUCCUCUGCCAGGCGCAUGGCUUGACGCCAGAACAGG | |
| | UCGUGGCGAUUGCCUCACACGAUGGGGGAAAGCAGGCUCU | |
| | GGAAACAGUGCAGAGAUUGUUACCUGUGCUCUGUCAAGCC | |
| | CACGGCUUGACUCCAGAACAGGUCGUAGCAAUUGCCUCCC | |
| | ACGAUGGCGGAAAGCAGGCGUUAGAGACAGUGCAACGGCU | |
| | GCUCCCCGUGUUGUGUCAAGCACAUGGGCUGACACCGGAA | |
| | CAAGUGGUAGCCAUCGCGAGCAACAUUGGAGGGAAGCAAG | |
| | CGCUCGAGACAGUCCAACGGCUUCUGCCGGUGCUGUGUCA | |
| | GGCACAUGGCCUGACCCCUGAGCAAGUUGUCGCCAUUGCU | |
| | UCCCAUGAUGGAGGAAAGCAAGCCCUAGAGACUGUCCAAC | |
| | GCCUGUUGCCGGUACUCUGCCAAGCCCAUGGUUUGACUCC | |
| | GGAACAGGUGGUGGCAAUCGCUUCCAAUAUCGGCGGCAAG | |
| | CAAGCUCUCGAAACCGUCCAGCGACUCCUGCCCGUGCUUU | |
| | GUCAGGCGCACGGACUGACUCCCGAACAAGUCGUCGCAAU | |
| | UGCCAGCCAUGAUGGUGGAAAGCAGGCCUUGGAAACUGUC | |
| | CAGCGGCUCCUGCCAGUGCUUUGCCAAGCACACGGACUUA | |
| | CCCCCGAGCAAGUGGUCGCAAUCGCGUCACAUGACGGCGG | |
| | GAAACAGGCGCUGGAAACCGUUCAGAGACUCUUGCCGGUG | |
| | UUGUGCCAGGCACACGGGCUCACCCCAGAACAGGUCGUCG | |
| | CCAUCGCCAGCCACGACGGUGGAAAACAAGCUCUUGAAAC | |
| | GGUGCAGCGCCUCCUUCCGGUGCUCUGUCAAGCUCAUGGG | |
| | CUUACUCCAGAGCAAGUAGUCGCUAUCGCCUCGCAUGACG | |
| | GUGGACGACCAGCCCUGGAAUCCAUCGUGGCCCAGCUCUC | |
| | CAGACCGGACCCUGCUCUCGCUGCCCUGACCAACGACCACU | |
| | UGGUGGCCCUGGCCUGUCUGGGUGGUAGACCGGCGUUGGA | |
| | CGCUGUCAAGAAGGGACUCCCUCACGCACCGGCACUUAUU | |
| | AAGCGGACCAACAGGCGGAUCCCUGAGCGCACUUCCCAUC | |
| | GCGUGGCAUCUGGGAGCGGAGGAGGAUCCGGCGGAGCCAG | |
| | AGACUCCAAGGUCGAGAACAAGACCAAGAAGCUGCGGGUG | |
| | UUCGAGGCCUUUGCCGGGAUCGGAGCACAGCGGAAGGCCU | |
| | UGGAGAAGGUCCGCAAGGACGAAUACGAAAUCGUGGGCCU | |
| | UGCCGAGUGGUACGUGCCAGCCAUCGUGAUGUACCAGGCU | |
| | AUCCACAACAACUUCCACACCAAGCUGGAGUACAAGUCCG | |
| | UGAGCAGAGAAGAGAUGAUCGACUACCUCGAAAACAAGAC | |
| | CCUGUCCUGGAACUCCAAGAAUCCCGUGUCGAACGGUUAC | |
| | UGGAAGCGCAAGAAGGAUGAUGAACUGAAGAUUAUCUACA | |
| | ACGCGAUUAAGCUGUCCGAAAAGGAAGGAAACAUUUUCGA | |
| | CAUUCGCGAUCUGUACAAGAGAACCCUCAAGAACAUUGAU | |
| | CUGCUUACCUACUCCUUCCCGUGCCAAGAUCUCAGCCAGCA | |
| | GGGGAUUCAGAAGGGGAUGAAGCGGGGUUCAGGGACCAGA | |
| | UCAGGCCUGCUCUGGGAGAUCGAAAGAGCCCUGGACUCCA | |
| | CCGAGAAGAACGAUCUCCCGAAAUACCUUCUGAUGGAGAA | |
| | CGUCGGCGCUCUCCUGCACAAGAAGAACGAAGAAGAACUG | |
| | AACCAGUGGAAGCAGAAGCUGGAGUCACUCGGAUACCAGA | |
| | AUUCUAUUGAGGUGCUGAACGCGGCCGAUUUCGGAUCCUC | |
| | GCAAGCCAGGCGGAGAGUGUUCAUGAUUUCGACCCUGAAC | |
| | GAGUUCGUGGAGCUGCCUAAGGGCGACAAGAAGCCCAAGU | |
| | CCAUCAAGAAGGUCCUGAACAAGAUUGUCAGCGAAAAGGA | |
| | CAUCCUCAACAAUCUGCUUAAGUACAACCUGACCGAGUUU | |
| | AAAAAGACCAAAAGCAACAUCAACAAGGCCUCCCUGAUCG | |
| | GAUACUCCAAGUUCAAUUCCGAAGGCUACGUGUACGACCC | |
| | GGAAUUCACCGGACCAACCUUGACCGCCUCCGGUGCUAAC | |
| | UCCCGGAUCAAGAUCAAGGACGGAUCCAACAUCCGGAAGA | |
| | UGAACUCCGACGAGACUUUUCUGUACAUCGGUUUCGACUC | |
| | CCAAGACGGAAAGCGCGUGAACGAGAUAGAGUUCCUCACC | |
| | GAGAAUCAGAAGAUCUUUGUGUGCGGCAACUCCAUUAGCG | |
| | UGGAGGUGCUGGAGGCCAUCAUCGAUAAGAUCGGUGGCCC | |
| | UUCCUCCGGUGGAAAGCGCCCAGCGGCUACAAAGAAGGCC | |
| | GGACAGGCCAAGAAGAAGAAGGGUUCGUACCCCUACGACG | |
| | UGCCUGACUAUGCCUGAGCGGCCGCUUAAUUAAGCUGCCU | |
| | UCUGCGGGGCUUGCCUUCUGGCCAUGCCCUUCUUCUCUCCC | |
| | UUGCACCUGUACCUCUUGGUCUUUGAAUAAAGCCUGAGUA | |
| | GGAAGUCUAGAAAAAAAAAAAAAAAAAAAAAAAAAAAAA | |
| | AAAAAAAAAAAAAAAAAAAAAAAAAAAAAAAAAAAAAAA | |
| | AAAAAAAAAAAAAAAAAAAAAAAAAAAAAAAAAAAAAAA | |
| | AAAA | |
| TAL01-MQ1 | ATGGCCCCAAAGAAGAAGCGGAAAGTCGGAGGTTCAGGCG | 42 |
| DNA | GCTCGCATATGGTGGATCTGAGAACCCTGGGTTACAGCCAG | |
| sequence | CAGCAGCAGGAAAAGATTAAGCCGAAGGTCCGCTCAACCGT | |
| (ORF) | GGCCCAACACCACGAGGCCTTGGTCGGACATGGGTTCACTC | |
| | ACGCCCACATCGTGGCGCTCTCGCAACACCCGGCAGCCCTG | |
| | GGAACCGTGGCTGTGAAGTACCAAGACATGATCGCGGCCCT | |
| | GCCTGAAGCAACGCATGAGGCCATTGTCGGCGTGGGAAAGC | |
| | GCGGAGCTGGAGCCAGAGCGCTGGAAGCCCTGCTGACCGTC | |
| | GCTGGGGAACTGCGGGGACCACCTCTGCAACTGGACACTGG | |
| | ACAACTGCTCAAGATCGCTAAGCGCGGGGGAGTGACTGCCG | |
| | TGGAGGCGGTGCATGCCTGGAGGAACGCCCTGACTGGAGCA | |
| | CCTCTGAATCTGACGCCCGAACAGGTGGTGGCCATTGCGTCC | |
| | AACAACGGAGGTAAACAGGCCCTTGAGACTGTGCAACGGTT | |
| | GCTGCCAGTCCTGTGCCAGGCCCATGGCCTTACACCAGAACA | |
| | AGTGGTCGCTATTGCCAGCAACGGAGGAGGCAAGCAGGCAC | |
| | TGGAGACTGTTCAACGCCTCCTGCCGGTCCTGTGCCAGGCCC | |
| | ATGGATTGACTCCTGAGCAAGTCGTGGCTATCGCGTCGCACG | |
| | ATGGAGGCAAACAGGCTCTCGAAACGGTGCAAAGACTTCTG | |
| | CCCGTCCTGTGTCAAGCGCATGGCCTCACCCCTGAACAAGTA | |
| | GTGGCCATAGCCTCCCATGACGGGGGAAAACAGGCTTTGGA | |
| | GACTGTGCAGAGGCTGCTGCCTGTCCTGTGCCAGGCCCACGG | |
| | TTTGACCCCGGAACAGGTTGTGGCCATTGCCTCGAATATCGG | |
| | AGGGAAACAAGCGCTAGAAACCGTGCAGAGACTTCTTCCTG | |
| | TCCTCTGTCAGGCTCACGGTCTGACTCCTGAACAGGTGGTGG | |
| | CTATTGCTTCTAACAACGGTGGGAAGCAGGCCCTAGAAACT | |
| | GTGCAAAGGCTGCTTCCCGTGCTATGCCAGGCTCATGGACTC | |
| | ACCCCGGAGCAAGTCGTGGCCATTGCATCCAATGGAGGGGG | |
| | GAAGCAGGCCCTGGAAACTGTGCAGCGCTTGCTGCCTGTGCT | |
| | GTGCCAAGCTCACGGCCTAACCCCCGAACAGGTGGTGGCCA | |
| | TCGCCTCCAACATCGGCGGGAAGCAGGCCCTCGAAACAGTC | |
| | CAGAGATTGCTCCCGGTGCTGTGTCAGGCCCACGGACTCACG | |
| | CCGGAACAAGTGGTGGCGATTGCCTCACACGATGGTGGCAA | |
| | ACAAGCACTGGAAACGGTCCAGAGACTGCTCCCGGTCCTCT | |
| | GCCAGGCGCATGGCTTGACGCCAGAACAGGTCGTGGCGATT | |
| | GCCTCACACGATGGGGGAAAGCAGGCTCTGGAAACAGTGCA | |
| | GAGATTGTTACCTGTGCTCTGTCAAGCCCACGGCTTGACTCC | |
| | AGAACAGGTCGTAGCAATTGCCTCCCACGATGGCGGAAAGC | |
| | AGGCGTTAGAGACAGTGCAACGGCTGCTCCCCGTGTTGTGTC | |
| | AAGCACATGGGCTGACACCGGAACAAGTGGTAGCCATCGCG | |
| | AGCAACATTGGAGGGAAGCAAGCGCTCGAGACAGTCCAACG | |
| | GCTTCTGCCGGTGCTGTGTCAGGCACATGGCCTGACCCCTGA | |
| | GCAAGTTGTCGCCATTGCTTCCCATGATGGAGGAAAGCAAG | |
| | CCCTAGAGACTGTCCAACGCCTGTTGCCGGTACTCTGCCAAG | |
| | CCCATGGTTTGACTCCGGAACAGGTGGTGGCAATCGCTTCCA | |
| | ATATCGGCGGCAAGCAAGCTCTCGAAACCGTCCAGCGACTC | |
| | CTGCCCGTGCTTTGTCAGGCGCACGGACTGACTCCCGAACAA | |
| | GTCGTCGCAATTGCCAGCCATGATGGTGGAAAGCAGGCCTT | |
| | GGAAACTGTCCAGCGGCTCCTGCCAGTGCTTTGCCAAGCACA | |
| | CGGACTTACCCCCGAGCAAGTGGTCGCAATCGCGTCACATG | |
| | ACGGCGGGAAACAGGCGCTGGAAACCGTTCAGAGACTCTTG | |
| | CCGGTGTTGTGCCAGGCACACGGGCTCACCCCAGAACAGGT | |
| | CGTCGCCATCGCCAGCCACGACGGTGGAAAACAAGCTCTTG | |
| | AAACGGTGCAGCGCCTCCTTCCGGTGCTCTGTCAAGCTCATG | |
| | GGCTTACTCCAGAGCAAGTAGTCGCTATCGCCTCGCATGACG | |
| | GTGGACGACCAGCCCTGGAATCCATCGTGGCCCAGCTCTCCA | |
| | GACCGGACCCTGCTCTCGCTGCCCTGACCAACGACCACTTGG | |
| | TGGCCCTGGCCTGTCTGGGTGGTAGACCGGCGTTGGACGCTG | |
| | TCAAGAAGGGACTCCCTCACGCACCGGCACTTATTAAGCGG | |
| | ACCAACAGGCGGATCCCTGAGCGCACTTCCCATCGCGTGGC | |
| | ATCTGGGAGCGGAGGAGGATCCGGCGGAGCCAGAGACTCCA | |
| | AGGTCGAGAACAAGACCAAGAAGCTGCGGGTGTTCGAGGCC | |
| | TTTGCCGGGATCGGAGCACAGCGGAAGGCCTTGGAGAAGGT | |
| | CCGCAAGGACGAATACGAAATCGTGGGCCTTGCCGAGTGGT | |
| | ACGTGCCAGCCATCGTGATGTACCAGGCTATCCACAACAACT | |
| | TCCACACCAAGCTGGAGTACAAGTCCGTGAGCAGAGAAGAG | |
| | ATGATCGACTACCTCGAAAACAAGACCCTGTCCTGGAACTCC | |
| | AAGAATCCCGTGTCGAACGGTTACTGGAAGCGCAAGAAGGA | |
| | TGATGAACTGAAGATTATCTACAACGCGATTAAGCTGTCCGA | |
| | AAAGGAAGGAAACATTTTCGACATTCGCGATCTGTACAAGA | |
| | GAACCCTCAAGAACATTGATCTGCTTACCTACTCCTTCCCGT | |
| | GCCAAGATCTCAGCCAGCAGGGGATTCAGAAGGGGATGAAG | |
| | CGGGGTTCAGGGACCAGATCAGGCCTGCTCTGGGAGATCGA | |
| | AAGAGCCCTGGACTCCACCGAGAAGAACGATCTCCCGAAAT | |
| | ACCTTCTGATGGAGAACGTCGGCGCTCTCCTGCACAAGAAG | |
| | AACGAAGAAGAACTGAACCAGTGGAAGCAGAAGCTGGAGT | |
| | CACTCGGATACCAGAATTCTATTGAGGTGCTGAACGCGGCC | |
| | GATTTCGGATCCTCGCAAGCCAGGCGGAGAGTGTTCATGATT | |
| | TCGACCCTGAACGAGTTCGTGGAGCTGCCTAAGGGCGACAA | |
| | GAAGCCCAAGTCCATCAAGAAGGTCCTGAACAAGATTGTCA | |
| | GCGAAAAGGACATCCTCAACAATCTGCTTAAGTACAACCTG | |
| | ACCGAGTTTAAAAAGACCAAAAGCAACATCAACAAGGCCTC | |
| | CCTGATCGGATACTCCAAGTTCAATTCCGAAGGCTACGTGTA | |
| | CGACCCGGAATTCACCGGACCAACCTTGACCGCCTCCGGTGC | |
| | TAACTCCCGGATCAAGATCAAGGACGGATCCAACATCCGGA | |
| | AGATGAACTCCGACGAGACTTTTCTGTACATCGGTTTCGACT | |
| | CCCAAGACGGAAAGCGCGTGAACGAGATAGAGTTCCTCACC | |
| | GAGAATCAGAAGATCTTTGTGTGCGGCAACTCCATTAGCGTG | |
| | GAGGTGCTGGAGGCCATCATCGATAAGATCGGTGGCCCTTC | |
| | CTCCGGTGGAAAGCGCCCAGCGGCTACAAAGAAGGCCGGAC | |
| | AGGCCAAGAAGAAGAAGGGTTCGTACCCCTACGACGTGCCT | |
| | GACTATGCCTGA | |
| TAL01-MQ1 | MAPKKKRKVGGSGGSHMVDLRTLGYSQQQQEKIKPKVRSTVA | 43 |
| amino acid | QHHEALVGHGFTHAHIVALSQHPAALGTVAVKYQDMIAALPE | |
| sequence | ATHEAIVGVGKRGAGARALEALLTVAGELRGPPLQLDTGQLLK | |
| | IAKRGGVTAVEAVHAWRNALTGAPLNLTPEQVVAIASNNGGK | |
| | QALETVQRLLPVLCQAHGLTPEQVVAIASNGGGKQALETVQRL | |
| | LPVLCQAHGLTPEQVVAIASHDGGKQALETVQRLLPVLCQAHG | |
| | LTPEQVVAIASHDGGKQALETVQRLLPVLCQAHGLTPEQVVAI | |
| | ASNIGGKQALETVQRLLPVLCQAHGLTPEQVVAIASNNGGKQA | |
| | LETVQRLLPVLCQAHGLTPEQVVAIASNGGGKQALETVQRLLP | |
| | VLCQAHGLTPEQVVAIASNIGGKQALETVQRLLPVLCQAHGLT | |
| | PEQVVAIASHDGGKQALETVQRLLPVLCQAHGLTPEQVVAIAS | |
| | HDGGKQALETVQRLLPVLCQAHGLTPEQVVAIASHDGGKQAL | |
| | ETVQRLLPVLCQAHGLTPEQVVAIASNIGGKQALETVQRLLPVL | |
| | CQAHGLTPEQVVAIASHDGGKQALETVQRLLPVLCQAHGLTPE | |
| | QVVAIASNIGGKQALETVQRLLPVLCQAHGLTPEQVVAIASHD | |
| | GGKQALETVQRLLPVLCQAHGLTPEQVVAIASHDGGKQALET | |
| | VQRLLPVLCQAHGLTPEQVVAIASHDGGKQALETVQRLLPVLC | |
| | QAHGLTPEQVVAIASHDGGRPALESIVAQLSRPDPALAALTND | |
| | HLVALACLGGRPALDAVKKGLPHAPALIKRTNRRIPERTSHRV | |
| | ASGSGGGSGGARDSKVENKTKKLRVFEAFAGIGAQRKALEKV | |
| | RKDEYEIVGLAEWYVPAIVMYQAIHNNFHTKLEYKSVSREEMI | |
| | DYLENKTLSWNSKNPVSNGYWKRKKDDELKIIYNAIKLSEKEG | |
| | NIFDIRDLYKRTLKNIDLLTYSFPCQDLSQQGIQKGMKRGSGTR | |
| | SGLLWEIERALDSTEKNDLPKYLLMENVGALLHKKNEEELNQ | |
| | WKQKLESLGYQNSIEVLNAADFGSSQARRRVFMISTLNEFVELP | |
| | KGDKKPKSIKKVLNKIVSEKDILNNLLKYNLTEFKKTKSNINKA | |
| | SLIGYSKFNSEGYVYDPEFTGPTLTASGANSRIKIKDGSNIRKMN | |
| | SDETFLYIGFDSQDGKRVNEIEFLTENQKIFVCGNSISVEVLEAII | |
| | DKIGGPSSGGKRPAATKKAGQAKKKKGSYPYDVPDYA | |
| TAL01-MQ1 | CAUAUGGUGGAUCUGAGAACCCUGGGUUACAGCCAGCAGC | 44 |
| DNA-binding | AGCAGGAAAAGAUUAAGCCGAAGGUCCGCUCAACCGUGGC | |
| domain | CCAACACCACGAGGCCUUGGUCGGACAUGGGUUCACUCAC | |
| (DBD) | GCCCACAUCGUGGCGCUCUCGCAACACCCGGCAGCCCUGGG | |
| mRNA | AACCGUGGCUGUGAAGUACCAAGACAUGAUCGCGGCCCUG | |
| sequence | CCUGAAGCAACGCAUGAGGCCAUUGUCGGCGUGGGAAAGC | |
| | GCGGAGCUGGAGCCAGAGCGCUGGAAGCCCUGCUGACCGU | |
| | CGCUGGGGAACUGCGGGGACCACCUCUGCAACUGGACACU | |
| | GGACAACUGCUCAAGAUCGCUAAGCGCGGGGGAGUGACUG | |
| | CCGUGGAGGCGGUGCAUGCCUGGAGGAACGCCCUGACUGG | |
| | AGCACCUCUGAAUCUGACGCCCGAACAGGUGGUGGCCAUU | |
| | GCGUCCAACAACGGAGGUAAACAGGCCCUUGAGACUGUGC | |
| | AACGGUUGCUGCCAGUCCUGUGCCAGGCCCAUGGCCUUAC | |
| | ACCAGAACAAGUGGUCGCUAUUGCCAGCAACGGAGGAGGC | |
| | AAGCAGGCACUGGAGACUGUUCAACGCCUCCUGCCGGUCC | |
| | UGUGCCAGGCCCAUGGAUUGACUCCUGAGCAAGUCGUGGC | |
| | UAUCGCGUCGCACGAUGGAGGCAAACAGGCUCUCGAAACG | |
| | GUGCAAAGACUUCUGCCCGUCCUGUGUCAAGCGCAUGGCC | |
| | UCACCCCUGAACAAGUAGUGGCCAUAGCCUCCCAUGACGG | |
| | GGGAAAACAGGCUUUGGAGACUGUGCAGAGGCUGCUGCCU | |
| | GUCCUGUGCCAGGCCCACGGUUUGACCCCGGAACAGGUUG | |
| | UGGCCAUUGCCUCGAAUAUCGGAGGGAAACAAGCGCUAGA | |
| | AACCGUGCAGAGACUUCUUCCUGUCCUCUGUCAGGCUCAC | |
| | GGUCUGACUCCUGAACAGGUGGUGGCUAUUGCUUCUAACA | |
| | ACGGUGGGAAGCAGGCCCUAGAAACUGUGCAAAGGCUGCU | |
| | UCCCGUGCUAUGCCAGGCUCAUGGACUCACCCCGGAGCAA | |
| | GUCGUGGCCAUUGCAUCCAAUGGAGGGGGGAAGCAGGCCC | |
| | UGGAAACUGUGCAGCGCUUGCUGCCUGUGCUGUGCCAAGC | |
| | UCACGGCCUAACCCCCGAACAGGUGGUGGCCAUCGCCUCCA | |
| | ACAUCGGCGGGAAGCAGGCCCUCGAAACAGUCCAGAGAUU | |
| | GCUCCCGGUGCUGUGUCAGGCCCACGGACUCACGCCGGAA | |
| | CAAGUGGUGGCGAUUGCCUCACACGAUGGUGGCAAACAAG | |
| | CACUGGAAACGGUCCAGAGACUGCUCCCGGUCCUCUGCCA | |
| | GGCGCAUGGCUUGACGCCAGAACAGGUCGUGGCGAUUGCC | |
| | UCACACGAUGGGGGAAAGCAGGCUCUGGAAACAGUGCAGA | |
| | GAUUGUUACCUGUGCUCUGUCAAGCCCACGGCUUGACUCC | |
| | AGAACAGGUCGUAGCAAUUGCCUCCCACGAUGGCGGAAAG | |
| | CAGGCGUUAGAGACAGUGCAACGGCUGCUCCCCGUGUUGU | |
| | GUCAAGCACAUGGGCUGACACCGGAACAAGUGGUAGCCAU | |
| | CGCGAGCAACAUUGGAGGGAAGCAAGCGCUCGAGACAGUC | |
| | CAACGGCUUCUGCCGGUGCUGUGUCAGGCACAUGGCCUGA | |
| | CCCCUGAGCAAGUUGUCGCCAUUGCUUCCCAUGAUGGAGG | |
| | AAAGCAAGCCCUAGAGACUGUCCAACGCCUGUUGCCGGUA | |
| | CUCUGCCAAGCCCAUGGUUUGACUCCGGAACAGGUGGUGG | |
| | CAAUCGCUUCCAAUAUCGGCGGCAAGCAAGCUCUCGAAAC | |
| | CGUCCAGCGACUCCUGCCCGUGCUUUGUCAGGCGCACGGA | |
| | CUGACUCCCGAACAAGUCGUCGCAAUUGCCAGCCAUGAUG | |
| | GUGGAAAGCAGGCCUUGGAAACUGUCCAGCGGCUCCUGCC | |
| | AGUGCUUUGCCAAGCACACGGACUUACCCCCGAGCAAGUG | |
| | GUCGCAAUCGCGUCACAUGACGGCGGGAAACAGGCGCUGG | |
| | AAACCGUUCAGAGACUCUUGCCGGUGUUGUGCCAGGCACA | |
| | CGGGCUCACCCCAGAACAGGUCGUCGCCAUCGCCAGCCACG | |
| | ACGGUGGAAAACAAGCUCUUGAAACGGUGCAGCGCCUCCU | |
| | UCCGGUGCUCUGUCAAGCUCAUGGGCUUACUCCAGAGCAA | |
| | GUAGUCGCUAUCGCCUCGCAUGACGGUGGACGACCAGCCC | |
| | UGGAAUCCAUCGUGGCCCAGCUCUCCAGACCGGACCCUGC | |
| | UCUCGCUGCCCUGACCAACGACCACUUGGUGGCCCUGGCCU | |
| | GUCUGGGUGGUAGACCGGCGUUGGACGCUGUCAAGAAGGG | |
| | ACUCCCUCACGCACCGGCACUUAUUAAGCGGACCAACAGG | |
| | CGGAUCCCUGAGCGCACUUCCCAUCGCGUG | |
| TAL01-MQ1 | HMVDLRTLGYSQQQQEKIKPKVRSTVAQHHEALVGHGFTHAH | 45 |
| DBD amino | IVALSQHPAALGTVAVKYQDMIAALPEATHEAIVGVGKRGAG | |
| acid sequence | ARALEALLTVAGELRGPPLQLDTGQLLKIAKRGGVTAVEAVHA | |
| | WRNALTGAPLNLTPEQVVAIASNNGGKQALETVQRLLPVLCQ | |
| | AHGLTPEQVVAIASNGGGKQALETVQRLLPVLCQAHGLTPEQV | |
| | VAIASHDGGKQALETVQRLLPVLCQAHGLTPEQVVAIASHDGG | |
| | KQALETVQRLLPVLCQAHGLTPEQVVAIASNIGGKQALETVQR | |
| | LLPVLCQAHGLTPEQVVAIASNNGGKQALETVQRLLPVLCQAH | |
| | GLTPEQVVAIASNGGGKQALETVQRLLPVLCQAHGLTPEQVVA | |
| | IASNIGGKQALETVQRLLPVLCQAHGLTPEQVVAIASHDGGKQ | |
| | ALETVQRLLPVLCQAHGLTPEQVVAIASHDGGKQALETVQRLL | |
| | PVLCQAHGLTPEQVVAIASHDGGKQALETVQRLLPVLCQAHGL | |
| | TPEQVVAIASNIGGKQALETVQRLLPVLCQAHGLTPEQVVAIAS | |
| | HDGGKQALETVQRLLPVLCQAHGLTPEQVVAIASNIGGKQALE | |
| | TVQRLLPVLCQAHGLTPEQVVAIASHDGGKQALETVQRLLPVL | |
| | CQAHGLTPEQVVAIASHDGGKQALETVQRLLPVLCQAHGLTPE | |
| | QVVAIASHDGGKQALETVQRLLPVLCQAHGLTPEQVVAIASHD | |
| | GGRPALESIVAQLSRPDPALAALTNDHLVALACLGGRPALDAV | |
| | KKGLPHAPALIKRTNRRIPERTSHRV | |
| TAL01-MQ1 | UCCAAGGUCGAGAACAAGACCAAGAAGCUGCGGGUGUUCG | 46 |
| effector | AGGCCUUUGCCGGGAUCGGAGCACAGCGGAAGGCCUUGGA | |
| mRNA | GAAGGUCCGCAAGGACGAAUACGAAAUCGUGGGCCUUGCC | |
| sequence | GAGUGGUACGUGCCAGCCAUCGUGAUGUACCAGGCUAUCC | |
| | ACAACAACUUCCACACCAAGCUGGAGUACAAGUCCGUGAG | |
| | CAGAGAAGAGAUGAUCGACUACCUCGAAAACAAGACCCUG | |
| | UCCUGGAACUCCAAGAAUCCCGUGUCGAACGGUUACUGGA | |
| | AGCGCAAGAAGGAUGAUGAACUGAAGAUUAUCUACAACGC | |
| | GAUUAAGCUGUCCGAAAAGGAAGGAAACAUUUUCGACAUU | |
| | CGCGAUCUGUACAAGAGAACCCUCAAGAACAUUGAUCUGC | |
| | UUACCUACUCCUUCCCGUGCCAAGAUCUCAGCCAGCAGGG | |
| | GAUUCAGAAGGGGAUGAAGCGGGGUUCAGGGACCAGAUCA | |
| | GGCCUGCUCUGGGAGAUCGAAAGAGCCCUGGACUCCACCG | |
| | AGAAGAACGAUCUCCCGAAAUACCUUCUGAUGGAGAACGU | |
| | CGGCGCUCUCCUGCACAAGAAGAACGAAGAAGAACUGAAC | |
| | CAGUGGAAGCAGAAGCUGGAGUCACUCGGAUACCAGAAUU | |
| | CUAUUGAGGUGCUGAACGCGGCCGAUUUCGGAUCCUCGCA | |
| | AGCCAGGCGGAGAGUGUUCAUGAUUUCGACCCUGAACGAG | |
| | UUCGUGGAGCUGCCUAAGGGCGACAAGAAGCCCAAGUCCA | |
| | UCAAGAAGGUCCUGAACAAGAUUGUCAGCGAAAAGGACAU | |
| | CCUCAACAAUCUGCUUAAGUACAACCUGACCGAGUUUAAA | |
| | AAGACCAAAAGCAACAUCAACAAGGCCUCCCUGAUCGGAU | |
| | ACUCCAAGUUCAAUUCCGAAGGCUACGUGUACGACCCGGA | |
| | AUUCACCGGACCAACCUUGACCGCCUCCGGUGCUAACUCCC | |
| | GGAUCAAGAUCAAGGACGGAUCCAACAUCCGGAAGAUGAA | |
| | CUCCGACGAGACUUUUCUGUACAUCGGUUUCGACUCCCAA | |
| | GACGGAAAGCGCGUGAACGAGAUAGAGUUCCUCACCGAGA | |
| | AUCAGAAGAUCUUUGUGUGCGGCAACUCCAUUAGCGUGGA | |
| | GGUGCUGGAGGCCAUCAUCGAUAAGAUCGGUGGC | |
| TAL01-MQ1 | SKVENKTKKLRVFEAFAGIGAQRKALEKVRKDEYEIVGLAEW | 47 |
| effector | YVPAIVMYQAIHNNFHTKLEYKSVSREEMIDYLENKTLSWNSK | |
| amino acid | NPVSNGYWKRKKDDELKIIYNAIKLSEKEGNIFDIRDLYKRTLK | |
| sequence | NIDLLTYSFPCQDLSQQGIQKGMKRGSGTRSGLLWEIERALDST | |
| | EKNDLPKYLLMENVGALLHKKNEEELNQWKQKLESLGYQNSI | |
| | EVLNAADFGSSQARRRVFMISTLNEFVELPKGDKKPKSIKKVLN | |
| | KIVSEKDILNNLLKYNLTEFKKTKSNINKASLIGYSKFNSEGYVY | |
| | DPEFTGPTLTASGANSRIKIKDGSNIRKMNSDETFLYIGFDSQDG | |
| | KRVNEIEFLTENQKIFVCGNSISVEVLEAIIDKIGG | |
| TAL01-MQ1 | GTCCAGTACCCACACCCC | 48 |
| target DNA | | |
| sequence | | |
| TAL02-MQ1 | GGGAAAUAAGAGAGAAAAGAAGAGUAAGAAGAAAUAUAA | 49 |
| full-length | GAGCCACCAUGGCCCCAAAGAAGAAGCGGAAAGUCGGAGG | |
| mRNA | UUCAGGCGGCUCGCACAUGGUGGAUCUGCGCACCCUGGGG | |
| sequence | UACUCGCAGCAGCAGCAGGAAAAGAUUAAGCCGAAGGUCC | |
| | GGUCCACUGUCGCCCAGCACCACGAAGCGCUCGUCGGACAC | |
| | GGUUUCACUCACGCACACAUAGUCGCACUGAGCCAGCACC | |
| | CUGCGGCACUGGGAACCGUGGCCGUGAAGUACCAGGAUAU | |
| | GAUCGCAGCCCUUCCUGAGGCCACCCACGAAGCCAUCGUG | |
| | GGCGUGGGAAAGAGAGGUGCCGGAGCAAGGGCUCUUGAGG | |
| | CCCUGCUGACUGUCGCGGGAGAACUCAGGGGACCGCCUCU | |
| | GCAACUGGACACCGGACAGCUGCUGAAAAUUGCCAAGCGG | |
| | GGCGGUGUGACUGCCGUGGAAGCCGUGCACGCCUGGAGAA | |
| | ACGCACUCACUGGCGCUCCUCUGAACCUGACCCCGGAACAG | |
| | GUCGUCGCGAUUGCGUCGAACAAUGGAGGGAAACAGGCGU | |
| | UGGAAACUGUCCAACGGUUGCUCCCGGUGCUAUGUCAAGC | |
| | CCAUGGACUGACGCCCGAACAAGUCGUAGCCAUCGCGUCA | |
| | AACAUCGGCGGAAAACAGGCGCUCGAAACGGUCCAGCGCU | |
| | UACUCCCCGUGCUGUGUCAAGCGCAUGGAUUGACUCCGGA | |
| | GCAAGUCGUCGCUAUCGCCAGCCACGACGGAGGAAAGCAA | |
| | GCUUUGGAAACAGUGCAACGACUGCUGCCCGUCUUGUGUC | |
| | AAGCACACGGGUUGACCCCAGAGCAAGUGGUGGCCAUUGC | |
| | CUCCCAUGAUGGGGGAAAGCAGGCCCUGGAGACUGUGCAG | |
| | CGCCUGUUGCCGGUGUUGUGCCAAGCACAUGGGCUGACAC | |
| | CCGAACAGGUCGUGGCGAUUGCCUCCAACGGUGGCGGCAA | |
| | ACAAGCACUCGAGACAGUGCAGCGACUCCUCCCGGUUCUG | |
| | UGCCAGGCUCACGGCCUCACUCCCGAACAGGUGGUGGCAA | |
| | UAGCCUCCAACAACGGAGGCAAACAGGCUUUAGAAACGGU | |
| | UCAGCGCUUGCUGCCAGUCCUGUGCCAAGCGCACGGAUUG | |
| | ACCCCGGAGCAAGUGGUGGCCAUAGCCAGCAACGGCGGAG | |
| | GGAAGCAGGCCUUGGAAACCGUACAGAGACUCCUCCCCGU | |
| | GUUAUGCCAGGCACACGGCCUGACACCAGAACAGGUGGUG | |
| | GCGAUUGCUUCCAACGGAGGCGGAAAGCAGGCCCUCGAAA | |
| | CAGUCCAGCGACUUCUGCCGGUGCUGUGCCAGGCUCACGG | |
| | UUUGACUCCAGAGCAAGUUGUAGCUAUUGCCAGCAACAUC | |
| | GGUGGGAAGCAGGCGCUGGAAACUGUGCAACGGCUCCUGC | |
| | CCGUCCUCUGCCAAGCUCAUGGCCUCACCCCCGAACAGGUC | |
| | GUGGCCAUCGCGAGCCACGAUGGAGGAAAACAAGCCCUGG | |
| | AAACGGUGCAGAGGUUGCUGCCCGUACUUUGUCAGGCCCA | |
| | CGGGCUUACUCCCGAGCAAGUGGUGGCCAUCGCCUCCAAC | |
| | AUUGGCGGCAAGCAGGCACUGGAAACUGUGCAGAGACUGC | |
| | UUCCCGUGCUCUGCCAGGCACACGGACUCACCCCUGAACAG | |
| | GUUGUGGCCAUAGCCUCAAACGGAGGAGGAAAGCAGGCCC | |
| | UUGAAACUGUCCAGAGACUGUUGCCCGUACUGUGUCAGGC | |
| | CCACGGCCUUACUCCUGAACAGGUAGUGGCAAUCGCAUCG | |
| | AACGGCGGUGGGAAACAAGCUCUCGAGACUGUCCAGCGGC | |
| | UGCUACCUGUGUUGUGUCAGGCUCAUGGACUUACGCCGGA | |
| | ACAAGUAGUGGCCAUUGCUUCCCACGACGGGGGUAAACAG | |
| | GCUCUGGAAACCGUCCAAAGGCUGCUGCCAGUGUUGUGCC | |
| | AGGCGCAUGGCCUAACCCCAGAACAAGUGGUCGCAAUCGC | |
| | CUCGCACGAUGGUGGUAAACAAGCGCUAGAAACCGUGCAG | |
| | AGGCUUCUCCCAGUGCUCUGUCAGGCCCACGGUCUGACUC | |
| | CAGAACAAGUGGUCGCCAUCGCAUCCCAUGACGGGGGGAA | |
| | GCAAGCCUUGGAGACAGUCCAACGGCUGCUUCCGGUGUUA | |
| | UGUCAGGCGCACGGACUGACUCCUGAGCAAGUCGUGGCUA | |
| | UCGCGUCGCAUGAUGGGGGCAAGCAAGCCCUGGAAACGGU | |
| | GCAACGCCUGCUGCCUGUCCUUUGCCAGGCACACGGGCUG | |
| | ACUCCGGAACAGGUGGUGGCUAUUGCCUCGCAUGACGGAG | |
| | GUAGGCCCGCACUCGAAAGCAUUGUCGCACAGCUGUCCCG | |
| | GCCAGAUCCUGCUUUGGCCGCCCUGACCAACGAUCACCUCG | |
| | UCGCUCUGGCCUGCCUGGGUGGAAGGCCGGCUCUGGAUGC | |
| | CGUGAAGAAGGGGCUGCCUCAUGCCCCUGCCCUGAUUAAG | |
| | AGAACUAACCGGCGCAUUCCGGAGAGAACCUCCCACAGAG | |
| | UGGCAUCUGGGAGCGGAGGAGGAUCCGGCGGAGCCAGAGA | |
| | CUCCAAGGUCGAGAACAAGACCAAGAAGCUGCGGGUGUUC | |
| | GAGGCCUUUGCCGGGAUCGGAGCACAGCGGAAGGCCUUGG | |
| | AGAAGGUCCGCAAGGACGAAUACGAAAUCGUGGGCCUUGC | |
| | CGAGUGGUACGUGCCAGCCAUCGUGAUGUACCAGGCUAUC | |
| | CACAACAACUUCCACACCAAGCUGGAGUACAAGUCCGUGA | |
| | GCAGAGAAGAGAUGAUCGACUACCUCGAAAACAAGACCCU | |
| | GUCCUGGAACUCCAAGAAUCCCGUGUCGAACGGUUACUGG | |
| | AAGCGCAAGAAGGAUGAUGAACUGAAGAUUAUCUACAACG | |
| | CGAUUAAGCUGUCCGAAAAGGAAGGAAACAUUUUCGACAU | |
| | UCGCGAUCUGUACAAGAGAACCCUCAAGAACAUUGAUCUG | |
| | CUUACCUACUCCUUCCCGUGCCAAGAUCUCAGCCAGCAGG | |
| | GGAUUCAGAAGGGGAUGAAGCGGGGUUCAGGGACCAGAUC | |
| | AGGCCUGCUCUGGGAGAUCGAAAGAGCCCUGGACUCCACC | |
| | GAGAAGAACGAUCUCCCGAAAUACCUUCUGAUGGAGAACG | |
| | UCGGCGCUCUCCUGCACAAGAAGAACGAAGAAGAACUGAA | |
| | CCAGUGGAAGCAGAAGCUGGAGUCACUCGGAUACCAGAAU | |
| | UCUAUUGAGGUGCUGAACGCGGCCGAUUUCGGAUCCUCGC | |
| | AAGCCAGGCGGAGAGUGUUCAUGAUUUCGACCCUGAACGA | |
| | GUUCGUGGAGCUGCCUAAGGGCGACAAGAAGCCCAAGUCC | |
| | AUCAAGAAGGUCCUGAACAAGAUUGUCAGCGAAAAGGACA | |
| | UCCUCAACAAUCUGCUUAAGUACAACCUGACCGAGUUUAA | |
| | AAAGACCAAAAGCAACAUCAACAAGGCCUCCCUGAUCGGA | |
| | UACUCCAAGUUCAAUUCCGAAGGCUACGUGUACGACCCGG | |
| | AAUUCACCGGACCAACCUUGACCGCCUCCGGUGCUAACUCC | |
| | CGGAUCAAGAUCAAGGACGGAUCCAACAUCCGGAAGAUGA | |
| | ACUCCGACGAGACUUUUCUGUACAUCGGUUUCGACUCCCA | |
| | AGACGGAAAGCGCGUGAACGAGAUAGAGUUCCUCACCGAG | |
| | AAUCAGAAGAUCUUUGUGUGCGGCAACUCCAUUAGCGUGG | |
| | AGGUGCUGGAGGCCAUCAUCGAUAAGAUCGGUGGCCCUUC | |
| | CUCCGGUGGAAAGCGCCCAGCGGCUACAAAGAAGGCCGGA | |
| | CAGGCCAAGAAGAAGAAGGGUUCGUACCCCUACGACGUGC | |
| | CUGACUAUGCCUGAGCGGCCGCUUAAUUAAGCUGCCUUCU | |
| | GCGGGGCUUGCCUUCUGGCCAUGCCCUUCUUCUCUCCCUU | |
| | GCACCUGUACCUCUUGGUCUUUGAAUAAAGCCUGAGUAGG | |
| | AAGUCUAGAAAAAAAAAAAAAAAAAAAAAAAAAAAAAAA | |
| | AAAAAAAAAAAAAAAAAAAAAAAAAAAAAAAAAAAAAAA | |
| | AAAAAAAAAAAAAAAAAAAAAAAAAAAAAAAAAAAAAAA | |
| | AA | |
| TAL02-MQ1 | ATGGCCCCAAAGAAGAAGCGGAAAGTCGGAGGTTCAGGCG | 50 |
| DNA | GCTCGCACATGGTGGATCTGCGCACCCTGGGGTACTCGCAGC | |
| sequence | AGCAGCAGGAAAAGATTAAGCCGAAGGTCCGGTCCACTGTC | |
| (ORF) | GCCCAGCACCACGAAGCGCTCGTCGGACACGGTTTCACTCA | |
| | CGCACACATAGTCGCACTGAGCCAGCACCCTGCGGCACTGG | |
| | GAACCGTGGCCGTGAAGTACCAGGATATGATCGCAGCCCTT | |
| | CCTGAGGCCACCCACGAAGCCATCGTGGGCGTGGGAAAGAG | |
| | AGGTGCCGGAGCAAGGGCTCTTGAGGCCCTGCTGACTGTCG | |
| | CGGGAGAACTCAGGGGACCGCCTCTGCAACTGGACACCGGA | |
| | CAGCTGCTGAAAATTGCCAAGCGGGGCGGTGTGACTGCCGT | |
| | GGAAGCCGTGCACGCCTGGAGAAACGCACTCACTGGCGCTC | |
| | CTCTGAACCTGACCCCGGAACAGGTCGTCGCGATTGCGTCGA | |
| | ACAATGGAGGGAAACAGGCGTTGGAAACTGTCCAACGGTTG | |
| | CTCCCGGTGCTATGTCAAGCCCATGGACTGACGCCCGAACA | |
| | AGTCGTAGCCATCGCGTCAAACATCGGCGGAAAACAGGCGC | |
| | TCGAAACGGTCCAGCGCTTACTCCCCGTGCTGTGTCAAGCGC | |
| | ATGGATTGACTCCGGAGCAAGTCGTCGCTATCGCCAGCCAC | |
| | GACGGAGGAAAGCAAGCTTTGGAAACAGTGCAACGACTGCT | |
| | GCCCGTCTTGTGTCAAGCACACGGGTTGACCCCAGAGCAAG | |
| | TGGTGGCCATTGCCTCCCATGATGGGGGAAAGCAGGCCCTG | |
| | GAGACTGTGCAGCGCCTGTTGCCGGTGTTGTGCCAAGCACAT | |
| | GGGCTGACACCCGAACAGGTCGTGGCGATTGCCTCCAACGG | |
| | TGGCGGCAAACAAGCACTCGAGACAGTGCAGCGACTCCTCC | |
| | CGGTTCTGTGCCAGGCTCACGGCCTCACTCCCGAACAGGTGG | |
| | TGGCAATAGCCTCCAACAACGGAGGCAAACAGGCTTTAGAA | |
| | ACGGTTCAGCGCTTGCTGCCAGTCCTGTGCCAAGCGCACGGA | |
| | TTGACCCCGGAGCAAGTGGTGGCCATAGCCAGCAACGGCGG | |
| | AGGGAAGCAGGCCTTGGAAACCGTACAGAGACTCCTCCCCG | |
| | TGTTATGCCAGGCACACGGCCTGACACCAGAACAGGTGGTG | |
| | GCGATTGCTTCCAACGGAGGCGGAAAGCAGGCCCTCGAAAC | |
| | AGTCCAGCGACTTCTGCCGGTGCTGTGCCAGGCTCACGGTTT | |
| | GACTCCAGAGCAAGTTGTAGCTATTGCCAGCAACATCGGTG | |
| | GGAAGCAGGCGCTGGAAACTGTGCAACGGCTCCTGCCCGTC | |
| | CTCTGCCAAGCTCATGGCCTCACCCCCGAACAGGTCGTGGCC | |
| | ATCGCGAGCCACGATGGAGGAAAACAAGCCCTGGAAACGGT | |
| | GCAGAGGTTGCTGCCCGTACTTTGTCAGGCCCACGGGCTTAC | |
| | TCCCGAGCAAGTGGTGGCCATCGCCTCCAACATTGGCGGCA | |
| | AGCAGGCACTGGAAACTGTGCAGAGACTGCTTCCCGTGCTCT | |
| | GCCAGGCACACGGACTCACCCCTGAACAGGTTGTGGCCATA | |
| | GCCTCAAACGGAGGAGGAAAGCAGGCCCTTGAAACTGTCCA | |
| | GAGACTGTTGCCCGTACTGTGTCAGGCCCACGGCCTTACTCC | |
| | TGAACAGGTAGTGGCAATCGCATCGAACGGCGGTGGGAAAC | |
| | AAGCTCTCGAGACTGTCCAGCGGCTGCTACCTGTGTTGTGTC | |
| | AGGCTCATGGACTTACGCCGGAACAAGTAGTGGCCATTGCTT | |
| | CCCACGACGGGGGTAAACAGGCTCTGGAAACCGTCCAAAGG | |
| | CTGCTGCCAGTGTTGTGCCAGGCGCATGGCCTAACCCCAGAA | |
| | CAAGTGGTCGCAATCGCCTCGCACGATGGTGGTAAACAAGC | |
| | GCTAGAAACCGTGCAGAGGCTTCTCCCAGTGCTCTGTCAGGC | |
| | CCACGGTCTGACTCCAGAACAAGTGGTCGCCATCGCATCCCA | |
| | TGACGGGGGGAAGCAAGCCTTGGAGACAGTCCAACGGCTGC | |
| | TTCCGGTGTTATGTCAGGCGCACGGACTGACTCCTGAGCAAG | |
| | TCGTGGCTATCGCGTCGCATGATGGGGGCAAGCAAGCCCTG | |
| | GAAACGGTGCAACGCCTGCTGCCTGTCCTTTGCCAGGCACAC | |
| | GGGCTGACTCCGGAACAGGTGGTGGCTATTGCCTCGCATGA | |
| | CGGAGGTAGGCCCGCACTCGAAAGCATTGTCGCACAGCTGT | |
| | CCCGGCCAGATCCTGCTTTGGCCGCCCTGACCAACGATCACC | |
| | TCGTCGCTCTGGCCTGCCTGGGTGGAAGGCCGGCTCTGGATG | |
| | CCGTGAAGAAGGGGCTGCCTCATGCCCCTGCCCTGATTAAG | |
| | AGAACTAACCGGCGCATTCCGGAGAGAACCTCCCACAGAGT | |
| | GGCATCTGGGAGCGGAGGAGGATCCGGCGGAGCCAGAGACT | |
| | CCAAGGTCGAGAACAAGACCAAGAAGCTGCGGGTGTTCGAG | |
| | GCCTTTGCCGGGATCGGAGCACAGCGGAAGGCCTTGGAGAA | |
| | GGTCCGCAAGGACGAATACGAAATCGTGGGCCTTGCCGAGT | |
| | GGTACGTGCCAGCCATCGTGATGTACCAGGCTATCCACAAC | |
| | AACTTCCACACCAAGCTGGAGTACAAGTCCGTGAGCAGAGA | |
| | AGAGATGATCGACTACCTCGAAAACAAGACCCTGTCCTGGA | |
| | ACTCCAAGAATCCCGTGTCGAACGGTTACTGGAAGCGCAAG | |
| | AAGGATGATGAACTGAAGATTATCTACAACGCGATTAAGCT | |
| | GTCCGAAAAGGAAGGAAACATTTTCGACATTCGCGATCTGT | |
| | ACAAGAGAACCCTCAAGAACATTGATCTGCTTACCTACTCCT | |
| | TCCCGTGCCAAGATCTCAGCCAGCAGGGGATTCAGAAGGGG | |
| | ATGAAGCGGGGTTCAGGGACCAGATCAGGCCTGCTCTGGGA | |
| | GATCGAAAGAGCCCTGGACTCCACCGAGAAGAACGATCTCC | |
| | CGAAATACCTTCTGATGGAGAACGTCGGCGCTCTCCTGCACA | |
| | AGAAGAACGAAGAAGAACTGAACCAGTGGAAGCAGAAGCT | |
| | GGAGTCACTCGGATACCAGAATTCTATTGAGGTGCTGAACG | |
| | CGGCCGATTTCGGATCCTCGCAAGCCAGGCGGAGAGTGTTC | |
| | ATGATTTCGACCCTGAACGAGTTCGTGGAGCTGCCTAAGGGC | |
| | GACAAGAAGCCCAAGTCCATCAAGAAGGTCCTGAACAAGAT | |
| | TGTCAGCGAAAAGGACATCCTCAACAATCTGCTTAAGTACA | |
| | ACCTGACCGAGTTTAAAAAGACCAAAAGCAACATCAACAAG | |
| | GCCTCCCTGATCGGATACTCCAAGTTCAATTCCGAAGGCTAC | |
| | GTGTACGACCCGGAATTCACCGGACCAACCTTGACCGCCTCC | |
| | GGTGCTAACTCCCGGATCAAGATCAAGGACGGATCCAACAT | |
| | CCGGAAGATGAACTCCGACGAGACTTTTCTGTACATCGGTTT | |
| | CGACTCCCAAGACGGAAAGCGCGTGAACGAGATAGAGTTCC | |
| | TCACCGAGAATCAGAAGATCTTTGTGTGCGGCAACTCCATTA | |
| | GCGTGGAGGTGCTGGAGGCCATCATCGATAAGATCGGTGGC | |
| | CCTTCCTCCGGTGGAAAGCGCCCAGCGGCTACAAAGAAGGC | |
| | CGGACAGGCCAAGAAGAAGAAGGGTTCGTACCCCTACGACG | |
| | TGCCTGACTATGCCTGA | |
| TAL02-MQ1 | MAPKKKRKVGGSGGSHMVDLRTLGYSQQQQEKIKPKVRSTVA | 51 |
| amino acid | QHHEALVGHGFTHAHIVALSQHPAALGTVAVKYQDMIAALPE | |
| sequence | ATHEAIVGVGKRGAGARALEALLTVAGELRGPPLQLDTGQLLK | |
| | IAKRGGVTAVEAVHAWRNALTGAPLNLTPEQVVAIASNNGGK | |
| | QALETVQRLLPVLCQAHGLTPEQVVAIASNIGGKQALETVQRL | |
| | LPVLCQAHGLTPEQVVAIASHDGGKQALETVQRLLPVLCQAHG | |
| | LTPEQVVAIASHDGGKQALETVQRLLPVLCQAHGLTPEQVVAI | |
| | ASNGGGKQALETVQRLLPVLCQAHGLTPEQVVAIASNNGGKQ | |
| | ALETVQRLLPVLCQAHGLTPEQVVAIASNGGGKQALETVQRLL | |
| | PVLCQAHGLTPEQVVAIASNGGGKQALETVQRLLPVLCQAHGL | |
| | TPEQVVAIASNIGGKQALETVQRLLPVLCQAHGLTPEQVVAIAS | |
| | HDGGKQALETVQRLLPVLCQAHGLTPEQVVAIASNIGGKQALE | |
| | TVQRLLPVLCQAHGLTPEQVVAIASNGGGKQALETVQRLLPVL | |
| | CQAHGLTPEQVVAIASNGGGKQALETVQRLLPVLCQAHGLTPE | |
| | QVVAIASHDGGKQALETVQRLLPVLCQAHGLTPEQVVAIASHD | |
| | GGKQALETVQRLLPVLCQAHGLTPEQVVAIASHDGGKQALET | |
| | VQRLLPVLCQAHGLTPEQVVAIASHDGGKQALETVQRLLPVLC | |
| | QAHGLTPEQVVAIASHDGGRPALESIVAQLSRPDPALAALIND | |
| | HLVALACLGGRPALDAVKKGLPHAPALIKRTNRRIPERTSHRV | |
| | ASGSGGGSGGARDSKVENKTKKLRVFEAFAGIGAQRKALEKV | |
| | RKDEYEIVGLAEWYVPAIVMYQAIHNNFHTKLEYKSVSREEMI | |
| | DYLENKTLSWNSKNPVSNGYWKRKKDDELKIIYNAIKLSEKEG | |
| | NIFDIRDLYKRTLKNIDLLTYSFPCQDLSQQGIQKGMKRGSGTR | |
| | SGLLWEIERALDSTEKNDLPKYLLMENVGALLHKKNEEELNQ | |
| | WKQKLESLGYQNSIEVLNAADFGSSQARRRVFMISTLNEFVELP | |
| | KGDKKPKSIKKVLNKIVSEKDILNNLLKYNLTEFKKTKSNINKA | |
| | SLIGYSKFNSEGYVYDPEFTGPTLTASGANSRIKIKDGSNIRKMN | |
| | SDETFLYIGFDSQDGKRVNEIEFLTENQKIFVCGNSISVEVLEAII | |
| | DKIGGPSSGGKRPAATKKAGQAKKKKGSYPYDVPDYA* | |
| TAL02-MQ1 | CACAUGGUGGAUCUGCGCACCCUGGGGUACUCGCAGCAGC | 52 |
| DBD mRNA | AGCAGGAAAAGAUUAAGCCGAAGGUCCGGUCCACUGUCGC | |
| sequence | CCAGCACCACGAAGCGCUCGUCGGACACGGUUUCACUCAC | |
| | GCACACAUAGUCGCACUGAGCCAGCACCCUGCGGCACUGG | |
| | GAACCGUGGCCGUGAAGUACCAGGAUAUGAUCGCAGCCCU | |
| | UCCUGAGGCCACCCACGAAGCCAUCGUGGGCGUGGGAAAG | |
| | AGAGGUGCCGGAGCAAGGGCUCUUGAGGCCCUGCUGACUG | |
| | UCGCGGGAGAACUCAGGGGACCGCCUCUGCAACUGGACAC | |
| | CGGACAGCUGCUGAAAAUUGCCAAGCGGGGCGGUGUGACU | |
| | GCCGUGGAAGCCGUGCACGCCUGGAGAAACGCACUCACUG | |
| | GCGCUCCUCUGAACCUGACCCCGGAACAGGUCGUCGCGAU | |
| | UGCGUCGAACAAUGGAGGGAAACAGGCGUUGGAAACUGUC | |
| | CAACGGUUGCUCCCGGUGCUAUGUCAAGCCCAUGGACUGA | |
| | CGCCCGAACAAGUCGUAGCCAUCGCGUCAAACAUCGGCGG | |
| | AAAACAGGCGCUCGAAACGGUCCAGCGCUUACUCCCCGUG | |
| | CUGUGUCAAGCGCAUGGAUUGACUCCGGAGCAAGUCGUCG | |
| | CUAUCGCCAGCCACGACGGAGGAAAGCAAGCUUUGGAAAC | |
| | AGUGCAACGACUGCUGCCCGUCUUGUGUCAAGCACACGGG | |
| | UUGACCCCAGAGCAAGUGGUGGCCAUUGCCUCCCAUGAUG | |
| | GGGGAAAGCAGGCCCUGGAGACUGUGCAGCGCCUGUUGCC | |
| | GGUGUUGUGCCAAGCACAUGGGCUGACACCCGAACAGGUC | |
| | GUGGCGAUUGCCUCCAACGGUGGCGGCAAACAAGCACUCG | |
| | AGACAGUGCAGCGACUCCUCCCGGUUCUGUGCCAGGCUCA | |
| | CGGCCUCACUCCCGAACAGGUGGUGGCAAUAGCCUCCAAC | |
| | AACGGAGGCAAACAGGCUUUAGAAACGGUUCAGCGCUUGC | |
| | UGCCAGUCCUGUGCCAAGCGCACGGAUUGACCCCGGAGCA | |
| | AGUGGUGGCCAUAGCCAGCAACGGCGGAGGGAAGCAGGCC | |
| | UUGGAAACCGUACAGAGACUCCUCCCCGUGUUAUGCCAGG | |
| | CACACGGCCUGACACCAGAACAGGUGGUGGCGAUUGCUUC | |
| | CAACGGAGGCGGAAAGCAGGCCCUCGAAACAGUCCAGCGA | |
| | CUUCUGCCGGUGCUGUGCCAGGCUCACGGUUUGACUCCAG | |
| | AGCAAGUUGUAGCUAUUGCCAGCAACAUCGGUGGGAAGCA | |
| | GGCGCUGGAAACUGUGCAACGGCUCCUGCCCGUCCUCUGC | |
| | CAAGCUCAUGGCCUCACCCCCGAACAGGUCGUGGCCAUCGC | |
| | GAGCCACGAUGGAGGAAAACAAGCCCUGGAAACGGUGCAG | |
| | AGGUUGCUGCCCGUACUUUGUCAGGCCCACGGGCUUACUC | |
| | CCGAGCAAGUGGUGGCCAUCGCCUCCAACAUUGGCGGCAA | |
| | GCAGGCACUGGAAACUGUGCAGAGACUGCUUCCCGUGCUC | |
| | UGCCAGGCACACGGACUCACCCCUGAACAGGUUGUGGCCA | |
| | UAGCCUCAAACGGAGGAGGAAAGCAGGCCCUUGAAACUGU | |
| | CCAGAGACUGUUGCCCGUACUGUGUCAGGCCCACGGCCUU | |
| | ACUCCUGAACAGGUAGUGGCAAUCGCAUCGAACGGCGGUG | |
| | GGAAACAAGCUCUCGAGACUGUCCAGCGGCUGCUACCUGU | |
| | GUUGUGUCAGGCUCAUGGACUUACGCCGGAACAAGUAGUG | |
| | GCCAUUGCUUCCCACGACGGGGGUAAACAGGCUCUGGAAA | |
| | CCGUCCAAAGGCUGCUGCCAGUGUUGUGCCAGGCGCAUGG | |
| | CCUAACCCCAGAACAAGUGGUCGCAAUCGCCUCGCACGAU | |
| | GGUGGUAAACAAGCGCUAGAAACCGUGCAGAGGCUUCUCC | |
| | CAGUGCUCUGUCAGGCCCACGGUCUGACUCCAGAACAAGU | |
| | GGUCGCCAUCGCAUCCCAUGACGGGGGGAAGCAAGCCUUG | |
| | GAGACAGUCCAACGGCUGCUUCCGGUGUUAUGUCAGGCGC | |
| | ACGGACUGACUCCUGAGCAAGUCGUGGCUAUCGCGUCGCA | |
| | UGAUGGGGGCAAGCAAGCCCUGGAAACGGUGCAACGCCUG | |
| | CUGCCUGUCCUUUGCCAGGCACACGGGCUGACUCCGGAAC | |
| | AGGUGGUGGCUAUUGCCUCGCAUGACGGAGGUAGGCCCGC | |
| | ACUCGAAAGCAUUGUCGCACAGCUGUCCCGGCCAGAUCCU | |
| | GCUUUGGCCGCCCUGACCAACGAUCACCUCGUCGCUCUGGC | |
| | CUGCCUGGGUGGAAGGCCGGCUCUGGAUGCCGUGAAGAAG | |
| | GGGCUGCCUCAUGCCCCUGCCCUGAUUAAGAGAACUAACC | |
| | GGCGCAUUCCGGAGAGAACCUCCCACAGAGUG | |
| TAL02 DNA | HMVDLRTLGYSQQQQEKIKPKVRSTVAQHHEALVGHGFTHAH | 53 |
| bindingn | IVALSQHPAALGTVAVKYQDMIAALPEATHEAIVGVGKRGAG | |
| domain | ARALEALLTVAGELRGPPLQLDTGQLLKIAKRGGVTAVEAVHA | |
| amino acid | WRNALTGAPLNLTPEQVVAIASNNGGKQALETVQRLLPVLCQ | |
| sequence | AHGLTPEQVVAIASNIGGKQALETVQRLLPVLCQAHGLTPEQV | |
| | VAIASHDGGKQALETVQRLLPVLCQAHGLTPEQVVAIASHDGG | |
| | KQALETVQRLLPVLCQAHGLTPEQVVAIASNGGGKQALETVQR | |
| | LLPVLCQAHGLTPEQVVAIASNNGGKQALETVQRLLPVLCQAH | |
| | GLTPEQVVAIASNGGGKQALETVQRLLPVLCQAHGLTPEQVVA | |
| | IASNGGGKQALETVQRLLPVLCQAHGLTPEQVVAIASNIGGKQ | |
| | ALETVQRLLPVLCQAHGLTPEQVVAIASHDGGKQALETVQRLL | |
| | PVLCQAHGLTPEQVVAIASNIGGKQALETVQRLLPVLCQAHGL | |
| | TPEQVVAIASNGGGKQALETVQRLLPVLCQAHGLTPEQVVAIA | |
| | SNGGGKQALETVQRLLPVLCQAHGLTPEQVVAIASHDGGKQA | |
| | LETVQRLLPVLCQAHGLTPEQVVAIASHDGGKQALETVQRLLP | |
| | VLCQAHGLTPEQVVAIASHDGGKQALETVQRLLPVLCQAHGLT | |
| | PEQVVAIASHDGGKQALETVQRLLPVLCQAHGLTPEQVVAIAS | |
| | HDGGRPALESIVAQLSRPDPALAALTNDHLVALACLGGRPALD | |
| | AVKKGLPHAPALIKRTNRRIPERTSHRV | |
| 3′ UTR AU | (AAUAAA)ₙ | 54 |
| sequence | | |
| Linker | ASGSGGGSGG | 55 |
| TAL02-MQ1 | GACCTGTTACATTCCCCC | 56 |
| target DNA | | |
| sequence | | |
| TAL03-MQ1 | GGGAAAUAAGAGAGAAAAGAAGAGUAAGAAGAAAUAUAA | 57 |
| full-length | GAGCCACCAUGGCCCCAAAGAAGAAGCGGAAAGUCGGAGG | |
| mRNA | UUCAGGCGGCUCGCACAUGGUGGACCUGAGAACUCUGGGU | |
| sequence | UACUCCCAGCAACAGCAAGAAAAAAUCAAGCCAAAAGUGC | |
| | GCAGCACUGUCGCGCAGCACCACGAGGCCCUUGUGGGGCA | |
| | CGGGUUCACCCACGCUCACAUCGUGGCCCUGAGCCAGCAUC | |
| | CGGCAGCCCUGGGAACCGUCGCAGUGAAGUACCAAGACAU | |
| | GAUCGCCGCCUUGCCGGAAGCCACUCACGAAGCAAUUGUG | |
| | GGAGUGGGGAAGAGAGGAGCCGGCGCAAGAGCCCUCGAAG | |
| | CGCUUCUGACUGUGGCCGGAGAAUUGAGAGGCCCCCCACU | |
| | GCAGCUGGAUACCGGACAACUGCUGAAGAUCGCCAAACGG | |
| | GGGGGAGUGACCGCUGUGGAAGCCGUGCACGCAUGGAGGA | |
| | ACGCUCUCACCGGCGCACCGUUAAACCUUACGCCCGAACAG | |
| | GUUGUGGCCAUAGCCUCCAACAUUGGCGGGAAACAGGCAU | |
| | UGGAGACUGUCCAACGGUUGCUUCCAGUGCUUUGCCAAGC | |
| | CCACGGAUUGACACCAGAACAGGUCGUUGCAAUCGCCAGC | |
| | CACGAUGGGGGUAAACAAGCGCUGGAGACAGUCCAACGGC | |
| | UGCUCCCCGUGCUGUGCCAGGCCCAUGGUCUGACUCCGGA | |
| | ACAGGUGGUGGCCAUCGCCAGCAACGGUGGAGGCAAACAA | |
| | GCCCUCGAAACGGUACAAAGACUUUUGCCCGUGUUGUGCC | |
| | AGGCGCAUGGACUUACCCCUGAGCAAGUGGUAGCUAUUGC | |
| | CAGCCAUGAUGGGGGAAAGCAAGCCUUGGAGACAGUGCAA | |
| | CGCCUGCUCCCUGUGCUGUGUCAGGCGCACGGCCUCACUCC | |
| | GGAACAGGUCGUCGCUAUCGCUUCCCAUGACGGCGGCAAG | |
| | CAGGCUCUGGAAACAGUGCAGCGGUUGCUGCCCGUGCUCU | |
| | GCCAAGCACAUGGGCUGACUCCUGAACAAGUCGUGGCAAU | |
| | UGCCAGCAACAUUGGAGGAAAGCAGGCCUUGGAAACUGUC | |
| | CAGCGGCUCCUUCCGGUCCUCUGUCAAGCACACGGACUGA | |
| | CCCCGGAACAAGUGGUGGCGAUUGCUUCGAACAUCGGUGG | |
| | CAAGCAGGCACUGGAAACGGUGCAGAGGCUGCUGCCAGUG | |
| | CUGUGCCAGGCACACGGGCUUACUCCAGAACAGGUUGUCG | |
| | CUAUUGCCUCCAACGGAGGAGGCAAGCAGGCGCUCGAAAC | |
| | CGUACAACGCUUGCUGCCGGUCCUUUGUCAAGCGCACGGG | |
| | CUCACUCCAGAGCAAGUGGUCGCGAUUGCCUCCCACGAUG | |
| | GAGGGAAACAAGCUCUUGAGACUGUGCAGAGACUCUUGCC | |
| | UGUGCUCUGUCAGGCCCACGGUCUGACCCCUGAACAGGUG | |
| | GUUGCGAUUGCAAGCCACGACGGUGGUAAACAGGCCCUGG | |
| | AAACUGUGCAGCGCCUCCUGCCUGUCCUGUGCCAAGCGCA | |
| | UGGCCUCACCCCGGAACAAGUGGUGGCGAUUGCGAGCAAC | |
| | GGAGGGGGCAAACAGGCGCUGGAAACGGUGCAGAGGCUGC | |
| | UCCCGGUGCUUUGCCAAGCCCACGGACUUACACCCGAACA | |
| | GGUCGUGGCCAUCGCCAGUCAUGACGGGGGCAAGCAAGCU | |
| | UUGGAAACCGUGCAGCGGCUGUUGCCAGUGUUGUGUCAAG | |
| | CUCACGGGCUGACGCCUGAACAAGUCGUCGCCAUCGCUUC | |
| | ACACGACGGAGGAAAGCAGGCCUUGGAAACUGUCCAGAGG | |
| | CUCUUGCCGGUCCUAUGUCAGGCACAUGGACUGACUCCCG | |
| | AACAAGUGGUGGCAAUCGCCUCGAACGGUGGUGGGAAGCA | |
| | GGCCCUCGAGACUGUGCAACGGCUUCUGCCUGUGUUAUGC | |
| | CAGGCUCAUGGCCUGACCCCCGAGCAAGUAGUCGCCAUCG | |
| | CCUCACACGAUGGCGGAAAACAGGCUCUCGAAACAGUCCA | |
| | AAGACUGCUGCCCGUCCUGUGUCAAGCCCAUGGACUCACC | |
| | CCCGAACAAGUGGUGGCUAUCGCGUCGAAUAUCGGCGGGA | |
| | AGCAGGCCCUUGAAACCGUCCAGAGACUUCUGCCGGUGCU | |
| | CUGCCAAGCUCACGGCCUGACGCCAGAACAAGUAGUGGCC | |
| | AUUGCAUCUAACAACGGGGGGAAGCAAGCUCUCGAAACUG | |
| | UGCAAAGGCUCCUGCCCGUCUUGUGUCAGGCUCACGGUCU | |
| | GACCCCUGAACAGGUGGUCGCCAUUGCGUCACGCCAUGGA | |
| | GGACGCCCUGCCCUCGAGUCCAUUGUGGCCCAGCUGUCAC | |
| | GCCCAGAUCCUGCACUGGCCGCUCUUACCAACGAUCACCUG | |
| | GUCGCUCUCGCGUGCCUGGGAGGAAGGCCUGCCCUGGAUG | |
| | CCGUCAAGAAGGGACUGCCUCAUGCCCCUGCUUUGAUUAA | |
| | GAGGACUAAUCGGCGCAUCCCCGAACGGACCAGCCACAGG | |
| | GUGGCAUCUGGGAGCGGAGGAGGAUCCGGCGGAGCCAGAG | |
| | ACUCCAAGGUCGAGAACAAGACCAAGAAGCUGCGGGUGUU | |
| | CGAGGCCUUUGCCGGGAUCGGAGCACAGCGGAAGGCCUUG | |
| | GAGAAGGUCCGCAAGGACGAAUACGAAAUCGUGGGCCUUG | |
| | CCGAGUGGUACGUGCCAGCCAUCGUGAUGUACCAGGCUAU | |
| | CCACAACAACUUCCACACCAAGCUGGAGUACAAGUCCGUG | |
| | AGCAGAGAAGAGAUGAUCGACUACCUCGAAAACAAGACCC | |
| | UGUCCUGGAACUCCAAGAAUCCCGUGUCGAACGGUUACUG | |
| | GAAGCGCAAGAAGGAUGAUGAACUGAAGAUUAUCUACAAC | |
| | GCGAUUAAGCUGUCCGAAAAGGAAGGAAACAUUUUCGACA | |
| | UUCGCGAUCUGUACAAGAGAACCCUCAAGAACAUUGAUCU | |
| | GCUUACCUACUCCUUCCCGUGCCAAGAUCUCAGCCAGCAG | |
| | GGGAUUCAGAAGGGGAUGAAGCGGGGUUCAGGGACCAGAU | |
| | CAGGCCUGCUCUGGGAGAUCGAAAGAGCCCUGGACUCCAC | |
| | CGAGAAGAACGAUCUCCCGAAAUACCUUCUGAUGGAGAAC | |
| | GUCGGCGCUCUCCUGCACAAGAAGAACGAAGAAGAACUGA | |
| | ACCAGUGGAAGCAGAAGCUGGAGUCACUCGGAUACCAGAA | |
| | UUCUAUUGAGGUGCUGAACGCGGCCGAUUUCGGAUCCUCG | |
| | CAAGCCAGGCGGAGAGUGUUCAUGAUUUCGACCCUGAACG | |
| | AGUUCGUGGAGCUGCCUAAGGGCGACAAGAAGCCCAAGUC | |
| | CAUCAAGAAGGUCCUGAACAAGAUUGUCAGCGAAAAGGAC | |
| | AUCCUCAACAAUCUGCUUAAGUACAACCUGACCGAGUUUA | |
| | AAAAGACCAAAAGCAACAUCAACAAGGCCUCCCUGAUCGG | |
| | AUACUCCAAGUUCAAUUCCGAAGGCUACGUGUACGACCCG | |
| | GAAUUCACCGGACCAACCUUGACCGCCUCCGGUGCUAACU | |
| | CCCGGAUCAAGAUCAAGGACGGAUCCAACAUCCGGAAGAU | |
| | GAACUCCGACGAGACUUUUCUGUACAUCGGUUUCGACUCC | |
| | CAAGACGGAAAGCGCGUGAACGAGAUAGAGUUCCUCACCG | |
| | AGAAUCAGAAGAUCUUUGUGUGCGGCAACUCCAUUAGCGU | |
| | GGAGGUGCUGGAGGCCAUCAUCGAUAAGAUCGGUGGCCCU | |
| | UCCUCCGGUGGAAAGCGCCCAGCGGCUACAAAGAAGGCCG | |
| | GACAGGCCAAGAAGAAGAAGGGUUCGUACCCCUACGACGU | |
| | GCCUGACUAUGCCUGAGCGGCCGCUUAAUUAAGCUGCCUU | |
| | CUGCGGGGCUUGCCUUCUGGCCAUGCCCUUCUUCUCUCCCU | |
| | UGCACCUGUACCUCUUGGUCUUUGAAUAAAGCCUGAGUAG | |
| | GAAGUCUAGAAAAAAAAAAAAAAAAAAAAAAAAAAAAAA | |
| | AAAAAAAAAAAAAAAAAAAAAAAAAAAAAAAAAAAAAAA | |
| | AAAAAAAAAAAAAAAAAAAAAAAAAAAAAAAAAAAAAAA | |
| | AAA | |
| TAL03-MQ1 | ATGGCCCCAAAGAAGAAGCGGAAAGTCGGAGGTTCAGGCG | 58 |
| DNA | GCTCGCACATGGTGGACCTGAGAACTCTGGGTTACTCCCAGC | |
| sequence | AACAGCAAGAAAAAATCAAGCCAAAAGTGCGCAGCACTGTC | |
| (ORF) | GCGCAGCACCACGAGGCCCTTGTGGGGCACGGGTTCACCCA | |
| | CGCTCACATCGTGGCCCTGAGCCAGCATCCGGCAGCCCTGG | |
| | GAACCGTCGCAGTGAAGTACCAAGACATGATCGCCGCCTTG | |
| | CCGGAAGCCACTCACGAAGCAATTGTGGGAGTGGGGAAGAG | |
| | AGGAGCCGGCGCAAGAGCCCTCGAAGCGCTTCTGACTGTGG | |
| | CCGGAGAATTGAGAGGCCCCCCACTGCAGCTGGATACCGGA | |
| | CAACTGCTGAAGATCGCCAAACGGGGGGGAGTGACCGCTGT | |
| | GGAAGCCGTGCACGCATGGAGGAACGCTCTCACCGGCGCAC | |
| | CGTTAAACCTTACGCCCGAACAGGTTGTGGCCATAGCCTCCA | |
| | ACATTGGCGGGAAACAGGCATTGGAGACTGTCCAACGGTTG | |
| | CTTCCAGTGCTTTGCCAAGCCCACGGATTGACACCAGAACAG | |
| | GTCGTTGCAATCGCCAGCCACGATGGGGGTAAACAAGCGCT | |
| | GGAGACAGTCCAACGGCTGCTCCCCGTGCTGTGCCAGGCCC | |
| | ATGGTCTGACTCCGGAACAGGTGGTGGCCATCGCCAGCAAC | |
| | GGTGGAGGCAAACAAGCCCTCGAAACGGTACAAAGACTTTT | |
| | GCCCGTGTTGTGCCAGGCGCATGGACTTACCCCTGAGCAAGT | |
| | GGTAGCTATTGCCAGCCATGATGGGGGAAAGCAAGCCTTGG | |
| | AGACAGTGCAACGCCTGCTCCCTGTGCTGTGTCAGGCGCACG | |
| | GCCTCACTCCGGAACAGGTCGTCGCTATCGCTTCCCATGACG | |
| | GCGGCAAGCAGGCTCTGGAAACAGTGCAGCGGTTGCTGCCC | |
| | GTGCTCTGCCAAGCACATGGGCTGACTCCTGAACAAGTCGTG | |
| | GCAATTGCCAGCAACATTGGAGGAAAGCAGGCCTTGGAAAC | |
| | TGTCCAGCGGCTCCTTCCGGTCCTCTGTCAAGCACACGGACT | |
| | GACCCCGGAACAAGTGGTGGCGATTGCTTCGAACATCGGTG | |
| | GCAAGCAGGCACTGGAAACGGTGCAGAGGCTGCTGCCAGTG | |
| | CTGTGCCAGGCACACGGGCTTACTCCAGAACAGGTTGTCGCT | |
| | ATTGCCTCCAACGGAGGAGGCAAGCAGGCGCTCGAAACCGT | |
| | ACAACGCTTGCTGCCGGTCCTTTGTCAAGCGCACGGGCTCAC | |
| | TCCAGAGCAAGTGGTCGCGATTGCCTCCCACGATGGAGGGA | |
| | AACAAGCTCTTGAGACTGTGCAGAGACTCTTGCCTGTGCTCT | |
| | GTCAGGCCCACGGTCTGACCCCTGAACAGGTGGTTGCGATTG | |
| | CAAGCCACGACGGTGGTAAACAGGCCCTGGAAACTGTGCAG | |
| | CGCCTCCTGCCTGTCCTGTGCCAAGCGCATGGCCTCACCCCG | |
| | GAACAAGTGGTGGCGATTGCGAGCAACGGAGGGGGCAAAC | |
| | AGGCGCTGGAAACGGTGCAGAGGCTGCTCCCGGTGCTTTGC | |
| | CAAGCCCACGGACTTACACCCGAACAGGTCGTGGCCATCGC | |
| | CAGTCATGACGGGGGCAAGCAAGCTTTGGAAACCGTGCAGC | |
| | GGCTGTTGCCAGTGTTGTGTCAAGCTCACGGGCTGACGCCTG | |
| | AACAAGTCGTCGCCATCGCTTCACACGACGGAGGAAAGCAG | |
| | GCCTTGGAAACTGTCCAGAGGCTCTTGCCGGTCCTATGTCAG | |
| | GCACATGGACTGACTCCCGAACAAGTGGTGGCAATCGCCTC | |
| | GAACGGTGGTGGGAAGCAGGCCCTCGAGACTGTGCAACGGC | |
| | TTCTGCCTGTGTTATGCCAGGCTCATGGCCTGACCCCCGAGC | |
| | AAGTAGTCGCCATCGCCTCACACGATGGCGGAAAACAGGCT | |
| | CTCGAAACAGTCCAAAGACTGCTGCCCGTCCTGTGTCAAGCC | |
| | CATGGACTCACCCCCGAACAAGTGGTGGCTATCGCGTCGAA | |
| | TATCGGCGGGAAGCAGGCCCTTGAAACCGTCCAGAGACTTC | |
| | TGCCGGTGCTCTGCCAAGCTCACGGCCTGACGCCAGAACAA | |
| | GTAGTGGCCATTGCATCTAACAACGGGGGGAAGCAAGCTCT | |
| | CGAAACTGTGCAAAGGCTCCTGCCCGTCTTGTGTCAGGCTCA | |
| | CGGTCTGACCCCTGAACAGGTGGTCGCCATTGCGTCACGCCA | |
| | TGGAGGACGCCCTGCCCTCGAGTCCATTGTGGCCCAGCTGTC | |
| | ACGCCCAGATCCTGCACTGGCCGCTCTTACCAACGATCACCT | |
| | GGTCGCTCTCGCGTGCCTGGGAGGAAGGCCTGCCCTGGATG | |
| | CCGTCAAGAAGGGACTGCCTCATGCCCCTGCTTTGATTAAGA | |
| | GGACTAATCGGCGCATCCCCGAACGGACCAGCCACAGGGTG | |
| | GCATCTGGGAGCGGAGGAGGATCCGGCGGAGCCAGAGACTC | |
| | CAAGGTCGAGAACAAGACCAAGAAGCTGCGGGTGTTCGAGG | |
| | CCTTTGCCGGGATCGGAGCACAGCGGAAGGCCTTGGAGAAG | |
| | GTCCGCAAGGACGAATACGAAATCGTGGGCCTTGCCGAGTG | |
| | GTACGTGCCAGCCATCGTGATGTACCAGGCTATCCACAACA | |
| | ACTTCCACACCAAGCTGGAGTACAAGTCCGTGAGCAGAGAA | |
| | GAGATGATCGACTACCTCGAAAACAAGACCCTGTCCTGGAA | |
| | CTCCAAGAATCCCGTGTCGAACGGTTACTGGAAGCGCAAGA | |
| | AGGATGATGAACTGAAGATTATCTACAACGCGATTAAGCTG | |
| | TCCGAAAAGGAAGGAAACATTTTCGACATTCGCGATCTGTA | |
| | CAAGAGAACCCTCAAGAACATTGATCTGCTTACCTACTCCTT | |
| | CCCGTGCCAAGATCTCAGCCAGCAGGGGATTCAGAAGGGGA | |
| | TGAAGCGGGGTTCAGGGACCAGATCAGGCCTGCTCTGGGAG | |
| | ATCGAAAGAGCCCTGGACTCCACCGAGAAGAACGATCTCCC | |
| | GAAATACCTTCTGATGGAGAACGTCGGCGCTCTCCTGCACAA | |
| | GAAGAACGAAGAAGAACTGAACCAGTGGAAGCAGAAGCTG | |
| | GAGTCACTCGGATACCAGAATTCTATTGAGGTGCTGAACGC | |
| | GGCCGATTTCGGATCCTCGCAAGCCAGGCGGAGAGTGTTCA | |
| | TGATTTCGACCCTGAACGAGTTCGTGGAGCTGCCTAAGGGCG | |
| | ACAAGAAGCCCAAGTCCATCAAGAAGGTCCTGAACAAGATT | |
| | GTCAGCGAAAAGGACATCCTCAACAATCTGCTTAAGTACAA | |
| | CCTGACCGAGTTTAAAAAGACCAAAAGCAACATCAACAAGG | |
| | CCTCCCTGATCGGATACTCCAAGTTCAATTCCGAAGGCTACG | |
| | TGTACGACCCGGAATTCACCGGACCAACCTTGACCGCCTCCG | |
| | GTGCTAACTCCCGGATCAAGATCAAGGACGGATCCAACATC | |
| | CGGAAGATGAACTCCGACGAGACTTTTCTGTACATCGGTTTC | |
| | GACTCCCAAGACGGAAAGCGCGTGAACGAGATAGAGTTCCT | |
| | CACCGAGAATCAGAAGATCTTTGTGTGCGGCAACTCCATTAG | |
| | CGTGGAGGTGCTGGAGGCCATCATCGATAAGATCGGTGGCC | |
| | CTTCCTCCGGTGGAAAGCGCCCAGCGGCTACAAAGAAGGCC | |
| | GGACAGGCCAAGAAGAAGAAGGGTTCGTACCCCTACGACGT | |
| | GCCTGACTATGCCTGA | |
| TAL03-MQ1 | MAPKKKRKVGGSGGSHMVDLRTLGYSQQQQEKIKPKVRSTVA | 59 |
| amino acid | QHHEALVGHGFTHAHIVALSQHPAALGTVAVKYQDMIAALPE | |
| sequence | ATHEAIVGVGKRGAGARALEALLTVAGELRGPPLQLDTGQLLK | |
| | IAKRGGVTAVEAVHAWRNALTGAPLNLTPEQVVAIASNIGGKQ | |
| | ALETVQRLLPVLCQAHGLTPEQVVAIASHDGGKQALETVQRLL | |
| | PVLCQAHGLTPEQVVAIASNGGGKQALETVQRLLPVLCQAHGL | |
| | TPEQVVAIASHDGGKQALETVQRLLPVLCQAHGLTPEQVVAIA | |
| | SHDGGKQALETVQRLLPVLCQAHGLTPEQVVAIASNIGGKQAL | |
| | ETVQRLLPVLCQAHGLTPEQVVAIASNIGGKQALETVQRLLPVL | |
| | CQAHGLTPEQVVAIASNGGGKQALETVQRLLPVLCQAHGLTPE | |
| | QVVAIASHDGGKQALETVQRLLPVLCQAHGLTPEQVVAIASHD | |
| | GGKQALETVQRLLPVLCQAHGLTPEQVVAIASNGGGKQALET | |
| | VQRLLPVLCQAHGLTPEQVVAIASHDGGKQALETVQRLLPVLC | |
| | QAHGLTPEQVVAIASHDGGKQALETVQRLLPVLCQAHGLTPEQ | |
| | VVAIASNGGGKQALETVQRLLPVLCQAHGLTPEQVVAIASHDG | |
| | GKQALETVQRLLPVLCQAHGLTPEQVVAIASNIGGKQALETVQ | |
| | RLLPVLCQAHGLTPEQVVAIASNNGGKQALETVQRLLPVLCQA | |
| | HGLTPEQVVAIASRHGGRPALESIVAQLSRPDPALAALTNDHLV | |
| | ALACLGGRPALDAVKKGLPHAPALIKRTNRRIPERTSHRVASGS | |
| | GGGSGGARDSKVENKTKKLRVFEAFAGIGAQRKALEKVRKDE | |
| | YEIVGLAEWYVPAIVMYQAIHNNFHTKLEYKSVSREEMIDYLE | |
| | NKTLSWNSKNPVSNGYWKRKKDDELKIIYNAIKLSEKEGNIFDI | |
| | RDLYKRTLKNIDLLTYSFPCQDLSQQGIQKGMKRGSGTRSGLL | |
| | WEIERALDSTEKNDLPKYLLMENVGALLHKKNEEELNQWKQK | |
| | LESLGYQNSIEVLNAADFGSSQARRRVFMISTLNEFVELPKGDK | |
| | KPKSIKKVLNKIVSEKDILNNLLKYNLTEFKKTKSNINKASLIGY | |
| | SKFNSEGYVYDPEFTGPTLTASGANSRIKIKDGSNIRKMNSDETF | |
| | LYIGFDSQDGKRVNEIEFLTENQKIFVCGNSISVEVLEAIIDKIGG | |
| | PSSGGKRPAATKKAGQAKKKKGSYPYDVPDYA* | |
| TAL03-MQ1 | CACAUGGUGGACCUGAGAACUCUGGGUUACUCCCAGCAAC | 60 |
| DBD mRNA | AGCAAGAAAAAAUCAAGCCAAAAGUGCGCAGCACUGUCGC | |
| sequence | GCAGCACCACGAGGCCCUUGUGGGGCACGGGUUCACCCAC | |
| | GCUCACAUCGUGGCCCUGAGCCAGCAUCCGGCAGCCCUGG | |
| | GAACCGUCGCAGUGAAGUACCAAGACAUGAUCGCCGCCUU | |
| | GCCGGAAGCCACUCACGAAGCAAUUGUGGGAGUGGGGAAG | |
| | AGAGGAGCCGGCGCAAGAGCCCUCGAAGCGCUUCUGACUG | |
| | UGGCCGGAGAAUUGAGAGGCCCCCCACUGCAGCUGGAUAC | |
| | CGGACAACUGCUGAAGAUCGCCAAACGGGGGGGAGUGACC | |
| | GCUGUGGAAGCCGUGCACGCAUGGAGGAACGCUCUCACCG | |
| | GCGCACCGUUAAACCUUACGCCCGAACAGGUUGUGGCCAU | |
| | AGCCUCCAACAUUGGCGGGAAACAGGCAUUGGAGACUGUC | |
| | CAACGGUUGCUUCCAGUGCUUUGCCAAGCCCACGGAUUGA | |
| | CACCAGAACAGGUCGUUGCAAUCGCCAGCCACGAUGGGGG | |
| | UAAACAAGCGCUGGAGACAGUCCAACGGCUGCUCCCCGUG | |
| | CUGUGCCAGGCCCAUGGUCUGACUCCGGAACAGGUGGUGG | |
| | CCAUCGCCAGCAACGGUGGAGGCAAACAAGCCCUCGAAAC | |
| | GGUACAAAGACUUUUGCCCGUGUUGUGCCAGGCGCAUGGA | |
| | CUUACCCCUGAGCAAGUGGUAGCUAUUGCCAGCCAUGAUG | |
| | GGGGAAAGCAAGCCUUGGAGACAGUGCAACGCCUGCUCCC | |
| | UGUGCUGUGUCAGGCGCACGGCCUCACUCCGGAACAGGUC | |
| | GUCGCUAUCGCUUCCCAUGACGGCGGCAAGCAGGCUCUGG | |
| | AAACAGUGCAGCGGUUGCUGCCCGUGCUCUGCCAAGCACA | |
| | UGGGCUGACUCCUGAACAAGUCGUGGCAAUUGCCAGCAAC | |
| | AUUGGAGGAAAGCAGGCCUUGGAAACUGUCCAGCGGCUCC | |
| | UUCCGGUCCUCUGUCAAGCACACGGACUGACCCCGGAACA | |
| | AGUGGUGGCGAUUGCUUCGAACAUCGGUGGCAAGCAGGCA | |
| | CUGGAAACGGUGCAGAGGCUGCUGCCAGUGCUGUGCCAGG | |
| | CACACGGGCUUACUCCAGAACAGGUUGUCGCUAUUGCCUC | |
| | CAACGGAGGAGGCAAGCAGGCGCUCGAAACCGUACAACGC | |
| | UUGCUGCCGGUCCUUUGUCAAGCGCACGGGCUCACUCCAG | |
| | AGCAAGUGGUCGCGAUUGCCUCCCACGAUGGAGGGAAACA | |
| | AGCUCUUGAGACUGUGCAGAGACUCUUGCCUGUGCUCUGU | |
| | CAGGCCCACGGUCUGACCCCUGAACAGGUGGUUGCGAUUG | |
| | CAAGCCACGACGGUGGUAAACAGGCCCUGGAAACUGUGCA | |
| | GCGCCUCCUGCCUGUCCUGUGCCAAGCGCAUGGCCUCACCC | |
| | CGGAACAAGUGGUGGCGAUUGCGAGCAACGGAGGGGGCAA | |
| | ACAGGCGCUGGAAACGGUGCAGAGGCUGCUCCCGGUGCUU | |
| | UGCCAAGCCCACGGACUUACACCCGAACAGGUCGUGGCCA | |
| | UCGCCAGUCAUGACGGGGGCAAGCAAGCUUUGGAAACCGU | |
| | GCAGCGGCUGUUGCCAGUGUUGUGUCAAGCUCACGGGCUG | |
| | ACGCCUGAACAAGUCGUCGCCAUCGCUUCACACGACGGAG | |
| | GAAAGCAGGCCUUGGAAACUGUCCAGAGGCUCUUGCCGGU | |
| | CCUAUGUCAGGCACAUGGACUGACUCCCGAACAAGUGGUG | |
| | GCAAUCGCCUCGAACGGUGGUGGGAAGCAGGCCCUCGAGA | |
| | CUGUGCAACGGCUUCUGCCUGUGUUAUGCCAGGCUCAUGG | |
| | CCUGACCCCCGAGCAAGUAGUCGCCAUCGCCUCACACGAUG | |
| | GCGGAAAACAGGCUCUCGAAACAGUCCAAAGACUGCUGCC | |
| | CGUCCUGUGUCAAGCCCAUGGACUCACCCCCGAACAAGUG | |
| | GUGGCUAUCGCGUCGAAUAUCGGCGGGAAGCAGGCCCUUG | |
| | AAACCGUCCAGAGACUUCUGCCGGUGCUCUGCCAAGCUCA | |
| | CGGCCUGACGCCAGAACAAGUAGUGGCCAUUGCAUCUAAC | |
| | AACGGGGGGAAGCAAGCUCUCGAAACUGUGCAAAGGCUCC | |
| | UGCCCGUCUUGUGUCAGGCUCACGGUCUGACCCCUGAACA | |
| | GGUGGUCGCCAUUGCGUCACGCCAUGGAGGACGCCCUGCC | |
| | CUCGAGUCCAUUGUGGCCCAGCUGUCACGCCCAGAUCCUG | |
| | CACUGGCCGCUCUUACCAACGAUCACCUGGUCGCUCUCGCG | |
| | UGCCUGGGAGGAAGGCCUGCCCUGGAUGCCGUCAAGAAGG | |
| | GACUGCCUCAUGCCCCUGCUUUGAUUAAGAGGACUAAUCG | |
| | GCGCAUCCCCGAACGGACCAGCCACAGGGUG | |
| TAL03-MQ1 | HMVDLRTLGYSQQQQEKIKPKVRSTVAQHHEALVGHGFTHAH | 61 |
| DBD amino | IVALSQHPAALGTVAVKYQDMIAALPEATHEAIVGVGKRGAG | |
| acid sequence | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASHDGGKQALETVQRLLPVLCQAHGLTPEQVV | |
| | AIASNGGGKQALETVQRLLPVLCQAHGLTPEQVVAIASHDGGK | |
| | QALETVQRLLPVLCQAHGLTPEQVVAIASHDGGKQALETVQRL | |
| | LPVLCQAHGLTPEQVVAIASNIGGKQALETVQRLLPVLCQAHG | |
| | LTPEQVVAIASNIGGKQALETVQRLLPVLCQAHGLTPEQVVAIA | |
| | SNGGGKQALETVQRLLPVLCQAHGLTPEQVVAIASHDGGKQA | |
| | LETVQRLLPVLCQAHGLTPEQVVAIASHDGGKQALETVQRLLP | |
| | VLCQAHGLTPEQVVAIASNGGGKQALETVQRLLPVLCQAHGLT | |
| | PEQVVAIASHDGGKQALETVQRLLPVLCQAHGLTPEQVVAIAS | |
| | HDGGKQALETVQRLLPVLCQAHGLTPEQVVAIASNGGGKQAL | |
| | ETVQRLLPVLCQAHGLTPEQVVAIASHDGGKQALETVQRLLPV | |
| | LCQAHGLTPEQVVAIASNIGGKQALETVQRLLPVLCQAHGLTP | |
| | EQVVAIASNNGGKQALETVQRLLPVLCQAHGLTPEQVVAIASR | |
| | HGGRPALESIVAQLSRPDPALAALINDHLVALACLGGRPALDA | |
| | VKKGLPHAPALIKRTNRRIPERTSHRV | |
| Linker | ASGSGGGSGGARD | 62 |
| Gly-ser linker | G4S | 63 |
| TAL03-MQ1 | ACTCCAATCCTCCTCAGG | 64 |
| target DNA | | |
| sequence | | |
| mPCSK9 | GTTTAGGAGAAGTTAGTTAATATTGTTG | 65 |
| forward | | |
| primer | | |
| mPCSK9 | ATATCTCTAAAAAACCAAAAACTAACTC | 66 |
| reverse | | |
| primer | | |
| DBD473- | HMVDLRTLGYSQQQQEKIKPKVRSTVAQHHEALVGHGFTHAH | 81 |
| PCSK9 | IVALSQHPAALGTVAVKYQDMIAALPEATHEAIVGVGKRGAG | |
| TAL04 | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASNNGGKQALETVQRLLPVLCQAHGLTPEQVV | |
| | AIASNIGGKQALETVQRLLPVLCQAHGLTPEQVVAIASHDGGK | |
| | QALETVQRLLPVLCQAHGLTPEQVVAIASNGGGKQALETVQRL | |
| | LPVLCQAHGLTPEQVVAIASNGGGKQALETVQRLLPVLCQAHG | |
| | LTPEQVVAIASHDGGKQALETVQRLLPVLCQAHGLTPEQVVAI | |
| | ASNGGGKQALETVQRLLPVLCQAHGLTPEQVVAIASNNGGKQ | |
| | ALETVQRLLPVLCQAHGLTPEQVVAIASNIGGKQALETVQRLLP | |
| | VLCQAHGLTPEQVVAIASNIGGKQALETVQRLLPVLCQAHGLT | |
| | PEQVVAIASNGGGKQALETVQRLLPVLCQAHGLTPEQVVAIAS | |
| | HDGGKQALETVQRLLPVLCQAHGLTPEQVVAIASNIGGKQALE | |
| | TVQRLLPVLCQAHGLTPEQVVAIASNIGGKQALETVQRLLPVLC | |
| | QAHGLTPEQVVAIASNGGGKQALETVQRLLPVLCQAHGLTPEQ | |
| | VVAIASHDGGKQALETVQRLLPVLCQAHGLTPEQVVAIASHDG | |
| | GRPALESIVAQLSRPDPALAALTNDHLVALACLGGRPALDAVK | |
| | KGLPHAPALIKRTNRRIPERTSHRV | |
| DBD474- | HMVDLRTLGYSQQQQEKIKPKVRSTVAQHHEALVGHGFTHAH | 82 |
| PCSK9 | IVALSQHPAALGTVAVKYQDMIAALPEATHEAIVGVGKRGAG | |
| TAL05 | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASNNGGKQALETVQRLLPVLCQAHGLTPEQVV | |
| | AIASNIGGKQALETVQRLLPVLCQAHGLTPEQVVAIASNIGGKQ | |
| | ALETVQRLLPVLCQAHGLTPEQVVAIASNNGGKQALETVQRLL | |
| | PVLCQAHGLTPEQVVAIASNGGGKQALETVQRLLPVLCQAHGL | |
| | TPEQVVAIASHDGGKQALETVQRLLPVLCQAHGLTPEQVVAIA | |
| | SNGGGKQALETVQRLLPVLCQAHGLTPEQVVAIASHDGGKQA | |
| | LETVQRLLPVLCQAHGLTPEQVVAIASNIGGKQALETVQRLLPV | |
| | LCQAHGLTPEQVVAIASHDGGKQALETVQRLLPVLCQAHGLTP | |
| | EQVVAIASNGGGKQALETVQRLLPVLCQAHGLTPEQVVAIASN | |
| | NGGKQALETVQRLLPVLCQAHGLTPEQVVAIASRHGGKQALET | |
| | VQRLLPVLCQAHGLTPEQVVAIASNGGGKQALETVQRLLPVLC | |
| | QAHGLTPEQVVAIASHDGGKQALETVQRLLPVLCQAHGLTPEQ | |
| | VVAIASNIGGKQALETVQRLLPVLCQAHGLTPEQVVAIASNNG | |
| | GRPALESIVAQLSRPDPALAALTNDHLVALACLGGRPALDAVK | |
| | KGLPHAPALIKRTNRRIPERTSHRV | |
| DBD475- | HMVDLRTLGYSQQQQEKIKPKVRSTVAQHHEALVGHGFTHAH | 83 |
| PCSK9 | IVALSQHPAALGTVAVKYQDMIAALPEATHEAIVGVGKRGAG | |
| TAL06 | ARALEALLTVAGELRGPPLQLDTGQLLKIAKRGGVTAVEAVHA | |
| | WRNALTGAPLNLTPEQVVAIASNNGGKQALETVQRLLPVLCQ | |
| | AHGLTPEQVVAIASRHGGKQALETVQRLLPVLCQAHGLTPEQV | |
| | VAIASNGGGKQALETVQRLLPVLCQAHGLTPEQVVAIASNNGG | |
| | KQALETVQRLLPVLCQAHGLTPEQVVAIASRHGGKQALETVQR | |
| | LLPVLCQAHGLTPEQVVAIASNIGGKQALETVQRLLPVLCQAH | |
| | GLTPEQVVAIASHDGGKQALETVQRLLPVLCQAHGLTPEQVVA | |
| | IASHDGGKQALETVQRLLPVLCQAHGLTPEQVVAIASHDGGKQ | |
| | ALETVQRLLPVLCQAHGLTPEQVVAIASNIGGKQALETVQRLLP | |
| | VLCQAHGLTPEQVVAIASNNGGKQALETVQRLLPVLCQAHGLT | |
| | PEQVVAIASRHGGKQALETVQRLLPVLCQAHGLTPEQVVAIAS | |
| | NIGGKQALETVQRLLPVLCQAHGLTPEQVVAIASNIGGKQALET | |
| | VQRLLPVLCQAHGLTPEQVVAIASNIGGKQALETVQRLLPVLC | |
| | QAHGLTPEQVVAIASHDGGKQALETVQRLLPVLCQAHGLTPEQ | |
| | VVAIASNIGGKQALETVQRLLPVLCQAHGLTPEQVVAIASHDG | |
| | GRPALESIVAQLSRPDPALAALTNDHLVALACLGGRPALDAVK | |
| | KGLPHAPALIKRTNRRIPERTSHRV | |
| DBD476- | HMVDLRTLGYSQQQQEKIKPKVRSTVAQHHEALVGHGFTHAH | 84 |
| PCSK9 | IVALSQHPAALGTVAVKYQDMIAALPEATHEAIVGVGKRGAG | |
| TAL07 | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASNIGGKQALETVQRLLPVLCQAHGLTPEQVV | |
| | AIASNNGGKQALETVQRLLPVLCQAHGLTPEQVVAIASRHGGK | |
| | QALETVQRLLPVLCQAHGLTPEQVVAIASNGGGKQALETVQRL | |
| | LPVLCQAHGLTPEQVVAIASNNGGKQALETVQRLLPVLCQAHG | |
| | LTPEQVVAIASRHGGKQALETVQRLLPVLCQAHGLTPEQVVAI | |
| | ASNIGGKQALETVQRLLPVLCQAHGLTPEQVVAIASHDGGKQA | |
| | LETVQRLLPVLCQAHGLTPEQVVAIASHDGGKQALETVQRLLP | |
| | VLCQAHGLTPEQVVAIASHDGGKQALETVQRLLPVLCQAHGLT | |
| | PEQVVAIASNIGGKQALETVQRLLPVLCQAHGLTPEQVVAIASN | |
| | NGGKQALETVQRLLPVLCQAHGLTPEQVVAIASRHGGKQALET | |
| | VQRLLPVLCQAHGLTPEQVVAIASNIGGKQALETVQRLLPVLC | |
| | QAHGLTPEQVVAIASNIGGKQALETVQRLLPVLCQAHGLTPEQ | |
| | VVAIASNIGGKQALETVQRLLPVLCQAHGLTPEQVVAIASHDG | |
| | GRPALESIVAQLSRPDPALAALTNDHLVALACLGGRPALDAVK | |
| | KGLPHAPALIKRTNRRIPERTSHRV | |
| DBD477- | HMVDLRTLGYSQQQQEKIKPKVRSTVAQHHEALVGHGFTHAH | 85 |
| PCSK9 | IVALSQHPAALGTVAVKYQDMIAALPEATHEAIVGVGKRGAG | |
| TAL08 | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASHDGGKQALETVQRLLPVLCQAHGLTPEQVV | |
| | AIASNGGGKQALETVQRLLPVLCQAHGLTPEQVVAIASNNGGK | |
| | QALETVQRLLPVLCQAHGLTPEQVVAIASRHGGKQALETVQRL | |
| | LPVLCQAHGLTPEQVVAIASNIGGKQALETVQRLLPVLCQAHG | |
| | LTPEQVVAIASNNGGKQALETVQRLLPVLCQAHGLTPEQVVAI | |
| | ASRHGGKQALETVQRLLPVLCQAHGLTPEQVVAIASNIGGKQA | |
| | LETVQRLLPVLCQAHGLTPEQVVAIASNGGGKQALETVQRLLP | |
| | VLCQAHGLTPEQVVAIASHDGGKQALETVQRLLPVLCQAHGLT | |
| | PEQVVAIASNIGGKQALETVQRLLPVLCQAHGLTPEQVVAIASN | |
| | NGGKQALETVQRLLPVLCQAHGLTPEQVVAIASRHGGKQALET | |
| | VQRLLPVLCQAHGLTPEQVVAIASNGGGKQALETVQRLLPVLC | |
| | QAHGLTPEQVVAIASNGGGKQALETVQRLLPVLCQAHGLTPEQ | |
| | VVAIASNGGGKQALETVQRLLPVLCQAHGLTPEQVVAIASHDG | |
| | GRPALESIVAQLSRPDPALAALINDHLVALACLGGRPALDAVK | |
| | KGLPHAPALIKRTNRRIPERTSHRV | |
| DBD478- | HMVDLRTLGYSQQQQEKIKPKVRSTVAQHHEALVGHGFTHAH | 86 |
| PCSK9 | IVALSQHPAALGTVAVKYQDMIAALPEATHEAIVGVGKRGAG | |
| TAL09 | ARALEALLTVAGELRGPPLQLDTGQLLKIAKRGGVTAVEAVHA | |
| | WRNALTGAPLNLTPEQVVAIASNNGGKQALETVQRLLPVLCQ | |
| | AHGLTPEQVVAIASNGGGKQALETVQRLLPVLCQAHGLTPEQV | |
| | VAIASNGGGKQALETVQRLLPVLCQAHGLTPEQVVAIASNGGG | |
| | KQALETVQRLLPVLCQAHGLTPEQVVAIASNIGGKQALETVQR | |
| | LLPVLCQAHGLTPEQVVAIASNIGGKQALETVQRLLPVLCQAH | |
| | GLTPEQVVAIASNGGGKQALETVQRLLPVLCQAHGLTPEQVVA | |
| | IASHDGGKQALETVQRLLPVLCQAHGLTPEQVVAIASNIGGKQ | |
| | ALETVQRLLPVLCQAHGLTPEQVVAIASNNGGKQALETVQRLL | |
| | PVLCQAHGLTPEQVVAIASNIGGKQALETVQRLLPVLCQAHGL | |
| | TPEQVVAIASNGGGKQALETVQRLLPVLCQAHGLTPEQVVAIA | |
| | SNIGGKQALETVQRLLPVLCQAHGLTPEQVVAIASNNGGKQAL | |
| | ETVQRLLPVLCQAHGLTPEQVVAIASRHGGKQALETVQRLLPV | |
| | LCQAHGLTPEQVVAIASNIGGKQALETVQRLLPVLCQAHGLTP | |
| | EQVVAIASNGGGKQALETVQRLLPVLCQAHGLTPEQVVAIASH | |
| | DGGRPALESIVAQLSRPDPALAALINDHLVALACLGGRPALDA | |
| | VKKGLPHAPALIKRTNRRIPERTSHRV | |
| DBD479- | HMVDLRTLGYSQQQQEKIKPKVRSTVAQHHEALVGHGFTHAH | 87 |
| PCSK9 | IVALSQHPAALGTVAVKYQDMIAALPEATHEAIVGVGKRGAG | |
| TAL10 | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASNGGGKQALETVQRLLPVLCQAHGLTPEQVV | |
| | AIASHDGGKQALETVQRLLPVLCQAHGLTPEQVVAIASNGGGK | |
| | QALETVQRLLPVLCQAHGLTPEQVVAIASNNGGKQALETVQRL | |
| | LPVLCQAHGLTPEQVVAIASNIGGKQALETVQRLLPVLCQAHG | |
| | LTPEQVVAIASNGGGKQALETVQRLLPVLCQAHGLTPEQVVAI | |
| | ASNGGGKQALETVQRLLPVLCQAHGLTPEQVVAIASNIGGKQA | |
| | LETVQRLLPVLCQAHGLTPEQVVAIASNIGGKQALETVQRLLPV | |
| | LCQAHGLTPEQVVAIASNIGGKQALETVQRLLPVLCQAHGLTP | |
| | EQVVAIASHDGGKQALETVQRLLPVLCQAHGLTPEQVVAIASN | |
| | IGGKQALETVQRLLPVLCQAHGLTPEQVVAIASNGGGKQALET | |
| | VQRLLPVLCQAHGLTPEQVVAIASNGGGKQALETVQRLLPVLC | |
| | QAHGLTPEQVVAIASNIGGKQALETVQRLLPVLCQAHGLTPEQ | |
| | VVAIASNIGGKQALETVQRLLPVLCQAHGLTPEQVVAIASHDG | |
| | GRPALESIVAQLSRPDPALAALTNDHLVALACLGGRPALDAVK | |
| | KGLPHAPALIKRTNRRIPERTSHRV | |
| DBD480- | HMVDLRTLGYSQQQQEKIKPKVRSTVAQHHEALVGHGFTHAH | 88 |
| PCSK9 | IVALSQHPAALGTVAVKYQDMIAALPEATHEAIVGVGKRGAG | |
| TAL11 | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASNIGGKQALETVQRLLPVLCQAHGLTPEQVV | |
| | AIASNIGGKQALETVQRLLPVLCQAHGLTPEQVVAIASHDGGK | |
| | QALETVQRLLPVLCQAHGLTPEQVVAIASHDGGKQALETVQRL | |
| | LPVLCQAHGLTPEQVVAIASNGGGKQALETVQRLLPVLCQAHG | |
| | LTPEQVVAIASNGGGKQALETVQRLLPVLCQAHGLTPEQVVAI | |
| | ASHDGGKQALETVQRLLPVLCQAHGLTPEQVVAIASNGGGKQ | |
| | ALETVQRLLPVLCQAHGLTPEQVVAIASNIGGKQALETVQRLLP | |
| | VLCQAHGLTPEQVVAIASNNGGKQALETVQRLLPVLCQAHGLT | |
| | PEQVVAIASRHGGKQALETVQRLLPVLCQAHGLTPEQVVAIAS | |
| | RHGGKQALETVQRLLPVLCQAHGLTPEQVVAIASNGGGKQAL | |
| | ETVQRLLPVLCQAHGLTPEQVVAIASNNGGKQALETVQRLLPV | |
| | LCQAHGLTPEQVVAIASNGGGKQALETVQRLLPVLCQAHGLTP | |
| | EQVVAIASNNGGKQALETVQRLLPVLCQAHGLTPEQVVAIASR | |
| | HGGRPALESIVAQLSRPDPALAALTNDHLVALACLGGRPALDA | |
| | VKKGLPHAPALIKRTNRRIPERTSHRV | |
| DBD481- | HMVDLRTLGYSQQQQEKIKPKVRSTVAQHHEALVGHGFTHAH | 89 |
| PCSK9 | IVALSQHPAALGTVAVKYQDMIAALPEATHEAIVGVGKRGAG | |
| TAL12 | ARALEALLTVAGELRGPPLQLDTGQLLKIAKRGGVTAVEAVHA | |
| | WRNALTGAPLNLTPEQVVAIASNIGGKQALETVQRLLPVLCQA | |
| | HGLTPEQVVAIASNIGGKQALETVQRLLPVLCQAHGLTPEQVV | |
| | AIASHDGGKQALETVQRLLPVLCQAHGLTPEQVVAIASHDGGK | |
| | QALETVQRLLPVLCQAHGLTPEQVVAIASNGGGKQALETVQRL | |
| | LPVLCQAHGLTPEQVVAIASNGGGKQALETVQRLLPVLCQAHG | |
| | LTPEQVVAIASHDGGKQALETVQRLLPVLCQAHGLTPEQVVAI | |
| | ASNGGGKQALETVQRLLPVLCQAHGLTPEQVVAIASNIGGKQA | |
| | LETVQRLLPVLCQAHGLTPEQVVAIASNNGGKQALETVQRLLP | |
| | VLCQAHGLTPEQVVAIASRHGGKQALETVQRLLPVLCQAHGLT | |
| | PEQVVAIASRHGGKQALETVQRLLPVLCQAHGLTPEQVVAIAS | |
| | NGGGKQALETVQRLLPVLCQAHGLTPEQVVAIASNNGGKQAL | |
| | ETVQRLLPVLCQAHGLTPEQVVAIASNGGGKQALETVQRLLPV | |
| | LCQAHGLTPEQVVAIASNNGGKQALETVQRLLPVLCQAHGLTP | |
| | EQVVAIASRHGGKQALETVQRLLPVLCQAHGLTPEQVVAIASR | |
| | HGGRPALESIVAQLSRPDPALAALTNDHLVALACLGGRPALDA | |
| | VKKGLPHAPALIKRTNRRIPERTSHRV | |
| DBD482- | HMVDLRTLGYSQQQQEKIKPKVRSTVAQHHEALVGHGFTHAH | 90 |
| PCSK9 | IVALSQHPAALGTVAVKYQDMIAALPEATHEAIVGVGKRGAG | |
| TAL13 | ARALEALLTVAGELRGPPLQLDTGQLLKIAKRGGVTAVEAVHA | |
| | WRNALTGAPLNLTPEQVVAIASHDGGKQALETVQRLLPVLCQ | |
| | AHGLTPEQVVAIASNIGGKQALETVQRLLPVLCQAHGLTPEQV | |
| | VAIASHDGGKQALETVQRLLPVLCQAHGLTPEQVVAIASHDGG | |
| | KQALETVQRLLPVLCQAHGLTPEQVVAIASHDGGKQALETVQR | |
| | LLPVLCQAHGLTPEQVVAIASNIGGKQALETVQRLLPVLCQAH | |
| | GLTPEQVVAIASHDGGKQALETVQRLLPVLCQAHGLTPEQVVA | |
| | IASNIGGKQALETVQRLLPVLCQAHGLTPEQVVAIASHDGGKQ | |
| | ALETVQRLLPVLCQAHGLTPEQVVAIASHDGGKQALETVQRLL | |
| | PVLCQAHGLTPEQVVAIASHDGGKQALETVQRLLPVLCQAHGL | |
| | TPEQVVAIASNGGGKQALETVQRLLPVLCQAHGLTPEQVVAIA | |
| | SNIGGKQALETVQRLLPVLCQAHGLTPEQVVAIASNNGGKQAL | |
| | ETVQRLLPVLCQAHGLTPEQVVAIASNIGGKQALETVQRLLPVL | |
| | CQAHGLTPEQVVAIASNIGGKQALETVQRLLPVLCQAHGLTPE | |
| | QVVAIASNNGGKQALETVQRLLPVLCQAHGLTPEQVVAIASRH | |
| | GGRPALESIVAQLSRPDPALAALTNDHLVALACLGGRPALDAV | |
| | KKGLPHAPALIKRTNRRIPERTSHRV | |
| DBD483- | HMVDLRTLGYSQQQQEKIKPKVRSTVAQHHEALVGHGFTHAH | 91 |
| PCSK9 | IVALSQHPAALGTVAVKYQDMIAALPEATHEAIVGVGKRGAG | |
| TAL14 | ARALEALLTVAGELRGPPLQLDTGQLLKIAKRGGVTAVEAVHA | |
| | WRNALTGAPLNLTPEQVVAIASNNGGKQALETVQRLLPVLCQ | |
| | AHGLTPEQVVAIASNIGGKQALETVQRLLPVLCQAHGLTPEQV | |
| | VAIASNIGGKQALETVQRLLPVLCQAHGLTPEQVVAIASNGGG | |
| | KQALETVQRLLPVLCQAHGLTPEQVVAIASHDGGKQALETVQR | |
| | LLPVLCQAHGLTPEQVVAIASNIGGKQALETVQRLLPVLCQAH | |
| | GLTPEQVVAIASNIGGKQALETVQRLLPVLCQAHGLTPEQVVAI | |
| | ASNGGGKQALETVQRLLPVLCQAHGLTPEQVVAIASHDGGKQ | |
| | ALETVQRLLPVLCQAHGLTPEQVVAIASHDGGKQALETVQRLL | |
| | PVLCQAHGLTPEQVVAIASNGGGKQALETVQRLLPVLCQAHGL | |
| | TPEQVVAIASNIGGKQALETVQRLLPVLCQAHGLTPEQVVAIAS | |
| | HDGGKQALETVQRLLPVLCQAHGLTPEQVVAIASNGGGKQAL | |
| | ETVQRLLPVLCQAHGLTPEQVVAIASNNGGKQALETVQRLLPV | |
| | LCQAHGLTPEQVVAIASNGGGKQALETVQRLLPVLCQAHGLTP | |
| | EQVVAIASNNGGKQALETVQRLLPVLCQAHGLTPEQVVAIASR | |
| | HGGRPALESIVAQLSRPDPALAALINDHLVALACLGGRPALDA | |
| | VKKGLPHAPALIKRTNRRIPERTSHRV | |
| DBD484- | HMVDLRTLGYSQQQQEKIKPKVRSTVAQHHEALVGHGFTHAH | 92 |
| PCSK9 | IVALSQHPAALGTVAVKYQDMIAALPEATHEAIVGVGKRGAG | |
| TAL15 | ARALEALLTVAGELRGPPLQLDTGQLLKIAKRGGVTAVEAVHA | |
| | WRNALTGAPLNLTPEQVVAIASNNGGKQALETVQRLLPVLCQ | |
| | AHGLTPEQVVAIASNIGGKQALETVQRLLPVLCQAHGLTPEQV | |
| | VAIASNGGGKQALETVQRLLPVLCQAHGLTPEQVVAIASHDGG | |
| | KQALETVQRLLPVLCQAHGLTPEQVVAIASHDGGKQALETVQR | |
| | LLPVLCQAHGLTPEQVVAIASNGGGKQALETVQRLLPVLCQAH | |
| | GLTPEQVVAIASNIGGKQALETVQRLLPVLCQAHGLTPEQVVAI | |
| | ASNGGGKQALETVQRLLPVLCQAHGLTPEQVVAIASHDGGKQ | |
| | ALETVQRLLPVLCQAHGLTPEQVVAIASNGGGKQALETVQRLL | |
| | PVLCQAHGLTPEQVVAIASNNGGKQALETVQRLLPVLCQAHGL | |
| | TPEQVVAIASNIGGKQALETVQRLLPVLCQAHGLTPEQVVAIAS | |
| | NGGGKQALETVQRLLPVLCQAHGLTPEQVVAIASNGGGKQAL | |
| | ETVQRLLPVLCQAHGLTPEQVVAIASNIGGKQALETVQRLLPVL | |
| | CQAHGLTPEQVVAIASNIGGKQALETVQRLLPVLCQAHGLTPE | |
| | QVVAIASNIGGKQALETVQRLLPVLCQAHGLTPEQVVAIASHD | |
| | GGRPALESIVAQLSRPDPALAALTNDHLVALACLGGRPALDAV | |
| | KKGLPHAPALIKRTNRRIPERTSHRV | |
| DBD485- | HMVDLRTLGYSQQQQEKIKPKVRSTVAQHHEALVGHGFTHAH | 93 |
| PCSK9 | IVALSQHPAALGTVAVKYQDMIAALPEATHEAIVGVGKRGAG | |
| TAL16 | ARALEALLTVAGELRGPPLQLDTGQLLKIAKRGGVTAVEAVHA | |
| | WRNALTGAPLNLTPEQVVAIASHDGGKQALETVQRLLPVLCQ | |
| | AHGLTPEQVVAIASNIGGKQALETVQRLLPVLCQAHGLTPEQV | |
| | VAIASHDGGKQALETVQRLLPVLCQAHGLTPEQVVAIASNIGG | |
| | KQALETVQRLLPVLCQAHGLTPEQVVAIASHDGGKQALETVQR | |
| | LLPVLCQAHGLTPEQVVAIASHDGGKQALETVQRLLPVLCQAH | |
| | GLTPEQVVAIASHDGGKQALETVQRLLPVLCQAHGLTPEQVVA | |
| | IASNGGGKQALETVQRLLPVLCQAHGLTPEQVVAIASNIGGKQ | |
| | ALETVQRLLPVLCQAHGLTPEQVVAIASNNGGKQALETVQRLL | |
| | PVLCQAHGLTPEQVVAIASNIGGKQALETVQRLLPVLCQAHGL | |
| | TPEQVVAIASNIGGKQALETVQRLLPVLCQAHGLTPEQVVAIAS | |
| | NNGGKQALETVQRLLPVLCQAHGLTPEQVVAIASRHGGKQAL | |
| | ETVQRLLPVLCQAHGLTPEQVVAIASNGGGKQALETVQRLLPV | |
| | LCQAHGLTPEQVVAIASNGGGKQALETVQRLLPVLCQAHGLTP | |
| | EQVVAIASNGGGKQALETVQRLLPVLCQAHGLTPEQVVAIASH | |
| | DGGRPALESIVAQLSRPDPALAALINDHLVALACLGGRPALDA | |
| | VKKGLPHAPALIKRTNRRIPERTSHRV | |
| DBD486- | HMVDLRTLGYSQQQQEKIKPKVRSTVAQHHEALVGHGFTHAH | 94 |
| PCSK9 | IVALSQHPAALGTVAVKYQDMIAALPEATHEAIVGVGKRGAG | |
| TAL17 | ARALEALLTVAGELRGPPLQLDTGQLLKIAKRGGVTAVEAVHA | |
| | WRNALTGAPLNLTPEQVVAIASNNGGKQALETVQRLLPVLCQ | |
| | AHGLTPEQVVAIASNIGGKQALETVQRLLPVLCQAHGLTPEQV | |
| | VAIASNNGGKQALETVQRLLPVLCQAHGLTPEQVVAIASRHGG | |
| | KQALETVQRLLPVLCQAHGLTPEQVVAIASRHGGKQALETVQR | |
| | LLPVLCQAHGLTPEQVVAIASNIGGKQALETVQRLLPVLCQAH | |
| | GLTPEQVVAIASNIGGKQALETVQRLLPVLCQAHGLTPEQVVAI | |
| | ASNNGGKQALETVQRLLPVLCQAHGLTPEQVVAIASNIGGKQA | |
| | LETVQRLLPVLCQAHGLTPEQVVAIASNIGGKQALETVQRLLPV | |
| | LCQAHGLTPEQVVAIASNIGGKQALETVQRLLPVLCQAHGLTP | |
| | EQVVAIASNIGGKQALETVQRLLPVLCQAHGLTPEQVVAIASNI | |
| | GGKQALETVQRLLPVLCQAHGLTPEQVVAIASNIGGKQALETV | |
| | QRLLPVLCQAHGLTPEQVVAIASHDGGKQALETVQRLLPVLCQ | |
| | AHGLTPEQVVAIASNGGGKQALETVQRLLPVLCQAHGLTPEQV | |
| | VAIASHDGGKQALETVQRLLPVLCQAHGLTPEQVVAIASHDGG | |
| | RPALESIVAQLSRPDPALAALTNDHLVALACLGGRPALDAVKK | |
| | GLPHAPALIKRTNRRIPERTSHRV | |
| dCas9 amino | DKKYSIGLAIGTNSVGWAVITDEYKVPSKKFKVLGNTDRHSIK | 95 |
| acid sequence | KNLIGALLFDSGETAEATRLKRTARRRYTRRKNRICYLQEIFSN | |
| | EMAKVDDSFFHRLEESFLVEEDKKHERHPIFGNIVDEVAYHEK | |
| | YPTIYHLRKKLVDSTDKADLRLIYLALAHMIKFRGHFLIEGDLN | |
| | PDNSDVDKLFIQLVQTYNQLFEENPINASGVDAKAILSARLSKS | |
| | RRLENLIAQLPGEKKNGLFGNLIALSLGLTPNFKSNFDLAEDAK | |
| | LQLSKDTYDDDLDNLLAQIGDQYADLFLAAKNLSDAILLSDILR | |
| | VNTEITKAPLSASMIKRYDEHHQDLTLLKALVRQQLPEKYKEIF | |
| | FDQSKNGYAGYIDGGASQEEFYKFIKPILEKMDGTEELLVKLNR | |
| | EDLLRKQRTFDNGSIPHQIHLGELHAILRRQEDFYPFLKDNREKI | |
| | EKILTFRIPYYVGPLARGNSRFAWMTRKSEETITPWNFEEVVDK | |
| | GASAQSFIERMTNFDKNLPNEKVLPKHSLLYEYFTVYNELTKV | |
| | KYVTEGMRKPAFLSGEQKKAIVDLLFKTNRKVTVKQLKEDYF | |
| | KKIECFDSVEISGVEDRFNASLGTYHDLLKIIKDKDFLDNEENED | |
| | ILEDIVLTLTLFEDREMIEERLKTYAHLFDDKVMKQLKRRRYTG | |
| | WGRLSRKLINGIRDKQSGKTILDFLKSDGFANRNFMQLIHDDSL | |
| | TFKEDIQKAQVSGQGDSLHEHIANLAGSPAIKKGILQTVKVVDE | |
| | LVKVMGRHKPENIVIEMARENQTTQKGQKNSRERMKRIEEGIK | |
| | ELGSQILKEHPVENTQLQNEKLYLYYLQNGRDMYVDQELDINR | |
| | LSDYDVAAIVPQSFLKDDSIDNKVLTRSDKARGKSDNVPSEEV | |
| | VKKMKNYWRQLLNAKLITQRKFDNLTKAERGGLSELDKAGFI | |
| | KRQLVETRQITKHVAQILDSRMNTKYDENDKLIREVKVITLKSK | |
| | LVSDFRKDFQFYKVREINNYHHAHDAYLNAVVGTALIKKYPKL | |
| | ESEFVYGDYKVYDVRKMIAKSEQEIGKATAKYFFYSNIMNFFK | |
| | TEITLANGEIRKRPLIETNGETGEIVWDKGRDFATVRKVLSMPQ | |
| | VNIVKKTEVQTGGFSKESILPKRNSDKLIARKKDWDPKKYGGF | |
| | DSPTVAYSVLVVAKVEKGKSKKLKSVKELLGITIMERSSFEKNP | |
| | IDFLEAKGYKEVKKDLIIKLPKYSLFELENGRKRMLASAGELQK | |
| | GNELALPSKYVNFLYLASHYEKLKGSPEDNEQKQLFVEQHKHY | |
| | LDEIIEQISEFSKRVILADANLDKVLSAYNKHRDKPIREQAENIIH | |
| | LFTLTNLGAPAAFKYFDTTIDRKRYTSTKEVLDATLIHQSITGLY | |
| | ETRIDLSQLGGD | |
| dCas9 | GACAAGAAGTACAGCATCGGCCTGGCCATCGGCACCAACAG | 96 |
| nucleotide | CGTGGGCTGGGCCGTGATCACCGACGAGTACAAGGTGCCCA | |
| sequence | GCAAGAAGTTCAAGGTGCTGGGCAACACCGACCGGCACAGC | |
| | ATCAAGAAGAACCTGATCGGCGCCCTGCTGTTCGACAGCGG | |
| | CGAGACCGCCGAGGCCACCCGGCTGAAGCGGACCGCCCGGC | |
| | GGCGGTACACCCGGCGGAAGAACCGGATCTGCTACCTGCAG | |
| | GAGATCTTCAGCAACGAGATGGCCAAGGTGGACGACAGCTT | |
| | CTTCCACCGGCTGGAGGAGAGCTTCCTGGTGGAGGAGGACA | |
| | AGAAGCACGAGCGGCACCCCATCTTCGGCAACATCGTGGAC | |
| | GAGGTGGCCTACCACGAGAAGTACCCCACCATCTACCACCT | |
| | GCGGAAGAAGCTGGTGGACAGCACCGACAAGGCCGACCTGC | |
| | GGCTGATCTACCTGGCCCTGGCCCACATGATCAAGTTCCGGG | |
| | GCCACTTCCTGATCGAGGGCGACCTGAACCCCGACAACAGC | |
| | GACGTGGACAAGCTGTTCATCCAGCTGGTGCAGACCTACAA | |
| | CCAGCTGTTCGAGGAGAACCCCATCAACGCCAGCGGCGTGG | |
| | ACGCCAAGGCCATCCTGAGCGCCCGGCTGAGCAAGAGCCGG | |
| | CGGCTGGAGAACCTGATCGCCCAGCTGCCCGGCGAGAAGAA | |
| | GAACGGCCTGTTCGGCAACCTGATCGCCCTGAGCCTGGGCCT | |
| | GACCCCCAACTTCAAGAGCAACTTCGACCTGGCCGAGGACG | |
| | CCAAGCTGCAGCTGAGCAAGGACACCTACGACGACGACCTG | |
| | GACAACCTGCTGGCCCAGATCGGCGACCAGTACGCCGACCT | |
| | GTTCCTGGCCGCCAAGAACCTGAGCGACGCCATCCTGCTGA | |
| | GCGACATCCTGCGGGTGAACACCGAGATCACCAAGGCCCCC | |
| | CTGAGCGCCAGCATGATCAAGCGGTACGACGAGCACCACCA | |
| | GGACCTGACCCTGCTGAAGGCCCTGGTGCGGCAGCAGCTGC | |
| | CCGAGAAGTACAAGGAGATCTTCTTCGACCAGAGCAAGAAC | |
| | GGCTACGCCGGCTACATCGACGGCGGCGCCAGCCAGGAGGA | |
| | GTTCTACAAGTTCATCAAGCCCATCCTGGAGAAGATGGACG | |
| | GCACCGAGGAGCTGCTGGTGAAGCTGAACCGGGAGGACCTG | |
| | CTGCGGAAGCAGCGGACCTTCGACAACGGCAGCATCCCCCA | |
| | CCAGATCCACCTGGGCGAGCTGCACGCCATCCTGCGGCGGC | |
| | AGGAGGACTTCTACCCCTTCCTGAAGGACAACCGGGAGAAG | |
| | ATCGAGAAGATCCTGACCTTCCGGATCCCCTACTACGTGGGC | |
| | CCCCTGGCCCGGGGCAACAGCCGGTTCGCCTGGATGACCCG | |
| | GAAATCCGAGGAGACCATCACCCCCTGGAACTTCGAGGAGG | |
| | TGGTGGACAAGGGCGCCAGCGCCCAGAGCTTCATCGAGCGG | |
| | ATGACCAACTTCGACAAGAACCTGCCCAACGAGAAGGTGCT | |
| | GCCCAAGCACAGCCTGCTGTACGAGTACTTCACCGTGTACAA | |
| | CGAGCTGACCAAGGTGAAGTACGTGACCGAGGGCATGCGGA | |
| | AGCCCGCCTTCCTGAGCGGCGAGCAGAAGAAGGCCATCGTG | |
| | GACCTGCTGTTCAAGACCAACCGGAAGGTGACCGTGAAGCA | |
| | GCTGAAGGAGGACTACTTCAAGAAGATCGAGTGCTTCGACA | |
| | GCGTGGAGATCAGCGGCGTGGAGGACCGGTTCAACGCCAGC | |
| | CTGGGCACCTACCACGACCTGCTGAAGATCATCAAGGACAA | |
| | GGACTTCCTGGACAACGAGGAGAACGAGGACATCCTGGAGG | |
| | ACATCGTGCTGACCCTGACCCTGTTCGAGGACCGGGAGATG | |
| | ATCGAGGAGCGGCTGAAAACCTACGCCCACCTGTTCGACGA | |
| | CAAGGTGATGAAGCAGCTGAAGCGGCGGCGGTACACCGGCT | |
| | GGGGCCGGCTGAGCCGGAAGCTGATCAACGGCATCCGGGAC | |
| | AAGCAGAGCGGCAAGACCATCCTGGACTTCCTGAAATCCGA | |
| | CGGCTTCGCCAACCGGAACTTCATGCAGCTGATCCACGACG | |
| | ACAGCCTGACCTTCAAGGAGGACATCCAGAAGGCCCAGGTG | |
| | AGCGGCCAGGGCGACAGCCTGCACGAGCACATCGCCAACCT | |
| | GGCCGGCAGCCCCGCCATCAAGAAGGGCATCCTGCAGACCG | |
| | TGAAGGTGGTGGACGAGCTGGTGAAGGTGATGGGCCGGCAC | |
| | AAGCCCGAGAACATCGTGATCGAGATGGCCCGGGAGAACCA | |
| | GACCACCCAGAAGGGCCAGAAGAACAGCCGGGAGCGGATG | |
| | AAGCGGATCGAGGAGGGCATCAAGGAGCTGGGCAGCCAGA | |
| | TCCTGAAGGAGCACCCCGTGGAGAACACCCAGCTGCAGAAC | |
| | GAGAAGCTGTACCTGTACTACCTGCAGAACGGCCGGGACAT | |
| | GTACGTGGACCAGGAGCTGGACATCAACCGGCTGAGCGACT | |
| | ACGACGTGGCCGCCATCGTGCCCCAGAGCTTCCTGAAGGAC | |
| | GACAGCATCGACAACAAGGTGCTGACCCGGAGCGACAAGGC | |
| | CCGGGGCAAGAGCGACAACGTGCCCAGCGAGGAGGTGGTG | |
| | AAGAAGATGAAGAACTACTGGCGGCAGCTGCTGAACGCCAA | |
| | GCTGATCACCCAGCGGAAGTTCGACAACCTGACCAAGGCCG | |
| | AGCGGGGCGGCCTGAGCGAGCTGGACAAGGCCGGCTTCATC | |
| | AAGCGGCAGCTGGTGGAGACCCGGCAGATCACCAAGCACGT | |
| | GGCCCAGATCCTGGACAGCCGGATGAACACCAAGTACGACG | |
| | AGAACGACAAGCTGATCCGGGAGGTGAAGGTGATCACCCTG | |
| | AAATCCAAGCTGGTGAGCGACTTCCGGAAGGACTTCCAGTT | |
| | CTACAAGGTGCGGGAGATCAACAACTACCACCACGCCCACG | |
| | ACGCCTACCTGAACGCCGTGGTGGGCACCGCCCTGATCAAG | |
| | AAGTACCCCAAGCTGGAGAGCGAGTTCGTGTACGGCGACTA | |
| | CAAGGTGTACGACGTGCGGAAGATGATCGCCAAGAGCGAGC | |
| | AGGAGATCGGCAAGGCCACCGCCAAGTACTTCTTCTACAGC | |
| | AACATCATGAACTTCTTCAAGACCGAGATCACCCTGGCCAAC | |
| | GGCGAGATCCGGAAGCGGCCCCTGATCGAGACCAACGGCGA | |
| | GACCGGCGAGATCGTGTGGGACAAGGGCCGGGACTTCGCCA | |
| | CCGTGCGGAAGGTGCTGAGCATGCCCCAGGTGAACATCGTG | |
| | AAGAAAACCGAGGTGCAGACCGGCGGCTTCAGCAAGGAGA | |
| | GCATCCTGCCCAAGCGGAACAGCGACAAGCTGATCGCCCGG | |
| | AAGAAGGACTGGGACCCCAAGAAGTACGGCGGCTTCGACAG | |
| | CCCCACCGTGGCCTACAGCGTGCTGGTGGTGGCCAAGGTGG | |
| | AGAAGGGCAAGAGCAAGAAGCTGAAATCCGTGAAGGAGCT | |
| | GCTGGGCATCACCATCATGGAGCGGAGCAGCTTCGAGAAGA | |
| | ACCCCATCGACTTCCTGGAGGCCAAGGGCTACAAGGAGGTG | |
| | AAGAAGGACCTGATCATCAAGCTGCCCAAGTACAGCCTGTT | |
| | CGAGCTGGAGAACGGCCGGAAGCGGATGCTGGCCAGCGCCG | |
| | GCGAGCTGCAGAAGGGCAACGAGCTGGCCCTGCCCAGCAAG | |
| | TACGTGAACTTCCTGTACCTGGCCAGCCACTACGAGAAGCTG | |
| | AAGGGCAGCCCCGAGGACAACGAGCAGAAGCAGCTGTTCGT | |
| | GGAGCAGCACAAGCACTACCTGGACGAGATCATCGAGCAGA | |
| | TCAGCGAGTTCAGCAAGCGGGTGATCCTGGCCGACGCCAAC | |
| | CTGGACAAGGTGCTGAGCGCCTACAACAAGCACCGGGACAA | |
| | GCCCATCCGGGAGCAGGCCGAGAACATCATCCACCTGTTCA | |
| | CCCTGACCAACCTGGGCGCCCCCGCCGCCTTCAAGTACTTCG | |
| | ACACCACCATCGACCGGAAGCGGTACACCAGCACCAAGGAG | |
| | GTGCTGGACGCCACCCTGATCCACCAGAGCATCACCGGCCT | |
| | GTACGAGACCCGGATCGACCTGAGCCAGCTGGGCGGCGAC | |
| MQ1 amino | SKVENKTKKLRVFEAFAGIGAQRKALEKVRKDEYEIVGLAEW | 97 |
| acid sequence | YVPAIVMYQAIHNNFHTKLEYKSVSREEMIDYLENKTLSWNSK | |
| 1 | NPVSNGYWKRKKDDELKIIYNAIKLSEKEGNIFDIRDLYKRTLK | |
| | NIDLLTYSFPCQDLSQQGIQKGMKRGSGTRSGLLWEIERALDST | |
| | EKNDLPKYLLMENVGALLHKKNEEELNQWKQKLESLGYQNSI | |
| | EVLNAADFGSSQARRRVFMISTLNEFVELPKGDKKPKSIKKVLN | |
| | KIVSEKDILNNLLKYNLTEFKKTKSNINKASLIGYSKFNSEGYVY | |
| | DPEFTGPTLTASGANSRIKIKDGSNIRKMNSDETFLYIGFDSQDG | |
| | KRVNEIEFLTENQKIFVCGNSISVEVLEAIIDKIGG | |
| MQ1 | AGCAAGGTGGAGAACAAGACCAAGAAGCTGCGGGTGTTCGA | 98 |
| nucleotide | GGCCTTCGCCGGCATCGGCGCCCAGCGGAAGGCCCTGGAGA | |
| sequence 1 | AGGTGCGGAAGGACGAGTACGAGATCGTGGGCCTGGCCGAG | |
| | TGGTACGTGCCCGCCATCGTGATGTACCAGGCCATCCACAAC | |
| | AACTTCCACACCAAGCTGGAGTACAAGAGCGTGAGCCGGGA | |
| | GGAGATGATCGACTACCTGGAGAACAAGACCCTGAGCTGGA | |
| | ACAGCAAGAACCCCGTGAGCAACGGCTACTGGAAGCGGAAG | |
| | AAGGACGACGAGCTGAAGATCATCTACAACGCCATCAAGCT | |
| | GAGCGAGAAGGAGGGCAACATCTTCGACATCCGGGACCTGT | |
| | ACAAGCGGACCCTGAAGAACATCGACCTGCTGACCTACAGC | |
| | TTCCCCTGCCAGGACCTGAGCCAGCAGGGCATCCAGAAGGG | |
| | CATGAAGCGGGGCAGCGGCACCCGGAGCGGCCTGCTGTGGG | |
| | AGATCGAGCGGGCCCTGGACAGCACCGAGAAGAACGACCTG | |
| | CCCAAGTACCTGCTGATGGAGAACGTGGGCGCCCTGCTGCA | |
| | CAAGAAGAACGAGGAGGAGCTGAACCAGTGGAAGCAGAAG | |
| | CTGGAGAGCCTGGGCTACCAGAACAGCATCGAGGTGCTGAA | |
| | CGCCGCCGACTTCGGCAGCAGCCAGGCCCGGCGGCGGGTGT | |
| | TCATGATCAGCACCCTGAACGAGTTCGTGGAGCTGCCCAAG | |
| | GGCGACAAGAAGCCCAAGAGCATCAAGAAGGTGCTGAACA | |
| | AGATCGTGAGCGAGAAGGACATCCTGAACAACCTGCTGAAG | |
| | TACAACCTGACCGAGTTCAAGAAAACCAAGAGCAACATCAA | |
| | CAAGGCCAGCCTGATCGGCTACAGCAAGTTCAACAGCGAGG | |
| | GCTACGTGTACGACCCCGAGTTCACCGGCCCCACCCTGACCG | |
| | CCAGCGGCGCCAACAGCCGGATCAAGATCAAGGACGGCAGC | |
| | AACATCCGGAAGATGAACAGCGACGAGACCTTCCTGTACAT | |
| | CGGCTTCGACAGCCAGGACGGCAAGCGGGTGAACGAGATCG | |
| | AGTTCCTGACCGAGAACCAGAAGATCTTCGTGTGCGGCAAC | |
| | AGCATCAGCGTGGAGGTGCTGGAGGCCATCATCGACAAGAT | |
| | CGGCGGC | |
| MQ1 amino | MSKVENKTKKLRVFEAFAGIGAQRKALEKVRKDEYEIVGLAE | 99 |
| acid sequence | WYVPAIVMYQAIHNNFHTKLEYKSVSREEMIDYLENKTLSWNS | |
| 2 | KNPVSNGYWKRKKDDELKIIYNAIKLSEKEGNIFDIRDLYKRTL | |
| | KNIDLLTYSFPCQDLSQQGIQKGMKRGSGTRSGLLWEIERALDS | |
| | TEKNDLPKYLLMENVGALLHKKNEEELNQWKQKLESLGYQNS | |
| | IEVLNAADFGSSQARRRVFMISTLNEFVELPKGDKKPKSIKKVL | |
| | NKIVSEKDILNNLLKYNLTEFKKTKSNINKASLIGYSKFNSEGYV | |
| | YDPEFTGPTLTASGANSRIKIKDGSNIRKMNSDETFLYIGFDSQD | |
| | GKRVNEIEFLTENQKIFVCGNSISVEVLEAIIDKIGG | |
| MQ1 | AGCAAGGUGGAGAACAAGACCAAGAAGCUGCGGGUGUUCG | 100 |
| nucleotide | AGGCCUUCGCCGGCAUCGGCGCCCAGCGGAAGGCCCUGGA | |
| sequence 2 | GAAGGUGCGGAAGGACGAGUACGAGAUCGUGGGCCUGGCC | |
| | GAGUGGUACGUGCCCGCCAUCGUGAUGUACCAGGCCAUCC | |
| | ACAACAACUUCCACACCAAGCUGGAGUACAAGAGCGUGAG | |
| | CCGGGAGGAGAUGAUCGACUACCUGGAGAACAAGACCCUG | |
| | AGCUGGAACAGCAAGAACCCCGUGAGCAACGGCUACUGGA | |
| | AGCGGAAGAAGGACGACGAGCUGAAGAUCAUCUACAACGC | |
| | CAUCAAGCUGAGCGAGAAGGAGGGCAACAUCUUCGACAUC | |
| | CGGGACCUGUACAAGCGGACCCUGAAGAACAUCGACCUGC | |
| | UGACCUACAGCUUCCCCUGCCAGGACCUGAGCCAGCAGGG | |
| | CAUCCAGAAGGGCAUGAAGCGGGGCAGCGGCACCCGGAGC | |
| | GGCCUGCUGUGGGAGAUCGAGCGGGCCCUGGACAGCACCG | |
| | AGAAGAACGACCUGCCCAAGUACCUGCUGAUGGAGAACGU | |
| | GGGCGCCCUGCUGCACAAGAAGAACGAGGAGGAGCUGAAC | |
| | CAGUGGAAGCAGAAGCUGGAGAGCCUGGGCUACCAGAACA | |
| | GCAUCGAGGUGCUGAACGCCGCCGACUUCGGCAGCAGCCA | |
| | GGCCCGGCGGCGGGUGUUCAUGAUCAGCACCCUGAACGAG | |
| | UUCGUGGAGCUGCCCAAGGGCGACAAGAAGCCCAAGAGCA | |
| | UCAAGAAGGUGCUGAACAAGAUCGUGAGCGAGAAGGACAU | |
| | CCUGAACAACCUGCUGAAGUACAACCUGACCGAGUUCAAG | |
| | AAaACCAAGAGCAACAUCAACAAGGCCAGCCUGAUCGGCUA | |
| | CAGCAAGUUCAACAGCGAGGGCUACGUGUACGACCCCGAG | |
| | UUCACCGGCCCCACCCUGACCGCCAGCGGCGCCAACAGCCG | |
| | GAUCAAGAUCAAGGACGGCAGCAACAUCCGGAAGAUGAAC | |
| | AGCGACGAGACCUUCCUGUACAUCGGCUUCGACAGCCAGG | |
| | ACGGCAAGCGGGUGAACGAGAUCGAGUUCCUGACCGAGAA | |
| | CCAGAAGAUCUUCGUGUGCGGCAACAGCAUCAGCGUGGAG | |
| | GUGCUGGAGGCCAUCAUCGACAAGAUCGGCGGC | |
| KRAB amino | DAKSLTAWSRTLVTFKDVFVDFTREEWKLLDTAQQILYRNVM | 101 |
| acid | LENYKNLVSLGYQLTKPDVILRLEKGEEPWLVEREIHQETHPDS | |
| | ETAFEIKSSV | |
| KRAB | GACGCCAAGAGCCTGACCGCCTGGAGCCGGACCCTGGTGAC | 102 |
| nucleotide | CTTCAAGGACGTGTTCGTGGACTTCACCCGGGAGGAGTGGA | |
| | AGCTGCTGGACACCGCCCAGCAGATCCTGTACCGGAACGTG | |
| | ATGCTGGAGAACTACAAGAACCTGGTGAGCCTGGGCTACCA | |
| | GCTGACCAAGCCCGACGTGATCCTGCGGCTGGAGAAGGGCG | |
| | AGGAGCCCTGGCTGGTGGAGCGGGAGATCCACCAGGAGACC | |
| | CACCCCGACAGCGAGACCGCCTTCGAGATCAAGAGCAGCGT | |
| | G | |
| human | VDLRTLDVFSGCGGLSEGFHQAGISDTLWAIEMWDPAAQAFRL | 103 |
| DNMT1 | NNPGSTVFTEDCNILLKLVMAGETTNSRGQRLPQKGDVEMLCG | |
| amino acid | GPPCQGFSGMNRFNSRTYSKFKNSLWSFLSYCDYYRPRFFLLEN | |
| | VRNFVSFKRSMVLKLTLRCLVRMGYQCTFGVLQAGQYGVAQT | |
| | RRRAIILAAAPGEKLPLFPEPLHVFAPRACQLSVVVDDKKFVSNI | |
| | TRLSSGPFRTITVRDTMSDLPEVRNGASALEISYNGEPQSWFQR | |
| | QLRGAQYQPILRDHICKDMSALVAARMRHIPLAPGSDWRDLPN | |
| | IEVRLSDGTMARKLRYTHHDRKNGRSSSGALRGVCSCVEAGK | |
| | ACDPAARQFNTLIPWCLPHTGNRHNHWAGLYGRLEWDGFFST | |
| | TVTNPEPMGKQGRVLHPEQHRVVSVRECARSQGFPDTYRLFGN | |
| | ILDKHRQVGNAVPPPLAKAIGLEIKLCMLAKARESASAKIKEEE | |
| | AAKD | |
| human | GTGGATCTGAGGACACTCGACGTGTTTAGCGGATGCGGCGG | 104 |
| DNMT1 | ACTCTCCGAAGGCTTCCACCAAGCCGGAATTTCCGACACACT | |
| nucleotide | CTGGGCCATTGAGATGTGGGACCCCGCCGCTCAAGCCTTCAG | |
| | ACTGAATAATCCCGGCTCCACCGTGTTCACCGAGGACTGCAA | |
| | CATTCTGCTGAAGCTGGTGATGGCTGGCGAAACCACCAACTC | |
| | TAGAGGCCAGAGGCTGCCCCAGAAGGGAGATGTGGAAATGC | |
| | TCTGTGGAGGCCCTCCTTGCCAAGGCTTCTCCGGCATGAACA | |
| | GGTTCAACTCTAGAACATACAGCAAGTTCAAGAACTCTCTGG | |
| | TCGTGAGCTTTCTGAGCTACTGCGACTACTATAGACCTAGGT | |
| | TCTTTCTGCTGGAGAACGTGAGAAATTTCGTGTCCTTCAAGA | |
| | GGAGCATGGTGCTGAAGCTGACACTGAGGTGTCTGGTGAGG | |
| | ATGGGCTACCAGTGCACATTCGGAGTGCTGCAAGCTGGCCA | |
| | GTACGGCGTGGCCCAGACCAGAAGGAGGGCCATCATTCTGG | |
| | CTGCTGCCCCCGGCGAGAAACTCCCTCTGTTCCCCGAGCCCC | |
| | TCCACGTGTTCGCCCCTAGAGCTTGCCAGCTGAGCGTGGTGG | |
| | TCGACGATAAGAAGTTCGTGAGCAACATCACAAGGCTGTCC | |
| | AGCGGACCCTTCAGAACCATTACCGTGAGGGATACCATGTC | |
| | CGACCTCCCCGAGGTGAGGAATGGCGCCAGCGCTCTGGAGA | |
| | TTTCCTACAACGGCGAACCTCAGAGCTGGTTCCAAAGGCAG | |
| | CTGAGAGGCGCTCAGTATCAGCCCATTCTGAGGGACCACAT | |
| | CTGCAAAGATATGAGCGCTCTGGTGGCCGCTAGAATGAGAC | |
| | ATATTCCTCTGGCCCCCGGCAGCGACTGGAGAGATCTGCCCA | |
| | ATATTGAGGTGAGACTCAGCGACGGAACAATGGCTAGAAAA | |
| | CTGAGGTACACCCATCATGATAGAAAGAACGGAAGGAGCAG | |
| | CAGCGGCGCTCTGAGAGGAGTGTGTAGCTGCGTGGAAGCTG | |
| | GCAAGGCTTGCGATCCCGCCGCTAGGCAGTTCAATACCCTCA | |
| | TCCCTTGGTGTCTGCCTCACACCGGCAACAGACACAATCATT | |
| | GGGCTGGACTGTATGGAAGGCTCGAATGGGACGGCTTTTTC | |
| | AGCACCACCGTGACCAATCCCGAACCTATGGGCAAGCAAGG | |
| | AAGGGTGCTCCACCCCGAGCAGCATAGAGTCGTGTCCGTGA | |
| | GAGAATGCGCTAGAAGCCAAGGCTTCCCCGACACCTATAGA | |
| | CTGTTCGGCAACATTCTGGATAAGCACAGACAAGTGGGAAA | |
| | TGCTGTCCCTCCTCCTCTGGCCAAGGCTATCGGACTGGAGAT | |
| | CAAGCTGTGTATGCTCGCCAAAGCTAGGGAGAGCGCTTCCG | |
| | CCAAGATTAAGGAGGAGGAGGCCGCCAAGGAC | |

1. An expression repressor targeting PCSK9 comprising
(i) a DNA targeting moiety that binds to a target sequence, wherein the target sequence comprises a sequence having at least 90% identity to a sequence selected from SEQ ID NOs: 67-80; and
(ii) an effector domain.

2. The expression repressor of claim 1, wherein the target sequence comprises a sequence selected from SEQ ID NOs: 67-80.

3. The expression repressor of claim 1, wherein the target sequence consists of a sequence selected from SEQ ID NOs: 67-80

4. The expression repressor of one of claims 1-3, wherein the DNA-targeting moiety comprises a zinc finger (ZF) domain.

5. The expression repressor of one of claims 1-3, wherein the DNA-targeting moiety comprises a transcription activator-like effector (TALE) domain.

6. The expression repressor of one of claims 1-3, wherein the DNA-targeting moiety comprises a nuclease inactive Cas polypeptide (dCas9) and a gRNA comprising a sequence complementary to the target sequence.

7. An expression repressor targeting PCSK9 comprising
(i) a DNA targeting moiety that binds to a target sequence comprising an amino acid sequence having at least 90% identity to a sequence selected from 81-94; and
(ii) an effector domain.

8. The expression repressor of any one of claims 1-7, wherein the effector domain comprises a transcriptional repressor moiety.

9. The expression repressor of claim 9, wherein the transcriptional repressor moiety comprises a Kruppel associated box (KRAB) domain or a functional variant or fragment thereof.

10. The expression repressor of claim 9, wherein the transcriptional repressor moiety comprises a histone modifying enzyme selected from a histone methyltransferase, a histone deacetylase, and a histone demethylase.

11. The expression repressor of claim 10, wherein the histone modifying enzyme is a histone deacetylase.

12. The expression repressor of claim 11, wherein the histone deacetylase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment thereof.

13. The expression repressor of claim 10, wherein the histone modifying enzyme is a histone methyltransferase.

14. The expression repressor of claim 18, wherein the histone methyltransferase is SETDB1, SETDB2, EHMT2, EHMT1, SUV39H1, EZH2, EZH1, SUV39H2, SETD8, SUV420H1, SUV420H2, or a functional variant or fragment thereof.

15. The expression repressor of claim 8, wherein the transcriptional repressor moiety comprises a DNA methyltransferase.

16. The expression repressor of claim 15, wherein the DNA methyltransferase is MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment thereof.

17. The expression repressor of claim 15, wherein the DNA methyltransferase is MQ1, or a functional variant or fragment thereof.

18. A nucleic acid comprising a nucleotide sequence encoding the expression repressor of any one of claims 1-17.

19. A recombinant expression vector comprising the nucleic acid of claim 18.

20. A messenger RNA (mRNA) encoding the expression repressor of any one of claims 1-17.

21. A lipid nanoparticle (LNP) comprising the expression repressor of any one of claims 1-17, the nucleic acid of claim 18, the recombinant expression vector of claim 19, or the mRNA of claim 20.

22. A pharmaceutical composition comprising the expression repressor of any one of claims 1-17, the nucleic acid of claim 18, the recombinant expression vector of claim 19, the mRNA of claim 20, or the LNP of claim 21, and a pharmaceutically acceptable carrier.

23. A method of altering expression of PCSK9 in a cell, comprising contacting the cell with the expression repressor of any one of claims 1-17, the nucleic acid of claim 18, the recombinant expression vector of claim 19, the mRNA of claim 20, the LNP of claim 21, or the pharmaceutical composition of claim 22.

24. The method of claim 23, wherein expression of PCSK9 is decreased.

25. A method of introducing one or more epigenetic modifications to PCSK9 in a cell, comprising contacting the cell with the expression repressor of any one of claims 1-17, the nucleic acid of claim 18, the recombinant expression vector of claim 19, the mRNA of claim 20, the LNP of claim 21, or the pharmaceutical composition of claim 22.

26. The method of claim 25, wherein the epigenetic modification is DNA methylation or histone methylation.

27. A method of treating a condition associated with PCSK9 expression in a subject, comprising administering to the subject the expression repressor of any one of claims 1-17, the nucleic acid of claim 18, the recombinant expression vector of claim 19, the mRNA of claim 20, the LNP of claim 21, or the pharmaceutical composition of claim 22.

28. The method of claim 27, wherein the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

29. A method of increasing LDL receptor-mediated clearance of LDL cholesterol in a subject, comprising administering to the subject the expression repressor of any one of claims 1-17, the nucleic acid of claim 18, the recombinant expression vector of claim 19, the mRNA of claim 20, the LNP of claim 21, or the pharmaceutical composition of claim 22.

30. A method of decreasing a circulating cholesterol level in a subject, comprising administering to the subject the expression repressor of any one of claims 1-17, the nucleic acid of claim 18, the recombinant expression vector of claim 19, the mRNA of claim 20, the LNP of claim 21, or the pharmaceutical composition of claim 22.

31. A kit comprising a container comprising a pharmaceutical composition comprising the expression repressor of any one of claims 1-17, the nucleic acid of claim 18, the recombinant expression vector of claim 19, the mRNA of claim 20, or the LNP of claim 21, and a pharmaceutically acceptable carrier, and instructions for use in treating a condition associated with PCSK9 expression in a subject.

32. The kit of claim 31, wherein the condition is selected from hypercholesterolemia, elevated total cholesterol level, elevated low-density lipoprotein (LDL) level, elevated LDL-cholesterol level, reduced high-density lipoprotein level, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and a system thereof.

33. A kit comprising a container comprising a pharmaceutical composition comprising the expression repressor of any one of claims 1-17, the nucleic acid of claim 18, the recombinant expression vector of claim 19, the mRNA of claim 20, or the LNP of claim 21, and a pharmaceutically acceptable carrier, and instructions for use in increasing LDL receptor-mediated clearance of LDL cholesterol and/or decreasing a circulating cholesterol level in a subject.
